(19) United States
(12) Patent Application Publication
(10) Pub. No.: US 2024/0307460 A1
(43) Pub. Date: Sep. 19, 2024

(54) COMPOSITIONS AND METHODS FOR DECOLONIZING ANTIBIOTIC-RESISTANT BACTERIA IN THE GUT

(71) Applicant: Finch Therapeutics Holdings LLC, Boston, MA (US)

(72) Inventors: Marina SANTIAGO, Boston, MA (US); Kevin ROELOFS, Boston, MA (US)

(73) Assignee: Finch Therapeutics Holdings LLC, Boston, MA (US)

(21) Appl. No.: 18/590,022

(22) Filed: Feb. 28, 2024

Related U.S. Application Data
(63) Continuation of application No. 16/707,277, filed on Dec. 9, 2019, now abandoned.
Application No. 16/707,277 is a continuation of application No. PCT/US2018/045592, filed on Aug. 7, 2018.
(60) Provisional application No. 62/542,033, filed on Aug. 7, 2017.

(51) Int. Cl. A61K 35/741; A61P 1/00; A61P 31/04

(52) U.S. Cl. CPC A61K 35/741; A61P 1/00; A61P 31/04

(57) ABSTRACT

The present invention relates to, in part, compositions and methods for delivery of novel mixtures of bacterial strains for the decolonization and/or eradication of various pathogenic bacteria and, particularly, antibiotic-resistant bacteria (ARB).

### PRIORITY

This application is a continuation of U.S. application Ser. No. 16/707,277 filed Dec. 9, 2019, which is a continuation of International Application No. PCT/US2018/045592 filed Aug. 7, 2018, which claims the benefit of U.S. Provisional Application No. 62/542,033, filed Aug. 7, 2017, the contents of which are hereby incorporated by reference in their entireties.

### FIELD OF THE INVENTION

The present invention relates to, in part, compositions and methods for the delivery of novel mixtures of bacterial strains useful for decolonizing and/or eradicating infectious pathogens, e.g., antibiotic-resistant bacteria.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: FIN-003PC_ST25; date recorded: Aug. 7, 2018; file size: 6.07 MB).

### BACKGROUND

Antibiotic-Resistant Bacteria (ARB) are a major health care challenge in the US and around the world. Currently, the Centers for Disease Control and Prevention (CDC) estimates that more than two million people in the US develop antibiotic-resistant infections every year, resulting in more than 23,000 deaths. The economic burden of healthcare institution-acquired infections alone in the US is more than $28 billion.

Many ARBs infect and/or colonize the human gastrointestinal (GI) tract. In fact, gut colonization may be central to the problem of ARB infections. In particular, patients who spend time in healthcare institutions are at a high risk for GI colonization by ARB, especially if they are immunocompromised and/or are being treated with antibiotics and/or treated with an anti-cancer therapy. Such treatments disrupt the gut ecosystem's natural and critical ability to resist infection and colonization by infectious agents, thereby creating an optimal environment for ARB infection, growth, and colonization. These can result in ARB domination of the gut ecosystem. GI colonization by an ARB may be asymptomatic, but colonized patients may shed the ARB into the environment, thereby transmitting the ARB to other patients and to healthcare workers. Moreover, a patients whose gut is colonized and/or dominated by antibiotic-resistant pathogens is him/herself at a higher risk for developing life-threatening bloodstream infections (BSIs) should the ARB translocate across the gut epithelia into the bloodstream; s/he is also at high risk for developing catheter or intravascular-line infections (e.g., central-line infections), for developing chronic inflammatory diseases, for developing meningitis, for developing pneumonia, e.g., ventilator-associated pneumonia, for developing skin and soft tissue infections, for developing surgical-site infections, for developing urinary tract infections (e.g., antibiotic-resistant urinary tract infections and catheter-associated urinary tract infections), for developing wound infections, and for developing other well-known infections: antibiotic-resistant infections and antibiotic-sensitive infections.

Some healthcare institutions perform active surveillance, including testing patients for ARB infection or colonization upon admission and throughout a patient's stay, however, such active surveillance can be costly; thus, few institutions can or have implemented such surveillance. ARB-colonized or infected patients must be put under contact precautions or isolation to prevent transmission of the ARB to other patients and to healthcare workers. Yet, isolation of ARB-colonized or infected patients does nothing to reduce the patient's risk for BSI and other related infections (as known in the art and/or as described herein), and studies have shown isolated patients receive worse care than non-isolated patients; additionally, isolation itself causes emotional distress and is deleterious to the patient's outcome. Unfortunately, there are currently no therapies for decolonizing and/or eradicating ARB from GI-colonized patients.

Accordingly, there remains an unmet need for effective therapies that decolonize and/or eradicate and/or reduce the load of ARB from GI colonized patients.

### SUMMARY OF THE INVENTION

The present invention is based, in part, on the discovery that certain mixtures of commensal bacterial strains can decolonize and/or eradicate and/or reduce the load of antibiotic-resistant enteric pathogens in the gut. Thus, the present invention decreases the economic burden of antibiotic resistance through reduced patient isolation, minimizes a patient's length of stay at a medical treatment center, reduces the number of antibiotic-resistant infections, and lessens the need for antibiotic prescriptions. Additionally, the present invention diminishes the likelihood of transmission of antibiotic-resistant bacteria to other patients and diminishes the risk of bloodstream infection in the original patient, which is a condition with a high mortality rate. Additionally, the present invention improves antimicrobial stewardship by decreasing emergence and prevalence of antibiotic-resistant infections.

In various aspects, the present invention relates to a pharmaceutical composition comprising a bacterial mixture. The bacterial mixture includes at least one bacterial strain comprising a 16S rRNA sequence that is greater than about 97% identical (e.g., about 98%, 99%, 99.5%, and 100% identical) to the 16S rRNA sequence of any one of the operational taxonomic units (OTUs) recited in Table 5 and/or to the 16S rRNA sequence of any one OTU of a genus recited in Table 6.

In various embodiments, the bacterial mixture includes at least two bacterial strains (e.g., at least about five, ten, twenty, thirty, forty, fifty, and one-hundred bacterial strains), wherein a plurality of the bacterial strains comprises a 16S rRNA sequence that is greater than about 97% identical (e.g., about 98%, 99%, 99.5%, and 100% identical) to the 16S rRNA sequence of any one of the OTUs recited in Table 5 and/or to the 16S rRNA sequence of any one OTU of a genus recited in Table 6. In embodiments, the bacterial mixture includes at least two bacterial strains e.g., at least about five, ten, twenty, thirty, forty, fifty, and one-hundred bacterial strains), wherein each bacterial strain comprises a 16S rRNA sequence that is greater than about 97% identical (e.g., about 98%, 99%, 99.5%, and 100% identical) to the 16S rRNA sequence of one of the OTUs recited in Table 5 and/or to the 16S rRNA sequence of any one OTU of a genus recited in Table 6.

In various embodiments, the bacterial mixture includes between about five and one hundred bacterial strains (e.g., between about ten and about seventy-five, between about fifteen and about fifty, between about twenty and about forty-five, between about twenty-five and about forty bacterial, and between about thirty and about thirty-five bacterial strains), wherein a plurality of the bacterial strains comprise a 16S rRNA sequence that is greater than about 97% identical (e.g., about 98%, 99%, 99.5%, and 100% identical) to the 16S rRNA sequence of any one of the OTUs recited in Table 5 and/or to the 16S rRNA sequence of any one OTU of a genus recited in Table 6. In embodiments, the bacterial mixture includes between about five and about one hundred bacterial strains (e.g., between about ten and about seventy-five, between about fifteen and about fifty, between about twenty and about forty-five, between about twenty-five and about forty bacterial, and between about thirty and about thirty-five bacterial strains), wherein each bacterial strain comprises a 16S rRNA sequence that is greater than about 97% identical (e.g., about 98%, 99%, 99.5%, and 100% identical) to the 16S rRNA sequence of one of the OTUs recited in Table 5 and/or to the 16S rRNA sequence of any one OTU of a genus recited in Table 6.

In various embodiments, the bacterial mixture comprises a fecal microbiota preparation that comprises a donor's entire or substantially complete microbiota. In one aspect, a fecal microbiota preparation comprises a non-selected fecal microbiota. In another aspect, a fecal microbiota preparation comprises an isolated or purified population of live non-pathogenic fecal bacteria. In a further aspect, a fecal microbiota preparation comprises a non-selected and substantially complete fecal microbiota preparation from a single donor. In such embodiments, the bacterial mixture includes at least one bacterial strain comprising a 16S rRNA sequence that is greater than about 97% identical (e.g., about 98%, 99%, 99.5%, and 100% identical) to the 16S rRNA sequence of any one of the operational taxonomic units (OTUs) recited in Table 5 and/or to the 16S rRNA sequence of any one OTU of a genus recited in Table 6.

In various embodiments, at least one bacterial strain in a bacterial mixture is a commensal bacterial strain.

In various embodiments, at least one bacterial strain in a bacterial mixture is obtained from one or more human beings, e.g., a human being who is healthy and/or satisfies at least one selection criterion.

In various embodiments, at least one bacterial strain in a bacterial mixture is obtained from a laboratory stock or bacterial cell bank.

In various embodiments, at least one bacterial strain in a bacterial mixture is isolated, cultured, and/or purified from its source material prior to forming the bacterial mixture. In various embodiments, at least one bacterial strain in a bacterial mixture is not isolated, cultured, and/or purified from its source material prior to forming the bacterial mixture.

In various embodiments, at least one bacterial strain is included in a bacterial mixture due to its ability to directly inhibit an antibiotic-resistant bacterium (ARB), for example through production of a secreted product and/or its capability to directly compete with the ARB for a niche and/or for nutrients.

In various embodiments, a pharmaceutical composition further includes a pharmaceutically acceptable excipient. In various embodiments, a pharmaceutical composition is formulated for oral administration and/or for delivery of the bacterial mixture to an intestine, e.g., the small intestine and/or the large intestine (e.g., including the cecum). In various embodiments, delivery of a pharmaceutical composition is substantially completed prior to the rectum. In various embodiments, a pharmaceutical composition is formulated as a capsule, e.g., a capsule including a delayed-release coating.

In various embodiments, a pharmaceutical composition is formulated for vaginal administration and/or for delivery of the bacterial mixture to the female reproductive system.

In various embodiments, a pharmaceutical composition includes a plurality of the bacterial strains that are live, vegetative cells, and/or lyophilized cells. A plurality of the bacterial strains in the bacterial mixture can be spores and/or spore-forming. In various embodiments, a plurality of the bacterial strains (e.g., all the bacterial strains) in the bacterial mixture can be non-pathogenic bacteria.

In various embodiments, a pharmaceutical composition is capable of decolonizing and/or eradicating a pathogen (e.g., an antibiotic-resistant bacterium (ARB)) in the gastrointestinal (GI) tract of a subject, in the genitourinary tract of the subject, and/or in the bloodstream of the subject, e.g., a human subject.

In various aspects, the present invention relates to a method for treating an infection by one or more pathogens. The method includes administering to a subject (e.g., a human subject) in need thereof an effective amount of a pharmaceutical composition of any herein-disclosed aspect or embodiment.

In various embodiments, the one or more pathogens has infected and/or colonized the GI tract of the subject, the genitourinary tract of the subject, and/or the bloodstream of the subject. In various embodiments, administering an effective amount of the pharmaceutical composition decolonizes the one or more pathogens and/or eradicates the one or more pathogens. In various embodiments, the one or more pathogens treated by the method includes one or more of Aeromonas hydrophila, Bacillus, e.g., Bacillus cereus, Bifidobacterium, Bordetella, Borrelia, Brucella, Burkholderia, C. difficile, Campylobacter, e.g., Campylobacter fetus and Campylobacter jejuni, Chlamydia, Chlamydophila, Clostridium, e.g., Clostridium botulinum, Clostridium difficile, and Clostridium perfringens, Corynebacterium, Coxiella, Ehrlichia, Enterobacteriaceae, e.g., Carbapenem-resistant Enterobacteriaceae (CRE) and Extended Spectrum Beta-Lactamase producing Enterobacteriaceae (ESBL-E), fluoroquinolone-resistant Enterobacteriaceae, Enterococcus, e.g., vancomycin-resistant Enterococcus spp., extended spectrum beta-lactam resistant Enterococci (ESBL), and vancomycin-resistant Enterococci (VRE), Escherichia, e.g., enteroaggregative Escherichia coli, enterohemorrhagic Escherichia coli, enteroinvasive Escherichia coli, enteropathogenic E. coli, enterotoxigenic Escherichia coli (such as but not limited to LT and/or ST), Escherichia coli 0157:H7, and multi-drug resistant bacteria E. coli, Francisella, Haemophilus, Helicobacter, e.g., Helicobacter pylori, Klebsiella, e.g., Klebsiella pneumonia and multi-drug resistant bacteria Klebsiella, Legionella, Leptospira, Listeria, e.g., Lysteria monocytogenes, Morganella, Mycobacterium, Mycoplasma, Neisseria, Orientia, Plesiomonas shigelloides, Antibiotic-resistant Proteobacteria, Proteus, Pseudomonas, Rickettsia, Salmonella, e.g., Salmonella paratyphi, Salmonella spp., and Salmonella typhi, Shigella, e.g., Shigella spp., Staphylococcus, e.g., Staphylococcus aureus and Staphylococcus spp., Streptococcus, Treponema, Vibrio, e.g., Vibrio cholerae, Vibrio parahaemolyticus, Vibrio spp., and Vibrio vulnificus, and Yersinia, e.g., Yersinia enterocolitica. At least one of the one or more pathogens can be an antibiotic-resistant bacterium (ARB), e.g., Antibiotic-resistant Proteobacteria, Vancomycin Resistant Enterococcus (VRE), Carbapenem Resistant Enterobacteriaceae (CRE), fluoroquinolone-resistant Enterobacteriaceae, or Extended Spectrum Beta-Lactamase producing Enterobacteriaceae (ESBL-E).

In various aspects, the present invention relates to a method for preventing an infection by one or more pathogens. The method includes administering to a subject (e.g., a human subject) in need thereof an effective amount of a pharmaceutical composition of any herein-disclosed aspect or embodiment.

In various embodiments, the one or more pathogens have not yet infected and/or colonized the GI tract of the subject, the genitourinary tract of the subject, and/or the bloodstream of the subject. In various embodiments, administering an effective amount of the pharmaceutical composition prevents infection and/or colonization. In various embodiments, the one or more pathogens have infected and/or colonized the GI tract of the subject and have not infected and/or colonized the genitourinary tract of the subject and/or the bloodstream of the subject. In various embodiments, administering an effective amount of the pharmaceutical composition prevents infection and/or colonization of the genitourinary tract of the subject and/or the bloodstream of the subject.

In various embodiments, the subject has been directly or indirectly in contact with a subject having an infection or colonization by the one or more pathogens.

In various embodiments, the one or more pathogens includes one or more of Aeromonas hydrophila, Bacillus, e.g., Bacillus cereus, Bifidobacterium, Bordetella, Borrelia, Brucella, Burkholderia, C. difficile, Campylobacter, e.g., Campylobacter fetus and Campylobacter jejuni, Chlamydia, Chlamydophila, Clostridium, e.g., Clostridium botulinum, Clostridium difficile, and Clostridium perfringens, Corynebacterium, Coxiella, Ehrlichia, Enterobacteriaceae, e.g., Carbapenem-resistant Enterobacteriaceae (CRE), fluoroquinolone-resistant Enterobacteriaceae, and Extended Spectrum Beta-Lactamase producing Enterobacteriaceae (ESBL-E), Enterococcus, e.g., vancomycin-resistant Enterococcus spp., extended spectrum beta-lactam resistant Enterococci (ESBL), and vancomycin-resistant Enterococci (VRE), Escherichia, e.g., enteroaggregative Escherichia coli, enterohemorrhagic Escherichia coli, enteroinvasive Escherichia coli, enteropathogenic E. coli, enterotoxigenic Escherichia coli (such as but not limited to LT and/or ST), Escherichia coli 0157:H7, and multi-drug resistant bacteria E. coli, Francisella, Haemophilus, Helicobacter, e.g., Helicobacter pylori, Klebsiella, e.g., Klebsiella pneumonia and multi-drug resistant bacteria Klebsiella, Legionella, Leptospira, Listeria, e.g., Lysteria monocytogenes, Morganella, Mycobacterium, Mycoplasma, Neisseria, Orientia, Plesiomonas shigelloides, Antibiotic-resistant Proteobacteria, Proteus, Pseudomonas, Rickettsia, Salmonella, e.g., Salmonella paratyphi, Salmonella spp., and Salmonella typhi, Shigella, e.g., Shigella spp., Staphylococcus, e.g., Staphylococcus aureus and Staphylococcus spp., Streptococcus, Treponema, Vibrio, e.g., Vibrio cholerae, Vibrio parahaemolyticus, Vibrio spp., and Vibrio vulnificus, and Yersinia, e.g., Yersinia enterocolitica. At least one of the one or more pathogens can be an antibiotic-resistant bacterium (ARB), e.g., Antibiotic-resistant Proteobacteria, Vancomycin Resistant Enterococcus (VRE), Carbapenem Resistant Enterobacteriaceae (CRE), fluoroquinolone-resistant Enterobacteriaceae, or Extended Spectrum Beta-Lactamase producing Enterobacteriaceae (ESBL-E).

In various embodiments, a subject in need thereof has chronic kidney disease, cancer, and/or received an organ transplant.

In various embodiments, a subject in need thereof has received, is receiving, or will receive an anti-cancer therapeutic agent and/or an anti-cancer therapy. Thus, the pharmaceutical compositions find use in reducing, treating, or preventing a side effect of an anti-cancer therapeutic agent and/or a side effect of an anti-cancer therapy and/or in increasing efficacy of an anti-cancer therapeutic agent and/or anti-cancer therapy. The anti-cancer therapy may be surgery, radiation therapy, chemotherapy (including hormonal therapy) and/or targeted therapy (including an immunotherapy). In embodiments, the subject in need thereof is suffering from a side effect of the anti-cancer therapy caused by or related to gut dysbiosis.

An aspect of the present invention provides methods for increasing efficacy of an anti-cancer therapeutic agent and/or anti-cancer therapy. The method comprises administering to a subject in need thereof an effective amount of a pharmaceutical composition comprising a bacterial mixture as disclosed herein. The pharmaceutical composition is administered before, after, and/or contemporaneously with the anti-cancer therapeutic agent and/or anti-cancer therapy.

The subject in need thereof can be in an outpatient setting, hospitalized and/or in a long-term care facility.

In various embodiments, a subject in need thereof has or is at risk for a bloodstream infection (BSI), catheter or intravascular-line infections (e.g., central-line infections), chronic inflammatory diseases, meningitis, pneumonia, e.g., ventilator-associated pneumonia, skin and soft tissue infections, surgical-site infections, urinary tract infections (e.g., antibiotic-resistant urinary tract infections and catheter-associated urinary tract infections), wound infections, and/or other well-known infections: antibiotic-resistant infections and antibiotic-sensitive infections.

Any aspect or embodiment disclosed herein can be combined with any other aspect or embodiment as disclosed herein.

### BRIEF DESCRIPTION OF THE FIGURES

FIG. 1 is a pie chart showing percentages of all operational taxonomic units (OTUs) in the GreenGenes database that are not found in the healthy human gut, that are found in a heathy human gut but not included in Table 5, and that are found in a heathy human gut and are included in Table 5.

### DETAILED DESCRIPTION

The present invention is based, in part, on the discovery that certain mixtures of commensal bacterial strains, including non-selected or substantially complete fecal microbiota preparations, can decolonize and/or eradicate antibiotic-resistant enteric pathogens, e.g., caused by inappropriate broad-spectrum antibiotic use, by a nosocomial infection, or by a previous or current anti-cancer therapy. Thus, instead of developing new antibiotics for decolonization, the present invention includes novel therapeutics that treat gut dysbiosis, in part, by restoring ARB-colonization resistance which is naturally provided by the human microbiome. Thus, the present invention is a more effective long-term strategy for combating antibiotic resistance than a search for and use of new antibiotics. Moreover, the bacterial mixtures can increase efficacy of an anti-cancer therapy, e.g., by simulating an immune response against a cancer cell, and can decrease the severity of a side effect or eliminate the side effect of an anti-cancer therapy, e.g., by promoting the decolonization and/or eradicating various pathogenic bacteria and/or repairing/repopulating his/her gut microbiome after receiving the anti-cancer therapy. Thus, the bacterial mixtures of the present invention are also useful in cancer-related applications.

### Mixtures of Bacterial Strains

The present invention relates to pharmaceutical compositions of mixtures of bacterial strains that are introduced into the gut and promote the decolonization and/or eradication of various pathogenic bacteria; in particular antibiotic-resistant colonizing pathogens including but not limited to: Antibiotic-resistant Proteobacteria, Vancomycin Resistant Enterococcus (VRE), Carbapenem Resistant Enterobacteriaceae (CRE), fluoroquinolone-resistant Enterobacteriaceae, and Extended Spectrum Beta-Lactamase producing Enterobacteriaceae (ESBL-E).

In embodiments, and without wishing to be bound by theory, the mixture of bacterial strains of the present invention acts to protect patients through one or more mechanisms. For example, a first mechanism is direct inhibition of the pathogenic bacteria through production of secreted products and a second mechanism is through competition for nutrients in the gut.

The present invention is also useful in patient populations such as chronic kidney disease on hemodialysis, oncology, and solid organ transplant patients that are at a high risk for ARB colonization and BSI. The present invention is also useful for patients who are in an outpatient setting, hospitalized, and/or in long-term care facilities.

The mixture of bacterial strains of the present invention can be delivered to patients in a variety of ways including orally (e.g., in a capsule), via ND/NG tube, intravaginally (e.g., as vaginal suppository), or colonoscopically. The mixture can also be formulated in a multitude of formulations including pure and/or isolated cultures, both lyophilized bacteria and aqueous solutions, spores, and as part of a broader community or mixture of bacteria (e.g., a mixture of natural communities, including bacteria contained in a source material, and including a substantially complete fecal microbiota of a single healthy stool donor).

In embodiments, the present mixtures of bacterial strains are substantially complete fecal microbiota preparations (e.g., from a single healthy donor). A substantially complete fecal microbiota preparation generally comprises a full complement of functional microorganisms found in feces of one or more healthy humans.

In embodiments, a present mixture of bacterial strains comprises a full complement of functional microorganisms found in feces of one healthy human or in feces of more than one healthy human donor. In embodiments, a present mixture of bacterial strains comprises a full complement of functional microorganisms found in feces of one healthy human or in feces of more than one healthy human donor and further comprises at least one bacterial strain comprising a 16S rRNA sequence that is greater than about 97% identical (e.g., about 98%, 99%, 99.5%, and 100% identical) to the 16S rRNA sequence of any one of the operational taxonomic units (OTUs) recited in Table 5 and/or to the 16S rRNA sequence of any one OTU of a genus recited in Table 6. In other words, a bacterial mixture comprising at least one bacterial strain in the bacterial mixture which comprises a 16S rRNA sequence that is greater than about 97% identical to the 16S rRNA sequence of any one OTU recited in Table 5 or any one OTU of a genus recited in Table 6 could further include a full complement of functional microorganisms, as disclosed herein.

In embodiments, a present mixture of bacterial strains comprises “less than the full complement” of functional microorganisms found in feces of one healthy human or in feces of more than one healthy human donor; here, at least one functional microorganism has been omitted from the full complement. In embodiments, a present mixture of bacterial strains comprises less than the full complement of functional microorganisms found in feces of one healthy human or in feces of more than one healthy human donor and further comprises at least one bacterial strain comprising a 16S rRNA sequence that is greater than about 97% identical (e.g., about 98%, 99%, 99.5%, and 100% identical) to the 16S rRNA sequence of any one of the operational taxonomic units (OTUs) recited in Table 5 and/or to the 16S rRNA sequence of any one OTU of a genus recited in Table 6. In other words, a bacterial mixture comprising at least one bacterial strain in the bacterial mixture which comprises a 16S rRNA sequence that is greater than about 97% identical to the 16S rRNA sequence of any one OTU recited in Table 5 or any one OTU of a genus recited in Table 6 could further include less than the full complement of functional microorganisms, as disclosed herein.

In various embodiments, the bacterial strains of the invention comprise bacteria isolated or purified from one or more humans. In various embodiments, the present mixtures of bacterial strains are isolated or purified from one or more humans. For instance, the isolation or purification may be from feces of the one or more humans. Further, the isolation or purification may be from aspirates of the fluid in the GI tract or mucosal biopsies from a site in the GI tract.

In various embodiments, the bacterial strains of the invention are isolated or purified from its source material, i.e., separated from at least some of the components with which they were associated when initially produced (e.g., when isolated from nature, such as from feces, the bacterial strains can be isolated from fiber and rough particulate matter; in other embodiments, when the bacterial strains are isolated in an experimental setting (e.g., from a laboratory stock, the bacteria can be isolated from associated media and minerals) and/or produced, prepared, purified, and/or manufactured by man. Bacterial strains may be separated from at least about 10%, or about 20%, or about 30%, or about 40%, or about 50%, or about 60%, or about 70%, or about 80%, or about 90%, or more of the other components with which they were initially associated. In some embodiments, bacterial strains are more than about 80%, or about 85%, or about 90%, or about 91%, or about 92%, or about 93%, or about 94%, or about 95%, or about 96%, or about 97%, or about 98%, or about 99%, or more than about 99% pure.

In embodiments, bacterial strains for a bacterial mixture are directly obtained from human feces. In these embodiments, fecal matter is collected from one or more humans and processed ultimately until a formulation suitable for oral delivery and/or delivery into the GI tract is prepared.

In various embodiments, fecal matter is collected from one or more humans and processed ultimately until a formulation suitable for vaginal delivery is prepared. An example of such formulation is a vaginal suppository.

In other embodiments, bacterial strains for a bacterial mixture are indirectly obtained from human feces and/or are obtained independent of human feces (e.g., from a bacterial cell bank or from a laboratory stock). When indirectly obtained, bacterial strains from human feces are cultured and the bacteria are expanded and then isolated and/or purified. The isolated/purified bacteria can be introduced into a bacterial mixture comprising bacterial strains directly obtained from human feces. Alternately, a plurality of isolated/purified bacteria can be combined into a defined bacterial mixture comprising only bacterial strains indirectly obtained from human feces or obtained independent of human feces.

In various embodiments, human feces are obtained from screened, qualified donors.

In embodiments, a qualified donor provides feces having a full complement of functional microorganisms found in feces of one or more healthy humans, as disclosed herein.

In embodiments, a qualified donor provides feces having at least one bacterial strain (e.g., a plurality of bacterial strains) comprising a 16S rRNA sequence that is greater than about 97% identical (e.g., about 98%, 99%, 99.5%, and 100% identical) to the 16S rRNA sequence of any one of the operational taxonomic units (OTUs) recited in Table 5 and/or to the 16S rRNA sequence of any one OTU of a genus recited in Table 6. Moreover, the qualified donor provides feces substantially lacking bacterial strains known to be associated with infectious diseases, as disclosed elsewhere herein.

In embodiments of the present invention, potential donors are screened via: (1) Initial Preliminary Screen. Prior to enrollment, potential donors (e.g., aged about 18 to about 50) undergo a preliminary screen comprising a subset of questions selected from a Donor Health Questionnaire (DHQ) to assess eligibility and/or (2) In-Person Donor Interview. If the potential donor passes the initial preliminary screen, he/she conducts in-person interview and clinical assessment with a healthcare professional. As part of this interview the potential donor completes informed consent and a donor affidavit attesting to provide true, accurate, and complete information. The DHQ, in-person interview, and clinical assessment determine the potential donor's eligibility as a donor.

The DHQ and clinical assessment identify relevant criteria which would preclude one from being a donor (e.g., temporarily and permanently). Three categories of criteria covered by a DHQ include: (1) Infectious risk factors, e.g., risk for factors for multi-drug resistance organisms (MDROs); high-risk sexual behaviors; social history, including illicit drug use; high-risk travel history (including a 12-month exclusion if a potential donor has traveled to a high-risk or very high-risk area, as defined by current International SOS (ISOS) guidelines); (2) potential microbiome-mediated conditions and general health status, e.g., gastrointestinal comorbidities; metabolic comorbidities; neurological comorbidities; psychiatric comorbidities; chronic pain syndromes; infectious diseases; autoimmune diseases; atopy, asthma and allergies (food and other); malignancy; surgeries/other medical history; current symptoms (including stool habits); medications including antimicrobial therapy; diet; and family history; and (3) pregnancy and breastfeeding status, for potential female donors. In embodiments, the clinical assessment includes, as examples, determination of vital signs including temperature, blood pressure, heart rate, respiratory rate, waist circumference, and body mass index (BMI).

In embodiments, the DHQ is analogous to that used by the Red Cross for screening potential blood donors (with fewer or additional questions, if desired).

Potential donors who are eligible to be donors based upon their DHQ, in-person interview results, and clinical assessment then undergo a series of serological, stool, and nasal swab screens/tests. Serological, stool, and nasal swab testing/screening are performed in conjunction with a diagnostic laboratory, e.g., a Clinical Laboratory Improvement Amendments (CLIA)-certified diagnostic laboratory.

Table 1 provides an overview of exemplary serological, stool, and nasal swab screens/tests conducted as part of the donor screening process of various embodiments. Screening/testing is performed under conditions well-known in the art, such as, by way of a non-limiting example: Hepatitis C may be detected by an immunoassay (IA), Shiga may be detected by enzyme immunoassay (EIA), and Clostridium difficile may be detected by real-time polymerase chain reaction (RT-PCR).

TABLE 1
Exemplary Serological, Stool, and Nasal Swab Screens/Tests

| | Pathogen | |
|---|---|---|
| Serological | HIV 1/2 | |
| Testing | Hepatitis A | |
| | Hepatitis B | |
| | Hepatitis C | |
| | Treponema pallidum | |
| | Strongyloides | |
| Stool | Multi-Drug Resistant | VRE |
| Testing | Organisms | CRE |
| | | FRE |
| | | ESBL |
| | Salmonella spp | |
| | Shigella spp | |
| | Campylobacter spp | |
| | Vibrio spp | |
| | Rotavirus A | |
| | Cryptosporidium spp | |
| | Shiga | |
| | Giardia lamblia | |
| | Adenovirus | |
| | Norovirus | |
| | Clostridium difficile (e.g., a producer of Toxin B) | |
| | Cryptosporidium spp | |
| | Helicobacter pylori | |
| | Ova and parasites | |
| | Cyclospora and Isospora | |
| | Microsporidia | |
| | Bristol Stool Type assessment | |
| Nasal Swab | Multi-Drug Resistant | VRE |
| | Organisms | CRE |
| | | FRE |
| | | MRSA |
| | | ESBL |

VRE = Vancomycin-resistant enterococci; CRE = carbapenem-resistant Enterobacteriaceae; ESBL = Extended-spectrum beta-lactamases; FRE = fluoroquinolone-resistant Enterobacteriaceae.

In some embodiments, a potential donor is excluded if he/she has a positive result in a test/screen for an infectious disease, e.g., caused by one of the pathogens listed in Table 1. In some embodiments, a potential donor who tests positive for HIV-1/2, Hepatitis B, or Hepatitis C is indefinitely excluded from donating.

In some embodiments, a potential donor who tests positive for Hepatitis A, Treponema pallidum, or Strongyloides is deferred from donating until eight weeks after a successful treatment has been completed, symptoms have resolved, and no recurrence of symptoms have occurred.

In some embodiments, a potential donor who tests positive for Adenovirus, Campylobacter spp, Clostridium difficile toxin B, Cryptosporidium spp, Cyclospora and Isospora, Giardia lamblia, Proteus, Morganella, Helicobacter pylori, Microsporidia, Norovirus, Ova and parasites, Salmonella spp, Shiga, Shigella spp, or Vibrio spp, is immediately placed on hold and deferred for eight weeks from symptom resolution, completion of treatment, and no recurrence. Screened donors deferred for eight weeks from symptom resolution, completion of treatment, and no recurrence due to any of the above may undergo a full repeat screen to evaluate for inclusion.

In some embodiments, a potential donor who tests positive for rotavirus is placed immediately on donation hold and undergoes repeat confirmatory testing. If confirmed positive, these donors are ineligible for donation for eight weeks. Screened donors deferred for eight weeks due to rotavirus may undergo a full repeat screen to evaluate for inclusion.

In some embodiments, a potential donor who tests positive for a Multi-Drug Resistant Organism (MDROs), e.g., Vancomycin-resistant Enterococcus (VRE), Carbapenem-resistant enterobacteriaceae (CRE), fluoroquinolone-resistant Enterobacteriaceae (FRE), and Extended-spectrum beta-lactamase (ESBL) is immediately placed on hold and deferred for eight weeks after successful treatment/decolonization with no symptoms or recurrence. Screened donors deferred for eight weeks after successful treatment/decolonization with no symptoms or recurrence due to any of the above may undergo a full repeat screen to evaluate for inclusion.

In some embodiments, a potential donor who tests positive for Methicillin-resistant Staphylococcus aureus (MRSA) is immediately placed on hold and deferred for eight weeks after successful treatment/decolonization with no symptoms or recurrence. Screened donors deferred for eight weeks after successful treatment/decolonization with no symptoms or recurrence due to any of the above may undergo a full repeat screen to evaluate for inclusion.

In some embodiments, potential donors may submit samples for additional screening which may include assays for Liver Function Panel, Alanine Aminotransferase (ALT), Aspartate Aminotransferase (AST), Alkaline Phosphatase (ALP), Albumin, Bilirubin (Total, direct, or indirect), and Complete Blood Count (CBC) with Differential. Donors whose results from these Additional Screening assays are outside the bounds of normal (see, e.g., Table 2) are ineligible to donate stool.

TABLE 2
Exemplary Low and High limit for Complete Blood
Count (CBC) and Hepatic Function Panel (HFP)

| Test | Category | Low | High | Units |
|---|---|---|---|---|
| CBC | WBC | 3.8 | 10.8 | ×10³/μL |
| CBC | RBC | 4.20 | 5.80 | ×10⁶/μL |
| CBC | Hemoglobin | 13.2 | 17.1 | g/dL |
| CBC | Hematocrit | 38.5 | 50.0 | % |
| CBC | MCV | 80 | 100 | fL |
| CBC | MCH | 27.0 | 33.0 | pg |
| CBC | MCHC | 32.0 | 36.0 | g/dL |
| CBC | RDW | 11 | 15 | % |
| CBC | Platelets | 140 | 400 | ×10³/μL |
| CBC | MPV | 7.5 | 11.5 | fL |
| CBC | Absolute Neutrophils | 1500 | 7800 | cells/μL |
| CBC | Absolute Lymphocytes | 850 | 3900 | cells/μL |
| CBC | Absolute Monocytes | 200 | 950 | cells/μL |
| CBC | Absolute Eosinophils | 15 | 500 | cells/μL |
| CBC | Absolute Basophils | 0 | 200 | cells/μL |
| HFP | Protein, Total, Serum | 6.1 | 8.1 | g/dL |
| HFP | Albumin, Serum | 3.6 | 5.1 | g/dL |
| HFP | Bilirubin, Total | 0.2 | 1.2 | mg/dL |
| HFP | Bilirubin, Direct | 0.00 | 0.20 | mg/dL |
| HFP | Bilirubin, Indirect | 0.2 | 1.2 | mg/dL |
| HFP | Alkaline Phosphatase, Serum | 40 | 115 | U/L |
| HFP | AST (SGOT) | 10 | 40 | U/L |
| HFP | ALT (SGPT) | 9 | 46 | U/L |

If the cause of abnormal assay results is found to be either infectious or may otherwise compromise the health of the donor or an FMT recipient, that donor may be excluded from donating stool for clinical use. If the cause of the abnormal reading is determined to be not clinically significant and to pose no threat to an FMT recipient, as examples, the result is an incidental artifact or due to Gilbert's syndrome, then the donor may be considered for enrollment/re-enrollment.

Other screens or tests may also be used to exclude or include potential donors.

In some embodiments, a potential donor may be positive for one or both of Cytomegalovirus (CMV) and Epstein-Barr Virus (EBV). There have not been any reported cases of CMV or EBV infection among those who have received FMT from adult donors (Wang et al. 2016), including a large series of immunocompromised patients (Kelly et al. 2014) and solid organ transplant patients (Fischer et al. 2017).

In some embodiments, a potential donor may be positive for Listeria monocytogenes. In embodiments, donated material and/or serological samples are not tested for L. monocytogenes unless the donor is symptomatic for a L. monocytogenes infection.

In some embodiments, before or after a stool donation event, the pre-screened donor again completes a DHQ. A donor's eligibility will be further evaluated should he/she have any positive responses in this questionnaire. If the donor's responses indicate any changes in health status that involve an exclusion criterion, the donated material is discarded. When the donor's DHQ results do not indicate exclusion, the container and the stool material contained therein is processed.

In some embodiments, a donor may complete an in-person clinical health check around the time of a stool donation to ensure the donor's health. If the donor does not have good/optimal health, the donated material may be discarded.

In some embodiments, a donor is generally of good health and has microbiota consistent with such good health. Often, the donor has not been administered an antibiotic compound within a certain period prior to a stool donation.

In some embodiments, the donor does not have irritable bowel disease (e.g., Crohn's disease and ulcerative colitis), irritable bowel syndrome, celiac disease, colorectal cancer, or a family history of these diseases.

In some embodiments, a donor is selected for the presence of certain genera and/or species that provide increased efficacy of therapeutic compositions containing these genera or species. In some embodiments, a preferred donor donates stool material having a relatively high concentration of spores. In some embodiments, a preferred donor donates stool material comprising spores having increased efficacy.

In some embodiments, a sample of a donated stool material or a donated stool may be used for Additional Screening. Additional Screening may include assays for Liver Function Panel, Alanine Aminotransferase (ALT), Aspartate Aminotransferase (AST), Alkaline Phosphatase (ALP), Albumin, Bilirubin (Total, direct, indirect), and Complete Blood Count (CBC) with Differential. Donors whose results from these Additional Screening assays are outside the bounds of normal (see, e.g., Table 2) the donated material may be discarded.

Other screens or tests may also be used to temporarily or permanently exclude donors.

In some embodiments, a donor who tests positive for Hepatitis A, Treponema pallidum, or Strongyloides is deferred from donating until eight weeks after a successful treatment has been completed, symptoms have resolved, and no recurrence of symptoms have occurred. Impacted donated material will be destroyed. Screened donors deferred for eight weeks from symptom resolution, completion of treatment, and no recurrence due to any of the above may undergo a full repeat screen to evaluate his/her return as a donor.

In some embodiments, a donor who tests positive for Adenovirus, Campylobacter spp, Clostridium difficile toxin B, Cryptosporidium spp, Cyclospora and Isospora, Giardia lamblia, Proteus, Morganella, Helicobacter pylori, Microsporidia, Norovirus, Ova and parasites, Salmonella spp, Shiga, Shigella spp, or Vibrio spp, is immediately placed on hold and deferred for eight weeks from symptom resolution, completion of treatment, and no recurrence. Impacted donated material will be discarded. Screened donors deferred for eight weeks from symptom resolution, completion of treatment, and no recurrence due to any of the above may undergo a full repeat screen to evaluate his/her return as a donor.

In some embodiments, a donor who tests positive for rotavirus will be placed immediately on donation hold and have repeat confirmatory testing performed. If confirmed positive, these donors will have their donated material discarded and will be ineligible for donation for eight weeks. Screened donors deferred for eight weeks due to rotavirus may undergo a full repeat screen to evaluate his/her return as a donor.

A donor who tests positive for a Multi-Drug Resistant Organism (MDROs), e.g., Vancomycin-resistant Enterococcus (VRE), Carbapenem-resistant enterobacteriaceae (CRE), fluoroquinolone-resistant Enterobacteriaceae (FRE) and Extended-spectrum beta-lactamase (ESBL) is immediately placed on hold and deferred for eight weeks after successful treatment/decolonization with no symptoms or recurrence. Impacted donated material will be discarded. Screened donors deferred for eight weeks after successful treatment/decolonization with no symptoms or recurrence due to any of the above may undergo a full repeat screen to evaluate his/her return as a donor.

In some embodiments, a donor who tests positive for Methicillin-resistant Staphylococcus aureus (MRSA) is immediately placed on hold and deferred for eight weeks after successful treatment/decolonization with no symptoms or recurrence. Impacted donated material will be discarded. Screened donors deferred for eight weeks after successful treatment/decolonization with no symptoms or recurrence due to any of the above may undergo a full repeat screen to evaluate his/her return as a donor.

In some embodiments, a donor may be positive for one or both of Cytomegalovirus (CMV) and Epstein-Barr Virus (EBV). There have not been any reported cases of CMV or EBV infection among those who have received FMT from adult donors (Wang et al., 2016), including a large series of immunocompromised patients (Kelly et al., 2014) and solid organ transplant patients (Fischer et al., 2017).

In some embodiments, a donor undergoes a blood test about twenty-one days, e.g., two weeks to a month, or longer, after his/her last donation to account for HIV seroconversion.

In some embodiments, a donor may be positive for Listeria monocytogenes. In embodiments, donated material and/or serological samples are not tested for L. monocytogenes unless the donor is symptomatic for a L. monocytogenes infection.

In embodiments, processing of a donated material begins within six hours of passage of stool material. Elapsed time prior to stool processing may be noted.

In some embodiments, donated material will be assessed using the Bristol stool scale and/or for hematochezia, melena, mucus, and/or steatorrhea. Collection of samples from the donated material may occur within the biosafety cabinet.

Stool below Bristol Type 3 and stool above Bristol Type 5 is discarded.

Stool exhibiting signs of hematochezia, melena, mucus, and/or steatorrhea is discarded.

In some embodiments, donated material is quarantined (i.e., not included in a drug substance and/or not included in a drug product) for a “collection window” of about sixty days, e.g., thirty to ninety days, and until the donor has passed a second DHQ, a second in-person clinical assessment, and/or a second set of serological, stool, and/or nasal swab tests (as described above). See, Table 3.

TABLE 3
Donor Screening/Testing

| | | Testing Time Points | |
|---|---|---|---|
| | | Start of | End of |
| | | collection | collection |
| Parameter | Acceptance Criteria | window | window |
| Questionnaire & | Pass | x | x |
| Interview | | | |
| Serological | Negative for a panel of | x | x |
| | Infectious Diseases | | |
| Stool | Negative for a panel | x | x |
| | of Viruses, Enteric | | |
| | Pathogens, Parasites, etc. | | |
| MDRO | Negative for a | x | x |
| | panel of Multi-Drug | | |
| | Resistant Organisms | | |
| Additional | “Normal” for a Liver | x | x |
| Screening | Function panel and | | |
| | Complete Blood | | |
| | Count & Differential ^{b} | | |
| Donor Health | No issues noted that | x | x |
| Questionnaire | involve Exclusion | | |
| (DHQ) completed | Criteria | | |
| at Delivery ^{a} | | | |

^{a} In addition to the DHQ, if a donor experiences any abnormal symptoms, including a change in bowel habits or change in other relevant clinical factors (e.g., medicines and medical history) donors should notify to the donation facility immediately. A full health assessment in conducted and if symptoms would lead to stool that may impact the health of an FMT recipient, donation is suspended until an examination of the underlying symptoms is initiated by clinical assessment and/or diagnostic tests on stool and/or blood. The impacted material may be discarded. In the event of transient, self-limiting, mild symptoms, donors may be eligible when symptoms resolve.
^{b} See, Table 2

TABLE 4
Physical Testing Conducted on Donated Material

| Parameter | Acceptance Criteria | Justification |
|---|---|---|
| Bristol | Bristol Stool Type | Bristol Stool Type of 2, 3, |
| Stool Type | must be | 4, and 5 are considered |
| | Type 3, 4, or 5 | healthy. Types above that |
| | | range (i.e. Type 6 and 7) |
| | | indicate diarrhea; these |
| | | Stool Types are not |
| | | processed. |
| | | Stool with a Bristol Stool |
| | | Type 1 or 2, which indicates |
| | | constipation, may be too |
| | | rigid or dense for readily |
| | | processing; these Stool |
| | | Types are not processed. |
| Screening | Hematochezia | The presence of fresh |
| of Stool for | Visually | blood in stool indicates |
| Hematochezia | Absent | lower gastrointestinal |
| | | pathology (e.g., |
| | | diverticulosis and inflammatory |
| | | bowel disease) or, |
| | | less commonly, a brisk |
| | | upper gastrointestinal |
| | | bleed. Stool with hematochezia |
| | | is not processed. |
| Screening | Melena Visually | The presence of melena in |
| of Stool for | Absent | stool indicates upper |
| Melena | | gastrointestinal bleeding |
| | | (e.g., peptic ulcer |
| | | disease, gastritis, and |
| | | esophageal varices). Stool |
| | | with melena is not processed. |
| Screening | Mucus Visually | Although small amounts |
| of Stool for | Absent | of mucus are normal, the |
| Mucus | | presence of mucus in |
| | | stool potentially indicates |
| | | gastrointestinal pathology |
| | | (e.g., inflammatory |
| | | bowel disease and |
| | | malignancy). Stool with |
| | | mucus is processed. |
| Screening | Steatorrhea | The presence of steatorrhea |
| of Stool for | Visually | in stool indicates fat |
| Steatorrhea | Absent | malabsorption (e.g., pancreatic |
| | | insufficiency). Stool |
| | | with steatorrhea is not processed. |

In some embodiments, the viability of the microbiota of the donated stool may be confirmed by culturing a sample of the donated stool, an otherwise purified form of the donated stool, a filtrate, a homogenized product, a thawed-frozen intermediate, a pooled material, and/or a drug substance. Methods for culturing microbiota from stool or from stool-derived products are well-known in the art. In some embodiments, microbiota are cultured using the Center for Disease Control (CDC) plate, commonly referred to as “CDC Anaerobe 5% Sheep Blood Agar plate, which allows for the isolation and cultivation of fastidious and slow-growing obligatory anaerobic bacteria, the Bacteroides Bile Esculin Agar (BBE) plate, which is a specific indicator species media for Bacteroides, or GIFU Anaerobic Medium Agar (GAA). In some embodiments, the number of viable, culturable cells within the stool or stool-derived products may be confirmed by the presence of a colony forming unit (CFU) counts, e.g., by the Drop Plate CFU Assay. The diversity of the living microbes in the stool or from stool-derived products may be assayed. The mix of microbes present, or diversity of microbes, is a further measure of the quality of the donated stool and the drug substance.

In some embodiments, the viability of the microbiota of the donated stool may be confirmed by PMAseq; Chu et al., “Using Propodium Monoazide Sequencing (PMA-Seq) to Develop Data-Driven Best Practices in Fecal Microbiota Transplantations.” Open Forum Infect Dis. Oxford University Press; 2015)]. Briefly, this approach provides a high-throughput, culture-independent measure of cell viability.

In some embodiments, the bacteria are live, vegetative cells. In some embodiments, the bacteria are capable of forming spores. In some embodiments, the bacteria are in the form of spores, e.g., viable spores. In some embodiments, the mixtures of bacterial strains as described herein comprise live, vegetative cells and spores. In some embodiments, the mixture of bacterial strains as described herein is substantially free of live, vegetative cells. In some embodiments, the mixture of bacterial strains as described herein is substantially free of spores. In some embodiments, the bacterial strains are in the form of live, vegetative cells. In some embodiments, the bacterial strains are in the form of spores. In some embodiments, the bacterial strains are in the form of lyophilized cells. In some embodiments, the bacterial mixture comprises one or more of live, vegetative cells; spores; and lyophilized cells.

In some embodiments, the bacterial strains are non-pathogenic. For instance, in some embodiments, the bacterial strains are substantially free of organisms or entities which are capable of causing or affecting a disease, disorder or condition of a host organism containing the organism or entity. Illustrative pathogenic bacteria are provided elsewhere herein.

In various embodiments, the mixture of bacterial strains includes one or more non-pathogenic bacterial strains that are able to engraft in a patient's GI tract. In some embodiments, the mixture of bacterial strains includes one or more non-pathogenic bacterial strains that are able to colonize a patient's mucosal barrier. In some embodiments, the mixture of bacterial strains includes one or more bacterial strains that preserve and/or enhance mucosal barrier integrity and function in a patient. In various embodiments, the mixture of bacterial strains includes one or more bacterial strains that decolonize pathogenic infectious agents. In some embodiments, the mixture of bacterial strains includes one or more bacterial strains that compete with pathogenic infectious agents for resources (e.g., niche and/or nutrients). In another embodiment, the mixture of bacterial strains includes one or more bacterial strains that enhance production of one or more of butyrate, acetate, and propionate in the gut. In various embodiments, the mixture of bacterial strains includes one or more bacterial strains that supplement SCFA production in the gut. In various embodiments, the mixture of bacterial strains induces proliferation and/or accumulation of Foxp3⁺ cells, e.g., regulatory T cells (T_{regs}) in the mucosal lining of the gut. In various embodiments, the mixture of bacterial strains induces proliferation and/or accumulation of interleukin-10 (IL-10) in the gut. In various embodiments, the mixture of bacterial strains reduces proliferation and/or accumulation of interleukin-12 (IL-12), interleukin-4 (IL-4), and/or and gamma interferon (IFN γ) in the gut.

Without wishing to be bound by theory, treatment of the gut microbiome with antibiotics can result in a reduction of butyrate levels in the gut. Butyrate serves as the primary energy source of colonocytes and with decreased butyrate levels, colonocytes switch to anaerobic/fermentative growth. Because this growth does not utilize oxygen, the gut barrier becomes increasingly oxygenated. Butyrate also normally inhibits nitric oxide synthase, so in the absence of normal butyrate levels, the concentration of host-derived nitrate in the gut increases. Enterobacteriaceae are often opportunistic pathogens, and blooms of these bacteria are a sign of dysbiosis and inflammation. Members of Enterobacteriaceae can use both nitrate and oxygen as electron acceptors during respiration. Therefore, antibiotic treatment can result in decreased butyrate production, leading to increased oxygen and nitrate content in the gut, which gives potentially pathogen facultative anaerobes like Enterobacteriaceae a growth advantage, resulting in an Enterobacteriaceae bloom.

In various embodiments, the present mixture of bacterial strains provides both systemic anti-inflammatory and immunoregulatory effects.

In various embodiments, the present mixture of bacterial strains includes one or more isolated or purified bacterial strains that individually or when together in a mixture have a cytotoxic or cytostatic effect on a pathogenic bacterium, as described elsewhere herein, e.g., can eradicate and/or decolonize pathogenic infectious bacteria. In various embodiments, the present mixture of bacterial strains exerts an inhibitory effect on a pathogenic bacterium present in or entering into the GI tract of a patient. In various embodiments, the present mixture of bacterial strains augments growth of at least one type of bacteria not detectably present in a patient's GI tract prior to administration. In various embodiments, the present mixture of bacterial strains includes one or more isolated or purified bacterial strains that interact synergistically to have a cytotoxic or cytostatic effect on a pathogenic bacterium.

Illustrative pathogenic bacteria include C. difficile, Salmonella spp., enteropathogenic E. coli, multi-drug resistant bacteria such as Klebsiella, and E. coli, Carbapenem-resistant Enterobacteriaceae (CRE), extended spectrum beta-lactam resistant Enterococci (ESBL), fluoroquinolone-resistant Enterobacteriaceae, and vancomycin-resistant Enterococci (VRE). Further illustrative bacteria include Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Clostridium, Chlamydia, Chlamydophila, Campylobacter, Burkholderia, Brucella, Borrelia, Bordetella, Bifidobacterium, Bacillus, Proteus, Morganella, multi-drug resistant bacteria, extended spectrum beta-lactam resistant Enterococci (ESBL), Carbapenem-resistant Enterobacteriaceae (CRE), fluoroquinolone-resistant Enterobacteriaceae, and vancomycin-resistant Enterococci (VRE). Illustrative pathogenic bacteria include Aeromonas hydrophila, Campylobacter fetus, Plesiomonas shigelloides, Bacillus cereus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enterotoxigenic Escherichia coli (such as, but not limited to, LT and/or ST), Escherichia coli 0157:H7, Helicobacter pylori, Klebsiella pneumonia, Lysteria monocytogenes, Plesiomonas shigelloides, Salmonella spp., Salmonella typhi, Salmonella paratyphi, Shigella spp., Staphylococcus spp., Staphylococcus aureus, vancomycin-resistant Enterococcus spp., Vibrio spp., Vibrio cholerae, Vibrio parahaemolyticus, Vibrio vulnificus, and Yersinia enterocolitica. Specifically-relevant pathogenic bacteria include Antibiotic-resistant Proteobacteria, Vancomycin Resistant Enterococcus (VRE), Carbapenem Resistant Enterobacteriaceae (CRE), fluoroquinolone-resistant Enterobacteriaceae, and Extended Spectrum Beta-Lactamase producing Enterobacteriaceae (ESBL-E).

In various embodiments, a bacterial strain is included in the pharmaceutical composition of the invention based upon its 16S rRNA sequence identity. In an embodiment, the pharmaceutical composition of the invention comprises one or more bacterial strains having a 16S rRNA sequence that is at least about 80% identical to the 16S rRNA sequence of any one of the operational taxonomic units (OTUs) provided in Table 5 or Table 6. For example, the pharmaceutical composition may comprise one or more bacterial strains having a 16S rRNA sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical with the 16S rRNA sequence of any one of the operational taxonomic units provided in Table 5 or Table 6. In an embodiment, the pharmaceutical composition may comprise one or more bacterial strains having a 16S rRNA sequence that is at least about 97%, at least about 98%, at least about 99%, or about 100% identical with the 16S rRNA sequence of any one of the operational taxonomic units provided in Table 5 or Table 6.

In various embodiments, the pharmaceutical composition of the invention comprises a bacterial mixture of at least about 50 different bacterial strains, or at least about 49 different bacterial strains, or at least about 48 different bacterial strains, or at least about 47 different bacterial strains, or at least about 46 different bacterial strains, or at least about 45 different bacterial strains, or at least about 44 different bacterial strains, or at least about 43 different bacterial strains, or at least about 42 different bacterial strains, or at least about 41 different bacterial strains, or at least about 40 different bacterial strains, or at least about 39 bacterial strains, or at least about 38 bacterial strains, or at least about 37 bacterial strains, or at least about 36 bacterial strains, or at least about 35 bacterial strains, or at least about 34 bacterial strains, or at least about 33 bacterial strains, or at least about 32 bacterial strains, or at least about 31 bacterial strains, or at least about 30 bacterial strains, or at least about 29 bacterial strains, or at least about 28 bacterial strains, or at least about 27 bacterial strains, or at least about 26 bacterial strains, or at least about 25 bacterial strains, or at least about 24 bacterial strains, or at least about 23 bacterial strains, or at least about 22 bacterial strains, or at least about 21 bacterial strains, or at least about 20 bacterial strains, or at least about 19 bacterial strains, or at least about 18 bacterial strains, or at least about 17 bacterial strains, or at least about 16 bacterial strains, or at least about 15 bacterial strains, or at least about 14 bacterial strains, or at least about 13 bacterial strains, or at least about 12 bacterial strains, or at least about 11 bacterial strains, or at least about 10 bacterial strains, or at least about 9 bacterial strains, or at least about 8 bacterial strains, or at least about 7 bacterial strains, or at least about 6 bacterial strains, or at least about 5 bacterial strains, or at least about 4 bacterial strains, or at least about 3 bacterial strains, or at least about 2 bacterial strains, or about 1 bacterial strain with reference to Table 5 or Table 6, e.g., as listed in Table 5 or Table 6 or having a 16S rRNA sequence that is, as examples, at least about 95%, at least about 96%, at least about 97%, at least about 98%, at least about 99%, or about 100% identical with the 16S rRNA sequence of any one of the strains listed in Table 5 or Table 6.

In various embodiments, the pharmaceutical composition of the invention comprises a bacterial mixture of about 50 or fewer different bacterial strains as described herein (e.g., with reference to Table 5 or Table 6).

In some embodiments, the pharmaceutical composition of the invention comprises greater than about 2, greater than about 5, or greater than about 10, or greater than about 15, or greater than about 20, or greater than about 25, or greater than about 30, or greater than about 35, or greater than about 40, or greater than about 45, or greater than about 50, greater than about 75, or greater than about 100 different bacterial strains as described herein (e.g., with reference to Table 5 or Table 6).

In some embodiments, the pharmaceutical composition of the invention comprises less than about 5, or less than about 10, or less than about 15, or less than about 20, or less than about 25, or less than about 30, or less than about 35, or less than about 40, or less than about 45, or less than about 50 different bacterial strains as described herein (e.g., with reference to Table 5 or Table 6).

In some embodiments, the pharmaceutical composition of the invention comprises about 10 to about 50 different bacterial strains as described herein (e.g., with reference to Table 5 or Table 6), including about 10 to about 45, or about 10 to about 40, or about 10 to about 30, or about 10 to about 20, or about 10 to about 15 different bacterial strains.

In some embodiments, the pharmaceutical composition of the invention comprises about 10 to about 20 different bacterial strains as described herein (e.g., with reference to Table 5 or Table 6).

In various embodiments, the mixtures of bacterial strains are selected from any of the bacterial strains listed in Table 5 or Table 6 below or the bacterial strains having a 16S rRNA sequence that is, as examples, at least about 95%, at least about 96%, at least about 97%, at least about 98%, at least about 99%, or about 100% identical with the 16S rRNA sequence of any one of the strains listed in Table 5 or Table 6 below.

In embodiments, the mixtures of bacterial strains are substantially complete or non-selected fecal microbiota preparations (e.g., from a single healthy stool donor), which generally comprises a full complement of functional microorganisms found in feces of one or more healthy humans. Such mixtures of bacterial strains may further comprise one or more strains listed in Table 5 or Table 6 below and/or one or more strains having a 16S rRNA sequence that is at least about 95% identical with the 16S rRNA sequence of any one of the strains listed in Table 5 or Table 6 below.

Table 5 lists OTUs by their GreenGenes identification number and the Latin taxonomic name associated with each OTU.

### Columns:

### A—GreenGenes ID for OTU

B—The number of stool donors that OTU is present in
C—The fraction of all stool donors that OTU is present in
D—Taxonomy for that OTU
E—SEQ ID NO associated with the OTU

TABLE 5
Exemplary OTUs Useful in the Present Invention

| A¹ | B | C | D² | E |
|---|---|---|---|---|
| 9710 | 6 | 0.095238095 | k: Bacteria; p: Proteobacteria; | 1 |
| | | | c: Gammaproteobacteria; | |
| | | | o: Enterobacteriales; | |
| | | | f: Enterobacteriaceae; g:; s: | |
| 14157 | 1 | 0.015873016 | k: Bacteria; p: Firmicutes; | 2 |
| | | | c: Clostridia; o: Clostridiales; | |
| | | | f: [Mogibacteriaceae]; g:; s: | |
| 14159 | 3 | 0.047619048 | k: Bacteria; p: Firmicutes; | 3 |
| | | | c: Clostridia; o: Clostridiales; | |
| | | | f: [Mogibacteriaceae]; g:; s: | |
| 15286 | 1 | 0.015873016 | k: Bacteria; p: Firmicutes; | 4 |
| | | | c: Bacilli; o: Lactobacillales; | |
| | | | f: Enterococcaceae; | |
| | | | g: Enterococcus; s: | |
| 16036 | 1 | 0.015873016 | k: Bacteria; p: Firmicutes; | 5 |
| | | | c: Clostridia; o: Clostridiales; | |
| | | | f: Ruminococcaceae; | |
| | | | g: Ruminococcus; s: flavefaciens | |
| 16054 | 33 | 0.523809524 | k: Bacteria; p: Firmicutes; | 6 |
| | | | c: Clostridia; o: Clostridiales; | |
| | | | f: Ruminococcaceae; | |
| | | | g: Ruminococcus; s: callidus | |
| 17311 | 9 | 0.142857143 | k: Bacteria; p: Firmicutes; | 7 |
| | | | c: Clostridia; o: Clostridiales; | |
| | | | f: Christensenellaceae; g:; s: | |
| 20321 | 5 | 0.079365079 | k: Bacteria; p: Proteobacteria; | 8 |
| | | | c: Gammaproteobacteria; | |
| | | | f: Enterobacteriaceae; g: Erwinia; s: | |
| 31319 | 4 | 0.063492063 | k: Bacteria; p: Firmicutes; | 9 |
| | | | c: Clostridia; o: Clostridiales; | |
| | | | f: Clostridiaceae; g: Clostridium; | |
| | | | s: subterminale | |
| 34789 | 1 | 0.015873016 | k: Bacteria; p: Firmicutes; | 10 |
| | | | c: Clostridia; o: Clostridiales; | |
| | | | f: Christennsenellaceae; g:; s: | |
| 35260 | 3 | 0.047619048 | k: Bacteria; p: Firmicutes; | 11 |
| | | | c: Clostridia; o: Clostridiales; | |
| | | | f: Christensenallaceae; g:; s: | |
| 36792 | 1 | 0.015873016 | k: Bacteria; p: Firmicutes; | 12 |
| | | | c: Clostridia; o: Clostridiales; | |
| | | | f: [Mogibacteriaceae]; g:; s: | |
| 41229 | 2 | 0.031746032 | k: Bacteria; p: Proteobacteria; | 13 |
| | | | c: Betaproteobacteria; | |
| | | | o: Burkholderiales; | |
| | | | f: Alcaligenaceae; | |
| | | | g: Sutterella; s: | |
| 42839 | 51 | 0.80952381 | k: Bacteria; p: Firmicutes; | 14 |
| | | | c: Clostridia; o: Clostridiales; | |
| | | | f: Ruminococcaceae; | |
| | | | g: Ruminococcus; s: | |
| 43950 | 6 | 0.095238095 | k: Bacteria; p: Firmicutes; | 15 |
| | | | c: Clostridia; o: Clostridiales; | |
| | | | f: Ruminococcaceae; | |
| | | | g: Oscillospira; s: | |
| 44827 | 7 | 0.111111111 | k: Bacteria; p: Firmicutes; | 16 |
| | | | c: Clostridia; o: Clostridiales; | |
| | | | f: Ruminococcaceae; | |
| | | | g: Ruminococcus; | |
| | | | s: flavefaciens | |
| 45074 | 1 | 0.015873016 | k: Bacteria; p: Firmicutes; | 17 |
| | | | c: Clostridia; o: Clostridiales; | |
| | | | f: Clostridiaceae; g: Clostridium; s: | |
| 45363 | 44 | 0.698412698 | k: Bacteria; p: Firmicutes; | 18 |
| | | | c: Erysipelotrichi; | |
| | | | o: Erysipelotrichales; | |
| | | | f: Erysipelotrichaceae; | |
| | | | g: Clostridium; | |
| | | | s: cocleatum | |
| 48444 | 1 | 0.015873016 | k: Bacteria; p: Firmicutes; | 19 |
| | | | c: Clostridia; o: Clostridiales; | |
| | | | f: Ruminococcaceae; | |
| | | | g: Ruminococcus; s: | |
| 66188 | 1 | 0.015873016 | k: Bacteria; p: Firmicutes; | 20 |
| | | | c: Clostridia; o: Clostridiales; | |
| | | | f: Ruminococcaceae; | |
| | | | g: Oscillospira; s: | |
| 68841 | 10 | 0.158730159 | k: Bacteria; p: Firmicutes; | 21 |
| | | | c: Clostridia; o: Clostridiales; | |
| | | | f: Lachnospiraceae; | |
| | | | g: Lachnospira; s: | |
| 72481 | 2 | 0.031746032 | k: Bacteria; p: Firmicutes; | 22 |
| | | | c: Clostridia; o: Clostridiales; | |
| | | | f: Lachnospiraceae; | |
| | | | g: Coprococcus; s: | |
| 73753 | 52 | 0.825396825 | k: Bacteria; p: Bacteroidetes; | 23 |
| | | | c: Bacteroidia; o: Bacteroidales; | |
| | | | f: [Odoribacteraceae]; | |
| | | | g: Odoribacter; s: | |
| 91359 | 1 | 0.015873016 | k: Bacteria; p: Firmicutes; | 24 |
| | | | c: Clostridia; o: Clostridiales; | |
| | | | f: Lachnospiraceae; | |
| | | | g: Lachnospira; s: | |
| 91557 | 1 | 0.015873016 | k: Bacteria; p: Proteobacteria; | 25 |
| | | | c: Gammaproteobacteria; | |
| | | | o: Enterobacteriales; f: | |
| | | | Enterobacteriaceae; g:; s: | |
| 91962 | 4 | 0.063492063 | k: Bacteria; p: Proteobacteria; | 26 |
| | | | c: Gammaproteobacteria; | |
| | | | o: Enterobacteriales; | |
| | | | f: Enterbacteriaceae; g:; s: | |
| 100506 | 1 | 0.015873016 | k: Bacteria; p: Firmicutes; | 27 |
| | | | c: Clostridia; o: Clostridiales; | |
| | | | f: Lachnospiraceae; | |
| | | | g: Coprococcus; s: | |
| 105538 | 3 | 0.047619048 | k: Bacteria; p: Firmicutes; | 28 |
| | | | c: Clostridia; o: Clostridiales; | |
| | | | f: Clostridiaceae; g: Clostridium; s: | |
| 105964 | 1 | 0.015873016 | k: Bacteria; p: Firmicutes; | 29 |
| | | | c: Clostridia; o: Clostridiales; | |
| | | | f: Ruminococcaceae; | |
| | | | g: Ruminococcus; | |
| | | | s: flavefaciens | |
| 107044 | 16 | 0.253968254 | k: Bacteria; p: Bacteroidetes; | 30 |
| | | | c: Bacteroidia; o: Bacteroidales; | |
| | | | f: Rikenellaceae; g: Alistipes; | |
| | | | s: massiliensis | |
| 110192 | 2 | 0.031746032 | k: Bacteria; p: Firmicutes; | 31 |
| | | | c: Clostridia; o: Clostridiales; | |
| | | | f: Ruminococcaceae; | |
| | | | g: Oscillospira; s: | |
| 110458 | 2 | 0.031746032 | k: Bacteria; p: Firmicutes; | 32 |
| | | | c: Clostridia; o: Clostridiales; | |
| | | | f: Christensenellaceae; g:; s: | |
| 110562 | 14 | 0.222222222 | k: Bacteria; p: Firmicutes; | 33 |
| | | | c: Clostridia; o: Clostridiales; | |
| | | | f: Ruminococcaceae; | |
| | | | g: Ruminococcus; s: | |
| 112720 | 1 | 0.015873016 | k: Bacteria; p: Bacteroidetes; | 34 |
| | | | c: Bacteroidia; o: Bacteroidales; | |
| | | | f: Rikenellaceae; g:; s: | |
| 112891 | 2 | 0.031746032 | k: Bacteria; p: Proteobacteria; | 35 |
| | | | c: Betaproteobacteria; | |
| | | | o: Burkholderiales; | |
| | | | f: Alcaligenaceae; | |
| | | | g: Sutterella; s: | |
| 113003 | 13 | 0.206349206 | k: Bacteria; p: Firmicutes; | 36 |
| | | | c: Clostridia; o: Clostridiales; | |
| | | | f: Ruminococcaceae; | |
| | | | g: Ruminococcus; s: | |
| 114462 | 1 | 0.015873016 | k: Bacteria; p: Proteobacteria; | 37 |
| | | | c: Gammaproteobacteria; | |
| | | | o: Enterobacteriales; f: | |
| | | | Enterobacteriaceae; g:; s: | |
| 115035 | 2 | 0.031746032 | k: Bacteria; p: Proteobacteria; | 39 |
| | | | c: Gammaproteobacteria; | |
| | | | o: Enterobacteriales; | |
| | | | f: Enterobacteriaceae; | |
| | | | g: Xenorhabdus; | |
| | | | s: bovienii | |
| 122656 | 7 | 0.111111111 | k: Bacteria; p: Firmicutes; | 40 |
| | | | c: Clostridia; o: Clostridiales; | |
| | | | f: Christensenellaceae; g:; s: | |
| 129401 | 7 | 0.111111111 | k: Bacteria; p: Firmicutes; | 41 |
| | | | c: Clostridia; o: Clostridiales; | |
| | | | f: Ruminococcaceae; | |
| | | | g: Oscillospira; s: | |
| 132784 | 1 | 0.015873016 | k: Bacteria; p: Firmicutes; | 42 |
| | | | c: Clostridia; o: Clostridiales; | |
| | | | f: Veillonellaceae; g: | |
| | | | Phascolarctobacterium; s: | |
| 132991 | 45 | 0.714285714 | k: Bacteria; p: Firmicutes; | 43 |
| | | | c: Clostridia; o: Clostridiales; | |
| | | | f: Ruminococcaceae; | |
| | | | g: Faecalibacterium; | |
| | | | s: prausnitzii | |
| 136127 | 9 | 0.142857143 | k: Bacteria; p: Actinobacteria; | 44 |
| | | | c: Coriobacteriia; | |
| | | | o: Coriobacteriales; | |
| | | | f: Coriobacteriaceae; g:; s: | |
| 136789 | 1 | 0.015873016 | k: Bacteria; p: Firmicutes; | 45 |
| | | | c: Clostridia; o: Clostridiales; | |
| | | | f: Ruminococcaceae; | |
| | | | g: Ruminococcus; s: | |
| 137353 | 13 | 0.206349206 | k: Bacteria; p: Firmicutes; | 46 |
| | | | c: Clostridia; o: Clostridiales; | |
| | | | f: Lachnospiraceae; | |
| | | | g: Coprococcus; s: | |
| 141145 | 2 | 0.031746032 | k: Bacteria; p: Proteobacteria; | 47 |
| | | | c: Gammaproteobacteria; | |
| | | | o: Enterobacteriales; f: | |
| | | | Enterobacteriaceae; g:; s: | |
| 145236 | 5 | 0.079365079 | k: Bacteria; p: Firmicutes; | 48 |
| | | | c: Clostridia; o: Clostridiales; | |
| | | | f: Christensenellaceae; g:; s: | |
| 145856 | 7 | 0.111111111 | k: Bacteria; p: Firmicutes; | 49 |
| | | | c: Clostridia; o: Clostridiales; | |
| | | | f: [Mogibacteriaceae]; g:; s: | |
| 145887 | 1 | 0.015873016 | k: Bacteria; p: Firmicutes; | 50 |
| | | | c: Clostridia; o: Clostridiales; | |
| | | | f: [Mogibacteriaceae]; g:; s: | |
| 147071 | 54 | 0.857142857 | k: Bacteria; p: Actinobacteria; | 51 |
| | | | c: Coriobacteriia; | |
| | | | o: Coriobacteriales; | |
| | | | f: Coriobacteriaceae; | |
| | | | g: Collinsella; s: | |
| 147195 | 10 | 0.158730159 | k: Bacteria; p: Firmicutes; | 52 |
| | | | c: Clostridia; o: Clostridiales; | |
| | | | f: Lachnospiraceae; | |
| | | | g: Coprococcus; s: | |
| 147484 | 7 | 0.111111111 | k: Bacteria; p: Firmicutes; | 53 |
| | | | c: Clostridia; o: Clostridiales; | |
| | | | f: [ Mogibacteriaceae]; g:; s: | |
| 148620 | 48 | 0.761904762 | k: Bacteria; p: Proteobacteria; | 54 |
| | | | c: Gammaproteobacteria; | |
| | | | o: Enterobacteriales; | |
| | | | f: Enterbacteriaceae; | |
| | | | g: Erwinia; s: | |
| 148925 | 5 | 0.079365079 | k: Bacteria; p: Firmicutes; | 55 |
| | | | c: Clostridia; o: Clostridiales; | |
| | | | f: Ruminococcaceae; | |
| | | | g: Ruminococcus; s: | |
| 149588 | 16 | 0.253968254 | k: Bacteria; p: Proteobacteria; | 56 |
| | | | c: Gammaproteobacteria; | |
| | | | o: Enterobacteriales; | |
| | | | f: Enterobacteriaceae; | |
| | | | g: Erwinia; s: | |
| 157162 | 1 | 0.015873016 | k: Bacteria; p: Firmicutes; | 57 |
| | | | c: Clostridia; o: Clostridiales; | |
| | | | f: Ruminococcaceae; | |
| | | | g: Oscillospira; s: | |
| 157224 | 11 | 0.174603175 | k: Bacteria; p: Firmicutes; | 58 |
| | | | c: Clostridia; o: Clostridiales; | |
| | | | f: Ruminococcaceae; g: | |
| | | | Faecalibacterium; s: prausnitzii | |
| 157573 | 1 | 0.015873016 | k: Bacteria; p: Bacteroidetes; | 59 |
| | | | c: Bacteroidia; o: Bacteroidales; | |
| | | | f: Rikenellaceae; g:; s: | |
| 157772 | 22 | 0.349206349 | k: Bacteria; p: Firmicutes; | 60 |
| | | | c: Clostridia; o: Clostridiales; | |
| | | | f: Ruminococcaceae; | |
| | | | g: Oscillospira; s: | |
| 158045 | 33 | 0.523809524 | k: Bacteria; p: Firmicutes; | 61 |
| | | | c: Clostridia; o: Clostridiales; | |
| | | | f: Ruminococcaceae; g: | |
| | | | Faecalibacterium; s: | |
| | | | prausnitzii | |
| 158113 | 3 | 0.047619048 | k: Bacteria; p: Proteobacteria; | 62 |
| | | | c: Betaproteobacteria; | |
| | | | o: Burkholderiales; | |
| | | | f: Alcaligenaceae; | |
| | | | g: Sutterella; s: | |
| 158183 | 5 | 0.079365079 | k: Bacteria; p: Firmicutes; | 63 |
| | | | c: Clostridia; o: Clostridiales; | |
| | | | f: Ruminococcaceae; | |
| | | | g: Oscillospira; s: | |
| 158297 | 1 | 0.015873016 | k: Bacteria; p: Firmicutes; | 64 |
| | | | c: Clostridia; o: Clostridiales; | |
| | | | f: Ruminococcaceae; | |
| | | | g: Ruminococcus; s: | |
| 158722 | 11 | 0.174603175 | k: Bacteria; p: Firmicutes; | 65 |
| | | | c: Clostridia; o: Clostridiales; | |
| | | | f: Ruminococcaceae; | |
| | | | g: Oscillospira; s: | |
| 160750 | 1 | 0.015873016 | k: Bacteria; p: Firmicutes; | 66 |
| | | | c: Clostridia; o: Clostridiales; | |
| | | | f: Lachnospiraceae; | |
| | | | g: Coprococcus; s: | |
| 161762 | 2 | 0.031746032 | k: Bacteria; p: Firmicutes; | 67 |
| | | | c: Clostridia; o: Clostridiales; | |
| | | | f: Ruminococcaceae; | |
| | | | g: Ruminococcus; s: | |
| 162215 | 1 | 0.015873016 | k: Bacteria; p: Proteobacteria; | 68 |
| | | | c: Gammaproteobacteria; | |
| | | | o: Enterbacteriales; f: | |
| | | | Enterobacteriaceae; g:; s: | |
| 162660 | 20 | 0.317460317 | k: Bacteria; p: Firmicutes; | 69 |
| | | | c: Clostridia; o: Clostridiales; | |
| | | | f: Ruminococcaceae; | |
| | | | g: Ruminococcus; s: | |
| 163243 | 10 | 0.158730159 | k: Bacteria; p: Firmicutes; | 70 |
| | | | c: Clostridia; o: Clostridiales; | |
| | | | f: Ruminococcaceae; | |
| | | | g: Ruminococcus; s: | |
| 163421 | 1 | 0.015873016 | k: Bacteria; p: Proteobacteria; | 71 |
| | | | c: Gammaproteobacteria; | |
| | | | o: Enterobacteriales; | |
| | | | f: Enterobacteriaceae; | |
| | | | g: Citrobacter; s: | |
| 165046 | 3 | 0.074619048 | k: Bacteria; p: Firmicutes; | 72 |
| | | | c: Clostridia; o: Clostridiales; | |
| | | | f: Ruminococcaceae; | |
| | | | g: Oscillospira; s: | |
| 165924 | 55 | 0.873015873 | k: Bacteria; p: Firmicutes; | 73 |
| | | | c: Clostridia; o: Clostridiales; | |
| | | | f: Ruminococcaceae; | |
| | | | g: Faecalibacterium; | |
| | | | s: prausnitzii | |
| 166156 | 3 | 0.047619048 | k: Bacteria; p: Firmicutes; | 74 |
| | | | c: Clostridia; o: Clostridiales; | |
| | | | f: Lachnospiraceae; | |
| | | | g: Coprococcus; s: | |
| 166164 | 4 | 0.063492063 | k: Bacteria; p: Firmicutes; | 75 |
| | | | c: Clostridia; o: Clostridiales; | |
| | | | f: Ruminococcaceae; | |
| | | | g: Ruminococcus; s: | |
| 166226 | 4 | 0.063492063 | k: Bacteria; p: Firmicutes; | 76 |
| | | | c: Clostridia; o: Clostridiales; | |
| | | | f: Ruminococcaceae; | |
| | | | g: Oscillospira; s: | |
| 166908 | 59 | 0.936507937 | k: Bacteria; p: Proteobacteria; | 77 |
| | | | c: Gammaproteobacteria; | |
| | | | o: Enterobacteriales; f: | |
| | | | Enterobacteriaceae; g:; s: | |
| 166913 | 43 | 0.682539683 | k: Bacteria; p: Firmicutes; | 78 |
| | | | c: Clostridia; f: Clostridiaceae; g:; s: | |
| 169031 | 1 | 0.015873016 | k: Bacteria; p: Firmicutes; | 79 |
| | | | c: Clostridia; o: Clostridiales; | |
| | | | f: Veillonellaceae; g: | |
| | | | Phascolarctobacterium; s: | |
| 169182 | 3 | 0.047619048 | k: Bacteria; p: Proteobacteria; | 80 |
| | | | c: Gammaproteobacteria; | |
| | | | o: Enterobacteriales; f: | |
| | | | Enterobacteriaceae; g:; s: | |
| 171184 | 1 | 0.015873016 | k: Bacteria; p: Firmicutes; | 81 |
| | | | c: Clostridia; o: Clostridiales; | |
| | | | f: Clostridiaceae; g:; s; | |
| 171437 | 2 | 0.031746032 | k: Bacteria; p: Firmicutes; | 82 |
| | | | c: Clostridia; o: Clostridiales; | |
| | | | f: Clostridiaceae; g:; s: | |
| 171772 | 12 | 0.19047619 | k: Bacteria; p: Bacteroidetes; | 83 |
| | | | c: Bacteroidia; o: Bacteroidales; | |
| | | | f: Rikenellaceae; g:; s: | |
| 173103 | 7 | 0.111111111 | k: Bacteria; p: Firmicutes; | 84 |
| | | | c: Clostridia; o: Clostridiales; | |
| | | | f: Clostridiaceae; g:; s: | |
| 173135 | 13 | 0.206349206 | k: Bacteria; p: Firmicutes; | 85 |
| | | | c: Clostridia; o: Clostridiales; | |
| | | | f: Ruminococcaceae; | |
| | | | g: Faecalibacterium; | |
| | | | s: prausnitzii | |
| 173654 | 1 | 0.015873016 | k: Bacteria; p: Proteobacteria; | 86 |
| | | | c: Gammaproteobacteria; | |
| | | | o: Enterobacteriales; f: | |
| | | | Enterobacteriaceae; g:; s: | |
| 173726 | 28 | 0.444444444 | k: Bacteria; p: Proteobacteria; | 87 |
| | | | c: Betaproteobacteria; | |
| | | | o: Burkholderiales; | |
| | | | f: Alcaligenaceae; | |
| | | | g: Sutterella; s: | |
| 173792 | 3 | 0.047619048 | k: Bacteria; p: Firmicutes; | 88 |
| | | | c: Clostridia; o: Clostridiales; | |
| | | | f: Ruminococcaceae; | |
| | | | g: Ruminococcus; s: | |
| 173969 | 1 | 0.015873016 | k: Bacteria; p: Firmicutes; | 89 |
| | | | c: Clostridia; o: Clostridiales; | |
| | | | f: Lachnospiraceae; | |
| | | | g: Coprococcus; s: | |
| 173986 | 9 | 0.142857143 | k: Bacteria; p: Firmicutes; | 90 |
| | | | c: Clostridia; o: Clostridiales; | |
| | | | f: Ruminococcaceae; | |
| | | | g: Faecalibactrium; | |
| | | | g: prausnitzii | |
| 174045 | 42 | 0.666666667 | k: Bacteria; p: Firmicutes; | 91 |
| | | | c: Clostridia; o: Clostridiales; | |
| | | | f: Lachnospiraceae; | |
| | | | g: Coprococcus; s: | |
| 174142 | 3 | 0.047619048 | k: Bacteria; p: Actinobacteria; | 92 |
| | | | c: Coriobacteriia; | |
| | | | o: Coriobacteriales; | |
| | | | f: Coriobacteriaceae; g:; s: | |
| 174233 | 1 | 0.015873016 | k: Bacteria; p: Firmicutes; | 93 |
| | | | c: Clostridia; o: Clostridiales; | |
| | | | f: Clostridiaceae; g:; s: | |
| 174403 | 2 | 0.031746032 | k: Bacteria; p: Firmicutes; | 94 |
| | | | c: Clostridia; o: Clostridiales; | |
| | | | f: Ruminococcaceae; | |
| | | | g: Faecalibacterium; | |
| | | | s: prausnitzii | |
| 174439 | 43 | 0.682539683 | k: Bacteria; p: Firmicutes; | 95 |
| | | | c: Clostridia; o: Clostridiales; | |
| | | | f: Ruminococcaceae; | |
| | | | g: Faecalibacterium; | |
| | | | s: prausnitzii | |
| 174497 | 9 | 0.142857143 | k: Bacteria; p: Firmicutes; | 96 |
| | | | c: Clostridia; o: Clostridiales; | |
| | | | f: Clostridiaceae; g: Clostridium; s: | |
| 174516 | 7 | 0.111111111 | k: Bacteria; p: Firmicutes; | 97 |
| | | | c: Clostridia; o: Clostridiales; | |
| | | | f: Clostridiaceae; g: Clostridium; s: | |
| 174607 | 1 | 0.015873016 | k: Bacteria; p: Firmicutes; | 98 |
| | | | c: Clostridia; o: Clostridiales; | |
| | | | f: Ruminococcaceae; | |
| | | | g: Ruminococcus; s: | |
| 174614 | 48 | 0.761904762 | k: Bacteria; p: Firmicutes; | 99 |
| | | | c: Clostridia; o: Clostridiales; | |
| | | | f: Ruminococcaceae; | |
| | | | g: Ruminococcus; s: | |
| 174625 | 9 | 0.142857143 | k: Bacteria; p: Firmicutes; | 100 |
| | | | c: Clostridia; o: Clostridiales; | |
| | | | f: Lachnospiraceae; g: Coproccus; s: | |
| 174654 | 1 | 0.015873016 | k: Bacteria; p: Firmicutes; | 101 |
| | | | c: Clostridia; o: Clostridiales; | |
| | | | f: Ruminococcaceae; | |
| | | | g: Ruminococcus; | |
| | | | s: bromii | |
| 174688 | 2 | 0.031746032 | k: Bacteria; p: Firmicutes; | 102 |
| | | | c: Clostridia; o: Clostridiales; | |
| | | | f: Clostridiaceae; g: Clostridium; s: | |
| 174172 | 6 | 0.095238095 | k: Bacteria; p: Firmicutes; | 103 |
| | | | c: Clostridia; o: Clostridiales; | |
| | | | f: Ruminococcaceae; | |
| | | | g: Ruminococcus; s: | |
| 174755 | 12 | 0.19047619 | k: Bacteria; p: Actinobacteria; | 104 |
| | | | c: Coriobacteriia; | |
| | | | o: Coriobacteriales; | |
| | | | f: Coriobacteriaceae; g: Collinsella; | |
| | | | s: aerofaciens | |
| 174960 | 7 | 0.111111111 | k: Bacteria; p: Firmicutes; | 105 |
| | | | c: Clostridia; o: Clostridiales; | |
| | | | f: Clostridiaceae; g:; s: | |
| 174974 | 12 | 0.19047619 | k: Bacteria; p: Firmicutes; | 106 |
| | | | c: Clostridia; o: Clostridiales; | |
| | | | f: Lachnospiraceae; | |
| | | | g: Lachnospira; s: | |
| 175037 | 3 | 0.047619048 | k: Bacteria; p: Firmicutes; | 107 |
| | | | c: Clostridia; o: Clostridiales; | |
| | | | f: Christensenellaceae; g:; s: | |
| 175375 | 2 | 0.031746032 | k: Bacteria; p: Bacteroidetes; | 108 |
| | | | c: Bacteroidia; o: Bacteroidales; | |
| | | | f: Rikenellaceae; g:; s: | |
| 175397 | 59 | 0.936507937 | k: Bacteria: p: Firmicutes; | 109 |
| | | | c: Clostridia; o: Clostridiales; | |
| | | | f: Lachnospiraceae; | |
| | | | g: Coprococcus; s: | |
| 175438 | 2 | 0.031746032 | k: Bacteria; p: Firmicutes; | 110 |
| | | | c: Clostridia; o: Clostridiales; | |
| | | | f: Lachnospiraceae; | |
| | | | g: Coprococcus; s: | |
| 175508 | 46 | 0.73015873 | k: Bacteria; p: Actinobacter; | 111 |
| | | | c: Coriobacteriia; | |
| | | | o: Coriobacteriales; | |
| | | | f: Coriobacteriaceae; g:; s: | |
| 175560 | 63 | 1 | k: Bacteria; p: Firmicutes; c:, | 112 |
| | | | Clostridia; o: Clostridiales; | |
| | | | f: Ruminococcaceae; | |
| | | | g: Faecalibacterium; | |
| | | | s: prausnitzii | |
| 175846 | 3 | 0.047619048 | k: Bacteria;; p: Firmicutes; | 113 |
| | | | c: Clostridia; o: Clostridiales; | |
| | | | f: Lachnospiraceae; | |
| | | | g: Coprococcus; s: | |
| 175883 | 6 | 0.095238095 | k: Bacteria; p: Firmicutes; | 114 |
| | | | c: Clostridia; o: Clostridiales; | |
| | | | f: Lachnospiraceae; | |
| | | | g: Coprococcus; s: | |
| 175898 | 3 | 0.047619048 | k: Bacteria; p: Firmicutes; | 115 |
| | | | c: Clostridia; o: Clostridiales; | |
| | | | f: Ruminococcaceae; | |
| | | | g: Oscillospira; s: | |
| 175922 | 4 | 0.063492063 | k: Bacteria; p: Firmicutes; | 116 |
| | | | c: Clostridia; o: Clostridiales; | |
| | | | f: Ruminococcaceae; | |
| | | | g: Oscillospira; s: | |
| 176094 | 2 | 0.031746032 | k: Bacteria; p: Firmicutes; | 117 |
| | | | c: Clostridia; o: Clostridiales; | |
| | | | f: Ruminococcaceae; | |
| | | | g: Ruminococcus; s: | |
| 176104 | 23 | 0.365079365 | k: Bacteria; p: Firmicutes; | 118 |
| | | | c: Clostridia; o: Clostridiales; | |
| | | | f: Ruminococcaceae; | |
| | | | g: Oscillospira; s: | |
| 176108 | 9 | 0.142857143 | k: Bacteria; p: Bacteroidetes; | 119 |
| | | | c: Bacteroidia; o: Bacteroidales; | |
| | | | f: Rikenellaceae; g:; s: | |
| 176115 | 45 | 0.714285714 | k: Bacteria; p: Firmicutes; | 120 |
| | | | c: Clostridia; o: Clostridiales; | |
| | | | f: Ruminococcaceae; | |
| | | | g: Faecalibacterium; | |
| | | | s: praesnitzii | |
| 176167 | 14 | 0.222222222 | k: Bacteria; p: Firmicutes; | 121 |
| | | | c: Clostridia; o: Clostridiales; | |
| | | | f: Ruminococcaceae; | |
| | | | g: Ruminococcus; s: | |
| 176201 | 30 | 0.476190476 | k: Bacteria; p: Firmicutes; | 122 |
| | | | c: Clostridia; o: Clostridiales; | |
| | | | f: Lachnospiraceae; | |
| | | | g: Coprococcus; s: | |
| 176300 | 4 | 0.063492063 | k: Bacteria; p: Firmicutes; | 123 |
| | | | c: Clostridia; o: Cloatridiales; | |
| | | | f: Lachnospiraceae; | |
| | | | g: Coprococcus; s: | |
| 176518 | 10 | 0.158730159 | k: Bacteria; p: Firmicutes; | 124 |
| | | | c: Clostridia; o: Clostridiales; | |
| | | | f: Lachnospiraceae; g: Coprococcus; | |
| | | | s: eutactus | |
| 176566 | 1 | 0.015873016 | k: Bacteria; p: Firmicutes; | 125 |
| | | | c: Clostridia; o: Clostridiales; | |
| | | | f: Ruminococcaceae; | |
| | | | g: Oscollospira; s: | |
| 176705 | 4 | 0.063492063 | k: Bacteria; p: Firmicutes; | 126 |
| | | | c: Clostridia; o: Clostridiales; | |
| | | | f: Veillonellaceae; g: | |
| | | | Phascolarctobacterium; s: | |
| 176775 | 47 | 0.746031746 | k: Bacteria; p: Firmicutes; | 127 |
| | | | c: Clostridia; o: Clostridiales; | |
| | | | f: Veillonellaceae; g: | |
| | | | Phascolarctobacterium; s: | |
| 177058 | 5 | 0.079365079 | k: Bacteria; p: Firmicutes; | 128 |
| | | | c: Clostridia; o: Clostridiales; | |
| | | | f: Lachnospiraceae; | |
| | | | g: Coprococcus; s: | |
| 177109 | 1 | 0.015873016 | k: Bacteria; p: Firmicutes; | 129 |
| | | | c: Clostridia; o: Clostridiales; | |
| | | | f: Ruminococcaceae; | |
| | | | g: Oscillospira; s: | |
| 177111 | 39 | 0.619047619 | k: Bacteria; p: Firmicutes; | 130 |
| | | | c: Clostridia; o: Clostridiales; | |
| | | | f: Lachnospiraceae; | |
| | | | g: Coprococcus; s: | |
| 177161 | 35 | 0.555555556 | k: Bacteria; p: Firmicutes; | 131 |
| | | | c: Clostridia; o: Clostridiales; | |
| | | | f: Clostridiaceae; g: Clostridium; s: | |
| 177172 | 57 | 0.904761905 | k: Bacteria; p: Firmicutes; | 132 |
| | | | c: Clostridia; o: Clostridiales; | |
| | | | f: Lachnospiraceae; | |
| | | | g: Coprococcus; s: | |
| 177217 | 1 | 0.015873016 | k: Bacteria; p: Firmicutes; | 133 |
| | | | c: Clostridia; o: Clostridiales; | |
| | | | f: Ruminococcaceae; | |
| | | | g: Faecalibacterium; | |
| | | | s: prausnitzii | |
| 177228 | 2 | 0.031746032 | k: Bacteria; p: Firmicutes; | 134 |
| | | | c: Clostridia; o: Clostridiales; | |
| | | | f: Clostridiaceae; g:; s: | |
| 177301 | 2 | 0.031746032 | k: Bacteria; p: Firmicutes; | 135 |
| | | | c: Clostridia; o: Clostridiales; | |
| | | | f: Lachnospiraceae; | |
| | | | g: Coprococcus; s: | |
| 177403 | 1 | 0.015873016 | k: Bacteria; p: Firmicutes; | 136 |
| | | | c: Clostridia; o: Clostridiales; | |
| | | | f: Ruminococcaceae; | |
| | | | g: Ruminococcus; s: | |
| 177463 | 8 | 0.126984127 | k: Bacteria; p: Firmicutes; | 137 |
| | | | c: Clostridia; o: Clostridiales; | |
| | | | f: Lachnospiraceae; | |
| | | | g: Coprococcus; s: | |
| 177478 | 38 | 0.603174603 | k: Bacteria; p: Actinobacteria; | 138 |
| | | | c: Coriobacteriia; | |
| | | | o: Coriobacteriales; | |
| | | | f: Coriobacteriaceae; g:; s: | |
| 177493 | 2 | 0.031746032 | k: Bacteria; p: Firmicutes; | 139 |
| | | | c: Clostridia; o: Clostridiales; | |
| | | | f: Lachnospiraceae; | |
| | | | g: Coprococcus; s: | |
| 177518 | 12 | 0.19047619 | k: Bacteria; p: Firmicutes; | 140 |
| | | | c: Clostridia; o: Clostridiales; | |
| | | | f: Lachnospiraceae; | |
| | | | g: Coproccus; s: | |
| 177586 | 6 | 0.095238095 | k: Bacteria; p: Firmicutes; | |
| | | | c: Clostridia; o: Clostridiales; | 141 |
| | | | f: Lachnospiraceae; | |
| | | | g: Coprococcus; s: | |
| 177593 | 13 | 0.206349206 | k: Bacteria; p: Firmicutes; | |
| | | | c: Clostridia; o: Clostridiales; | 142 |
| | | | f: Ruminococcaceae; | |
| | | | g: Ruminococcus; s: | |
| 177600 | 3 | 0.047619048 | k: Bacteria; p: Firmicutes; | 143 |
| | | | c: Cloastridia; o: Clostridiales; | |
| | | | f: Christensenellaceae; g:; s: | |
| 177623 | 5 | 0.079365079 | k: Bacteria; p: Firmicutes; | 144 |
| | | | c: Clostridia; o: Clostridiales; | |
| | | | f: Ruminococcaceae; | |
| | | | g: Ruminococcus; s: | |
| 177727 | 1 | 0.015873016 | k: Bacteria; p: Firmicutes; | 145 |
| | | | c: Clostridia; o: Clostridiales; | |
| | | | f: Ruminococcaceae; | |
| | | | g: Ruminococcus; s: | |
| 177754 | 11 | 0.174603175 | k: Bacteria; p: Firmicutes; | 146 |
| | | | c: Clostridia; o: Clostridiales; | |
| | | | f: Lachmospiraceae; | |
| | | | g: Coprococcus; s: | |
| 177775 | 1 | 0.015873016 | k: Bacteria; p: Firmicutes; | 147 |
| | | | c: Clostridia; o: Clostridiales; | |
| | | | f: Ruminococcaceae; | |
| | | | g: Faecalibacterium; | |
| | | | s: prausnitzii | |
| 177800 | 20 | 0.317460317 | k: Bacteria; p: Firmicutes; | 148 |
| | | | c: Clostridia; o: Clostridiales; | |
| | | | f: Ruminococcaceae; | |
| | | | g: Ruminococcus; s: | |
| 177933 | 1 | 0.015873016 | k: Bacteria; p: Firmicutes; | 149 |
| | | | c: Clostridia; o: Clostridiales; | |
| | | | f: Lachnospiraceae; | |
| | | | g: Coprococcus; s: | |
| 177987 | 52 | 0.825396825 | k: Bacteria; p: Firmicutes; | 150 |
| | | | c: Clostridia; o: Clostridiales; | |
| | | | f: Lachnospiraceae; | |
| | | | g: Lachnospira; s: | |
| 178001 | 21 | 0.333333333 | k: Bacteria; p: Fifmicutes; | 151 |
| | | | c: Clostridia; o: Clostridiales; | |
| | | | f: Lachnospiraceae; | |
| | | | g: Lachnospira; s: | |
| 178082 | 13 | 0.206349206 | k: Bacteria; p: Firmicutes; | 152 |
| | | | c: Clostridia; o: Clostridiales; | |
| | | | f: Lachnospiraceae; | |
| | | | g: Lachnospira; s: | |
| 178117 | 9 | 0.142857143 | k: Bacteria; p: Firmicutes; | 153 |
| | | | c: Clostridia; o: Clostridiales; | |
| | | | f: Ruminococcaceae; | |
| | | | g: Ruminococcus; s: | |
| 178146 | 1 | 0.015783016 | k: Bacteria; p: Firmicutes; | 154 |
| | | | c: Clostridia; o: Clostridiales; | |
| | | | f: Lachnospiraceae; | |
| | | | g: Coprococcus; s: | |
| 178151 | 10 | 0.158730159 | k: Bacteria; p: Firmicutes; | 155 |
| | | | c: Clostridia; o: Clostridiales; | |
| | | | f: Ruminococcaceae; | |
| | | | g: Ruminococcus; s: | |
| 178183 | 39 | 0.619047619 | k: Bacteria; p: Firmicutes; | 156 |
| | | | c: Clostridia; o: Clostridiales; | |
| | | | f: Clostridiaceae; g:; s; | |
| 178205 | 18 | 0.285714286 | k: Bacteria; p: Firmicutes; | 157 |
| | | | c: Clostridia; o: Clostridiales; | |
| | | | f: Lachnospiraceae; | |
| | | | g: Coprococcus; s: | |
| 178238 | 59 | 0.936507937 | k: Bacteria; p: Frimicutes; | 158 |
| | | | c: Clostridia, o: Clostridiales; | |
| | | | f: Lachnospiraceae; | |
| | | | g: Coprococcus; s: | |
| 178370 | 3 | 0.047619048 | k: Bacteria; p: Firmicutes; | 159 |
| | | | c: Clostridia; o: Clostridiales; | |
| | | | f: Clostridiaceae; g: Clostridium; s: | |
| 178385 | 13 | 0.206349206 | k: Bacteria; p: Firmicutes; | 160 |
| | | | c: Clostridia; o: Clastridiales; | |
| | | | f: Ruminococcaceae; | |
| | | | g: Faecalibacterium; | |
| | | | s: prausnitzii | |
| 178557 | 6 | 0.095238095 | k: Bacteria; p: Firmicutes; | 161 |
| | | | c: Clostridia; o: Clostridiales; | |
| | | | f: Lachnospiraceae; | |
| | | | g: Coprococcus; s: | |
| 178559 | 1 | 0.015873016 | k: Bacteria; p: Firmicutes; | 162 |
| | | | c: Clostridia; o: Clostridiales; | |
| | | | f: Lachnospiraceae; | |
| | | | g: Coprococcus; s: | |
| 178584 | 47 | 0.746031746 | k: Bacteria; p: Firmicutes; | 163 |
| | | | c: Clostridia; o: Clostridiales; | |
| | | | f: Clostridiaceae; g: Clostridium; s: | |
| 178629 | 45 | 0.714285714 | k: Bacteria; p: Firmicutes; | 164 |
| | | | c: Clostridia; o: Clostridiales; | |
| | | | f: Lachnospiraceae; | |
| | | | g: Coprococcus; s: | |
| 178738 | 25 | 0.966825397 | k: Bacteria; p: Firmicutes; | 165 |
| | | | c: Clostridia; o: Clostridiales; | |
| | | | f: Clostridiaceae; g:; s: | |
| 178839 | 5 | 0.079365079 | k: Bacteria; p: Firmicutes; | 166 |
| | | | c: Clostridia; o: Clostridiales; | |
| | | | f: [Mogibacteriaceae]; g:; s: | |
| 178852 | 31 | 0.492063492 | k: Bacteria; p: Firmicutes; | 167 |
| | | | c: Clostridia; o: Clostridiales; | |
| | | | f: Ruminococcaceae; | |
| | | | g: Oscillospira; s: | |
| 178882 | 1 | 0.015873016 | k: Bacteria; p: Firmicutes; | 168 |
| | | | c: Clostridia; o: Clostridiales; | |
| | | | f: Lachnospiraceae; | |
| | | | g: Coprococcus; s: | |
| 178961 | 18 | 0.285714286 | k: Bacteria; p: Firmicutes; | 169 |
| | | | c: Clostridia; o: Clostridiales; | |
| | | | f: Lachnospiraceae; | |
| | | | g: Coprococcus; s: | |
| 178984 | 1 | 0.015873016 | k: Bacteria; p: Firmicutes; | 170 |
| | | | c: Clostridia; o: Clostridiales; | |
| | | | f: Lachnospiraceae; | |
| | | | g: Coprococcus; s: | |
| 179116 | 1 | 0.015873016 | k: Bacteria; p: Firmicutes; | 171 |
| | | | c: Clostridia; o: Clostridiales; | |
| | | | f: Ruminococcaceae; | |
| | | | g: Oscillospira; s: | |
| 179137 | 1 | 0.015873016 | k: Bacteria; p: Firmicutes; | 172 |
| | | | c: Clostridia; o: Clostridiales; | |
| | | | f: Lachnospiraceae; | |
| | | | g: Coprococcus; s: | |
| 179267 | 25 | 0.396825397 | k: Bacteria; p: Firmicutes; | 173 |
| | | | c: Clostridia; o: Clostridiales; | |
| | | | f: Lachnospiraceae; | |
| | | | g: Coprococcus; s: | |
| 179291 | 2 | 0.031746032 | k: Bacteria; p: Firmicutles; | 174 |
| | | | c: Clostridia; o: Clostridiales; | |
| | | | f: Ruminococcaceae; | |
| | | | g: Faecalibacterium; | |
| | | | s: prausnitzii | |
| 179423 | 3 | 0.047619048 | k: Bacteria; p: Firmicutes; | 175 |
| | | | c: Cloatrdia; o: Clostridiale; | |
| | | | f: Clostridiaceae; g:; s: | |
| 179536 | 2 | 0.031746032 | k: Bacteria; p: Firmicutes; | 176 |
| | | | c: Clostridia; o: Clostridiales; | |
| | | | f: Clostridiaceae; g:; s: | |
| 179572 | 15 | 0.238095238 | k: Bacteria; p: Firmicutes; | 177 |
| | | | c: Clostridia; o: Clostridiales; | |
| | | | f: Ruminococcaceae; | |
| | | | g: Ruminococcus; s: | |
| 179573 | 7 | 0.111111111 | k: Bacteria; p: Firmicutes; | 178 |
| | | | c: Clostridia; o: Clostridiales; | |
| | | | f: Ruminococcaceae; | |
| | | | g: Ruminococcus; s: | |
| 179608 | 2 | 0.031746032 | k: Bacteria; p: Firmicutes; | 179 |
| | | | c: Clostridia; o: Clostridiales; | |
| | | | f: Christensenellaceae; g:; s: | |
| 179644 | 1 | 0.015873016 | k: Bacteria; p: Firmicutes; | 180 |
| | | | c: Clostridia; o: Clostridiales; | |
| | | | f: Ruminococcaceae; | |
| | | | g: Faecalibacterium; | |
| | | | s: prausnitzii | |
| 179647 | 29 | 0.46031746 | k: Bacteria; p: Firmicutes; | 181 |
| | | | c: Clostridia; o: Clostridiales; | |
| | | | f: Ruminococcaceae; | |
| | | | g: Ruminococcus; s: | |
| 179649 | 2 | 0.031746032 | k: Bacteria; p: Firmicutes; | 182 |
| | | | c: Clostridia; o: Clostridiales; | |
| | | | f: Ruminococcaceae; | |
| | | | g: Faecalibacterium; | |
| | | | s: prausnitzii | |
| 179677 | 33 | 0.523809524 | k: Bacteria; p: Firmicutes; | 183 |
| | | | c: Clostridia; o: Clostridiales; | |
| | | | f: Lachnospiraceae; | |
| | | | g: Coprococcus; s: | |
| 179834 | 1 | 0.015873016 | k: Bacteria; p: Firmicutes; | 184 |
| | | | c: Clostridia; o: Clostridiales; | |
| | | | f: Ruminococcaceae; | |
| | | | g: Ruminococcus; s: | |
| 179911 | 3 | 0.047619048 | k: Bacteria; p: Firmicutes; | 185 |
| | | | c: Clostridia; o: Clostridiales; | |
| | | | f: Lachnospiraceae; | |
| | | | g: Coprococcus; s: | |
| 179946 | 2 | 0.031746032 | k: Bacteria; p: Firmicutes; | 186 |
| | | | c: Clostridia; o: Clostridiales; | |
| | | | f: Lachnospiraceae; | |
| | | | g: Coprococcus; s: | |
| 180107 | 8 | 0.126984127 | k: Bacteria; p: Firmicutes; | 187 |
| | | | c: Clostridia; o: Clostridiales; | |
| | | | f: Ruminococcaceae; | |
| | | | g: Ruminococcus; s: | |
| 180130 | 15 | 0.238095238 | k: Bacteria; p: Firmicutes; | 188 |
| | | | c: Clostridia; o: Clostridiales; | |
| | | | f: Ruminococcaceae; | |
| | | | g: Ruminococcus; s: | |
| 180133 | 1 | 0.015873016 | k: Bacteria; p: Bacteriaoidetes; | 189 |
| | | | c: Bacteroidia; o: Bacteroidales; | |
| | | | [Barnesiellaceae]; g:; s: | |
| 180136 | 4 | 0.063492063 | k: Bacteria; p: Firmicutes; | 190 |
| | | | c: Clostridia; o: Clostridiales; | |
| | | | f: Ruminococcaceae; | |
| | | | g: Oscillospira; s: | |
| 180381 | 7 | 0.111111111 | k: Bacteria; p: Firmicutes; | 191 |
| | | | c: Clostridia; o: Clostridiales; | |
| | | | f: Ruminococcaceae; | |
| | | | g: Ruminococcus; s: | |
| 180402 | 47 | 0.746031746 | k: Bacteria; p: Firmicutes; | 192 |
| | | | c: Clostridia; o: Clostridiales; | |
| | | | f: [Mogibacteriaceae]; g:; s: | |
| 180468 | 11 | 0.174603175 | k: Bacteria; p: Firmicutes; | 193 |
| | | | c: Clostridia; o: Clostridiales; | |
| | | | f: Ruminococcaceae; | |
| | | | g: Oscillospira; s: | |
| 180509 | 34 | 0.53968254 | k: Bacteria; p: Firmicutes; | 194 |
| | | | c: Clostridia; o: Clostridiales; | |
| | | | f: Ruminococcaceae; | |
| | | | g: Ruminococcus; s: | |
| 180572 | 1 | 0.015873016 | k: Bacteria; p: Firmicutes; | 195 |
| | | | c: Clostridia; o: Clostridiales; | |
| | | | f: Ruminococcaceae; | |
| | | | g: Faecalibacterium; | |
| | | | s: prausnitzii | |
| 180659 | 22 | 0.349206349 | k: Bacteria; p: Firmicutes; | 196 |
| | | | c: Clostridia; o: Cloastridiales; | |
| | | | f: Ruminococcaceae; | |
| | | | g: Oscillospira; s: | |
| 180665 | 4 | 0.063492063 | k: Bacteria; p: Firmicutes; | 197 |
| | | | c: Clostridia; o: Clostridiales; | |
| | | | f: Ruminococcaceae; | |
| | | | g: Faecalibacterium; | |
| | | | s: prausnitzii | |
| 180738 | 13 | 0.206349206 | k: Bacteria; p: Firmicutes; | 198 |
| | | | c: Clostridia; o: Clostridiales; | |
| | | | f: Ruminococcaceae; | |
| | | | g: Ruminococcus; s: | |
| 180826 | 28 | 0.444444444 | k: Bacteria; p: Firmicutes; | 199 |
| | | | c: Clostridia; o: Clostridiales; | |
| | | | f: Ruminococcaceae; | |
| | | | g: Ruminococcus; s: | |
| 180857 | 1 | 0.015873016 | k: Bacteria; p: Firmicutes; | 200 |
| | | | c: Clostridia; o: Clostridiales; | |
| | | | f: Ruminococcaceae; | |
| | | | g: Ruminococcus; s: | |
| 180975 | 3 | 0.047619048 | k: Bacteria; p: Firmicutes; | 201 |
| | | | c: Clostridia; o: Clostridiales; | |
| | | | f: Ruminococcaceae; | |
| | | | g: Oscillospira; s: | |
| 181016 | 3 | 0.047619048 | k: Bacteria; p: Firmicutes; | 202 |
| | | | c: Clostridia; o: Clostridiales; | |
| | | | f: Ruminococcaceae; | |
| | | | g: Ruminococcus; s: | |
| 181025 | 40 | 0.634920635 | k: Bacteria; p: Firmicutes; | 203 |
| | | | c: Clostridia; o: Clostridiales; | |
| | | | f: Lachnospiraceae; | |
| | | | g: Coprococcus; s: | |
| 181047 | 23 | 0.365079365 | k: Bacteria; p: Firmicutes; | 204 |
| | | | c: Clostridia; o: Clostridiales; | |
| | | | f: Lachnospiraceae; | |
| | | | g: Coprococcus; s: | |
| 181056 | 1 | 0.015873016 | k: Bacteria; p: Firmicutes; | 205 |
| | | | c: Clostridia; o: Clostridiales; | |
| | | | f: Ruminococcaceae; | |
| | | | g: Faecalibacterium; | |
| | | | s: prausnitzii | |
| 181062 | 2 | 0.031746032 | k: Bacteria; p: Firmicutes; | 206 |
| | | | c: Clostridia; o: Clostridiales; | |
| | | | f: Ruminococcaceae; | |
| | | | g: Oscillospira; s: | |
| 181139 | 7 | 0.111111111 | k: Bacteria; p: Firmicutes; | 207 |
| | | | c: Clostridia; o: Clostridiales; | |
| | | | f: Ruminococcaceae; | |
| | | | g: Faecalibacterium; | |
| | | | s: prausnitzii | |
| 181160 | 31 | 0.492063492 | k: Bacteria; p: Firmicutes; | 208 |
| | | | c: Cloastridia; o: Clostridiales; | |
| | | | f: Clostridiaceae; g: Clostridium; s: | |
| 181176 | 25 | 0.396825397 | k: Bacteria; p: Firmicutes; | 209 |
| | | | c: Clostridia; o: Clostridiales; | |
| | | | f: Lachnospiraceae; | |
| | | | g: Coprococcus; s: | |
| 181269 | 14 | 0.222222222 | k: Bacteria; p: Firmicutes; | 210 |
| | | | c: Clostridia; o: Clostridiales; | |
| | | | f: Lachnospiraceae; | |
| | | | g: Coprococcus; s: | |
| 181422 | 1 | 0.015873016 | k: Bacteria; p: Firmicutes; | 211 |
| | | | c: Clostridia; o: Clostridiales; | |
| | | | f: Ruminococcaceae; | |
| | | | g: Faecalibacterium; | |
| | | | s: prausnitzii | |
| 181466 | 1 | 0.015873016 | k: Bacteria; p: Firmicutes; | 212 |
| | | | c: Clostridia; o: Clostridiales; | |
| | | | f: Clostridiaceae; g:; s: | |
| 181467 | 5 | 0.079365079 | k: Bacteria; p: Firmicutes; | 213 |
| | | | c: Clostridia; o: Clostridiales; | |
| | | | f: Clostridiaceae; g:; s: | |
| 181560 | 2 | 0.031746032 | k: Bacteria; p: Firmicutes; | 214 |
| | | | c: Clostridia; o: Clostridiales; | |
| | | | f: Lachnospiraceae; | |
| | | | g: Coprococcus; s: | |
| 181672 | 1 | 0.015873016 | k: Bacteria; p: Firmicutes; | 215 |
| | | | c: Clostridia; o: Clostridiales; | |
| | | | f: Lachnospiraceae; | |
| | | | g: Coprococcus; s: | |
| 181853 | 4 | 0.063492063 | k: Bacteria; p: Firmicutes; | 216 |
| | | | c: Clostridia; o: Clostridiales; | |
| | | | f: Lachnospiraceae; | |
| | | | g: Coprococcus; s: | |
| 181918 | 56 | 0.888888889 | k: Bacteria; p: Firmicutes; | 217 |
| | | | c: Clostridia; o: Clostridiales; | |
| | | | f: Lachnospiraceae; | |
| | | | g: Coprococcus; s: | |
| 181969 | 4 | 0.063492063 | k: Bacteria; p: Firmicutes; | 218 |
| | | | c: Clostridia; o: Clostridiales; | |
| | | | f: Ruminococcaceae; | |
| | | | g: Ruminococcus; s: | |
| 182054 | 58 | 0.920634921 | k: Bacteria; p: Firmicutes; | 219 |
| | | | c: Clostridia; o: Clostridiales; | |
| | | | f: Lachnospiraceae; | |
| | | | g: Coprococcus; s: | |
| 182149 | 10 | 0.158730159 | k: Bacteria; p: Firmicutes; | 220 |
| | | | c: Clostridia; o: Clostridiales; | |
| | | | f: Ruminococcaceae; | |
| | | | g: Faecalibacterium; | |
| | | | s: prausnitzii | |
| 182456 | 1 | 0.015873016 | k: Bacteria; p: Firmicutes; | 221 |
| | | | c: Clostridia; o: Clostridiales; | |
| | | | f: Lachnospiraceae; | |
| | | | g: Lachnospira; s: | |
| 182538 | 15 | 0.238095238 | k: Bacteria; p: Firmicutes; | 222 |
| | | | c: Clostridia; o: Clostridiales; | |
| | | | f: Lachnospiraceae; | |
| | | | g: Coprococcus; s: | |
| 182588 | 8 | 0.126984127 | k: Bacteria; p: Firmicutes; | 223 |
| | | | c: Clostridia; o: Clostridiale; | |
| | | | f: Ruminococcaceae; | |
| | | | g: Ruminococcus; s: | |
| 182600 | 1 | 0.015873016 | k: Bacteria; p: Firmicutes; | 224 |
| | | | c: Clostridia; o: Clostridiales; | |
| | | | f: Ruminococcaceae; | |
| | | | g: Oscillospira; s: | |
| 182643 | 2 | 0.031746032 | k: Bacteria; p: Firmicutes; | 225 |
| | | | c: Clostridia; o: Clostridiales; | |
| | | | f: Clostridiaceae; g:; s: | |
| 182799 | 56 | 0.888888889 | k: Bacteria; p: Firmicutes; | 226 |
| | | | c: Clostridia; o: Clostridiales; | |
| | | | f: Lachnospiraceae; | |
| | | | g: Lachnospira; s: | |
| 182891 | 1 | 0.015873016 | k: Bacteria; p: Firmicutes; | 227 |
| | | | c: Clostridia; o: Clostridiales; | |
| | | | f: Clostridiaceae; g:; s: | |
| 182986 | 10 | 0.158730159 | k: Bacteria; p: Firmicutes; | 228 |
| | | | c: Clostridia; o: Clostridiales; | |
| | | | f: Ruminococcaceae; | |
| | | | g: Faecalibacterium; | |
| | | | s: prausnitzii | |
| 183049 | 41 | 0.650793651 | k: Bacteria; p: Firmicutes; | 229 |
| | | | c: Clostridia; o: Clostridiales; | |
| | | | f: Ruminococcaceae; | |
| | | | g: Faecalibacterium; | |
| | | | s: prausnitzii | |
| 183089 | 22 | 0.349206349 | k: Bacteria; p: Firmicutes; | 230 |
| | | | c: Clostridia; o: Clostridiales; | |
| | | | f: Clostridiaceae; g:; s: | |
| 183104 | 6 | 0.095238095 | k: Bacteria; p: Actinobacter; | 231 |
| | | | c: Coriobacteriia; | |
| | | | o: Coriobacteriales; | |
| | | | f: Coriobacteriaceae; g:; s: | |
| 183157 | 5 | 0.079365079 | k: Bacteria; p: Firmicutes; | 232 |
| | | | c: Clostridia; o: Clostridiales; | |
| | | | f: Ruminococcaceae; | |
| | | | g: Faecalibacterium; | |
| | | | s: prausnitzii | |
| 183162 | 7 | 0.111111111 | k: Bacteria; p: Firmicutes; | 233 |
| | | | c: Clostridia; o: Clostridiales; | |
| | | | f: Ruminococcaceae; | |
| | | | g: Oscillospira; s: | |
| 183245 | 3 | 0.047619048 | k: Bacteria; p: Firmicutes; | 234 |
| | | | c: Clostridia; o: Clostridiales; | |
| | | | f: Ruminococcaceae; | |
| | | | g: Ruminococcus; s: | |
| 183395 | 2 | 0.031746032 | k: Bacteria; p: Bacteroidetes; | 235 |
| | | | c: Bacteroidia; o: Bacteroidales; | |
| | | | f: [Barnesiellaceae]; g:; s: | |
| 183680 | 2 | 0.031746032 | k: Bacteria; p: Firmicutes; | 236 |
| | | | c: Clostridia; o: Clostridiales; | |
| | | | f: Lachnospiraceae; | |
| | | | g: Coproccus; s: | |
| 183748 | 14 | 0.222222222 | k: Bacteria; p: Firmicutes; | 237 |
| | | | c: Clostridia; o: Clostridiales; | |
| | | | f: Christensenellaceae; g:; s: | |
| 183780 | 4 | 0.063492063 | k: Bacteria; p: Firmicutes; | 238 |
| | | | c: Clostridia; o: Clostridiales; | |
| | | | f: Ruminococcaceae; | |
| | | | g: Faecalibacterium; | |
| | | | s: prausnitzii | |
| 184000 | 47 | 0.746031746 | k: Bacteria; p: Firmicutes; | 239 |
| | | | c: Clostridia; o: Clostridiales; | |
| | | | f: Ruminnococcaceae; | |
| | | | g: Faecalibacterium; | |
| | | | s: prausnitzii | |
| 184009 | 19 | 0.301587302 | k: Bacteria; p: Firmicutes; | 240 |
| | | | c: Clostridia; o: Clostridiales; | |
| | | | f: Clostridiaceae; g:; s: | |
| 184013 | 8 | 0.126984127 | k: Bacteria; p: Firmicutes; | 241 |
| | | | c: Clostridia; o: Clostridiales; | |
| | | | f: Lachnospiraceae; | |
| | | | g: Coprococcus; s: | |
| 184025 | 4 | 0.063492063 | k: Bacteria; p: Firmicutes; | 242 |
| | | | c: Clostridia; o: Clostridiales; | |
| | | | f: Lachnospiraceae; | |
| | | | g: Coprococcus; s: | 243 |
| 184511 | 10 | 0.158730159 | k: Bacteria; p: Firmicutes; | |
| | | | c: Clostridia; o: Clostridiales; | |
| | | | f: Ruminococcaceae; | |
| | | | g: Faecalibacterium; | |
| | | | s: prausnitzii | |
| 184525 | 8 | 0.126984127 | k: Bacteria; p: Firmicutes; | 244 |
| | | | c: Clostridia; o: Clostridiales; | |
| | | | f: Lachnospiraceae; | |
| | | | g: Coprococcus; s: | |
| 184678 | 39 | 0.619047619 | k: Bacteria; p: Firmicutes; | 245 |
| | | | c: Clostridia; o: Clostridiales; | |
| | | | f: Ruminococcaceae; | |
| | | | g: Faecalibacterium; | |
| | | | s: prausnitzii | |
| 184725 | 4 | 0.063492063 | k: Bacteria; p: Bacteroidetes; | 246 |
| | | | c: Bacteroidia; o: Bacteroidales; | |
| | | | f: Rikenellaceae; g:; s: | |
| 184770 | 56 | 0.888888889 | k: Bacteria; p: Firmicutes; | 247 |
| | | | c: Clostridia; o: Clostridiales; | |
| | | | f: Lachnospiraceae; | |
| | | | g: Coprococcus; s: | |
| 184821 | 1 | 0.015873016 | k: Bacteria; p: Firmicutes; | 248 |
| | | | c: Clostridia; o: Clostridiales; | |
| | | | f: Lachnospiraceae; | |
| | | | g: Coprococcus; s: | |
| 184845 | 4 | 0.063492063 | k: Bacteria; p: Firmicutes; | 249 |
| | | | c: Clostridia; o: Clostridiales; | |
| | | | f: Ruminococcaceae; | |
| | | | g: Ruminococcus; s: | |
| 184876 | 7 | 0.111111111 | k: Bacteria; p: Firmicutes; | 250 |
| | | | c: Clostridia; o: Clostridiales; | |
| | | | f: Lachnospiraceae; | |
| | | | g: Lachnospira; s: | |
| 184897 | 47 | 0.746031746 | k: Bacteria; p: Firmicutes; | 251 |
| | | | c: Clostridia; o: Clostridiales; | |
| | | | f: Ruminococcaceae; | |
| | | | g: Faecalibacterium; | |
| | | | s: prausnitzii | |
| 184905 | 1 | 0.015873016 | k: Bacteria; p: Firmicutes; | 252 |
| | | | c: Clostridia; o: Clostridiales; | |
| | | | f: Lachnospiraceae; | |
| | | | g: Coprococcus; s: | |
| 184940 | 5 | 0.079365079 | k: Bacteria; p: Firmicutes; | 253 |
| | | | c: Clostridia; o: Clostridiales; | |
| | | | f: Christensenellaceae; g:; s: | |
| 184996 | 1 | 0.015873016 | k: Bacteria; p: Firmicutes; | 254 |
| | | | c: Clostridia; o: Clostridiales; | |
| | | | f: Ruminococcaceae; | |
| | | | g: Faecalibacterium; | |
| | | | s: prausnitzii | |
| 185042 | 2 | 0.031746032 | k: Bacteria; p: Firmicutes; | 255 |
| | | | c: Clostridia; o: Clostridiales; | |
| | | | f: Ruminococcaceae; | |
| | | | g: Oscillospira; s: | |
| 185159 | 4 | 0.063492063 | k: Bacteria; p: Firmicutes; | 256 |
| | | | c: Clostridia; o: Clostridiales; | |
| | | | f: Clostridiaceae; g:; s: | |
| 185175 | 3 | 0.047619048 | k: Bacteria; p: Firmicutes; | 257 |
| | | | c: Clostridia; o: Clostridiales; | |
| | | | f: Ruminococcaceae; | |
| | | | g: Ruminococcus; s: | |
| 185222 | 1 | 0.015873016 | k: Bacteria; p: Firmicutes; | 258 |
| | | | c: Clostridia; o: Clostridiales; | |
| | | | f: Clostridiaceae; g:; s: | |
| 185235 | 14 | 0.222222222 | k: Bacteria; p: Firmicutes; | 259 |
| | | | c: Clostridia; o: Clostridiales; | |
| | | | f: Lachnospiraceae; | |
| | | | g: Coprococcus; s: | |
| 185324 | 5 | 0.079365079 | k: Bacteria; p: Firmicutes; | 260 |
| | | | c: Clostridia; o: Clostridiales; | |
| | | | f: Ruminococcaceae; | |
| | | | g: Faecalibacterium; | |
| | | | s: prausnitzii | |
| 185392 | 1 | 0.015873016 | k: Bacteria; p: Firmicutes; | 261 |
| | | | c: Clostridia; o: Clostridiales; | |
| | | | f: Ruminococcaceae; | |
| | | | g: Ruminococcus; s: | |
| 185667 | 8 | 0.126984127 | k: Bacteria; p: Firmicutes; | 262 |
| | | | c: Clostridia; o: Clostridiales; | |
| | | | f: Lachnospiraceae; | |
| | | | g: Coprococcus; s: | |
| 185715 | 18 | 0.285714286 | k: Bacteria; p: Firmicutes; | 263 |
| | | | c: Clostridia; o: Clostridiales; | |
| | | | f: Lachnospiraceae; | |
| | | | g: Coprococcus; s: | |
| 185769 | 3 | 0.047619048 | k: Bacteria; p: Firmicutes; | 264 |
| | | | c: Clostridia; o: Clostridiales; | |
| | | | f: Ruminococcaceae; | |
| | | | g: Faecalibacterium; | |
| | | | s: prausnitzii | |
| 185821 | 12 | 0.19047619 | k: Bacteria; p: Firmicutes; | 265 |
| | | | c: Clostridia; o: Clostridiales; | |
| | | | f: Lachnospiraceae; | |
| | | | g: Coprococcus; | |
| | | | s: eutactus | |
| 185861 | 23 | 0.365079365 | k: Bacteria; p: Firmicutes; | 266 |
| | | | c: Clostridia; o: Clostridiales; | |
| | | | f: Lachnospiraceae; | |
| | | | g: Coprococcus; s: | |
| 186090 | 39 | 0.619047619 | k: Bacteria; p: Firmicutes; | 267 |
| | | | c: Clostridia; o: Clostridiales; | |
| | | | f: Ruminococcaceae; | |
| | | | g: Ruminococcus; s: | |
| 186092 | 17 | 0.26984127 | k: Bacteria; p: Firmicutes; | 268 |
| | | | c: Clostridia; o: Clostridiales; | |
| | | | f: Clostridiaceae; g:; s: | |
| 186133 | 15 | 0.238095238 | k: Bacteria; p: Firmicutes; | 269 |
| | | | c: Clostridia; o: Clostridiales; | |
| | | | f: Ruminococcaceae; | |
| | | | g: Ruminococcus; s: | |
| 186289 | 2 | 0.031746032 | k: Bacteria; p: Firmicutes; | 270 |
| | | | c: Clostridia; o: Clostridiales; | |
| | | | f: Clostridiaceae; g: Clostridium; s: | |
| 186319 | 3 | 0.047619048 | k: Bacteria; p: Firmicutes; | 271 |
| | | | c: Clostridia; o: Clostridiales; | |
| | | | f: Lachnospiraceae; | |
| | | | g: Coprococcus; s: | |
| 186328 | 2 | 0.031746032 | k: Bacteria; p: Firmicutes; | 272 |
| | | | c: Clostridia; o: Clostridiales; | |
| | | | f: Ruminococcaceae; | |
| | | | g: Faecalibacterium; | |
| | | | s: prausnitzii | |
| 186350 | 1 | 0.015873016 | k: Bacteria; p: Firmicutes; | 273 |
| | | | c: Clostridia; o: Clostridiales; | |
| | | | f: Ruminococcaceae; | |
| | | | g: Ruminococcus; s: | |
| 186389 | 1 | 0.015873016 | k: Bacterial; p: Firmicutes; | 274 |
| | | | c: Clostridia; o: Clostridiales; | |
| | | | f: Clostridiaceae; g: Clostridium; s: | |
| 186392 | 29 | 0.46031746 | k: Bacteria; p: Firmicutes; | 275 |
| | | | c: Clostridia; o: Clostridiales; | |
| | | | f: Ruminococcaceae; | |
| | | | g: Faecalibacterium; | |
| | | | s: prausnitzii | |
| 186554 | 1 | 0.015873016 | k: Bacteria; p: Firmicutes; | 276 |
| | | | c: Clostridia; o: Clostridiales; | |
| | | | f: Ruminococcaceae; | |
| | | | g: Ruminococccus; s: | |
| 186563 | 3 | 0.047619048 | k: Bacteria; p: Firmicutes; | 277 |
| | | | c: Clostridia; o: Clostridiales; | |
| | | | f: Ruminococcaceae; | |
| | | | g: Ruminococcus; | |
| | | | s: callidus | |
| 186592 | 1 | 0.015873016 | k: Bacteria; p: Firmicutes; | 278 |
| | | | c: Clostridia; o: Clostridiales; | |
| | | | f: Lachnospiraceae; | |
| | | | g: Coprococcus; s: | |
| 186614 | 60 | 0.952380952 | k: Bacteria; p: Firmicutes; | 279 |
| | | | c: Clostridia; o: Clostridiales; | |
| | | | f: Lachnospiraceae; | |
| | | | g: Coprococcus; s: | |
| 186703 | 15 | 0.238095238 | k: Bacteria; p: Actinobacteria; | 280 |
| | | | c: Coriobacteriia; | |
| | | | o: Coriobacteriales; | |
| | | | f: Coriobacterisceae; | |
| | | | g: Collinsella; | |
| | | | s: aerofaciens | |
| 183731 | 7 | 0.111111111 | k: Bacteria; p: Firmicutes; | 281 |
| | | | c: Clostridia; o: Clostridiales; | |
| | | | f: Lachnospiraceae; | |
| | | | g: Coprococcus; s: | |
| 186780 | 6 | 0.095238095 | k: Bacteria; p: Firmicutes; | 282 |
| | | | c: Clostridia; o: Clostridiales; | |
| | | | f: Lachnospiraceae; | |
| | | | g: Coprococcus; s: | |
| 186921 | 2 | 0.031746032 | k: Bacteria; p: Firmicutes; | 283 |
| | | | c: Clostridia; o: Clostridiales; | |
| | | | f: Lachnospiraceae; | |
| | | | g: Coprococcus; s: | |
| 186969 | 15 | 0.238095238 | k: Bacteria; p: Firmicutes; | 284 |
| | | | c: Clostridia; o: Clostridiales; | |
| | | | f: Lachnospiraceae; | |
| | | | g: Coprococcus; s: | |
| 186981 | 22 | 0.349206349 | k: Bacteria; p: Bacteroidetes; | 285 |
| | | | c: Bacteroidia; o: Bacteroidales; | |
| | | | f: [Barnesiellaceae]; g:; s: | |
| 187056 | 38 | 0.603174603 | k: Bacteria; p: Firmicutes; | 286 |
| | | | c: Clostridia; o: Clostridiales; | |
| | | | f: Ruminococcaceae; | |
| | | | g: Ruminococcus; s: | |
| 187181 | 9 | 0.142857143 | k: Bacteria; p: Firmicutes; | 287 |
| | | | c: Clostridia; o: Clostridiales; | |
| | | | f: Ruminococcaceae; | |
| | | | g: Ruminococcus; s: | |
| 187267 | 20 | 0.317460317 | k: Bacteria; p: Firmicutes; | 288 |
| | | | c: Clostridia; o: Clostridiales; | |
| | | | f: Ruminococcaceae; | |
| | | | g: Oscillospira; s: | |
| 187321 | 7 | 0.111111111 | k: Bacteria; p: Firmicutes; | 289 |
| | | | c: Clostridia; o: Clostridiales; | |
| | | | f: Ruminococcaceae; | |
| | | | g: Ruminococcus; s: | |
| 187356 | 60 | 0.952380952 | k: Bacteria; p: Firmicutes; | 290 |
| | | | c: Clostridia; o: Clostridiales; | |
| | | | f: Lachnospiraceae; | |
| | | | g: Coprococcus; s: | |
| 187409 | 1 | 0.015873016 | k: Bacteria; p: Firmicutes; | 291 |
| | | | c: Clostridia; o: Clostridiales; | |
| | | | f: Ruminococcaceae; | |
| | | | g: Oscillospira; s: | |
| 187466 | 5 | 0.079365079 | k: Bacteria; p: Firmicutes; | 292 |
| | | | c: Clostridia; o: Clostridiales; | |
| | | | f: Clostridiaceae; g:; s: | |
| 187470 | 2 | 0.031746032 | k: Bacteria; p: Firmicutes; | 293 |
| | | | c: Clostridia; o: Clostridiales; | |
| | | | f: Lachnospiraceae; | |
| | | | g: Coprococcus; s: | |
| 187490 | 49 | 0.777777778 | k: Bacteria; p: Actinobacteria; | 294 |
| | | | c: Coriobacteriia; | |
| | | | o: Coriobacteriales; | |
| | | | f: Coriobacteriaceae; | |
| | | | g: Collinsella; s: | |
| 187569 | 61 | 0.968253968 | k: Bacteria; p: Firmicutes; | 295 |
| | | | c: Clostridia; o: Clostridiales; | |
| | | | f: Lachnospiraceae; | |
| | | | g: Coprococcus; s: | |
| 187668 | 50 | 0.793650794 | k: Bactteria; p: Firmicutes; | 286 |
| | | | c: Clostridia; o: Clostridiales; | |
| | | | f: Clostridiaceae; | |
| | | | g: Clostridium; s: | |
| 187709 | 1 | 0.015873016 | k: Bacteria; p: Firmicutes; | 297 |
| | | | c: Clostridia; o: Clostridiales; | |
| | | | f: Ruminococcaceae; | |
| | | | g: Oscillospira; s: | |
| 187868 | 34 | 0.53968254 | k: Bacteria; p: Firmicutes; | 298 |
| | | | c: Clostridia; o: Clostridiales; | |
| | | | f: Lachnospiraceae; | |
| | | | g: Coprococcus; s: | |
| 187956 | 26 | 0.412698413 | k: Bacteria; p: Firmicutes; | 299 |
| | | | c: Clostridia; o: Clostridiales; | |
| | | | f: Clostridiaceae; g:; s: | |
| 188001 | 5 | 0.079365079 | k: Bacteria; p: Firmicutes; | 300 |
| | | | c: Clostridia; o: Clostridiales; | |
| | | | f: Ruminococcaceae; | |
| | | | g: Ruminococcus; s: | |
| 188047 | 62 | 0.984126984 | k: Bacteria; p: Firmicutes; | 301 |
| | | | c: Clostridia; o: Clostridiales; | |
| | | | f: Lachnospiraceae; | |
| | | | g: Coprococcus; s: | |
| 188079 | 36 | 0.571428571 | k: Bacteria; p: Firmicutes; | 302 |
| | | | c: Clostridia; o: Clostridiales; | |
| | | | f: Lachnospiraceae; | |
| | | | g: Coprococcus; s: | |
| 188126 | 1 | 0.015873016 | k: Bacteria; p: Firmicutes; | 303 |
| | | | c: Clostridia; o: Clostridiales; | |
| | | | f: Ruminococcaceae; | |
| | | | g: Faecalibacterium; | |
| | | | s: prausnitzii | |
| 188276 | 7 | 0.111111111 | k: Bacteria; p: Firmicutes; | 304 |
| | | | c: Clostridia; o: Clostridiales; | |
| | | | f: Ruminococcaceae; | |
| | | | g: Ruminococcus; s: | |
| 188316 | 8 | 0.126984127 | k: Bacteria; p: Firmicutes; | 305 |
| | | | c: Clostridia; o: Clostridiales; | |
| | | | f: Ruminococcaceae; | |
| | | | g: Oscillospira; s: | |
| 188329 | 3 | 0.047619048 | k: Bacteria; p: Firmicutes; | 306 |
| | | | c: Clostridia; o: Clostridiales; | |
| | | | f: Ruminococcaceae; | |
| | | | g: Faecalibacterium; | |
| | | | s: prausnitzii | |
| 188484 | 1 | 0.015873016 | k: Bacteria; p: Firmicutes; | 307 |
| | | | c: Clostridia; o: Clostridiales; | |
| | | | f: Ruminococcaceae; | |
| | | | g: Ruminococcus; s: | |
| 188625 | 19 | 0.301587302 | k: Bacteria; p: Firmicutes; | 308 |
| | | | c: Clostridia; o: Clostridiales; | |
| | | | f: Lachnospiraceae; | |
| | | | g: Coprococcus; s: | |
| 188789 | 1 | 0.015873016 | k: Bacteria; p: Firmicutes; | 309 |
| | | | c: Clostridia; o: Clostridiales; | |
| | | | f: Christensenellaceae; g:; s: | |
| 188881 | 9 | 0.142857143 | k: Bacteria; p: Firmicutes; | 310 |
| | | | c: Clostridia; o: Clostridiales; | |
| | | | f: Christensenellaceae; g:; s: | |
| 188918 | 30 | 0.476190476 | k: Bacteria; p: Firmicutes; | 311 |
| | | | c: Clostridia; o: Clostridiales; | |
| | | | f: Lachnospiraceae; | |
| | | | g: Coprococcus; s: | |
| 188966 | 39 | 0.619047619 | k: Bacteria; p: Actinobacteria; | 312 |
| | | | c: Coriobacteriia; | |
| | | | o: Coriobacteriales; | |
| | | | f: Coriobacteriaceae; g:; s: | |
| 188974 | 6 | 0.095238095 | k: Bacteria; p: Firmicutes; | 313 |
| | | | c: Clostridia; o: Clostridiales; | |
| | | | f: Lachnospiraceae; | |
| | | | g: Coprococcus; s: | |
| 189011 | 6 | 0.095238095 | k: Bacteria; p: Firmicutes; | 314 |
| | | | c: Clostridia; o: Clostridiales; | |
| | | | f: Ruminococcaceae; | |
| | | | g: Ruminococcus; s: | |
| 189035 | 22 | 0.349206349 | k: Bacteria; p: Firmicutes; | 315 |
| | | | c: Clostridia; o: Clostridiales; | |
| | | | f: Ruminococcaeae; | |
| | | | g: Ruminococcus; s: | |
| 189064 | 46 | 0.73015873 | k: Bacteria; p: Firmicutes; | 316 |
| | | | c: Clostridia; o: Clostridiales; | |
| | | | f: Ruminococcaceae; | |
| | | | g: Faecalibacterium; | |
| | | | s: prausnitzii | |
| 189092 | 4 | 0.063492063 | k: Bacteria; p: Firmicutes; | 317 |
| | | | c: Clostridia; o: Clostridiales; | |
| | | | f: Ruminococcaceae; | |
| | | | g: Faecalibacterium; | |
| | | | s: prausnitzii | |
| 189110 | 14 | 0.222222222 | k: Bacteria; p: Firmicutes; | 318 |
| | | | c: Clostridia; o: Clostridiales; | |
| | | | f: Ruminococcaceae; | |
| | | | g: Ruminococcus; s: | |
| 189116 | 2 | 0.031746032 | k: Bacteria; p: Firmicutes; | 319 |
| | | | c: Clostridia; o: Clostridiales; | |
| | | | f: Ruminococcaceae; | |
| | | | g: Ruminococcus; s: | |
| 189147 | 6 | 0.095238095 | k: Bacteria; p: Firmicutes; | 320 |
| | | | c: Clostridia; o: Clostridiales; | |
| | | | f: Ruminococcaceae; | |
| | | | g: Ruminococcus; s: | |
| 189150 | 31 | 0.492063492 | k: Bacteria; p: Firmicutes; | 321 |
| | | | c: Clostridia; o: Clostridiales; | |
| | | | f: Ruminococcaceae; | |
| | | | g: Ruminococcus; s: | |
| 189152 | 1 | 0.015873016 | k: Bacteria; p: Firmicutes; | 322 |
| | | | c: Clostridia; o: Clostridiales; | |
| | | | f: Lachnospiraceae; | |
| | | | g: Coprococcus; s: | |
| 189282 | 1 | 0.015873016 | k: Bacteria; p: Firmicutes; | 323 |
| | | | c: Clostridia; o: Clostridiales; | |
| | | | f: Ruminococcaceae; | |
| | | | g: Faecalibacterium; | |
| | | | s: prausnitzii | |
| 189294 | 43 | 0.682539683 | k: Bacteria; p: Actinobacteria; | 324 |
| | | | c: Coriobacteriia; | |
| | | | o: Coriobacteriales; | |
| | | | f: Coriobacteriaceae; | |
| | | | g: Collinsella; | |
| | | | s: aerofaciens | |
| 189407 | 6 | 0.095238095 | k: Bacteria; p: Firmicutes; | 325 |
| | | | c: Clostridia; o: Clostridiales; | |
| | | | f: [Mogibacteriaceae]; g:; s: | |
| 189459 | 12 | 0.19047619 | k: Bacteria; p: Firmicutes; | 326 |
| | | | c: Clostridia; o: Clostridiales; | |
| | | | f: Lachnospiraceae; | |
| | | | g: Coprococcus; s: | |
| 189478 | 1 | 0.015873016 | k: Bacteria; p: Firmicutes; | 327 |
| | | | c: Clostridia; o: Cloatridiales; | |
| | | | f: Lachnospiraceae; | |
| | | | g: Coprococcus; s: | |
| 189503 | 2 | 0.031746032 | k: Bacteria; p: Firmicutes; | 328 |
| | | | c: Clostridia; o: Clostridiales; | |
| | | | f: Clostridiaceae; g:; s: | |
| 189626 | 1 | 0.015873016 | k: Bacteria; p: Firmicutes; | 329 |
| | | | c: Clostridia; o: Clostridiales; | |
| | | | f: Lachnospiraceae; | |
| | | | g: Coprococcus; s: | |
| 189677 | 37 | 0.587301587 | k: Bacteria; p: Firmicutes; | 330 |
| | | | c: Clostridia; o: Clostridiales; | |
| | | | f: Lachnospiraceae; | |
| | | | g: Coprococcus; s: | |
| 189708 | 2 | 0.031746032 | k: Bacteria; p: Firmicutes; | 331 |
| | | | c: Clostridia; o: Clostridiales; | |
| | | | f: Ruminococcaceae; | |
| | | | g: Faecalibacterium; | |
| | | | s: prausnitzii | |
| 189820 | 5 | 0.079365079 | k: Bacteria; p: Firmicutes; | 332 |
| | | | c: Clostridia; o: Clostridiales; | |
| | | | f: Ruminococcaceae; | |
| | | | g: Ruminococcus; s: | |
| 189936 | 4 | 0.063492063 | k: Bacteria; p: Firmicutes; | 333 |
| | | | c: Clostridia; o: Clostridiales; | |
| | | | f: [Mogibacteriaceae]; g:; s: | |
| 189937 | 1 | 0.015873016 | k: Bacteria; p: Firmicutes; | 334 |
| | | | c: Clostridia; o: Clostridiales; | |
| | | | f: Ruminococcaceae; | |
| | | | g: Faecalibacterium; | |
| | | | s: prausnitzii | |
| 189960 | 6 | 0.095238095 | k: Bacteria; p: Bacteroidetes; | 335 |
| | | | c: Bacteroidia; o: Bacteroidales; | |
| | | | f: [Barnesiellaceae]; g:; s: | |
| 189975 | 3 | 0.047619048 | k: Bacteria; p: Firmicutes; | 336 |
| | | | c: Clostridia; o: Clostridiales; | |
| | | | f: Christensenellaceae; g:; s: | |
| 189997 | 49 | 0.777777778 | k: Bacteria; p: Actinobacteria; | 337 |
| | | | c: Coriobacteriia; | |
| | | | o: Coriobacteriales; | |
| | | | f: Coriobacteriaceae; | |
| | | | g: Collinsella; s: | |
| 190299 | 2 | 0.031746032 | k: Bacteria; p; Firmicutes; | 338 |
| | | | c: Clostridia; o: Clostridiales; | |
| | | | f: Lachnospiraceae; | |
| | | | g: Lachnospira; s: | |
| 190534 | 2 | 0.031746032 | k: Bacteria; p: Firmicutes; | 339 |
| | | | c: Clostridia; o: Clostridiales; | |
| | | | f: Lachnospiraceae; | |
| | | | g: Coprococcus; s: | |
| 190599 | 13 | 0.206349206 | k: Bacteria; p: Firmicutes; | 340 |
| | | | c: Clostridia; o: Clostridiales; | |
| | | | f: Lachnospiraceae; | |
| | | | g: Coprococcus; s: | |
| 190679 | 12 | 0.19047619 | k: Bacteria; p: Firmicutes; | 341 |
| | | | c: Clostridia; o: Clostridiales; | |
| | | | f: Lachnosoiraceae; | |
| | | | g: Coprococcus; | |
| | | | s: eutactus | |
| 190766 | 1 | 0.015873016 | k: Bacteria; p: Firmicutes; | 342 |
| | | | c: Clostridia; o: Clostridiales; | |
| | | | f: Clostridiaceae; g: Clostridium; s: | |
| 190844 | 1 | 0.015873016 | k: Bacteria; p: Bacteroidetes; | 343 |
| | | | c: Bacteroidia; o: Bacteroidales; | |
| | | | f: Rikenellaceae; g:; s: | |
| 190931 | 4 | 0.063492063 | k: Bacteria; p: Firmicutes; | 344 |
| | | | c: Clostridia; o: Clostridiales; | |
| | | | f: Ruminococcaceae; | |
| | | | g: Ruminococcus; s: | |
| 190975 | 1 | 0.015873016 | k: Bacteria; p: Bcateroidetes; | 345 |
| | | | c: Bacteroidia; o: Bacteroidales; | |
| | | | f: [Barnesiellaceae]; g:; s: | |
| 191238 | 7 | 0.111111111 | k: Bacteria; p: Firmicutes; | 346 |
| | | | c: Clostridia; o: Clostridiales; | |
| | | | f: Lachnospiraceae; | |
| | | | g: Coprococcus; s: | |
| 191270 | 46 | 0.73015873 | k: Bacteria; p: Firmicutes; | 347 |
| | | | c: Clostridia; o: Clostridiales; | |
| | | | f: Ruminococcaceae; | |
| | | | g: Oscillospira; s: | |
| 191389 | 7 | 0.111111111 | k: Bacteria; p: Firmicutes; | 348 |
| | | | c: Clostridia; o: Clostridiales; | |
| | | | f: [Mogibacteriaceae]; g:; s: | |
| 191547 | 1 | 0.015873016 | k: Bacteria; p: Firmicutes; | 349 |
| | | | c: Clostridia; o: Clostridiales; | |
| | | | f: Ruminococcaceae; | |
| | | | g: Faecalibacterium; | |
| | | | s; prausnitzii | |
| 191595 | 17 | 0.26984127 | k: Bacteria; p: Actinobacteria; | 350 |
| | | | c: Coriobacteriia; | |
| | | | o: Coriobacteriales; | |
| | | | f: Coriobacteriaceae; | |
| | | | g: Collinsella; | |
| | | | s: aerofaciens | |
| 191615 | 5 | 0.079365079 | k: Bacteria; p: Firmicutes; | 351 |
| | | | c: Clostridia; o: Clostridiales; | |
| | | | f: Ruminococcaceae; | |
| | | | g: Ruminococcus; s: | |
| 191660 | 3 | 0.047619048 | k: Bacteria; p: Firmicutes; | 352 |
| | | | c: Clostridia; o: Clostridiales; | |
| | | | f: Ruminococcaceae; | |
| | | | g: Faecalibacterium; | |
| | | | s: prausnitzii | |
| 191803 | 5 | 0.079365079 | k: Bacteria; p: Firmicutes; | 353 |
| | | | c: Clostridia; o: Clostridiales; | |
| | | | f: Clostridiaceae; g:; s: | |
| 191874 | 8 | 0.126984127 | k: Bacteria; p: Firmicutes; | 354 |
| | | | c: Clostridia; o: Clostridiales; | |
| | | | f: Ruminococcaceae; | |
| | | | g: Ruminococcus; s: | |
| 190248 | 4 | 0.063492063 | k: Bacteria; p: Firmicutes; | 355 |
| | | | c: Clostridia; o: Clostridiales; | |
| | | | f: Ruminococcaceae; | |
| | | | g: Faecalibacterium; | |
| | | | s: prausnitzii | |
| 192058 | 46 | 0.73015873 | k: Bacteria; p: Firmicutes; | 356 |
| | | | c: Clostridia; o: Clostridiales; | |
| | | | f: Ruminococcaceae; | |
| | | | g: Oscillospira; s: | |
| 192066 | 39 | 0.619047619 | k: Bacteria; p: Firmicutes; | 357 |
| | | | c: Clostridia; o: Clostridiales; | |
| | | | f: Lachnospiraceae; | |
| | | | g: Coprococcus; s: | |
| 192079 | 1 | 0.015873016 | k: Bacteria; p: Firmicutes; | 358 |
| | | | c: Clostridia; o: Clostridiales; | |
| | | | f: Ruminococcaceae; | |
| | | | g: Ruminococcus; s: | |
| 192218 | 12 | 0.19047619 | k: Bacteria; p: Firmicutes; | 359 |
| | | | c: Clostridia; o: Clostridiales; | |
| | | | f: Lachnospiraceae; | |
| | | | g: Coprococcus; s: | |
| 192231 | 1 | 0.015873016 | k: Bacteria; p: Firmicutes; | 360 |
| | | | c: Clostridia; o: Clostridiales; | |
| | | | f: Ruminococcaceae; | |
| | | | g: Faecalibacterium; | |
| | | | s: prausnitzii | |
| 192263 | 19 | 0.301587302 | k: Bacteria; p: Firmicutes; | 361 |
| | | | c: Clostridia; o: Clostridiales; | |
| | | | f: Lachnospiraceae; | |
| | | | g: Coprococcus; s: | |
| 192385 | 2 | 0.031746032 | k: Bacteria; p: Firmicutes; | 362 |
| | | | c: Clostridia; o: Clostridiales; | |
| | | | f: Christensenellaceae; g:; s: | |
| 192515 | 4 | 0.063492063 | k: Bacteria; p: Firmicutes; | 363 |
| | | | c: Clostridia; o: Clostridiales; | |
| | | | f: Ruminococcaceae; | |
| | | | g: Ruminococcus; | |
| | | | s: bromii | |
| 192566 | 25 | 0.396825397 | k: Bacteria; p: Firmicutes; | 364 |
| | | | c: Clostridia; o: Clostridiales; | |
| | | | f: Ruminococcaceae; | |
| | | | g: Faecalibacterium; | |
| | | | s: prausnitzii | |
| 192654 | 3 | 0.047619048 | k: Bacteria; p: Firmicutes; | 365 |
| | | | c: Clostridia; o: Clostridiales; | |
| | | | f: Ruminococcaceae; | |
| | | | g: Ruminococcus; s: | |
| 192893 | 2 | 0.031746032 | k: Bacteria; p: Firmicutes; | 366 |
| | | | c: Clostridia; o: Clostridiales; | |
| | | | f: Clostridiaceae; g: Clostridium; s: | |
| 192958 | 19 | 0.301587302 | k: Bacteria; p: Firmicutes; | 367 |
| | | | c: Clostridia; o: Clostridiales; | |
| | | | f: Lachnospiraceae; | |
| | | | g: Coprococcus; s: | |
| 193160 | 25 | 0.396825397 | k: Bacteria; p: Firmicutes; | 368 |
| | | | c: Clostridia; o: Clostridiales; | |
| | | | f: Lachnospiraceae; g: Coprococcus; | |
| | | | s: eutactus | |
| 193191 | 7 | 0.111111111 | k: Bacteria; p: Firmicutes; | 369 |
| | | | c: Clostridia; o: Clostridiales; | |
| | | | f: Lachnospiraceae; | |
| | | | g: Lachnospira; s: | |
| 193336 | 5 | 0.079365079 | k: Bacteria; p: Firmicutes; | 370 |
| | | | c: Clostridia; o: Clostridiales; | |
| | | | f: Ruminococcaceae; | |
| | | | g: Ruminococcus; s: | |
| 193436 | 9 | 0.142857143 | k: Bacteria; p: Actinobacteria; | 371 |
| | | | c: Coribacteriia; | |
| | | | o: Coriobacteriales; | |
| | | | f: Coriobacteriaceae; | |
| | | | g: Collinsella; s: | |
| 193551 | 13 | 0.206349206 | k: Bacteria; p: Firmicutes; | 372 |
| | | | c: Clostridia; o: Clostridiales; | |
| | | | f: Lachnospiraceae; | |
| | | | g: Coprococcus; s: | |
| 193575 | 39 | 0.619047619 | k: Bacteria; p: Actinobacteria; | 373 |
| | | | c: Coriobacteriia; | |
| | | | o: Coriobacteriales; | |
| | | | f: Coriobacteriaceae; | |
| | | | g: Collinsella; | |
| | | | s: aerofaciens | |
| 193621 | 21 | 0.333333333 | k: Bacteria; p: Firmicutes; | 374 |
| | | | c: Colstridia; o: Clostridiales; | |
| | | | f: Lachnospiraceae; | |
| | | | g: Coporococcus; s: | |
| 193831 | 13 | 0.206349206 | k: Bacteria; p: Firmicutes; | 375 |
| | | | c: Clostridia; o: Clostridiales; | |
| | | | f: Clostridiaceae; g: Clostridium; s: | |
| 193969 | 14 | 0.222222222 | k: Bacteria; p: Firmicutes; | 376 |
| | | | c: Clostridia; o: Clostridiales; | |
| | | | f: Lachnospiraceae; | |
| | | | g: Coprococcus; s: | |
| 194001 | 10 | 0.158730159 | k: Bacteria; p: Firmicutes; | 377 |
| | | | c: Clostridia; o: Clostridiales; | |
| | | | f: Lachnospiraceae; | |
| | | | g: Coprococcus; s: | |
| 194008 | 59 | 0.936507937 | k: Bacteria; p: Firmicutes; | 378 |
| | | | c: Clostridia; o: Clostridiales; | |
| | | | f: Lachnospiraceae; | |
| | | | g: Coprococcus; s: | |
| 194104 | 45 | 0.714285714 | k: Bacteria; p: Firmicutes; | 379 |
| | | | c: Clostridia; o: Clostridiales; | |
| | | | f: Lachnospiraceae; | |
| | | | g: Coprococcus; s: | |
| 194110 | 24 | 0.380952381 | k: Bacteria; p: Firmicutes; | 380 |
| | | | c: Clostridia; o: Clostridiales; | |
| | | | f: Lachnospiraceae; | |
| | | | g: Lachnospira; s: | |
| 194123 | 8 | 0.126984127 | k: Bacteria; p: Firmicutes; | 381 |
| | | | c: Clostridia; o: Clostridiales; | |
| | | | f: Lachnospiraceae; | |
| | | | g: Coprococcus; s: | |
| 194223 | 55 | 0.873015873 | k: Bacteria; p: Firmicutes; | 382 |
| | | | c: Clostridia; o: Costridiales; | |
| | | | f: Ruminococcaceae; | |
| | | | g: Ruminococcus; s: | |
| 194258 | 6 | 0.095238095 | k: Bacteria; p: Firmicutes; | 383 |
| | | | c: Clostridia; o: Clostridiales; | |
| | | | f: Ruminococcaceae; | |
| | | | g: Faecalibacterium; | |
| | | | s: prausnitzii | |
| 194297 | 12 | 0.19047619 | k: Bacteria; p: Firmicutes; | 384 |
| | | | c: Clostridia; o: Clostridiales; | |
| | | | f: Ruminococcaceae; | |
| | | | g: Ruminococcus; g: | |
| 194359 | 2 | 0.031746032 | k: Bacteria; p: Firmicutes; | 385 |
| | | | c: Clostridia; o; Clostridiales; | |
| | | | f: Lachnospiraceae; | |
| | | | g: Coprococcus; s: | |
| 194360 | 10 | 0.158730159 | k: Bacteria; p: Proteobacteria; | 386 |
| | | | c: Betaproteobacteria; | |
| | | | o: Burkholderiales; | |
| | | | f: Alcaligenaceae; g: Sutterella; s: | |
| 194383 | 3 | 0.047619048 | k: Bacteria; p: Firmicutes; | 387 |
| | | | c: Clostridia; o: Clostridiales; | |
| | | | f: Ruminococcaceae; | |
| | | | g: Faecalibacterium; | |
| | | | s: prausnitzii | |
| 194417 | 1 | 0.015873016 | k: Bacteria; p: Firmicutes; | 388 |
| | | | c: Clostridia; o: Clostridiales; | |
| | | | f: Lachnospiraceae; | |
| | | | g: Coprococcus; s: | |
| 194557 | 16 | 0.253968254 | k: Bacteria; p: Firmicutes; | 389 |
| | | | c: Clostridia; o: Clostridiales; | |
| | | | f: Lachnospiraceae; | |
| | | | g: Coprococcus; s: | |
| 194646 | 2 | 0.031746032 | k: Bacteria; p: Bacteroidetes; | 390 |
| | | | c: Bacteroidia; o: Bacteroidales; | |
| | | | f: [Odoribacteraceae]; | |
| | | | g: Odoribacter; s | |
| 194659 | 8 | 0.126984127 | k: Bacteria; p: Firmicutes; | 391 |
| | | | c: Clostridia; o: Clostridiales; | |
| | | | f: Clostridiaceae; g:; s: | |
| 194692 | 2 | 0.031746032 | k: Bacteria; p: Firmicutes; | 392 |
| | | | c: Clostridia; o: Clostridiales; | |
| | | | f: Ruminococcaceae; | |
| | | | g: Oscillospira; s: | |
| 194707 | 6 | 0.095238095 | k: Bacteria; p: Firmicutes; | 393 |
| | | | c: Clostridia; o: Clostridiales; | |
| | | | f: Lachnospiraceae; | |
| | | | g: Lachnospira; s: | |
| 194727 | 33 | 0.523809524 | k: Bacteria; p: Firmicutes; | 394 |
| | | | c: Clostridia; o: Clostridiales; | |
| | | | f: Lachnospiraceae; | |
| | | | g: Lachnospira; s: | |
| 194734 | 7 | 0.111111111 | k: Bacteria; p: Firmicutes; | 395 |
| | | | c: Clostridia; o: Clostridiales; | |
| | | | f: Lachnospiraceae; | |
| | | | g: Coprococcus; s: | |
| 194816 | 14 | 0.222222222 | k: Bacteria; p: Firmicutes; | 396 |
| | | | c: Clostridia; o: Clostridiales; | |
| | | | f: Ruminococcaceae; | |
| | | | g: Ruminococcus; s: | |
| 194868 | 25 | 0.396825397 | k: Bacteria; p: Firmicutes; | 397 |
| | | | c: Clostridia; o: Clostridiales; | |
| | | | f: Ruminococcaceae; | |
| | | | g: Ruminococcus; s: | |
| 194933 | 4 | 0.063492063 | k: Bacteria; p: Firmicutes; | 398 |
| | | | c: Clostridia; o: Clostridiales; | |
| | | | f: Ruminococcaceae; | |
| | | | g: Faecalibacterium; | |
| | | | s: prausnitzii | |
| 194947 | 3 | 0.047619048 | k: Bacteria; p: Firmicutes; | 399 |
| | | | c: Clostridia; o: Clostridiales; | |
| | | | f: Ruminococcaceea; | |
| | | | g: Ruminococcus; s: | |
| 195015 | 15 | 0.238095238 | k: Bacteria; p: Firmicutes; | 400 |
| | | | c: Clostridia; o: Clostridiales; | |
| | | | f: Lachnospiraceae; | |
| | | | g: Lachnospira; s: | |
| 195532 | 43 | 0.682539683 | k: Bacteria; p: Firmicutes; | 401 |
| | | | c: Clostridia; o: Clostridiales; | |
| | | | f: Lachnospiraceae; | |
| | | | g: Coprococcus; s: | |
| 195628 | 61 | 0.968253968 | k: Bacteria; p: Firmicutes; | 402 |
| | | | c: Clostridia; o: Clostridiales; | |
| | | | f: Lachnospiraceae; | |
| | | | g: Coprococcus; s: | |
| 195716 | 5 | 0.079365079 | k: Bacteria; p: Firmicutes; | 403 |
| | | | c: Clostridia; o: Clostridiales; | |
| | | | f: Lachnospiraceae; | |
| | | | g: Coprococcus; s: | |
| 195728 | 1 | 0.015873016 | k:Bacteria; p: Firmimcutes; | 404 |
| | | | c: Clostridia; o: Clostridiales; | |
| | | | f: Lachnospiraceae; | |
| | | | g: Coprococcus; s: | |
| 195752 | 28 | 0.444444444 | k: Bacteria; p: Firmicutes; | 405 |
| | | | c: Clostridia; o: Clostridiales; | |
| | | | f: Lachnospirsceae; | |
| | | | g: Lachnospira; s: | |
| 195801 | 18 | 0.258714286 | k: Bacteria; p: Firmicutes; | 406 |
| | | | c: Clostridia; o: Clostridiales; | |
| | | | f: Ruminococcaceae; | |
| | | | g: Faecalibacterium; | |
| | | | s: prausnitzii | |
| 195855 | 1 | 0.015873016 | k: Bacteria; p: Firmicutes; | 407 |
| | | | c: Clostridia; o: Clostridiales; | |
| | | | f: Ruminococcaceae; | |
| | | | g: Faecalibacterium; | |
| | | | s: prausnitzii | |
| 195876 | 14 | 0.222222222 | k: Bacteria; p: Firmicutes; | 408 |
| | | | c: Clostridia; o: Clostridiales; | |
| | | | f: Lachnospiraceae; | |
| | | | g: Coprococcus; s: | |
| 195929 | 18 | 0.285714286 | k: Bacteria; p: Firmicutes; | 409 |
| | | | c: Clostridia; o: Clostridiales; | |
| | | | f: Lachnospiraceae; | |
| | | | g: Coprococcus; s: | |
| 195933 | 11 | 0.174603175 | k: Bacteria; p: Firmicutes; | 410 |
| | | | c: Clostridia; o: Clostridiales; | |
| | | | f: Ruminococcaceae; | |
| | | | g: Oscillospira; s: | |
| 195947 | 7 | 0.111111111 | k: Bacteria; p: Firmicutes; | 411 |
| | | | c: Clostridia; o: Clostridiales; | |
| | | | f: Ruminococcaceae; | |
| | | | g: Ruminococcus; s: | |
| 196061 | 10 | 0.158730159 | k: Bacteria; p: Firmicutes; | 412 |
| | | | c: Clostridia; o: Clostridiales; | |
| | | | f: Ruminococcaceae; | |
| | | | g: Ruminococcus; s: | |
| 196112 | 5 | 0.079365079 | k: Bacteria; p: Firmicutes; | 413 |
| | | | c: Clostridia; o: Clostridiales; | |
| | | | f: Ruminococcaceae; | |
| | | | g: Ruminococcus; s: | |
| 196125 | 4 | 0.063492063 | k: Bacteria; p: Firmicutes; | 414 |
| | | | c: Clostridia; o: Clostridiales; | |
| | | | f: Ruminococcaceae; | |
| | | | g: Faecalibacterium; | |
| | | | s: prausnitzii | |
| 196131 | 1 | 0.015873016 | k: Bacteria; p: Bacteroidetes; | 415 |
| | | | c: Bacteroidia; o: Bacteroidales; | |
| | | | f: [Odoribacteraceae]; | |
| | | | g: Odoribacter; s: | |
| 196191 | 1 | 0.015873016 | k: Bacteria; p: Firmicutes; | 416 |
| | | | c: Clostridia; o: Clostridiales; | |
| | | | f: Ruminococcaceae; | |
| | | | g: Oscillospira; s: | |
| 196246 | 50 | 0.793650794 | k: Bacteria; p: Firmicutes; | 417 |
| | | | c: Clostridia; o: Clostridiales; | |
| | | | f: Ruminococcaceae; | |
| | | | g: Faecalibacterium; | |
| | | | s: prausnitzii | |
| 196386 | 3 | 0.047619048 | k: Bacteria; p: Bacteroidetes; | 418 |
| | | | c: Bacteroidia; o: Bacteroidales; | |
| | | | f: Rikenellaceae; g:; s: | |
| 196462 | 13 | 0.206349206 | k: Bacteria; p: Firmicutes; | 419 |
| | | | c: Clostridia; o: Clostridiales; | |
| | | | f: Christensenellaceae; g:; s: | |
| 196553 | 31 | 0.492063492 | k: Bacteria; p: Firmicutes; | 420 |
| | | | c: Clostridia; o: Clostridiales; | |
| | | | f: Ruminococcaceae; | |
| | | | g: Oscillospira; s: | |
| 196951 | 1 | 0.015873016 | k: Bacteria; p: Firmicutes; | 421 |
| | | | c: Clostridia; o: Clostridiales; | |
| | | | f: Ruminococcaceae; | |
| | | | g: Ruminococcus; s: | |
| 196991 | 8 | 0.126984127 | k: Bacteria; p: Firmicutes; | 422 |
| | | | c: Clostridia; o: Clostridiales; | |
| | | | f: Ruminococcaceae; | |
| | | | g: Oscillospira; s: | |
| 197003 | 14 | 0.222222222 | k: Bacteria; p: Firmicutes; | 423 |
| | | | c: Clostridia; o: Clostridiales; | |
| | | | f: Lachnospiraceae; | |
| | | | g: Coprocccus; s: | |
| 197022 | 23 | 0.365079365 | k: Bacteria; p: Firmicutes; | 424 |
| | | | c: Clostridia; o: Clostridiales; | |
| | | | f: Lachnospiraceae; | |
| | | | g: Coprococcus; s: | |
| 197099 | 16 | 0.253968254 | k: Bacteria; p: Firmicutes; | 425 |
| | | | c: Clostridia; o: Clostridiales; | |
| | | | f: Ruminococcaceae; | |
| | | | g: Oscillospira; s: | |
| 197107 | 1 | 0.015873016 | k: Bacteria; p: Firmicutes; | 426 |
| | | | c: Clostridia; o: Clostridiales; | |
| | | | f: Lachnospiraceae; | |
| | | | g: Lachnospira; s: | |
| 197112 | 1 | 0.015873016 | k: Baceria; p: Firmicutes; | 427 |
| | | | c: Clostridia; o: Clostridiales; | |
| | | | f: Lachnospiraceae; | |
| | | | g: Coprococcus; s: | |
| 197341 | 7 | 0.111111111 | k: Bacteria; p: Firmicutes; | 428 |
| | | | c: Clostridia; o: Clostridiales; | |
| | | | f: Lachnospiraceae; | |
| | | | g: Lachnospira; s: | |
| 197517 | 2 | 0.031746032 | k: Bacteria; p: Bacteroidetes; | 429 |
| | | | c: Bacteroidia; o: Bacteroidales; | |
| | | | f: [Barnediellaceae]; g:; s: | |
| 197581 | 29 | 0.46031746 | k: Bacteria; p: Firmicutes; | 430 |
| | | | c: Clostridia; o: Clostridiales; | |
| | | | f: Lachnospiraceae; | |
| | | | g: Lachnospira; s: | |
| 197603 | 3 | 0.047619048 | k: Bacteria; p: Firmicutes; | 431 |
| | | | c: Clostridia; o: Clostridiales; | |
| | | | f: Lachnospiraceae; | |
| | | | g: Coprococcus; s: | |
| 197649 | 7 | 0.111111111 | k: Bacteria; p: Firmicutes; | 432 |
| | | | c: Clostridia; o: Clostridiales; | |
| | | | f: Lachnospiraceae; | |
| | | | g: Lachnospira; s: | |
| 197675 | 4 | 0.063492063 | k: Bacteria; p: Firmicutes; | 433 |
| | | | c: Clostridia; o: Clostridiales; | |
| | | | f: Lachnospiraceae; | |
| | | | g: Coprococcus; s: | |
| 197708 | 33 | 0.523809524 | k: Bacteria; p: Firmicutes; | 434 |
| | | | c: Clostridia; o: Clostridiales; | |
| | | | f: Ruminococcaceae; | |
| | | | g: Faecalibacterium; | |
| | | | s: prausnitzii | |
| 197757 | 34 | 0.53968254 | k: Bacteria; p: Firmicutes; | 435 |
| | | | c: Clostridia; o: Clostridiales; | |
| | | | f: Ruminococcaceae; | |
| | | | g: Faecalibacterium; | |
| | | | s: prausnitzii | |
| 197807 | 8 | 0.126984127 | k: Bacteria; p: Firmicutes; | 436 |
| | | | c: Clostridia; o: Clostridiales; | |
| | | | f: Clostridiaceae; g: Clostridium; s: | |
| 197890 | 4 | 0.063492063 | k: Bacteria; p: Firmicutes; | 437 |
| | | | c: Clostridia; o: Clostridiales; | |
| | | | f: Ruminococcaceae; | |
| | | | g: Oscillospira; s: | |
| 197897 | 4 | 0.063492063 | k: Bacteria; p: Firmicutes; | 438 |
| | | | c: Clostridia; o: Clostridiales; | |
| | | | f: Lachnospiraceae; | |
| | | | g: Coprococcus; s: | |
| 197943 | 4 | 0.063492063 | k: Bacteria; p: Firmicutes; | 439 |
| | | | c: Clostridia; o: Clostridiales; | |
| | | | f: Ruminococceae; | |
| | | | g: Ruminococcus; s: | |
| 198034 | 2 | 0.031746032 | k: Bacteria; p: Firmicutes; | 440 |
| | | | c: Clostridia; o: Clostridiales; | |
| | | | f: Lachnospiraceae; | |
| | | | g: Lachnospira; s: | |
| 198085 | 4 | 0.063492063 | k: Bacteria; p: Firmicutes; | 441 |
| | | | c: Clostridia; o: Clostridiales; | |
| | | | f: Ruminococcaceae; | |
| | | | g: Faecalibacterium; | |
| | | | s: prausnitzii | |
| 198194 | 23 | 0.365079365 | k: Bacteria; p: Firmicutes; | 442 |
| | | | c: Clostridia; o: Clostridiales; | |
| | | | f: Ruminococcaceae; | |
| | | | g: Oscillospira; s: | |
| 198210 | 10 | 0.158730159 | k: Bacteria; p: Firmicutes; | 443 |
| | | | c: Clostridia; o: Clostridiales; | |
| | | | f: Ruminococcaceae; | |
| | | | g: Ruminococcus; s: | |
| 198555 | 12 | 0.19047619 | k: Bacteria; p: Firmicutes; | 444 |
| | | | c: Clostridia; o: Clostridiales; | |
| | | | f: Lachnospiraceae; | |
| | | | g: Coprococcus; s: | |
| 198909 | 26 | 0.412698413 | k: Bacteria; p: Firmicutes; | 445 |
| | | | c: Clostridia; o: Clostridiales; | |
| | | | f: Lachnospiraceae; | |
| | | | g: Lachnospira; s: | |
| 198980 | 23 | 0.365079365 | k: Bacteria; p: Firmicutes; | 446 |
| | | | c: Clostridia; o: Clostridiales; | |
| | | | f: Ruminococcaceae; | |
| | | | g: Ruminococcus; s: | |
| 199034 | 32 | 0.507936508 | k: Bacteria; p: Firmicutes; | 447 |
| | | | c: Clostridia; o: Clostridiales; | |
| | | | f: Clostridiaceae; g: Clostridium; s: | |
| 199077 | 7 | 0.111111111 | k: Bacteria; p: Firmicutes; | 448 |
| | | | c: Clostridia; o: Clostridiales; | |
| | | | f: Lachnospiraceae; | |
| | | | g: Coprococcus; s: | |
| 199081 | 12 | 0.19047619 | k: Bacteria; p: Firmicutes; | 449 |
| | | | c: Clostridia; o: Clostridiales; | |
| | | | f: Lachnospiraceae; | |
| | | | g: Coprococcus; s: | |
| 199145 | 11 | 0.174603175 | k: Bacteria; p: Firmicutes; | 450 |
| | | | c: Clostridia; o: Clostridiales; | |
| | | | f: Ruminococcaceae; | |
| | | | g: Faecalibacterium; | |
| | | | s: prausnitzii | |
| 199228 | 1 | 0.015873016 | k: Bacteria; p: Firmicutes; | 451 |
| | | | c: Clostridia; o: Clostridiales; | |
| | | | f: Lachnospiraceae; | |
| | | | g: Coprococcus; s: | |
| 199268 | 8 | 0.126984127 | k: Bacteria; p: Firmicutes; | 452 |
| | | | c: Clostridia; o: Clostridiales; | |
| | | | f: Clostridiaceae; g:; s: | |
| 199300 | 20 | 0.317460317 | k: Bacteria; p: Firmicutes; | 453 |
| | | | c: Clostridia; o: Clostridiales; | |
| | | | f: Lachnospiraceae; | |
| | | | g: Coprococcus; s: | |
| 199313 | 5 | 0.079365079 | k: Bacteria; p: Firmicutes; | 454 |
| | | | c: Clostridia; o: Clostridiales; | |
| | | | f: Lachnospiraceae; | |
| | | | g: Coprococcus; s: | |
| 199337 | 6 | 0.095238095 | k: Bacteria; p: Firmicutes; | 455 |
| | | | c: Clostridia; o: Clostridiales; | |
| | | | f: Ruminococcaceae; | |
| | | | g: Oscillospira; s: | |
| 199354 | 27 | 0.428571429 | k: Bacteria; p: Bacteoidetes; | 456 |
| | | | c: Bacteroidia; o: Bacteroidales; | |
| | | | f: [Barnesiellaceae]; g:; s: | |
| 199388 | 20 | 0.317460317 | k: Bacteria; p: Firmicutes; | 457 |
| | | | c: Clostridia; o: Clostridiales; | |
| | | | f: Clostridiaceae; g: Clostridium; s: | |
| 199421 | 33 | 0.523809524 | k: Bacteria; p: Firmicutes; | 458 |
| | | | c: Clostridia; o: Clostridiales; | |
| | | | f: Lachnospiraceae; | |
| | | | g: Lachnospira; s: | |
| 199430 | 1 | 0.015873016 | k: Bacteria; p: Firmicutes; | 459 |
| | | | c: Clostridia; o: Clostridiales; | |
| | | | f: Ruminococcaceae; | |
| | | | g: Faecalibacterium; | |
| | | | s: prausnitzii | |
| 199491 | 11 | 0.174603175 | k: Bacteria; p: Firmicutes; | 460 |
| | | | c: Clostridia; o: Clostridiales; | |
| | | | f: Lachnospiraceae; | |
| | | | g: Coprococcus; s: | |
| 199534 | 52 | 0.825396825 | k: Bacteria; p: Bacteroidetes; | 461 |
| | | | c: Bacteroidia; o: Bacteroidales; | |
| | | | f: Rikenellaceae; g:; s: | |
| 199543 | 2 | 0.031746032 | k: Bacteria; p: Firmicutes; | 462 |
| | | | c: Clostridia; o: Clostridiales; | |
| | | | f: Ruminococcaceae; | |
| | | | g: Ruminococcus; s: | |
| 199568 | 18 | 0.285714286 | k: Bacteria; p: Firmicutes; | 463 |
| | | | c: Clostridia; o: Clostridiales; | |
| | | | f: Clostridiaceae; g: Clostridium; s: | |
| 199686 | 7 | 0.111111111 | k: Bacteria; p: Actinobacteria; | 464 |
| | | | c: Coriobacteriia; | |
| | | | o: Coriobacteriales; | |
| | | | f: Coriobacteriaceae; g:; s: | |
| 199694 | 53 | 0.841269841 | k: Bacteria; p: Firmicutes; | 465 |
| | | | c: Clostridia; o: Clostridiales; | |
| | | | f: Clostridiaceae; g: Clostridium; s: | |
| 199702 | 1 | 0.015873016 | k: Bacteria; p: Firmicutes; | 466 |
| | | | c: Clostridia; o: Clostridiales; | |
| | | | f: Ruminococcaceae; | |
| | | | g: Faecalibacterium; | |
| | | | s: prausnitzii | |
| 202162 | 48 | 0.761904762 | k: Bacteria; p: Firmicutes; | 467 |
| | | | c: Clostridia; o: Clostridiales; | |
| | | | f: Ruminococcaceae; | |
| | | | g: Ruminococcus; s: | |
| 204126 | 10 | 0.158730159 | k: Bacteria; p: Firmicutes; | 468 |
| | | | c: Clostridia; o: Clostridiales; | |
| | | | f: Ruminococcaceae; | |
| | | | g: Oscillospira; s: | |
| 204352 | 6 | 0.095238095 | k: Bacteria; p: Firmicutes; | 469 |
| | | | c: Clsotridia; o: Clostridiales; | |
| | | | f: Ruminococcaceae; | |
| | | | g: Ruminococcus; s: | |
| 205613 | 4 | 0.063492063 | k: Bacteria; p: Firmicutes; | 470 |
| | | | c: Clostridia; o: Clostridiales; | |
| | | | f: Lachnospiraceae; | |
| | | | g: Coprococcus; s: | |
| 206513 | 23 | 0.365079365 | k: Bacteria; p: Firmicutes; | 471 |
| | | | c: Clostridia; o: Clostridiales; | |
| | | | f: Ruminococcaceae; | |
| | | | g: Oscillospira; s: | |
| 207252 | 17 | 0.26984127 | k: Bacteria; p: Firmicutes; | 472 |
| | | | c: Clostridia; o: Clostridiales; | |
| | | | f: Ruminococcaceae; | |
| | | | g: Oscillospira; s: | |
| 207340 | 3 | 0.047619048 | k: Bacteria; p: Firmicutes; | 473 |
| | | | c: Clostridia; o: Clostridiales; | |
| | | | f: [Mogibacteriaceae]; g:; s: | |
| 207487 | 33 | 0.523809524 | k: Bacteria; p: Firmicutes; | 474 |
| | | | c: Clostridia; o: Clostridiales; | |
| | | | f: Lachnospiraceae; | |
| | | | g: Lachnospira; s: | |
| 207994 | 19 | 0.301587302 | k: Bacteria; p: Firmicutes; | 475 |
| | | | c: Clostridia; o: Clostridiales; | |
| | | | f: Ruminococcaceae; | |
| | | | g: Ruminococcus; s: | |
| 208539 | 1 | 0.015873016 | k: Bacteria; p: Firmicutes; | 476 |
| | | | c: Clostridia; o: Clostridiales; | |
| | | | f: [Mogibacteriaceae]; g:; s: | |
| 208739 | 60 | 0.952380952 | k: Bacteria; p: Firmicutes; | 477 |
| | | | c: Clostridia; o: Clostridiales; | |
| | | | f: Ruminococcaceae; | |
| | | | g: Faecalibacterium; | |
| | | | s: prausnitzii | |
| 208843 | 2 | 0.031746032 | k: Bacteria; p: Bacteroidetes; | 478 |
| | | | c: Bacteroidia; o: Bacteroidales; | |
| | | | f: [Odoribacteraceae]; | |
| | | | g: Odoribacter; s: | |
| 208901 | 10 | 0.158730159 | k: Bacteria; p: Firmicutes; | 479 |
| | | | c: Clostridia; o: Clostridiales; | |
| | | | f: Lachnospiraceae; | |
| | | | g: Coprococcus; s: | |
| 209327 | 1 | 0.015873016 | k: Bacteria; p: Firmicutes; | 480 |
| | | | c: Clostridia; o: Clostridiales; | |
| | | | f: Ruminococcaceae; | |
| | | | g: Oscillospira; s: | |
| 209760 | 1 | 0.015873016 | k: Bacteria; p: Firmicutes; | 481 |
| | | | c: Clostridia; o: Clostridiales; | |
| | | | f: Ruminococcaceae; | |
| | | | g: Faecalibacterium; | |
| | | | s: prausnitzii | |
| 210095 | 14 | 0.222222222 | k: Bacteria; p: Firmicutes; | 482 |
| | | | c: Clostridia; o: Clostridiales; | |
| | | | f: Ruminococcaceae; | |
| | | | g: Ruminococccus; s: | |
| 210292 | 5 | 0.079365079 | k: Bacteria; p: Firmicutes; | 483 |
| | | | c: Clostridia; o: Clostridiales; | |
| | | | f: Ruminococcaceae; | |
| | | | g: Oscillospira; s: | |
| 210542 | 2 | 0.031746032 | k: Bacteria; p: Firmicutes; | 484 |
| | | | c: Clostridia; o: Clostridiales; | |
| | | | f: Lachnospiraceae; | |
| | | | g: Lachnospira; s: | |
| 210647 | 22 | 0.349206349 | k: Bacteria; p: Firmicutes; | 485 |
| | | | c: Clostridia; o: Clostridiales; | |
| | | | f: Ruminococcaceae; | |
| | | | g: Ruminococcus; s: | |
| 212686 | 6 | 0.095238095 | k: Bacteria; p: Firmicutes; | 486 |
| | | | c: Clostridia; o: Clostridiales; | |
| | | | f: Ruminococcaceae; | |
| | | | g: Oscillospira; s: | |
| 213394 | 16 | 0.253968254 | k: Bacteria; p: Firmicutes; | 487 |
| | | | c: Clostridia; o: Clostridiales; | |
| | | | f: Lachnospiraceae; | |
| | | | g: Lachnospira; s: | |
| 213671 | 1 | 0.015873016 | k: Bacteria; p: Bacteroidetes; | 488 |
| | | | c: Bacteroidia; o: Bacteroidales; | |
| | | | f: Rikenellaceae; g:; s: | |
| 214031 | 14 | 0.222222222 | k: Bacteria; p: Bacteroidetes; | 489 |
| | | | c: Bacteroidia; o: Bacteroidales; | |
| | | | f: Rikenellaceae; g:; s: | |
| 214036 | 44 | 0.698412698 | k: Bacteria; p: Firmicutes; | 490 |
| | | | c: Clostridia; o: Clostridiales; | |
| | | | f: [Mogibacteriaceae]; g:; s: | |
| 215097 | 9 | 0.142857143 | k: Bacteria; p: Proteobacteria; | 491 |
| | | | c: Betaproteobacteria; | |
| | | | o: Burkholderiales; | |
| | | | f: Alcaligenaceae; g: Sutterella; s: | |
| 215963 | 1 | 0.015873016 | k: Bacteria; p: Firmicutes; | 492 |
| | | | c: Clostridia; o: Clostridiales; | |
| | | | f: Clostridiaceae; g: Clostridium; s: | |
| 216010 | 21 | 0.333333333 | k: Bacteria; p: Firmicutes; | 493 |
| | | | c: Clostridia; o: Clostridiales; | |
| | | | f: [Mogibacteriaceae]; g:; s: | |
| 216550 | 2 | 0.031746032 | k: Bacteria; p: Bacteroidetes; | 494 |
| | | | c: Bacteroidia; o: Bacteroidales; | |
| | | | f: Rikenellaceae; g:; s: | |
| 216599 | 27 | 0.428571429 | k: Bacteria; p: Bacteroidetes; | 495 |
| | | | c: Bacteroidia; o: Bacteroidales; | |
| | | | f: Rikenellaceae; g:; s: | |
| 217109 | 8 | 0.126984127 | k: Bacteria; p: Firmicutes; | 496 |
| | | | c: Clostridia; o: Clostridiales; | |
| | | | f: Christensenellaceae; g:; s: | |
| 221361 | 15 | 0.238095238 | k: Bacteria; p: Proteobacteria; | 497 |
| | | | c: Gammaproteobacteria; | |
| | | | o: Enterobacteriales; f: | |
| | | | Enterobacteriaceae; g:; s: | |
| 221454 | 4 | 0.063492063 | k: Bacteria; p: Firmicutes; | 498 |
| | | | c: Clostridia; o: Clostridiales; | |
| | | | f: Ruminococcaceae; | |
| | | | g: Ruminococcus; s: | |
| 223059 | 1 | 0.015873016 | k: Bacteria; p: Firmicutes; | 499 |
| | | | c: Clostridia; o: Clostridiales; | |
| | | | f: Ruminococcaceae; | |
| | | | g: Ruminococcus; s: | |
| 227697 | 7 | 0.111111111 | k: Bacteria; p: Firmicutes; | 500 |
| | | | c: Clostridia; o: Clostridiales; | |
| | | | f: Ruminococcaceae; | |
| | | | g: Ruminococcus; s: | |
| 227819 | 15 | 0.238095238 | k: Bacteria; p: Actinobacteria; | 501 |
| | | | c: Coriobacteriia; | |
| | | | o: Coriobacteriales; | |
| | | | f: Coriobacteriaceae; g:; s: | |
| 228199 | 4 | 0.063492063 | k: Bacteria; p: Firmicutes; | 502 |
| | | | c: Clostridia; o: Clostridiales; | |
| | | | f: Ruminococcaceae; | |
| | | | g: Ruminococcus; s: | |
| 228748 | 5 | 0.079365079 | k: Bacteria; p: Firmicutes; | 503 |
| | | | c: Clostridia; o: Clostridiales; | |
| | | | f: Christensenellaceae; g:; s: | |
| 229455 | 2 | 0.031746032 | k: Bacteria; p: Firmicutes; | 504 |
| | | | c: Clostridia; o: Clostridiales; | |
| | | | f: Ruminococcaceae; | |
| | | | g: Ruminococcus; s: | |
| 229905 | 1 | 0.015873016 | k: Bacteria; p: Firmixutes; | 505 |
| | | | c: Clostridia; o: Clostridiales; | |
| | | | f: [Mogibacteriaceae]; g:; s: | |
| 230578 | 6 | 0.095238095 | k: Bacteria; p: Actinobacteria; | 506 |
| | | | c: Coriobacteriia; | |
| | | | o: Coriobacteriales; | |
| | | | f: Coriobacteriaceae; g:; s: | |
| 231787 | 2 | 0.031746032 | k: Bacteria; p: Proteobacteria; | 507 |
| | | | c: Gammaproteobacteria; | |
| | | | o: Enterobacteriales; f: | |
| | | | Enterobacteriaceae; g:; s: | |
| 232417 | 11 | 0.174603175 | k: Bacteria; p: Firmicutes; | 508 |
| | | | c: Clostridia; o: Clostridiales; | |
| | | | f: Ruminococcaceae; | |
| | | | g: Ruminococcus; s: | |
| 232900 | 7 | 0.111111111 | k: Bacteria; p: Actinobacteria; | 509 |
| | | | c: Coriobacteriia; | |
| | | | o: Coriobacteriales; | |
| | | | f: Coriobacteriaceae; g:; s: | |
| 232975 | 41 | 0.650793651 | k: Bacteria; p: Firmicutes; | 510 |
| | | | c: Clostridia; o: Clostridiales; | |
| | | | f: Ruminococcaceae; | |
| | | | g: Oscillospira; s: | |
| 233052 | 10 | 0.158730159 | k: Bacteria; p: Firmicutes; | 511 |
| | | | c: Clostridia; o: Clostridiales; | |
| | | | f: Christensenellaceae; g:; s: | |
| 233648 | 2 | 0.031746032 | k: Bacteria; p: Firimicutes; | 512 |
| | | | c: Clostridia; o: Clostridiales; | |
| | | | f: Lachnospiraceae; | |
| | | | g: Coprococcus; s: | |
| 234443 | 2 | 0.031746032 | k: Bacteria; p: Bacteroidetes; | 513 |
| | | | c: Bacteroidia; o: Bacteroidales; | |
| | | | f: Rikenellaceae; g:; s: | |
| 234447 | 1 | 0.015873016 | k: Bacteria; p: Firmicutes; | 514 |
| | | | c: Clostridia; o: Clostridiale; | |
| | | | f: Christensenellaceae; g:; s: | |
| 234912 | 1 | 0.015873016 | k: Bacteria; p: Firmicutes; | 515 |
| | | | c: Clostridia; o: Clostridiales; | |
| | | | f: Clostridiaceae; g: Clostridium; s: | |
| 235127 | 2 | 0.031746032 | k: Bacteria; p: Actinobacteria; | 516 |
| | | | c: Coriobacteriia; | |
| | | | o: Coriobacteriales; | |
| | | | f: Coriobacteriaceae; g:; s: | |
| 236811 | 2 | 0.031746032 | k: Bacteria; p: Proteobacteria; | 517 |
| | | | c: Gammaproteobacteria; | |
| | | | o: Enterobacteriales; | |
| | | | f: Enterobacteriaceae; | |
| | | | g: Enterobacter; s: | |
| 240271 | 8 | 0.126984127 | k: Bacteria; p: Firmicutes; | 518 |
| | | | c: Clostridia; o: Clostridiales; | |
| | | | f: Ruminococcaceae; | |
| | | | g: Oscillospira; s: | |
| 243150 | 4 | 0.063492063 | k: Bacteria; p: Bacteroidetes; | 519 |
| | | | c: Bacteroidia; o: Bacteroidales; | |
| | | | f: Rikenellaceae; g:; s: | |
| 245625 | 35 | 0.555555556 | k: Bacteria; p: Firmicutes; | 520 |
| | | | c: Clostridia; o: Clostridiales; | |
| | | | f: Clostridiaceae; g: Clostridium; s: | |
| 246330 | 1 | 0.015873016 | k: Bacteria; p: Proteobacteria; | 521 |
| | | | c: Gammaproteobacteria; | |
| | | | o: Enterobacteriales; f: | |
| | | | Enterobacteriaceae; g:; s: | |
| 251456 | 7 | 0.111111111 | k: Bacteria; p: Firmicutes; | 522 |
| | | | c: Clostridia; o: Clostridiales; | |
| | | | f: Ruminococcaceae; | |
| | | | g: Ruminococcus; | |
| | | | s: flavefaciens | |
| 259212 | 2 | 0.031746032 | k: Bacteria; p: Firmicutes; | 523 |
| | | | c: Closstridia; o: Clostridiales; | |
| | | | f: Clostridiaceae; g:; s: | |
| 259772 | 12 | 0.19047619 | k: Bacteria; p: Firmicutes; | 524 |
| | | | c: Clsotridia; o: Clostridiales; | |
| | | | f: Lachnospiraceae; | |
| | | | g: Coprococcus; s: | |
| 260559 | 11 | 0.174603175 | k: Bacteria; p: Firmicutes; | 252 |
| | | | c: Clostridia; o: Clostridiales; | |
| | | | f: Ruminococcaceae; | |
| | | | g: Ruminococcus; s: | |
| 260842 | 1 | 0.015873016 | k: Bacteria; p: Firmicutes; | 526 |
| | | | c: Clostridia; o: Clostridiales; | |
| | | | f: Ruminococcaceae; | |
| | | | g: Oscillospira; s: | |
| 261572 | 1 | 0.015873016 | k: Bacteria; p: Firmicutes; | 527 |
| | | | c: Clostridia; o: Clostridiales; | |
| | | | f: [Mogibacteriaceae]; g:; s: | |
| 262024 | 2 | 0.031746032 | k: Bacteria; p: Firmicutes; | 528 |
| | | | c: Clostridia; o: Clostridiales; | |
| | | | f: Ruminococcaceae; | |
| | | | g: Oscillospira; s: | |
| 262060 | 11 | 0.174603175 | k: Bacteria; p: Firmicutes; | 529 |
| | | | c: Clostridia; o: Clostridiales; | |
| | | | f: Clostridiaceae; g:; s: | |
| 262355 | 1 | 0.015873016 | k: Bacteria; p: firmicutes; | 530 |
| | | | c: Clostridia; o: Clostridiales; | |
| | | | f: Clostridiaceae; g:; s: | |
| 262668 | 19 | 0.301587302 | k: Bacteria; p: Firmicutes; | 531 |
| | | | c: Clostridia; o: Clostridiales; | |
| | | | f: Ruminococcaceae; | |
| | | | g: Ruminococcus; s: | |
| 263074 | 3 | 0.047619048 | k: Bacteria; p: Firmicutes; | 532 |
| | | | c: Clostridia; o: Clostridiales; | |
| | | | f: Lachnospiraceae; | |
| | | | g: Coprococcus; s: | |
| 263337 | 1 | 0.015873016 | k: Bacteria; p: Firmicutes; | 533 |
| | | | c: Clostridia; o: Clostridiales; | |
| | | | f: Ruminococcaceae; | |
| | | | g: Oscillospira; s: | |
| 264325 | 2 | 0.031746032 | k: Bacteria; p: Bacteroidetes; | 534 |
| | | | c: Bacteroidia; o: Bacteroidales; | |
| | | | f: Rikenellaceae; g:; s: | |
| 265363 | 1 | 0.015873016 | k: Bacteria; p: Firmicutes; | 535 |
| | | | c: Clostridia; o: Clostridiales; | |
| | | | f: Clostridiaceae; g:; s: | |
| 266726 | 1 | 0.015873016 | k: Bacteria; p: Frimicutes; | 536 |
| | | | c: Clostridia; o: Clostridiales; | |
| | | | f: Lachnospiraceae; | |
| | | | g: Coprococcus; s: | |
| 267042 | 2 | 0.031746032 | k: Bacteria; p: Firmicutes; | 537 |
| | | | c: Clostridia; o: Clostridiales; | |
| | | | f: Ruminococcaceae; | |
| | | | g: Ruminococcus; s: | |
| 267514 | 1 | 0.015873016 | k: Bacteria; p: Firmicutes; | 538 |
| | | | c: Clostridia; o: Clostridiales; | |
| | | | f: Lachnospiraceae; | |
| | | | g: Coprococcus; s: | |
| 268074 | 2 | 0.031746032 | k: Bacteria; p: Firmicutes; | 539 |
| | | | c: Clostridia; o: Clostridiales; | |
| | | | f: Clostridiaceae; g:; s: | |
| 268733 | 2 | 0.031746032 | k: Bacteria; p: Firmicutes; | 540 |
| | | | c: Clostridia; o: Clostridiales; | |
| | | | f: Clostridiaceae; g:; s: | |
| 269019 | 1 | 0.015873016 | k: Bacteria; p: Firmicutes; | 541 |
| | | | c: Clostridia; o: Clostridiales; | |
| | | | f: Ruminococcaceae; | |
| | | | g: Oscillospira; s: | |
| 269145 | 32 | 0.507936508 | k: Bacteria; p: Firmicutes; | 542 |
| | | | c: Clostridia; o: Clostridiales; | |
| | | | f: Ruminococcaceae; | |
| | | | g: Oscillospira; s: | |
| 269833 | 6 | 0.095238095 | k: Bacteria; p: Firmicutes; | 543 |
| | | | c: Clostridia; o: Clostridiales; | |
| | | | f: Ruminococcaceae; | |
| | | | g: Ruminococcus; | |
| | | | s: flavefaciens | |
| 270059 | 11 | 0.174603175 | k: Bacteria; p: Firmicutes; | 544 |
| | | | c: Clostridia; o: Clostridiales; | |
| | | | f: Ruminococcaceae; | |
| | | | g: Oscillospira; s: | |
| 270162 | 6 | 0.095238095 | k: Bacteria; p: Firmicutes; | 545 |
| | | | c: Clostridia; o: Clostridiales; | |
| | | | f: Veillonellaceae; g: | |
| | | | Phascolarctobacterium; s: | |
| 270200 | 3 | 0.047619048 | k: Bacteria; p: Firmicutes; | 546 |
| | | | c: Clostridia; o: Clostridiales; | |
| | | | f: Clostridiaceae; g:; s: | |
| 270519 | 1 | 0.015873016 | k: Bacteria; p: Firmicutes; | 547 |
| | | | c: Clostridia; o: Clostridiales; | |
| | | | f: Ruminococcaceae; | |
| | | | g: Oscillospira; s: | |
| 271430 | 6 | 0.095238095 | k: Bacteria ; p: Firmicutes; | 548 |
| | | | c: Clostridia; o: Clostridiales; | |
| | | | f: Ruminococcaceae; | |
| | | | g: Ruminococcus; s: | |
| 271563 | 11 | 0.174603175 | k: Bacteria; p: Firmicutes; | 549 |
| | | | c: Clostridia; o: Clostridiales; | |
| | | | f: Ruminococcaceae; | |
| | | | g: Oscillospira; s: | |
| 274083 | 17 | 0.26984127 | k: Bacteria; p: firmicutes; | 550 |
| | | | c: Clostridia; o: Clostridiales; | |
| | | | f: Ruminococcaceae; | |
| | | | g: Oscillospira; s: | |
| 274569 | 3 | 0.047619048 | k: Bacteria; p: Firmicutes; | 551 |
| | | | c: Clostridia; o: Clostridiales; | |
| | | | f: Ruminococcaceae; | |
| | | | g: Ruminococcus; s: | |
| 275097 | 3 | 0.047619048 | k: Bacteria; p: Proteobacteria; | 552 |
| | | | c: Gammaproteobacteria; | |
| | | | o: Enterobacteriales; f: | |
| | | | Enteorbacteriaceae; g:; s: | |
| 275237 | 1 | 0.015873016 | k: Bacteria; p: Firmicutes; | 553 |
| | | | c: Clostridia; o: Clostridiales; | |
| | | | f: Veillonellaceae; g: | |
| | | | Phascolarctobacterium; s: | |
| 275288 | 3 | 0.047619048 | k: Bacterium; p: Firmicutes; | 554 |
| | | | c: Clostridium; o: Clostridiales; | |
| | | | f: Ruminococcaceae; | |
| | | | g: Oscillospira; s: | |
| 275592 | 1 | 0.015873016 | k: Bacteria; p: Firmicutes; | 555 |
| | | | c: Clostridia; o: Clostridiales; | |
| | | | f: Clostridiaceae; g:; s: | |
| 276650 | 2 | 0.031746032 | k: Bacteria; p: Actinobacteria; | 556 |
| | | | c: Coriobacteriia; | |
| | | | o: Coriobacteriales; | |
| | | | f: Coriobacteriaceae; g:; s: | |
| 278098 | 2 | 0.031746032 | k: Bacteria; p: Firmicutes; | 557 |
| | | | c: Clostridia; o: Clostridiales; | |
| | | | f: Ruminococcaceae; | |
| | | | g: Ruminococcus; s: | |
| 278506 | 9 | 0.142857143 | k: Bacteria; p: Firmicutes; | 558 |
| | | | c: Clostridia; o: Clostridiales; | |
| | | | f: Ruminococcaceae; | |
| | | | g: Ruminococcus; s: | |
| 278609 | 1 | 0.015873016 | k: Bacteria; p: Firmicutes; | 559 |
| | | | c: Clostridia; o: Clostridiales; | |
| | | | f: Ruminococcaceae; | |
| | | | g: Oscillospira; s: | |
| 278758 | 3 | 0.047619048 | k: Bacteria; p: Firmicutes; | 560 |
| | | | c: Clostridia; o: Clostridiales; | |
| | | | f: Ruminococcaceae; | |
| | | | g: Oscillospira; s: | |
| 281015 | 8 | 0.126984127 | k: Bacteria; p: Proteobacteria; | 561 |
| | | | c: Gammaproteobacteria; | |
| | | | o: Enterobacteriales; f: | |
| | | | Enterobacteriaceae; g:; s: | |
| 284014 | 1 | 0.015873016 | k: Bacteria; p: Proteobacteria; | 562 |
| | | | c: Gammaproteobacteria; | |
| | | | o: Enterobacteriales; f: | |
| | | | Enterobacteriaceae; g:; s: | |
| 287514 | 38 | 0.603174603 | k: Bacteria; p: Actinobacteria; | 563 |
| | | | c: Coriobacteriia; | |
| | | | o: Coriobacteriales; | |
| | | | f: Coriobacteriaceae; g: Collinsella; | |
| | | | s: aerofaciens | |
| 287608 | 1 | 0.015873016 | k: Bacteria; p: Firmicutes; | 564 |
| | | | c: Clostridia; o: Clostridiales; | |
| | | | f: Ruminococcaceae; | |
| | | | g: Ruminococcus; s: | |
| 289110 | 5 | 0.079365079 | k: Bacteria; p: Firmicutes; | 565 |
| | | | c: Clostridia; o: Clostridiales; | |
| | | | f: Veillonellaceae; g: | |
| | | | Phascolarctobacterium; s: | |
| 289306 | 49 | 0.777777778 | k: Bacteria; p: Firmicutes; | 566 |
| | | | c: Clostridia; o: Clostridiales; | |
| | | | f: Ruminococcaceae; | |
| | | | g: Oscillospira; s: | |
| 289709 | 1 | 0.015873016 | k: Bacteria; p: Proteobacteria; | 567 |
| | | | c: Gammaproteobacteria; | |
| | | | o: Enterobacteriales; f: | |
| | | | Enterobacteriaceae; g:; s: | |
| 289752 | 6 | 0.095238095 | k: Bacteria; p: Firmicutes; | 568 |
| | | | c: Clostridia; o: Clostridiales; | |
| | | | f: Veillonellaceae; g: | |
| | | | Phascolarctobacterium; s: | |
| 290253 | 3 | 0.047619048 | k: Bacteria; p: Firmicutes; | 569 |
| | | | c: Clostridia; o: Clostridiales; | |
| | | | f: Ruminococcaceae; | |
| | | | g: Oscillospira; s: | |
| 290420 | 1 | 0.015873016 | k: Bacteria; p: Firmicutes; | 570 |
| | | | c: Clostridia; o: Clostridiales; | |
| | | | f: Ruminococcaceae; | |
| | | | g: Ruminococcus; s: | |
| 290442 | 8 | 0.126984127 | k: Bacteria; p: Firmicutes; | 571 |
| | | | c: Clostridia; o: Clostridiales; | |
| | | | f: Ruminococcaceae; | |
| | | | g: Ruminococcus; s: | |
| 291315 | 1 | 0.015873016 | k: Bacteria; p: Firmicutes; | 572 |
| | | | c: Clostridia; o: Clostridiales; | |
| | | | f: Veillonellaceae; g: | |
| | | | Phascolarctobacterium; s: | |
| 291512 | 19 | 0.301587302 | k: Bacteria; p: Firmicutes; | 573 |
| | | | c: Clostridia; o: Clostridiales; | |
| | | | f: Lachnospiraceae; | |
| | | | g: Coprococcus; s: | |
| 291644 | 14 | 0.222222222 | k: Bacteria; p: Firmicutes; | 574 |
| | | | c: Clostridia; o: Clostridiales; | |
| | | | f: Ruminococcaceae; | |
| | | | g: Ruminococcus; s: | |
| 291902 | 2 | 0.031746032 | k: Bacteria; p: Firmicutes; | 575 |
| | | | c: Clostridia; o: Clostridiales; | |
| | | | f: Ruminococcaceae; | |
| | | | g: Ruminococcus; s: | |
| 292091 | 5 | 0.079365079 | k: Bacteria; p: Firmicutes; | 576 |
| | | | c: Clostridia; o: Clostridiales; | |
| | | | f: Clostridiaceae; g:; s: | |
| 292289 | 1 | 0.015873016 | k: Bacteria; p: Proteobacteria; | 577 |
| | | | c: Gammeproteobacteria; | |
| | | | o: Enterobacteriales; f: | |
| | | | Enterobacteriaceae; g:; s: | |
| 292537 | 12 | 0.19047619 | k: Bacteria; p: Firmicutes; | 578 |
| | | | c: Clostridia; o: Clostridiales; | |
| | | | f: Ruminococcaceae; | |
| | | | g: Faecalibacterium; | |
| | | | s: prausnitzii | |
| 293508 | 1 | 0.015873016 | k: Bacteria; p: Firmicutes; | 579 |
| | | | c: Clostridia; o: Clostridiales; | |
| | | | f: Lachnospiraceae; | |
| | | | g: Coprococcus; s: | |
| 293883 | 6 | 0.095238095 | k: Bacteria; p: Firmicutes; | 580 |
| | | | c: Clostridia; o: Clostridiales; | |
| | | | f: Veillonellaceae; g: | |
| | | | Phascolarctobacterium; s: | |
| 294289 | 1 | 0.015873016 | k: Bacteria; p: Firmicutes; | 581 |
| | | | c: Clostridia; o: Clostridiales; | |
| | | | f: Ruminococcaceae; | |
| | | | g: Ruminococcus; s: | |
| 294791 | 6 | 0.095238095 | k: Bactceria; p: Firmicutes; | 582 |
| | | | c: Clostridia; o: Clostridiales; | |
| | | | f: Lachnospiraceae; | |
| | | | g: Coprococcus; s: | |
| 294909 | 1 | 0.015873016 | k: Bacteria; p: Bacteroidetes; | 583 |
| | | | c: Bacteroidia; o: Bacteroidales; | |
| | | | f: [Barnesiellaceae]; g:; s: | |
| 294922 | 1 | 0.015873016 | k: Bacteria; p: Firmicutes; | 584 |
| | | | c: Clostridia; o: Clostridiales; | |
| | | | f: Ruminococcaceae; | |
| | | | g: Faecalibacterium; | |
| | | | s: prausnitzii | |
| 295344 | 3 | 0.047619048 | k: Bacteria; p: Firmicutes; | 585 |
| | | | c: Clostridia; o: Clostridiales; | |
| | | | f: Ruminococcaceae; | |
| | | | g: Oscillospira; s: | |
| 295485 | 6 | 0.095238095 | k: Bacteria; p: Firmicutes; | 586 |
| | | | c: Clostridia; o: Clostridiales; | |
| | | | f: Ruminococcaceae; | |
| | | | g: Faecalibacterium; | |
| | | | s: prausnitzii | |
| 295629 | 4 | 0.063492063 | k: Bacteria; p: Firmicutes; | 587 |
| | | | c: Clostridia; o: Clostridiales; | |
| | | | f: Lachnospiraceae; | |
| | | | g: Coprococcus; s: | |
| 295974 | 1 | 0.015873016 | k: Bacteria; p: Firmicutes; | 588 |
| | | | c: Clostridiales; o: Clostridiales; | |
| | | | f: Clostridiaceae; g: Clostridium; | |
| | | | s: perfringens | |
| 296420 | 5 | 0.079365079 | k: Bacteria; p: Firmicutes; | 589 |
| | | | c: Clostridia; o: Clostridiales; | |
| | | | f: Lachnospiraceae; | |
| | | | g: Coprococcus; s: | |
| 296837 | 1 | 0.015873016 | k: Bacteria; p: Firmicutes; | 590 |
| | | | c: Clostridia; o: Clostridiales; | |
| | | | f: Clostridiaceae; g: Clostridium; s: | |
| 296945 | 25 | 0.396825397 | k: Bacteria; p: Proteobacteria; | 591 |
| | | | c: Betaproteobacteria; | |
| | | | o: Burkholderiales; | |
| | | | f: Alcaligenaceae; g: Sutterella; s: | |
| 296960 | 5 | 0.079365079 | k: Bacteria; p: Firmicutes; | 592 |
| | | | c: Clostridia; o: Clostridiales; | |
| | | | f: Lachnospiraceae; | |
| | | | g: Coprococcus; s: | |
| 297038 | 1 | 0.015873016 | k: Bacteria; p: Firmicutes; | 593 |
| | | | c: Clostridia; o: Clostridiales; | |
| | | | f: Ruminococcaceae; | |
| | | | g: Ruminococcus; s: | |
| 297160 | 1 | 0.015873016 | k: Bacteria; p: Firmicutes; | 594 |
| | | | c: Clostridia; o: Clostridiales; | |
| | | | f: Ruminococcaceae; | |
| | | | g: Ruminococcus; s: | |
| 297266 | 4 | 0.063492063 | k: Bacteria; p: Bacteroidetes; | 595 |
| | | | c: Bacteroidia; o: Bacteroidales; | |
| | | | f: Rikenellaceae; g:; s: | |
| 297385 | 2 | 0.031746032 | k: Bacteria; p: Firmicutes; | 596 |
| | | | c: Clostridia; o: Clostridiales; | |
| | | | f: Ruminococcaceae; | |
| | | | g: Oscillospira; s: | |
| 298325 | 13 | 0.206349206 | k: Bacteria; p: Firmicutes; | 597 |
| | | | c: Clostridia; o: Clostridiales; | |
| | | | f: Ruminococcaceae; | |
| | | | g: Ruminococcus; s: | |
| 298427 | 1 | 0.015873016 | k: Bacteria; p: Firmicutes; | 598 |
| | | | c: Bacilli; o: Lactobacillales; | |
| | | | f: Enterococcaceae; | |
| | | | g: Enterococcus; s: | |
| 299267 | 1 | 0.015873016 | k: Bacteria; p: Proteobacteria; | 599 |
| | | | c: Gammaproteobacteria; | |
| | | | o: Enterobacteriales; | |
| | | | f: Enterobacteriaceae; g:; s: | |
| 299474 | 10 | 0.158730159 | k: Bacteria; p: Firmicutes; | 600 |
| | | | c: Clostridia; o: Clostridiales; | |
| | | | f: Clostridiaceae; g:; s: | |
| 299820 | 31 | 0.492063492 | k: Bacteria; p: Firmicutes; | 601 |
| | | | c: Clostridia; o: Clostridiales; | |
| | | | f: Ruminococcaceae; | |
| | | | g: Oscillospira; s: | |
| 299837 | 4 | 0.063492063 | k: Bacteria; p: Firmicutes; | 602 |
| | | | c: Clostridia; o: Clostridiales; | |
| | | | f: Ruminococcaceae; | |
| | | | g: Faecalibacterium; | |
| | | | s: prausnitzii | |
| 300297 | 14 | 0.222222222 | k: Bacteria; p: Firmicutes; | 603 |
| | | | c: Clostridia; o: Clostridiales; | |
| | | | f: Lachnospiraceae; | |
| | | | g: Coprococcus; s: | |
| 300374 | 2 | 0.031746032 | k: Bacteria; p: Firmicutes; | 604 |
| | | | c: Clostridia; o: Clostridiales; | |
| | | | f: Ruminococcaceae; | |
| | | | g: Oscillospira; s: | |
| 300378 | 2 | 0.031746032 | k: Bacteria; p: Firmicutes; | 605 |
| | | | c: Clostridia; o: Clostridiales; | |
| | | | f: Clostridiaceae; g:; s: | |
| 300491 | 8 | 0.126984127 | k: Bacteria; p: Firmicutes; | 606 |
| | | | c: Clostridia; o: Clostridiales; | |
| | | | f: Ruminococcaceae; | |
| | | | g: Oscillospira; s: | |
| 300582 | 4 | 0.063492063 | k: Bacteria; p: Firmicutes; | 607 |
| | | | c: Clostridia; o: Clostridiales; | |
| | | | f: Clostridiaceae; g:; s: | |
| 300829 | 5 | 0.079365079 | k: Bacteria; p: Firmicutes; | 608 |
| | | | c: Clostridia; o: Clostridiales; | |
| | | | f: Clostridiaceae; | |
| | | | g: Clostridium; s: | |
| 300851 | 1 | 0.015873016 | k: Bacteria; p: Firmicutes; | 609 |
| | | | c: Clostridia; o: Clostridiales; | |
| | | | f: Ruminococcaceae; | |
| | | | g: Oscillospira; s: | |
| 300855 | 34 | 0.53968254 | k: Bacteria; p: Firmicutes; | 610 |
| | | | c: Clostridia; o: Clostridiales; | |
| | | | f: Clostridiaceae; g:; s: | |
| 300952 | 25 | 0.396825397 | k: Bacteria; p: Firmicutes; | 611 |
| | | | c: Clostridia; c: Clostridiales; | |
| | | | f: Lachnospiraceae; | |
| | | | g: Coprococcus; s: | |
| 301184 | 1 | 0.015873016 | k: Bacteria; p: Proteobacteria; | 612 |
| | | | c: Gammaproteobacteria; | |
| | | | o: Enterobacteriales; | |
| | | | f: Enterobacteriaceae; g:; s: | |
| 301449 | 1 | 0.015873016 | k: Bacteria; p: Firmicutes; | 613 |
| | | | c: Clostridia; o: Clostridiales; | |
| | | | f: Clostridiaceae; | |
| | | | g: Clostridium; s: | |
| 301910 | 10 | 0.158730159 | k: Bacteria; p: Firmicutes; | 614 |
| | | | c: Clostridia; o: Clostridiales; | |
| | | | f: Lachnospiraceae; | |
| | | | g: Coprococcus; s: | |
| 302545 | 1 | 0.015873016 | k: Bacteria; p: Actinobacteria; | 615 |
| | | | c: Coriobacteriia; | |
| | | | o: Coriobacteriales; | |
| | | | f: Coriobacteriaceae; g:; s: | |
| 302617 | 9 | 0.142857143 | k: Bacteria; p: Firmicutes; | 616 |
| | | | c: Clostridia; o: Clostridiales; | |
| | | | f: Lachnospiraceae; | |
| | | | g: Lachnospira; s: | |
| 302932 | 31 | 0.492063492 | k: Bacteria; p: Firmicutes; | 617 |
| | | | c: Clostridia; o: Clostridiales; | |
| | | | f: Clostridiaceae; g:; s: | |
| 303379 | 1 | 0.015873016 | k: Bacteria; p: Firmicutes; | 618 |
| | | | c: Clostridia; o: Clostridiales; | |
| | | | f: Clostridiaceae; g:; s: | |
| 303844 | 6 | 0.095238095 | k: Bacteria; p: Firmicutes; | 619 |
| | | | c: Clostridia; o: Clostridiales; | |
| | | | f: Lachnospiraceae; | |
| | | | g: Coprococcus; s: | |
| 303864 | 5 | 0.079365079 | k: Bacteria; p: Firmicutes; | 620 |
| | | | c: Clostridia; o: Clostridiales; | |
| | | | f: Lachnospiraceae; | |
| | | | g: Lachnospira; s: | |
| 304131 | 1 | 0.015873016 | k: Bacteria; p: Firmicutes; | 621 |
| | | | c: Clostridia; o: Clostridiales; | |
| | | | f: Ruminococcaceae; | |
| | | | g: Oscillospira; s: | |
| 304427 | 19 | 0.301587302 | k: Bacteria; p: Firmicutes; | 622 |
| | | | c: Clostridia; o: Clostridiales; | |
| | | | f: Ruminococcaceae; | |
| | | | g: Oscillospira; s: | |
| 304641 | 1 | 0.015873016 | k: Bacteria; p: Proteobacteria; | 623 |
| | | | c: Gammaproteobacteria; | |
| | | | o: Enterobacteriales; | |
| | | | f: Enterobacteriaceae; g:; s: | |
| 304777 | 6 | 0.095238095 | k: Bacteria; p: Firmicutes; | 624 |
| | | | c: Clostridia; o: Clostridiales; | |
| | | | f: Ruminococcaceae; | |
| | | | g: Oscillospira; s: | |
| 304779 | 9 | 0.14285143 | k: Bacteria; p: Firmicutes; | 625 |
| | | | c: Clostridia; o: Clostridiales; | |
| | | | f: Clostridiaceae; g: Clostridium; | |
| | | | s: perfringens | |
| 305016 | 1 | 0.015873016 | k: Bacteria; p: Firmicutes; | 626 |
| | | | c: Clostridia; o: Clostridiales; | |
| | | | f: Lachnospiraceae; | |
| | | | g: Lachnospira; s: | |
| 305141 | 6 | 0.095238095 | k: Bacteria; p: Actinobacteria; | 627 |
| | | | c: Coriobacteriia; | |
| | | | o: Coriobacteriales; | |
| | | | f: Coriobacteriaceae; g:; s: | |
| 305288 | 5 | 0.079365079 | k: Bacteria; p: Firmicutes; | 628 |
| | | | c: Clostridia; o: Clostridiales; | |
| | | | f: Clostridiaceae; | |
| | | | g: Clostridium; s: | |
| 305608 | 4 | 0.063492063 | k: Bacteria; p: Firmicutes; | 629 |
| | | | c: Clostridia; o: Clostridiales; | |
| | | | f: Veillonellaceae; g: | |
| | | | Phascolarctobacterium; s: | |
| 306315 | 4 | 0.063492063 | k: Bacteria; p: Firmicutes; | 630 |
| | | | c: Clostridia; o: Clostridiales; | |
| | | | f: Ruminococcaceae; | |
| | | | g: Oscillospira; s: | |
| 306413 | 8 | 0.126984127 | k: Bacteria; p: Firmicutes; | 631 |
| | | | c: Clostridia; o: Clostridiales; | |
| | | | f: Clostridiaceae; | |
| | | | g: Clostridium; s: | |
| 306704 | 1 | 0.015873016 | k: Bacteria; p: Firmicutes; | 632 |
| | | | c: Clostridia; o: Clostridiales; | |
| | | | f: Clostridiaceae; g:; s: | |
| 307127 | 5 | 0.079365079 | k: Bacteria; p: Firmicutes; | 633 |
| | | | c: Clostridia; o: Clostridiales; | |
| | | | f: Lachnospiraceae; | |
| | | | g: Lachnospira; s: | |
| 307238 | 18 | 0.285714286 | k: Bacteria; p: Firmicutes; | 634 |
| | | | c: Clostridia; o: Clostridiales; | |
| | | | f: Ruminococcaceae; | |
| | | | g: Oscillospira; s: | |
| 308912 | 1 | 0.015873016 | k: Bacteria; p: Firmicutes; | 635 |
| | | | c: Clostridia; o: Clostridiales; | |
| | | | f: Christenellaceae; g:; s: | |
| 308957 | 1 | 0.015873016 | k: Bacteria; p: Bacteroidetes; | 636 |
| | | | c: Bacteroidia; o: Bacteroidales; | |
| | | | f: Rikenellaceae; g:; s: | |
| 309127 | 1 | 0.01583016 | k: Bacteria; p: Firmicutes; | 637 |
| | | | c: Clostridia; o: Clostridiales; | |
| | | | f: Clostridiaceae; g:; s: | |
| 309433 | 3 | 0.047619048 | k: Bacteria; p: Fiirmicutes; | 638 |
| | | | c: Clostridia; o: Clostridiales; | |
| | | | f: Lachnospiraceae; | |
| | | | g: Lachnospira; s: | |
| 309658 | 2 | 0.031746032 | k: Bacteria; p: Frimicutes; | 639 |
| | | | c: Clostridia; o: Clostridiales; | |
| | | | f: Clostridiaceae; g: Clostridium; s: | |
| 309929 | 1 | 0.015873016 | k: Bacteria; p: Firmicutes; | 640 |
| | | | c: Clostridia; o: Clostridiales; | |
| | | | f: Ruminococcaceae; | |
| | | | g: Ruminococcus; s: | |
| 310301 | 1 | 0.015873016 | k: Bacteria; p: Firmicutes; | 641 |
| | | | c: Clostridia; o: Clostridiales; | |
| | | | f: Ruminococcaceae; | |
| | | | g: Faecalibacterium; | |
| | | | s: prausnitzii | |
| 310945 | 1 | 0.015873016 | k: Bacteria; p: Firmicutes; | 642 |
| | | | c: Clostridia; o: Clostridiales; | |
| | | | f: Ruminococcaceae; | |
| | | | g: Oscillospira; s: | |
| 312070 | 14 | 0.222222222 | k: Bacteria; p: Firmicutes; | 643 |
| | | | c: Clostridia; o: Clostridiales; | |
| | | | f: [Mogibacteriaceae]; g:; s: | |
| 312515 | 1 | 0.015873016 | k: Bacteria; p: Firmicutes; | 644 |
| | | | c: Clostridia; o: Clostridiales; | |
| | | | f: Ruminococcaceae; | |
| | | | g: Oscillospira; s: | |
| 313387 | 1 | 0.015873016 | k: Bacteria; p: Firmicutes; | 645 |
| | | | c: Clostridia; o: Clostridiales; | |
| | | | f: Lachnospiraceae; | |
| | | | g: Coprococcus; s: | |
| 313672 | 2 | 0.031746032 | k: Bacteria; p: Firmicutes; | 646 |
| | | | c: Clostridia; o: Clostridiales; | |
| | | | f: Lachnospiraceae; | |
| | | | g: Lachnospira; s: | |
| 314053 | 2 | 0.095238095 | k: Bacteria; p: Firmicutes; | 647 |
| | | | c: Claostridia; o: Clostridiales; | |
| | | | f: Clostridiaceae; g: Clostridium; s: | |
| 314582 | 2 | 0.031746032 | k: Bacteria; p: Firmicutes; | 648 |
| | | | c: Clostridia; o: Clostridiales; | |
| | | | f: Ruminococcaceae; | |
| | | | g: Oscillospira; s: | |
| 315200 | 3 | 0.047619048 | k: Bacteria; p: Firmicutes; | 649 |
| | | | c: Clostridia; o: Clostridiales; | |
| | | | f: Ruminococceae; | |
| | | | g: Ruminococcus; s: | |
| 315382 | 3 | 0.047619048 | k: Bacteria; p: Firmicutes; | 650 |
| | | | c: Clostridia; o: Clostridiales; | |
| | | | f: Clostridiaceae; g: Clostridium; s: | |
| 315391 | 20 | 0.317460317 | k: Bacteria; p: Firmicutes; | 651 |
| | | | c: Clostridia; o: Clostridiales; | |
| | | | f: Ruminococcaceae; | |
| | | | g: Faecalibacterium; | |
| | | | s: prausnitzii | |
| 315831 | 2 | 0.031746032 | k: Bacteria; p: Firmicutes; | 652 |
| | | | c: Clostridia; o: Clostridiales; | |
| | | | f: [Mogibacteriaceae]; g:; s: | |
| 315846 | 9 | 0.142857143 | k: Bacteria; p: Bacteroidetes; | 653 |
| | | | c: Bacteroidia; o: Bacteroidales; | |
| | | | f: [Barnesiellaceae]; g:; s: | |
| 316378 | 3 | 0.047619048 | k: Bacteria; p: Firmicutes; | 654 |
| | | | c: Clostridia; o: Clostridiales; | |
| | | | f: Clostridiaceae; g:; s: | |
| 316732 | 27 | 0.428571429 | k: Bacteria; p: Firmicutes; | 655 |
| | | | c: Clostridia; o: Clostridiales; | |
| | | | f: Lachnospiraceae; | |
| | | | g: Lachnospira; s: | |
| 317948 | 1 | 0.015873016 | k: Bacteria; p: Firmicutes; | 656 |
| | | | c: Clostridia; o: Clostridiales; | |
| | | | f: Ruminococcaceae; | |
| | | | g: Ruminococcus; s: | |
| 318777 | 13 | 0.206349206 | k: Bacteria; p: Firmicutes; | 657 |
| | | | c: Clostridia; o: Clostridiales; | |
| | | | f: Veillonellaceae; g: | |
| | | | Phascolarctobacterium; s: | |
| 318865 | 4 | 0.063492063 | k: Bacteria; p: Firmicutes; | 658 |
| | | | c: Clostridia; o: Clostridiales; | |
| | | | f: Lachnospiraceae; | |
| | | | g: Lachnospira; s: | |
| 318928 | 14 | 0.222222222 | k: Bacteria; p: Firmicutes; | 659 |
| | | | c: Clostridia; o: Clostridiales; | |
| | | | f: Ruminococcaceae; | |
| | | | g: Ruminococcus; s: | |
| 319260 | 3 | 0.047619048 | k: Bacteria; p: Firmicutes; | 660 |
| | | | c: Clostridia; o: Clostridiales; | |
| | | | f: [Mogibacteriaceae]; g:; s: | |
| 319275 | 51 | 0.80952381 | k: Bacteria; p: Firmicutes; | 661 |
| | | | c: Clostridia; o: Clostridiales; | |
| | | | f: Ruminococcaceae; | |
| | | | g: Faecalibacterium; | |
| | | | s: prausnitzii | |
| 319455 | 20 | 0.317460317 | k: Bacteria; p: Firmicutes; | 662 |
| | | | c: Clostridia; o: Clostridiales; | |
| | | | f: Christensenellaceae; g:; s: | |
| 320224 | 6 | 0.095238095 | k: Bacteria; p: Frimicutes; | 663 |
| | | | c: Clostridia; o: Clostridiales; | |
| | | | f: Ruminococcaceae; | |
| | | | g: Ruminococcus; s: | |
| 320322 | 29 | 0.46031746 | k: Bacteria; p: Firmicutes; | 664 |
| | | | c: Clostridia; o: Clostridiales; | |
| | | | f: Lachnospiraceae; | |
| | | | g: Lachnospira; s: | |
| 321096 | 1 | 0.015873016 | k: Bacteria; p: Firmicutes; | 665 |
| | | | c: Clostridia; o: Clostridiales; | |
| | | | f: Clostridaceae; g:; s: | |
| 321517 | 5 | 0.079365079 | k: Bacteria; p: Firmicutes; | 666 |
| | | | c: Clostridia; o: Clostridiales; | |
| | | | f: Ruminococcaceae; | |
| | | | g: Ruminococcus; s: | |
| 321774 | 16 | 0.253968254 | k: Bacteria; p: Firmicutes; | 667 |
| | | | c: Clostridia; o: Clostridiales; | |
| | | | f: Lachnospiraceae; | |
| | | | g: Coprococcus; s: | |
| 321907 | 4 | 0.063492063 | k: Bacteria; p: Firmicutes; | 668 |
| | | | c: Clostridia; o: Clostridiales; | |
| | | | f: Lachnospiraceae; | |
| | | | g: Coprococcus; s: | |
| 322223 | 2 | 0.031746032 | k: Bacteria; p: Bacteroidetes; | 669 |
| | | | c: Bacteroidia; o: Bacteroidales; | |
| | | | f: Rikenellaceae; g:; s: | |
| 322258 | 6 | 0.095238095 | k: Bacteria; p: Firmicutes; | 670 |
| | | | c: Clostridia; o: Clostridiales; | |
| | | | f: Ruminococcaceae; | |
| | | | g: Oscillospira; s: | |
| 322367 | 2 | 0.031746032 | k: Bacteria; p: Firmicutes; | 671 |
| | | | c: Clostridia; o: Clostridiales; | |
| | | | f: Clostridiaceae; g:; s: | |
| 322835 | 15 | 0.238095238 | k: Bacteria; p: Firmicutes; | 672 |
| | | | c: Clostridia; o: Clostridiales; | |
| | | | f: Lachnospiraceae; | |
| | | | g: Lachnospira; s: | |
| 323135 | 10 | 0.158730159 | k: Bacteria; p: Firmicutes; | 674 |
| | | | c: Clostridia; o: Clostridiales; | |
| | | | f: Ruminococcaceae; | |
| | | | g: Ruminococcus; s: | |
| 323403 | 1 | 0.015873016 | k: Bacteria; p: Firmicutes; | 675 |
| | | | c: Clostridia; o: Clostridiales; | |
| | | | f: Ruminococcaceae; | |
| | | | g: Oscillospira; s: | |
| 324214 | 7 | 0.111111111 | k: Bacteria; p: Firmicutes; | 676 |
| | | | c: Clostridia; o: Clostridiales; | |
| | | | f: Ruminococcaceae; | |
| | | | g: Oscillospira; s: | |
| 324315 | 1 | 0.015873016 | k: Bacteria; p: Firmicutes; | 677 |
| | | | c: Clostridia; o: Clostridiales; | |
| | | | f: Clostridiaceae; g: Clostridium; s: | |
| 324882 | 3 | 0.047619048 | k: Bacteria; p: Firmicutes; | 678 |
| | | | c: Clostridia; o: Clostridiales; | |
| | | | f: [Mogibacteriaceae]; g:; s: | |
| 325599 | 4 | 0.063492063 | k: Bacteria; p: Firmicutes; | 679 |
| | | | c: Clostridia; o: Clostridiales; | |
| | | | f: Lachnospiraceae; | |
| | | | g: Lachnospira; s: | |
| 326991 | 27 | 0.428571429 | k: Bacteria; p: Firmicutes; | 680 |
| | | | c: Clostridia; o: Clostridiales; | |
| | | | f: Clostridiaceae; g: Clostridium; s: | |
| 327298 | 3 | 0.047619048 | k: Bacteria; p: Firmicutes; | 681 |
| | | | c: Clostridia; o: Clostridiales; | |
| | | | f: Clostridiaceae; g: Clostridium; s: | |
| 327598 | 8 | 0.126984127 | k: Bacteria; p: Firmicutes; | 682 |
| | | | c: Clostridia; o: Clostridiales; | |
| | | | f: Christensenellaceae; g:; s: | |
| 328059 | 1 | 0.015873016 | k: Bacteria; p: Firmicutes; | 683 |
| | | | c: Clostridia; o: Clostridiales; | |
| | | | f: Clostridiaceae; g:; s: | |
| 328905 | 57 | 0.904761905 | k: Bacteria; p: Firmicutes; | 684 |
| | | | c: Clostridia; o: Clostridiales; | |
| | | | f: Ruminococcaceae; | |
| | | | g: Oscillospira; s: | |
| 328993 | 1 | 0.015873016 | k: Bacteria; p: Firmicutes; | 685 |
| | | | c: Clostridia; o: Clostridiales; | |
| | | | f: Lachnospiraceae; | |
| | | | g: Coprococcus; s: | |
| 329096 | 2 | 0.031746032 | k: Bacteria; p: Proteobacteria; | 686 |
| | | | c: Gammaproteobacteria; | |
| | | | o: Enterobacteriales; | |
| | | | f: Eneterobacteriaceae; g:; s: | |
| 329688 | 10 | 0.158730159 | k: Bacteria; p: Actinobacteria; | 687 |
| | | | c: Coriobacteriia; | |
| | | | o: Coriobacteriales; | |
| | | | f: Coriobacteriaceae; g: Collinsella; | |
| | | | s: stercoris | |
| 329798 | 6 | 0.095238095 | k: Bacteria; p: Firmicutes; | 688 |
| | | | c: Clostridia; o: Clostridiales; | |
| | | | f: Clostridiaceae; g:; s: | |
| 330714 | 2 | 0.031746032 | k: Bacteria; p: Firmicutes; | 689 |
| | | | c: Clostridia; o: Clostridiales; | |
| | | | f: Clostridiaceae; g: Clostridium; s: | |
| 331881 | 21 | 0.333333333 | k: Bacteria; p: Firmicutes; | 690 |
| | | | c: Clostridia; o: Clostridiales; | |
| | | | f: Clostridiaceae; g:; s: | |
| 332027 | 7 | 0.111111111 | k: Bacteria; p: Firmicutes; | 691 |
| | | | c: Clostridia; o: Clostridiales; | |
| | | | f: Christensenellaceae; g:; s: | |
| 332163 | 14 | 0.222222222 | k: Bacteria; p: Firmicutes; | 692 |
| | | | c: Clostridia; o: Clostridiales; | |
| | | | f: Lachnospiraceae; | |
| | | | g: Lachnospira; s: | |
| 332450 | 3 | 0.047619048 | k: Bacteria; p: Firmicutes; | 693 |
| | | | c: Clostridia; o: Clostridiales; | |
| | | | f: Lachnospiraceae; g: Coprococcus; | |
| | | | s: eutactus | |
| 333096 | 1 | 0.015873016 | k: Bacteria; p: Firmicutes; | 694 |
| | | | c: Clostridia; o: Clostridiales; | |
| | | | f: Ruminococcaceae; | |
| | | | g: Ruminococcus; s: | |
| 333189 | 2 | 0.031746032 | k: Bacteria; p: Firmicutes; | 695 |
| | | | c: Clostridia; o: Clostridiales; | |
| | | | f: Ruminococcaceae; | |
| | | | g: Oscillospira; s: | |
| 333380 | 40 | 0.063492063 | k: Bacteria; p: Proteobacteria; | 696 |
| | | | c: Betaproteobacteria; | |
| | | | o: Burkholderiales; | |
| | | | f: Alcaligenaceae; g: Sutterella; s: | |
| 333768 | 7 | 0.111111111 | k: Bacteria; p: Firmicutes; | 697 |
| | | | c: Clostridia; o: Clostridiales; | |
| | | | f: Ruminococcaceae; | |
| | | | g: Oscillospira; s: | |
| 334215 | 4 | 0.063492063 | k: Bacteria; p: Firmicutes; | 698 |
| | | | c: Clostridia; o: Clostridiales; | |
| | | | f: Ruminococcaceae; | |
| | | | g: Oscillospira; s: | |
| 334935 | 1 | 0.015873016 | k: Bacteria; p: Firmicutes; | 699 |
| | | | c: Clostridia; o: Clostridiales; | |
| | | | f: Ruminococcaceae; | |
| | | | g: Oscillospira; s: | |
| 335910 | 22 | 0.349206349 | k: Bacteria; p: Firmicutes; | 700 |
| | | | c: Clostridia; o: Clostridiales; | |
| | | | f: Lachnospiraceae; | |
| | | | g: Coprococcus; s: | |
| 336205 | 7 | 0.111111111 | k: Bacteria; p: Firmicutes; | 701 |
| | | | c: Clostridia; o: Clostridiales; | |
| | | | f: Lachnospiraceae; | |
| | | | g: Coprococcus; s: | |
| 336338 | 2 | 0.031746032 | k: Bacteria; p: Firmicutes; | 702 |
| | | | c: Clostridia; o: Clostridiales; | |
| | | | f: Clostridiaceae; g:; s: | |
| 337048 | 8 | 0.126984127 | k: Bacteria; p: Firmicutes; | 703 |
| | | | c: Clostridia; o: Clostridiales; | |
| | | | f: Clostridiaceae; g:; s: | |
| 337511 | 14 | 0.222222222 | k: Bacteria; p: Firmicutes; | 704 |
| | | | c: Clsotridia; o: Clostridiales; | |
| | | | f: Clostridiaceae; g:; s: | |
| 338195 | 3 | 0.047619048 | k: Bacteria; p: Firmicutes; | 705 |
| | | | c: Clostridia; o: Clostridiales; | |
| | | | f: Christensenellaceae; g:; s: | |
| 339087 | 2 | 0.031746032 | k: Bacteria; p: Firmicutes; | 706 |
| | | | c: Clostridia; o: Clostridiales; | |
| | | | f: Ruminococcaceae; | |
| | | | g: Faecaliibacterium; | |
| | | | s: prausnitzii | |
| 339494 | 50 | 0.793650794 | k: Bacteria; p: Firmicutes; | 707 |
| | | | c: Clostridia; o: Clostridiales; | |
| | | | f: Ruminococcaceae; | |
| | | | g: Faecalibacterium; | |
| | | | s: prausnitzii | |
| 339712 | 2 | 0.031746032 | k: Bacterium; p: Firmicutes; | 708 |
| | | | c: Clostridia; o: Clostridiales; | |
| | | | f: Clostridiaceae; g:; s: | |
| 340170 | 11 | 0.174603175 | k: Bacteria; p: Firmicutes; | 709 |
| | | | c: Clostridia; o: Clostridiales; | |
| | | | f: Ruminococcaceae; | |
| | | | g: Ruminococcus; s: | |
| 340219 | 1 | 0.015873016 | k: Bacteria; p: Firmicutes; | 710 |
| | | | c: Clostridia; o: Clostridiales; | |
| | | | f: Ruminococcaceae; | |
| | | | g: Faecalibacterium; | |
| | | | p: prausnitzii | |
| 340794 | 2 | 0.031746032 | k: Bacteria; p: Firmicutes; | 711 |
| | | | c: Clostridia; o: Clostridiales; | |
| | | | f: Ruminococcaceae; | |
| | | | g: Ruminococcus; s: | |
| 341004 | 4 | 0.063492063 | k: Bacteria; p: Firmicutes; | 712 |
| | | | c: Clostridia; o: Clostridiales; | |
| | | | f: Ruminococcaceae; | |
| | | | g: Oscillospira; s: | |
| 341489 | 5 | 0.079365079 | k: Bacteria; p: Firmicutes; | 713 |
| | | | c: Clostridia; o: Clostridiales; | |
| | | | f: Ruminococcaceae; | |
| | | | g: Ruminococcus; s: | |
| 341885 | 9 | 0.142857143 | k: Bacteria; p: Firmicutes; | 714 |
| | | | c: Clostridia; o: Clostridiales; | |
| | | | f: Clostridiaceae; g:; s: | |
| 341920 | 21 | 0.333333333 | k: Bacteria; p: Firmicutes; | 715 |
| | | | c: Clostridia; o: Clostridiales; | |
| | | | f: Ruminococcaceae; | |
| | | | g: Faecalibacterium; | |
| | | | s: prausnitzii | |
| 342268 | 1 | 0.015873016 | k: Bacteria; p: Firmicutes; | 716 |
| | | | c: Clostridia; o: Clostridiales; | |
| | | | f: Peptostreptococcaceae; | |
| | | | g: Clostridium; | |
| | | | s: metallolevans | |
| 342375 | 3 | 0.047619048 | k: Bacteria; p: Firmicutes; | 717 |
| | | | c: Clostridia; o: Clostridiales; | |
| | | | f: Lachnospiraceae; | |
| | | | g: Lachnospira; s: | |
| 342504 | 5 | 0.079365079 | k: Bacteria; p: Firmicutes; | 718 |
| | | | c: Clostridia; o: Clostridiales; | |
| | | | f: Clostridiaceae; g:; s: | |
| 342666 | 2 | 0.031746032 | k: Bacteria; p: Firmicutes; | 719 |
| | | | c: Clostridia; o: Clostridiales; | |
| | | | f: Clostridiaceae; g:; s: | |
| 343090 | 6 | 0.095238095 | k: Bacteria; p: Firmicutes; | 720 |
| | | | c: Clostridia; o: Clostridiales; | |
| | | | f: Clostridiaceae; g:; s: | |
| 343104 | 3 | 0.047619048 | k: Bacteria; p: Firmicutes; | 721 |
| | | | c: Clostridia; o: Clostridiales; | |
| | | | f: Clostridiaceae; g:; s: | |
| 344798 | 1 | 0.015873016 | k: Bacteria; p: Firmicutes; | 722 |
| | | | c: Clostridia; o: Clostridiales; | |
| | | | f: Clostridiaceae; g:; s: | |
| 345362 | 2 | 0.031746032 | k: Bacteria; p: Proteobacteria; | 723 |
| | | | c: Gammaproteobacteria; | |
| | | | o: Enterobacteriales; | |
| | | | f: Enterobacteriaceae; g:; s: | |
| 345540 | 10 | 0.158730159 | k: Bacteria; p: Proteobacteria; | 724 |
| | | | c: Gammaproteobacteria; | |
| | | | o: Enterobacteriales; | |
| | | | f: Enterobacteriaceae; g:; s: | |
| 346302 | 3 | 0.047619048 | k: Bacteria; p: Firmicutes; | 725 |
| | | | c: Clostridia; o: Clostridia; | |
| | | | f: Lachnocpiraceae; | |
| | | | g: Lachnospira; s: | |
| 346793 | 2 | 0.031746032 | k: Bacteria; p: Firmicutes; | 726 |
| | | | c: Clostridia; o: Clostridiales; | |
| | | | f: Veillonellaceae; | |
| | | | g: Phascolarctobacterium; s: | |
| 347690 | 32 | 0.507936508 | k: Bacteria; p: Firmicutes; | 727 |
| | | | c: Clostridia; o: Clostridiales; | |
| | | | f: Ruminococcaceae; | |
| | | | g: Oscillospira; s: | |
| 348009 | 2 | 0.031746032 | k: Bacteria; p: Firmicutes; | 728 |
| | | | c: Clostridia; o: Clostridiales; | |
| | | | f: Ruminococcaceae; | |
| | | | g: Oscillospira; s: | |
| 349257 | 27 | 0.428571429 | k: Bacteria; p: Firmicutes; | 729 |
| | | | c: Clostridia; o: Clostridiales; | |
| | | | f: Lachnospiraceae; | |
| | | | g: Lachnospira; s: | |
| 349431 | 18 | 0.285714286 | k: Bacteria; p: Firmicutes; | 730 |
| | | | c: Clostridia; o: Clostridiales; | |
| | | | f: Ruminococcaceae; | |
| | | | g: Ruminococcus; s: | |
| 349798 | 1 | 0.015873016 | k: Bacteria; p: Firmicutes; | 731 |
| | | | c: Clostridia; o: Clostridiales; | |
| | | | f: Ruminococcaceae; | |
| | | | g: Oscillospira; s: | |
| 349884 | 1 | 0.015873016 | k: Bacteria; p: Firmicutes; | 732 |
| | | | c: Clostridia; o: Clostridiales; | |
| | | | f: Ruminococcaceae; | |
| | | | g: Ruminococcus; s: | |
| 350224 | 1 | 0.015873016 | k: Bacteria; p: Firmicutes; | 733 |
| | | | c: Clostridia; o: Clostridiales; | |
| | | | f: Ruminococcaceae; | |
| | | | g: Ruminococcus; | |
| | | | s: flavefaciens | |
| 350865 | 18 | 0.285714286 | k: Bacteria; p: Firmicutes; | 734 |
| | | | c: Clostridia; o: Clostridiales; | |
| | | | f: Lachnospiraceae; | |
| | | | g: Lachnospira; s: | |
| 351357 | 6 | 0.095238095 | k: Bacteria; p: Firmicutes; | 735 |
| | | | c: Clostridia; o: Clostridiales; | |
| | | | f: Clostridiaceae; g:; s: | |
| 352747 | 39 | 0.619047619 | k: Bacteria; p: Firmicutes; | 736 |
| | | | c: Clostridia; o: Clostridiales; | |
| | | | f: Lachnospiraceae; | |
| | | | g: Coprococcus; s: | |
| 353632 | 2 | 0.031746032 | k: Bacteria; p: Firmicutes; | 737 |
| | | | c: Clostridia; o: Clsotridiales; | |
| | | | f: Ruminococcaceae; | |
| | | | g: Ruminococcus; s: | |
| 355298 | 1 | 0.015873016 | k: Bacteria; p: Firmicutes; | 738 |
| | | | c: Clostridia; f: Clostridiales | |
| | | | f: Clostridiaceae; g:; s: | |
| 355450 | 27 | 0.428571429 | k: Bacteria; p: Firmicutes; | 739 |
| | | | c: Clostridia; o: Clostridiales; | |
| | | | f: Ruminococcaceae; | |
| | | | g: Oscillospira; s: | |
| 355975 | 3 | 0.047619048 | k: Bacteria; p: Firmicutes; | 740 |
| | | | c: Clostridia; o: Clostridiales; | |
| | | | f: Lachnospiraceae; | |
| | | | g: Lachnospira; s: | |
| 356644 | 8 | 0.126984127 | k: Bacteria; p: Firmicutes; | 741 |
| | | | c: Clostridia; o: Clostridiales; | |
| | | | f: Lachnospiraceae; | |
| | | | g: Lachnospira; s: | |
| 356745 | 40 | 0.634920635 | k: Bacteria; p: Firmicutes; | 742 |
| | | | c: Clostridia; o: Clostridiales; | |
| | | | f: Ruminococcaceae; | |
| | | | g: Ruminococcus; s: | |
| 357046 | 59 | 0.936507937 | k: Bacteria; p: Bacteroidetes; | 743 |
| | | | c: Bacteroidia; o: Bacteroidales; | |
| | | | f: Rikenellaceae; g:; s: | |
| 357261 | 40 | 0.634920635 | k: Bacteria; p: Firmicutes; | 744 |
| | | | c: Clostridia; o: Clostridiales; | |
| | | | f: Ruminococcaceae; | |
| | | | g: Ruminococcus; s: | |
| 357449 | 1 | 0.015873016 | k: Bacteria; p: Firmicutes; | 745 |
| | | | c: Clostridia; o: Clostridiales; | |
| | | | f: Lachnospiraceae; | |
| | | | g: Coprococcus; s: | |
| 357529 | 21 | 0.333333333 | k: Bacteria; p: Firmicutes; | 746 |
| | | | c: Clostridia; o: Clostridiales; | |
| | | | f: Clostridiaceae; | |
| | | | g: Colstridium; s: | |
| 357724 | 48 | 0.761904762 | k: Bacteria; p: Firmicutes; | 747 |
| | | | c: Clostridia; o: Clostridiales; | |
| | | | f: Ruminococcaceae; | |
| | | | g: Ruminococcus; | |
| | | | s: bromii | |
| 357849 | 45 | 0.714285714 | k: Bacteria; p: Actinobacteria; | 748 |
| | | | c: Coriobacteriia; | |
| | | | o: Coriobacteriales; | |
| | | | f: Coriobacteriaceae; | |
| | | | g: Collinsella; | |
| | | | s: aerofaciens | |
| 358008 | 2 | 0.031746032 | k: Bacteria; p: Firmicutes; | 749 |
| | | | c: Clostridia; o: Clostridiales; | |
| | | | f: Ruminococcaceae; | |
| | | | g: Faecalibacterium; | |
| | | | s: prausnitzii | |
| 358834 | 16 | 0.253968254 | k: Bacteria; p: Firmicutes; | 750 |
| | | | c: Clostridia; o: Clostridiales; | |
| | | | f: Lachnospiraceae; | |
| | | | g: Coprococcus; s: | |
| 359563 | 4 | 0.063492063 | k: Bacteria; p: Firmicutes; | 751 |
| | | | c: Clostridia; o: Clostridiales; | |
| | | | f: Ruminococcaceae; | |
| | | | g: Oscillospira; s: | |
| 359950 | 4 | 0.063492063 | k: Bacteria; p: Firmicutes; | 752 |
| | | | c: Clostridia; o: Clostridiales; | |
| | | | f: Ruminococcaceae; | |
| | | | g: Ruminococcus; s: | |
| 359993 | 12 | 0.19047619 | k: Bacteria; p: Firmicutes; | 753 |
| | | | c: Clostridia; o: Clostridiales; | |
| | | | f: Lachnospiraceae; g: Coprococcus; | |
| | | | s: eutactus | |
| 360147 | 1 | 0.015873016 | k: Bacteria; p: Firmicutes; | 754 |
| | | | c: Clostridia; o: Clostridiales; | |
| | | | f: Lachnospiraceae; | |
| | | | g: Coprococcus; s: | |
| 361581 | 1 | 0.015873016 | k: Bacteria; p: Firmicutes; | 755 |
| | | | c: Clostridia; o: Clostridiales; | |
| | | | f: Ruminococcaceae; | |
| | | | g: Faecalibacterium; | |
| | | | s: prausnitzii | |
| 361702 | 55 | 0.873015873 | k: Bacteria; p: Firmicutes; | 756 |
| | | | c: Clostridia; o: Clostridiales; | |
| | | | f: Ruminococcaceae; | |
| | | | g: Ruminococcus; s: | |
| 361966 | 1 | 0.015873016 | k: Bacteria; p: Firmicutes; | 757 |
| | | | c: Clostridia; o: Clostridiales; | |
| | | | f: Ruminococcaceae; | |
| | | | g: Faecalibacterium; | |
| | | | s: prausnitzii | |
| 362094 | 2 | 0.031746032 | k: Bacteria; p: Firmicutes; | 758 |
| | | | c: Clostridia; o: Clostridiales; | |
| | | | f: Ruminococcaceae; | |
| | | | g: Faecalibacterium; | |
| | | | s: prausnitzii | |
| 362327 | 1 | 0.015873016 | k: Bacteria; p: Firmicutes; | 759 |
| | | | c: Clostridia; o: Clostridiales; | |
| | | | f: Ruminococcaceae; | |
| | | | g: Faecalibacterium; | |
| | | | s: prausnitzii | |
| 362342 | 1 | 0.015873016 | k: Bacteria; p: Firmicutes; | 760 |
| | | | c: Clostridia; o: Clostridiales; | |
| | | | f: Ruminococcaceae; | |
| | | | g: Ruminococcus; s: | |
| 362501 | 2 | 0.031746032 | k: Bacteria; p: Firmicytes; | 761 |
| | | | c: Clostridia; o: Clostridiales; | |
| | | | f: Lachnospiraceae; | |
| | | | g: Coprococcus; s: | |
| | | | eutactus | |
| 362765 | 4 | 0.063492063 | k: Bacteria; p: Firimcutes; | 762 |
| | | | c: Clostridia; o: Clostridiales; | |
| | | | f: Ruminococcaceae; | |
| | | | g: Ruminococcus; s: | |
| 362793 | 3 | 0.047619048 | k: Bacteria; p: Firmicutes; | 763 |
| | | | c: Clostridia; o: Clostridiales; | |
| | | | f: Ruminococcaceae; | |
| | | | g: Oscillspira; s: | |
| 362991 | 14 | 0.222222222 | k: Bacteria; p: Firmicutes; | 764 |
| | | | c: Clostridia; o: Clostridiales; | |
| | | | f: Ruminococcaceae; | |
| | | | g: Ruminococcus; s: | |
| 363358 | 23 | 0.365079365 | k: Bacteria; p: Firmicutes; | 765 |
| | | | c: Clostridia; o: Clostridiales; | |
| | | | f: Ruminococcaceae; | |
| | | | g: Faecalibacterium; | |
| | | | s: prausnitzii | |
| 363389 | 16 | 0.253968254 | k: Bacteria; p: Firmicutes; | 766 |
| | | | c: Clostridia; o: Clostridiales; | |
| | | | f: Clostridiaceae; g: Clostridium; s: | |
| 363442 | 3 | 0.047619048 | k: Bacteria; p: Firmicutes; | 767 |
| | | | c: Clostridia; o: Clostridiales; | |
| | | | f: Ruminococcaceae; | |
| | | | g: Faecalibacterium; | |
| | | | s: prausnitzii | |
| 363519 | 21 | 0.333333333 | k: Bacteria; p: Firmicutes; | 768 |
| | | | c: Clostridia; o: Clostridiales; | |
| | | | f: Christensenellaceae; g:; s: | |
| 363646 | 2 | 0.031746032 | k: Bacteria; p: Firmicutes; | 769 |
| | | | c: Clostridia; o: Clostridiales; | |
| | | | f: Ruminococcaceae; | |
| | | | g: Ruminococcus; s: | |
| 363794 | 22 | 0.349206349 | k: Bacteria; p: Actinobacteria; | 770 |
| | | | c: Coriobacteriia; | |
| | | | o: Coriobacteriales; | |
| | | | f: Coriobacteriaceae; | |
| | | | g: Collinsella; | |
| | | | s: aerofaciens | |
| 364341 | 15 | 0.238095238 | k: Bacteria; p: Firmicutes; | 771 |
| | | | c: Clostridia; o: Clostridiales; | |
| | | | f: Ruminococcaceae; | |
| | | | g: Oscillospira; s: | |
| 364563 | 6 | 0.095238095 | k: Bacteria; p: Firmicutes; | 772 |
| | | | c: Clostridia; o: Clostridiales; | |
| | | | f: Ruminococaceae; | |
| | | | g: Faecalibacterium; | |
| | | | s: prausnitzii | |
| 365118 | 6 | 0.095238095 | k: Bacteria; p: Firmicutes; | 773 |
| | | | c: Clostridia; o: Clostridiales; | |
| | | | f: Lachnospiraceae; | |
| | | | g: Coprococcus; s: | |
| 365181 | 12 | 0.19047619 | k: Bacteria; p: Actinobacteria; | 774 |
| | | | c: Coriobacteriia; | |
| | | | o: Coriobacteriales; | |
| | | | f: Coriobacteriaceae; | |
| | | | g: Collinsella; | |
| | | | s: aerofaciens | |
| 365628 | 24 | 0.380952381 | k: Bacteria; p: Firmicutes; | 775 |
| | | | c: Clostridia; o: Clostridiales; | |
| | | | f: Veillonellaceae; | |
| | | | g: Phascolarctobacterium; s: | |
| 365717 | 23 | 0.365079365 | k: Bacteria; p: Firmicutes; | 776 |
| | | | c: Clostridia; o: Clostridiales; | |
| | | | f: Ruminococcaceae; | |
| | | | g: Faecalibacterium; | |
| | | | s: prausnitzii | |
| 365842 | 1 | 0.015873016 | k: Bacteria; p: Firmicutes; | 777 |
| | | | c: Clostridia; o: Clostridiales; | |
| | | | f: Ruminococcaceae; | |
| | | | g: Faecalibacterium; | |
| | | | s: prausnitzii | |
| 365965 | 10 | 0.158730159 | k: Bacteria; p: Firmicutes; | 778 |
| | | | c: Clostridia; o: Clostridiales; | |
| | | | f: Ruminococcaceae; | |
| | | | g: Ruminococcus; s: | |
| 366068 | 3 | 0.047619048 | k: Bacteria; p: Firmicutes; | 779 |
| | | | c: Clostridia; o: Clostridiales; | |
| | | | f: Ruminococcaceae; | |
| | | | g: Faecalibacterium; | |
| | | | s: prausnitzii | |
| 366195 | 13 | 0.206349206 | k: Bacteria; p: Firmicutes; | 780 |
| | | | c: Clostridia; o: Clostridiales; | |
| | | | f: Ruminococcaceae; | |
| | | | g: Ruminococcus; | |
| | | | s: bromii | |
| 366451 | 1 | 0.015873016 | k: Bacteria; p: Proteobacteria; | 781 |
| | | | c: Gammaproteobacteria; | |
| | | | o: Enterobacteriales; | |
| | | | f: Enterobacteriaceae; g:; s: | |
| 366781 | 3 | 0.047619048 | k: Bacteria; p: Firmicutes; | 782 |
| | | | c: Clostridia; o: Clostridiales; | |
| | | | f: Clostridiaceae; g: Clostridium; s: | |
| 367176 | 20 | 0.317460317 | k: Bacteria; p: Firmicutes; | 783 |
| | | | c: Clostridia; o: Clostridialed; | |
| | | | f: Ruminococcaceae; | |
| | | | g: Oscillospira; s: | |
| 367232 | 39 | 0.619047619 | k: Bacteria; p: Firmicutes; | 784 |
| | | | c: Clostridia; o: Clostridiales; | |
| | | | f: Ruminococcaceae; | |
| | | | g: Faecalibacterium; | |
| | | | s: prausnitzii | |
| 367453 | 18 | 0.285714286 | k: Bacteria; p: Firmicutes; | 785 |
| | | | c: Clostridia; o: Costridiales; | |
| | | | f: Lachnospiraceae; | |
| | | | g: Coprococcus; s: | |
| 367688 | 56 | 0.888888889 | k: Bacteria; p: Firmicutes; | 786 |
| | | | c: Clostridia; o: Clostridiales; | |
| | | | f: Lachnospiraceae; | |
| | | | g: Coprococcus; s: | |
| 367889 | 52 | 0.825396825 | k: Bacteria; p: Firmicutes; | 787 |
| | | | c: Clostridia; o: Clostridiales; | |
| | | | f: Lachnospiraceae; | |
| | | | g: Coprococcus; s: | |
| 368127 | 3 | 0.047619048 | k: Bacteria; p: Firmicutes; | 788 |
| | | | c: Clostridia; o: Clostridiales; | |
| | | | f: Ruminococcaceae; | |
| | | | g: Faecalibacterium; | |
| | | | s: prausnitzii | |
| 368175 | 4 | 0.063492063 | k: Bacterial; p: Actinobacteria; | 789 |
| | | | c: Coriobacteriia; | |
| | | | o: Coriobacteriales; | |
| | | | f: Coriobacteriaceae; | |
| | | | g: Collinsella; | |
| | | | s: aerofaciens | |
| 368219 | 52 | 0.825396825 | k: Bacteria; p: Firmicutes; | 790 |
| | | | c: Clostridia; o: Clostridiales; | |
| | | | f: Ruminococcaceae; | |
| | | | g: Faecalibacterium; | |
| | | | s: prausnitzii | |
| 368711 | 10 | 0.158730159 | k: Bacteria; p: Firmicutes; | 791 |
| | | | c: Clostridia; o: Clostridiales; | |
| | | | f: Christensenellaceae; g:; s: | |
| 369354 | 1 | 0.015873016 | k: Bacteria; p: Actinobacteria; | 792 |
| | | | c: Coriobacteriia; | |
| | | | o: Coriobacteriales; | |
| | | | f: Coriobacteriaceae; g:; s: | |
| 369486 | 53 | 0.841269841 | k: Bacteria; p: Firmicutes; | 793 |
| | | | c: Clostridia; o: Clostridiales; | |
| | | | f: Lachnospiraceae; | |
| | | | g: Lachnospira; s: | |
| 369555 | 6 | 0.095238095 | k: Bacteria; p: Frimicutes; | 794 |
| | | | c: Clostridia; o: Clostridiales; | |
| | | | f: Ruminococcaceae; | |
| | | | g: Ruminococcus; s: | |
| 369602 | 1 | 0.015873016 | k: Bacteria; p: Firmicutes; | 795 |
| | | | c: Clostridia; o: Clostridiales; | |
| | | | f: Ruminococcaceae; | |
| | | | g: Ruminococcus; | |
| | | | s: bromii | |
| 369797 | 1 | 0.015873016 | k: Bacteria; p: Firmicutes; | 796 |
| | | | c: Clostridia; o: Clostridiales; | |
| | | | f: Ruminococcaceae; | |
| | | | g: Ruminococcus; s: | |
| 369996 | 2 | 0.031746032 | k: Bacteria; p: Firmicutes; | 797 |
| | | | c: Clostridia; o: Clostridiales; | |
| | | | f: Ruminococcaceae; | |
| | | | g: Faecalibacterium; | |
| | | | s: prausnitzii | |
| 370075 | 4 | 0.063492063 | k: Bacteria; p: Firmicutes; | 798 |
| | | | c: Clostridia; o: Clostridiales; | |
| | | | f: [Mogibaceriaceae]; g:; s: | |
| 370098 | 22 | 0.349206349 | k: Bacteria; p: Firmicutes; | 799 |
| | | | c: Clostridia; o: Clostridiales; | |
| | | | f: Lachnospiraceae; | |
| | | | g: Lachnospira; s: | |
| 370287 | 20 | 0.317460317 | k: Bacteria; p: Firmicutes; | 800 |
| | | | c: Clostridia; o: Clostridiales; | |
| | | | f: Ruminococcaceae; | |
| | | | g: Faecalibacterium; | |
| | | | s: prausnitzii | |
| 372146 | 4 | 0.063492063 | k: Bacteria; p: Firmicutes; | 801 |
| | | | c: Clostridia; o: Clostridiales; | |
| | | | f: Ruminococcaceae; | |
| | | | g: Oscillospira; s: | |
| 372348 | 3 | 0.047619048 | k: Bacteria; p: Firmicutes; | 802 |
| | | | c: Clostridia; o: Clostridiales; | |
| | | | f: Ruminococcaceae; | |
| | | | g: Ruminococcus; s: | |
| 405780 | 2 | 0.031746032 | k: Bacteria; p: Firmicutes; | 803 |
| | | | c: Clostridia; o: Clostridiales; | |
| | | | f: Ruminococcaceae; | |
| | | | g: Ruminococcus; s: | |
| 414949 | 4 | 0.063492063 | k: Bacteria; p: Actinobacteria; | 804 |
| | | | c: Coriobacteriia; | |
| | | | o: Coriobacteriales; | |
| | | | f: Coriobacteriaceae; | |
| | | | g: Collinsella; s: | |
| 415315 | 1 | 0.015873016 | k: Bacteria; p: Actinobacteria; | 805 |
| | | | c: Coriobacteriia; | |
| | | | o: Coriobacteriales; | |
| | | | f: Coriobacteriaceae; | |
| | | | g: Collinsella; s: | |
| 422283 | 1 | 0.015873016 | k: Bacteria; p: Firmicutes; | 806 |
| | | | c: Clostridia; o: Clostridiales; | |
| | | | f: Ruminococcaceae; | |
| | | | g: Oscillospira; s: | |
| 437137 | 6 | 0.095238095 | k: Bacteria; p: Proteobacteria; | 807 |
| | | | c: Betaproteoabacteria; | |
| | | | o: Burkholderiales; | |
| | | | f: Alcaligenaceae; | |
| | | | g: Sutterella; s: | |
| 465433 | 1 | 0.015873016 | k: Bacteria; p: Bacteroidetes; | 808 |
| | | | c: Bacteroidales; | |
| | | | f: Rikenellaceae; g:; s: | |
| 470117 | 17 | 0.26984127 | k: Bacteria; p: Firmicutes; | 809 |
| | | | c: Clostridia; o: Clostridiales; | |
| | | | f: Ruminococcaceae; | |
| | | | g: Clostridium; | |
| | | | s: methylpentosum | |
| 508897 | 2 | 0.031746032 | k: Bacteria; p: Firmicutes; | 810 |
| | | | c: Clostridia; o: Clostridiales; | |
| | | | f: Lachnospiraceae; | |
| | | | g: Coprococcus; s: | |
| 509516 | 1 | 0.015873016 | k: Bacteria; p: Firmicutes; | 811 |
| | | | c: Bacilli; o: Lactobacillales; | |
| | | | f: Enterococcaceae; | |
| | | | g: Enterococcus; s: | |
| 512494 | 1 | 0.015873016 | k: Bacteria; p: Firmicutes; | 812 |
| | | | c: Clostridia; o: Clostridiales | |
| | | | f: Veillonellaceae; | |
| | | | g: Phascolarctobacterium; s: | |
| 514523 | 50 | 0.793650794 | k: Bacteria; p: Firmicutes; | 813 |
| | | | c: Clostridia; o: Clostridiales; | |
| | | | f: Ruminococcaceae; | |
| | | | g: Faecalibacterium; | |
| | | | s: prausnitzii | |
| 515575 | 3 | 0.047619048 | k: Bacteria; p: Firmicutes; | 814 |
| | | | c: Clostridia; o: Clostridiales; | |
| | | | f: Ruminococcaceae; | |
| | | | g: Faecalibacterium; | |
| | | | s: prausnitzii | |
| 518002 | 2 | 0.031746032 | k: Bacteria; p: Proteobacteria; | 815 |
| | | | c: Gammaproteobacteria; | |
| | | | o: Enterobacteriales; | |
| | | | f: Enterobacteriaceae; g:; s: | |
| 518460 | 9 | 0.142857143 | k: Bacteria; p: Firmicutes; | 816 |
| | | | c: Clostridia; o: Clostridiales; | |
| | | | f: Lachnospiraceae; | |
| | | | g: Coprococcus; s: | |
| 520413 | 2 | 0.031746032 | k: Bacteria; p: Firmicutes; | 817 |
| | | | c: Clostridia; o: Clostridiales; | |
| | | | f: Ruminococcaceae; | |
| | | | g: Faecalibacterium; | |
| | | | s: prausnitzii | |
| 521982 | 1 | 0.015873016 | k: Bacteria; p: Bacteroidetes; | 818 |
| | | | c: Bacteroidia; o: Bacteroidales; | |
| | | | f: Rikenellaceae; g:; s: | |
| 523140 | 16 | 0.253968254 | k: Bacteria; p: Firmicutes; | 819 |
| | | | c: Clostridia; o: Clostridiales; | |
| | | | f: Ruminococcaceae; | |
| | | | g: Ruminococcus; s: | |
| 525215 | 2 | 0.031746032 | k: Bacteria; p: Firmicutes; | 820 |
| | | | c: Clostridia; o: Clostridiales; | |
| | | | f: Ruminococcaceae; | |
| | | | g: Faecalibacterium; | |
| | | | s: prausnitzii | |
| 525698 | 39 | 0.619047619 | k: Bacteria; p: Firmicutes; | 821 |
| | | | c: Clostridia; o: Clostridiales; | |
| | | | f: Ruminococcaceae; | |
| | | | g: Faecalibacterium; | |
| | | | s: prausnitzii | |
| 526665 | 1 | 0.015873016 | k: Bacteria; p: Firmicutes; | 822 |
| | | | c: Clostridia; c: Clostridiales; | |
| | | | f: Ruminococcaceae; | |
| | | | g: Oscillospira; s: | |
| 528652 | 1 | 0.015873016 | k: Bacteria; p: Firmicutes; | 823 |
| | | | c: Clostridia; o: Clostridiales; | |
| | | | f: Ruminococcaceae; | |
| | | | g: Faecalibacterium; | |
| | | | s: prausnitzii | |
| 529652 | 3 | 0.047619048 | k: Bacteria; p: Proteobacteria; | 824 |
| | | | c: Betaproteobacteria; | |
| | | | o: Burkholderiales; | |
| | | | f: Alcaligenaceae; g: Sutterella; s: | |
| 529789 | 4 | 0.063492063 | k: Bacteria; p: Firmicutes; | 825 |
| | | | c: Clostridia; o: Clostridiales; | |
| | | | f: Ruminococacceae; | |
| | | | g: Ruminococcus; s: | |
| 533847 | 2 | 0.031746032 | k: Bacteria; p: Firmicutes; | 826 |
| | | | c: Clostridia; o: Clostridiales; | |
| | | | f: Ruminococcaceae; | |
| | | | g: Oscillospira; s: | |
| 534965 | 1 | 0.015873016 | k: Bacteria; p: Proteobacteria; | 827 |
| | | | c: Gammaproteobacteria; | |
| | | | o: Enterobacterials; | |
| | | | f: Enterobacteriaceae; g:; s: | |
| 535399 | 2 | 0.031746032 | k: Bacteria; p: Firmicutes; | 828 |
| | | | c: Clostridia; o: Clostridiales; | |
| | | | f: Ruminococcaceae; | |
| | | | g: Oscillospira; s: | |
| 536584 | 2 | 0.031746032 | k: Bacteria; p: Firmicutes; | 829 |
| | | | c: Clostridia; o: Clostridiales; | |
| | | | f: Christensenellaceae; g:; s: | |
| 539820 | 2 | 0.031746032 | k: Bacteria; p: Firmicutes; | 830 |
| | | | c: Clostridia; o: Clostridiales; | |
| | | | f: Ruminococcaceae; | |
| | | | g: Oscillospira; s: | |
| 540402 | 1 | 0.015873016 | k: Bacteria; p: Proteobacteria; | 831 |
| | | | c: Gammaproteobacteria; | |
| | | | o: Enterobacteriales; | |
| | | | f: Enterobacteriaceae; g:; s: | |
| 541119 | 2 | 0.031746032 | k: Bacteria; p: Proteobacteria; | 832 |
| | | | c: Gammaproteobacteria; | |
| | | | o: Enterobacteriales; | |
| | | | f: Enterobacteriaceae; g:; s: | |
| 543824 | 2 | 0.031746032 | k: Bacteria; p: Firmicutes; | 833 |
| | | | c: Bacilli; o: Lactobacillales; | |
| | | | f: Enterorococcaceae; | |
| | | | g: Enterococcus; s: | |
| 544996 | 1 | 0.015873016 | k: Bacteria; p: Firmicutes; | 834 |
| | | | c: Clostridia; o: Clostridiales; | |
| | | | f: Ruminococcaceae; | |
| | | | g: Oscillospira; s: | |
| 548032 | 52 | 0.825396825 | k: Bacteria; p: Firmicutes; | 835 |
| | | | c: Clostridia; o: Clostridiales; | |
| | | | f: Ruminococcaceae; | |
| | | | g: Oscillospira; s: | |
| 551822 | 1 | 0.015873016 | k: Bacteria; p: Firmicutes; | 836 |
| | | | c: Clostridia; o: Clostridiales; | |
| | | | f: Clostridiaceae; g:; s: | |
| 553150 | 15 | 0.238095238 | k: Bacteria; p: Firmicutes; | 837 |
| | | | c: Clostridia; o: Clostridiales; | |
| | | | f: Clostridiaceae; g:; | |
| | | | Coprococcus; s: | |
| 555547 | 17 | 0.26984127 | k: Bacteria; p: Firmicutes; | 838 |
| | | | c: Clostridia; o: Clostridiales; | |
| | | | f: Christensenellaceae; g:; s: | |
| 556240 | 5 | 0.079365079 | k: Bacteria; p: Firmicutes; | 839 |
| | | | c: Clostridia; o: Clostridiales; | |
| | | | f: LAchnospiraceae; g:; | |
| | | | Coprococcus; s: | |
| 556835 | 13 | 0.206349206 | k: Bacteria; p: Firmicutes; | 840 |
| | | | c: Clostridia; o: Clostridiales; | |
| | | | f: Phascolarctobacterium; s: | |
| 559204 | 2 | 0.031746032 | k: Bacteria; p: Proteobacteria; | 841 |
| | | | c: Gammaproteobacteria; | |
| | | | o: Enterobacteriales; | |
| | | | f: Enterobacteriaceae; g:; s: | |
| 562244 | 4 | 0.063492063 | k: Bacteria; p: Proteobacteria; | 842 |
| | | | c: Betaproteobacteria; | |
| | | | o: Burkholderiales; | |
| | | | f: Alcaligenaceae; g: Sutterella; s: | |
| 562410 | 8 | 0.126984127 | k: Bacteria; p: Firmicutes; | 843 |
| | | | c: Clostridia; o: Clostridiales; | |
| | | | f: Christensenellaceae; g:; s: | |
| 564334 | 1 | 0.015873016 | k: Bacteria; p: Firmicutes; | |
| | | | c: Clostridia; o: Clostridiales; f: | 844 |
| | | | Ruminococcaceae; | |
| | | | g: Oscillospira; s: | |
| 566976 | 23 | 0.365079365 | k: Bacteria; p: Proteobacteria; | 845 |
| | | | c: Gammaproteobacteria; | |
| | | | o: Enterobacteriales; | |
| | | | f: Enterobacteriaceae; g:; s: | |
| 571242 | 5 | 0.079365079 | k: Bacteria; p: Firmicutes; | 846 |
| | | | c: Clostridia; o: Clostridiales; | |
| | | | f: Ruminococcaceae; | |
| | | | g: Ruminococcus; s: | |
| 574122 | 5 | 0.079365079 | k: Bacteria; p: Firmicutes; | 847 |
| | | | c: Clostridia; o: Clostridiales; | |
| | | | f: Ruminococcaceae; | |
| | | | g: Oscillospira; s: | |
| 575041 | 6 | 0.095238095 | k: Bacteria; p: Bacteriodetes; | 848 |
| | | | c: Bacteroidia; o: Bacteroidales; | |
| | | | f: Rikenellaceae; g:; s: | |
| 578511 | 3 | 0.147619048 | k: Bacteria; p: Firmicutes; | 849 |
| | | | c: Clostridia; o: Clostridiales; | |
| | | | f: Lachnospiraceae; | |
| | | | g: Coprococcus; s: | |
| 579244 | 3 | 0.047619048 | k: Bacteria; p: Firmicutes; | 850 |
| | | | c: Clostridia; o: Clostridiales; | |
| | | | f: Ruminococcaceae; | |
| | | | g: Oscillospira; s: | |
| 581265 | 1 | 0.015873016 | k: Bacteria; p: Firmicutes; | 851 |
| | | | c: Clostridia; o: Clostridiales; | |
| | | | f: Ruminococcaceae; | |
| | | | g: Oscillospira; s: | |
| 584978 | 19 | 0.301587302 | k: Bacteria; p: Firmicutes; | 852 |
| | | | c: Clostridia; o: Clostridiales; | |
| | | | f: Ruminococcaceae; | |
| | | | g: Ruminococcus; s: | |
| 585227 | 1 | 0.015873016 | k: Bacteria; p: Firmicutes; | 853 |
| | | | c: Clostridia; o: Clostridiales; | |
| | | | f: Ruminococcaceae; | |
| | | | g: Oscillospira; s: | |
| 585989 | 4 | 0.063492063 | k: Bacteria; p: Firmicutes; | 854 |
| | | | c: Clostridia; o: Clostridiales; | |
| | | | f: Veillonellaceae; g: | |
| | | | Phascolarctobacterium; s: | |
| 586453 | 19 | 0.301587302 | k: Bacteria; p: Firmicutes; | 855 |
| | | | c: Clostridia; o: Clostridiales; | |
| | | | f: Christensenellaceae; g; s: | |
| 589076 | 4 | 0.063492063 | k: Bacteria; p: Firmicutes; | 856 |
| | | | c: Clostridia; o: Clostridiales; | |
| | | | f: Ruminococcaceae; | |
| | | | g: Oscillospira; s: | |
| 591439 | 10 | 0.158730159 | k: Bacteria; p: Firmicutes; | 857 |
| | | | c: Clostridia; o: Clostridiales; | |
| | | | f: [Mogibacteriaceae]; g:; s: | |
| 591540 | 7 | 0.111111111 | k: Bacteria; p: Firmicutes; | 858 |
| | | | c: Clostridia; o: Clostridiales; | |
| | | | f: Lachnospiraceae; | |
| | | | g: Coprococcus; s: | |
| 593686 | 3 | 0.047619048 | k: Bacteria; p: Firmicutes; | 859 |
| | | | c: Clostridia; o: Clostridiales; | |
| | | | f: Lachnospiraceae; | |
| | | | g: Coprococcus; s: | |
| 608244 | 5 | 0.079365079 | k: Bacteria; p: Firmicutes; | 860 |
| | | | c: Clostridia; o: Clostridiales; | |
| | | | f: Ruminococcaceae; | |
| | | | g: Ruminococcus; s: | |
| 622303 | 2 | 0.031746032 | k: Bacteria; p: Firmicutes; | 861 |
| | | | c: Clostridia; o: Clostridiales; | |
| | | | f: Clostridiaceae; g: Clostridium; s: | |
| 644244 | 3 | 0.047619048 | k: Bacteria; p: Firmicutes; | 862 |
| | | | c: Clostridia; o: Clostridiales; | |
| | | | f: Christensenellaceae; g:; s: | |
| 663500 | 2 | 0.031746032 | k: Bacteria; p: Firmicutes; | 863 |
| | | | c: Clostridia; o: Clostridiales; | |
| | | | f: Veillonellaceae; | |
| | | | g: Phascolarctobacterium; s: | |
| 681779 | 2 | 0.031746032 | k: Bacteria; p: Proteobacteria; | 864 |
| | | | c: Gammaproteobacteria; | |
| | | | o: Enterobacteriales; | |
| | | | f: Enterobacteriaceae; g:; s: | |
| 686711 | 3 | 0.047619048 | k: Bacteria; p: Firmicutes; | 865 |
| | | | c: Clostridia; o: Clostridiales; | |
| | | | f: Christensenellaceae; g:; s: | |
| 686972 | 1 | 0.015873016 | k: Bacteria; p: Proteobacteria; | 866 |
| | | | c: Gammaproteobacteria; | |
| | | | o: Enterobacteriales; | |
| | | | f: Enterobacteriaceae; g:; s: | |
| 687940 | 1 | 0.015873016 | k: Bacteria; p: Proteobacteria; | 867 |
| | | | c: Gammaproteobacteria; | |
| | | | o: Enterobacteriales; | |
| | | | f: Enterobacteriaceae; | |
| | | | g: Enterobacter; s: arachidis | |
| 691423 | 1 | 0.015873016 | k: Bacteria; p: Proteobacteria; | 868 |
| | | | c: Gammaproteobacteria; | |
| | | | o: Enterobacteriales; | |
| | | | f: Enterobacteriaceae; g:; s: | |
| 708285 | 20 | 0.317460317 | k: Bacteria; p: Firmicutes; | 869 |
| | | | c: Clostridia; o: Clostridiales; | |
| | | | f: Clostridiaceae; g:; s: | |
| 731422 | 3 | 0.047619048 | k: Bacteria; p: Firmicutes; | 870 |
| | | | c: Clostridia; o: Clostridiales; | |
| | | | f: Veillonellaceae; g: | |
| | | | Phascolarctobacterium; s: | |
| 740158 | 3 | 0.047619048 | k: Bacteria; p: Firmicutes; | 871 |
| | | | c: Clostridia; o: Clostridiales; | |
| | | | f: Veillonellaceae; g: | |
| | | | Phascolarctobacterium; s: | |
| 742358 | 1 | 0.015873016 | k: Bacteria; p: Actinobacteria; | 872 |
| | | | c: Coriobacteriia; | |
| | | | o: Coriobacteriales; | |
| | | | f: Coriobacteriaceae; g:; s: | |
| 743082 | 2 | 0.031746032 | k: Bacteria; p: Firmicutes; | 873 |
| | | | c: Clostridia; o: Clostridiales; | |
| | | | f: Clostridiaceae; g:; s: | |
| 760594 | 3 | 0.047619048 | k: Bacteria; p: Firmicutes; | 874 |
| | | | c: Clostridia; o: Clostridiales; | |
| | | | f: Ruminococcaceae; | |
| | | | g: Ruminococcus; s: | |
| 766768 | 1 | 0.015873016 | k: Bacteria; p: Firmicutes; | 875 |
| | | | c: Bacilli; o: | |
| | | | Lactobacillococcaceae; | |
| | | | g: Enterococcus; s: | |
| 772282 | 3 | 0.047619048 | k: Bacteria; p: Bacteroidetes; | 876 |
| | | | c: Bacteroidia; o: Bacteroidales; | |
| | | | f: Rikenellaceae; g:; s: | |
| 786708 | 3 | 0.047619048 | k: Bacteria; p: Proteobacteria; | 877 |
| | | | c: Gammaproteobacteria; | |
| | | | o: Enterobacteriales; | |
| | | | f: Enterobacteriaceae; g:; s: | |
| 790466 | 1 | 0.015873016 | k: Bacteria; p: Firmicutes; | 878 |
| | | | c: Clostridia; o: Clostridiales; | |
| | | | f: [Mogibacteriaceae]; g:; s: | |
| 797229 | 1 | 0.015873016 | k: Bacteria; p: Proteobacteria; | 879 |
| | | | c: Gammaproteobacteria; | |
| | | | o: Enterobacteriales; | |
| | | | f: Enterobacteriaceae; g:; s: | |
| 798581 | 3 | 0.047619048 | k: Bacteria; p: Firmicutes; | 880 |
| | | | c: Clostridia; o: Clostridiales; | |
| | | | f: Ruminococcaceae; | |
| | | | g: Ruminococcus; | |
| | | | s: bromii | |
| 801210 | 1 | 0.015873016 | k: Bacteria; p: Proteobacteria; | 881 |
| | | | c: Betaproteobacteria; | |
| | | | o: Burkholderiales; | |
| | | | f: Alcaligenaceae; g: Sutterella; s: | 882 |
| 808609 | 1 | 0.015873016 | k: Bacteria; p: Firmicutes; | |
| | | | c: Clostridia; o: Clostridiales; | |
| | | | f: Ruminococcaceae; | |
| | | | g: Ruminococcus; s: | |
| 811453 | 2 | 0.031746032 | k: Bacteria; p: Firmicutes; | 883 |
| | | | c: Clostridia; o: Clostridiales; | |
| | | | f: Ruminococcaceae; | |
| | | | g: Oscillospira; s: | |
| 816299 | 1 | 0.015873016 | k: Bacteria; p: Actinobacteria; | 884 |
| | | | c: Coriobacteriia; | |
| | | | o: Coriobacteriales; | |
| | | | f: Coriobacteriaceae; g:; s: | |
| 818516 | 1 | 0.015873016 | k: Bacteria; p: Firmicutes; | 885 |
| | | | c: Clostridia; o: Clostridiales; | |
| | | | f: Clostridiaceae; g: Clostridium; | |
| | | | s: botulinum | |
| 823634 | 6 | 0.095238095 | k: Bacteria; p: Firmicutes; | 886 |
| | | | c: Clostridia; o: Clostridiales; | |
| | | | f: Ruminococcaceae; | |
| | | | g: Ruminococcus; s: | |
| 828776 | 1 | 0.015873016 | k: Bacteria; p: Firmicutes; | 887 |
| | | | c: Clostridia; o: Clostridiales; | |
| | | | f: Lachnospiraceae; | |
| | | | g: Coprococcus; s: | |
| 832089 | 10 | 0.158730159 | k: Bacteria; p: Firmicutes; | 888 |
| | | | c: Clostridia; o: Clostridiales; | |
| | | | f: [Mogibacteriaceae]; g:; s. | |
| 835346 | 1 | 0.015873016 | k: Bacteria; p: Proteobacteria; | 889 |
| | | | c: Gammaproteobacteria; | |
| | | | o: Enterobacteriales; | |
| | | | f: Enterobacteriaceae; | |
| | | | g: Erwinia; s: | |
| 835771 | 5 | 0.079365079 | k: Bacteria; p: Proteobacteria; | 890 |
| | | | c: Gammaproteobacteria; | |
| | | | o: Enterobacteriales; | |
| | | | f: Enterobacteriaceae; | |
| | | | g: Plesiomonas; s: shigelloides | |
| 839964 | 2 | 0.031746032 | k: Bacteria; p: Firmicutes; | 891 |
| | | | c: Clostridia; o: Clostridiales; | |
| | | | f: Ruminococcaceae; | |
| | | | g: Oscillospira; s: | |
| 840279 | 1 | 0.015873016 | k: Bacteria; p: Bacteroidetes; | 892 |
| | | | c: Bacteroidia; o: Bacteroidales; | |
| | | | f: [Barnesiellaceae]; g:; s: | |
| 841635 | 20 | 0.317460317 | k: Bacteria; p: Proteobacteria; | 893 |
| | | | c: Betaproteobacteria; | |
| | | | o: Burkholderiales; | |
| | | | f: Alcaligenaceae; g: Sutterella; s: | |
| 842596 | 45 | 0.714285714 | k: Bacteria; p: Firmicutes; | 894 |
| | | | c: Clostridia; o: Clostridiales; | |
| | | | f: Lachnospiraceae; | |
| | | | g: Coprococcus; s: | |
| 843553 | 4 | 0.063492063 | k: Bacteria; p: Firmicutes; | 895 |
| | | | c: Clostridia; o: Clostridiales; | |
| | | | f: Lachnospiraceae; | |
| | | | g: Lachnospira; s: | |
| 846798 | 8 | 0.126984127 | k: Bacteria; p: Bacteroidetes; | 896 |
| | | | c: Bacteroidia; o: Bacteroidales; | |
| | | | f: [Barnesiellaceae]; g:; s: | |
| 847427 | 1 | 0.015873016 | k: Bacteria; p: Bacteroidetes; | 897 |
| | | | c: Bacteroidia; o: Bacteroidales; | |
| | | | f: [Odoribacteraceae]; | |
| | | | g: Odoribacter; s: | |
| 847670 | 15 | 0.238095238 | k: Bacteria; p: Firmicutes; | 898 |
| | | | c: Clostridia; o: Clostridiales; | |
| | | | f: Clostridiaceae; g:; s: | |
| 847711 | 1 | 0.015873016 | k: Bacteria; p: Firmicutes; | 899 |
| | | | c: Clostridia; o: Clostridiales; | |
| | | | f: Christensenellaceae; g:; s: | |
| 847728 | 1 | 0.015873016 | k: Bacteria; p: Firmicutes; | 900 |
| | | | c: Clostridia; o: Clostridiales; | |
| | | | f: Ruminococcaceae; | |
| | | | g: Ruminococcus; s: | |
| 848669 | 11 | 0.174603175 | k: Bacteria; p: Proteobacteria; | 901 |
| | | | c: Betaproteobacteria; | |
| | | | o: Burkholderiales; | |
| | | | f: Alcaligenaceae; g: Sutterella; s: | |
| 848915 | 2 | 0.031746032 | k: Bacteria; p: Firmicutes; | 902 |
| | | | c: Clostridia; o: Clostridiales; | |
| | | | f: Ruminococcaceae; | |
| | | | g: Oscillospira; s: | |
| 916143 | 2 | 0.031746032 | k: Bacteria; p: Firmicutes; | 903 |
| | | | c: Clostridia; o: Clostridiales; | |
| | | | f: Veillonellaceae; g: | |
| | | | Phascolarctobacterium; s: | |
| 953855 | 18 | 0.285714286 | k: Bacteria; p: Bacteroidetes; | 904 |
| | | | c: Bacteroidia; o: Bacteroidales; | |
| | | | f: Rikenellaceae; g:; s: | |
| 1010876 | 1 | 0.015873016 | k: Bacteria; p: Firmicutes; | 905 |
| | | | c: Clostridia; o: Clostridiales; | |
| | | | f: Ruminococcaceae; | |
| | | | g: Oscillospira; s: | |
| 1026524 | 1 | 0.015873016 | k: Bacteria; p: Firmicutes; | 906 |
| | | | c: Clostridia; o: Clostridiales; | |
| | | | f: Ruminococcaceae; | |
| | | | g: Oscillospira; s: | |
| 1029949 | 18 | 0.285714286 | k: Bacteria; p: Firmicutes; | 907 |
| | | | c: Clostridia; o: Clostridiales; | |
| | | | f: Lachnospiraceae; | |
| | | | g: Lachnospira; s: | |
| 1034960 | 2 | 0.031746032 | k: Bacteria; p: Actinobacteria; | 908 |
| | | | c: Coriobacteriia; | |
| | | | o: Coriobacteriales; | |
| | | | f: Coriobacteriaceae; g:; s: | |
| 1068836 | 2 | 0.031746032 | k: Bacteria; p: Firmicutes; | 909 |
| | | | c: Clostridia; o: Clostridiales; | |
| | | | f: Ruminococcaceae; | |
| | | | g: Ruminococcus; s: | |
| 1105343 | 6 | 0.095238095 | k: Bacteria; p: Firmicutes; | 910 |
| | | | c: Clostridia; o: Clostridiales; | |
| | | | f: Clostridiaceae; g: Clostridium; s: | |
| 1105376 | 2 | 0.031746032 | k: Bacteria; p: Proteobacteria; | 911 |
| | | | c: Betaproteobacteria; | |
| | | | o: Burkholderiales; | |
| | | | f: Alcaligenaceae; g: Sutterella; s: | |
| 1106006 | 1 | 0.015873016 | k: Bacteria; p: Firmicutes; | 912 |
| | | | c: Clostridia; o: Clostridiales; | |
| | | | f: Ruminococcaceae; | |
| | | | g: Ruminococcus; s: | |
| 1106362 | 1 | 0.015873016 | k: Bacteria; p: Firmicutes; | 913 |
| | | | c: Clostridia; o: Clostridiales; | |
| | | | f: Clostridiaceae; g: Clostridium; s: | |
| 1108470 | 2 | 0.031746032 | k: Bacteria; p: Firmicutes; | 914 |
| | | | c: Clostridia; o: Clostridiales; | |
| | | | f: Ruminococcaceae; | |
| | | | g: Ruminococcus; s: | |
| 1108656 | 2 | 0.031746032 | k: Bacteria; p: Proteobacteria; | 915 |
| | | | c: Gammaproteobacteria; | |
| | | | o: Enterobacteriales; | |
| | | | f: Enterobacteriaceae; g:; s: | |
| 1108745 | 3 | 0.047619048 | k: Bacteria; p: Firmicutes; | 916 |
| | | | c: Clostridia; o: Clostridiales; | |
| | | | f: [Mogibacteriaceae]; g:; s: | |
| 1112762 | 2 | 0.031746032 | k: Bacteria; p: Firmicutes; | 917 |
| | | | c: Clostridia; o: Clostridiales; | |
| | | | f: Ruminococcaceae; | |
| | | | g: Oscillospira; s: | |
| 1123070 | 1 | 0.015873016 | k: Bacteria; p: Firmicutes; | 918 |
| | | | c: Clostridia; o: Clostridiales; | |
| | | | f: Ruminococcaceae; | |
| | | | g: Ruminococcus; s: | |
| 1128398 | 2 | 0.031746032 | k: Bacteria; p: Proteobacteria; | 919 |
| | | | c: Gammaproteobacteria; | |
| | | | o: Enterobacteriales; | |
| | | | f: Enterobacteriaceae; g:; s: | |
| 1129461 | 1 | 0.015873016 | k: Bacteria; p: Firmicutes; | 920 |
| | | | c: Bacilli; o: | |
| | | | Lactobacillococcaceae; | |
| | | | g: Enterococcus; s: | |
| | | | haemoperoxidus | |
| 1135616 | 2 | 0.031746032 | k: Bacteria; p: Firmicutes; | 921 |
| | | | c: Bacilli; o: | |
| | | | Lactobacillusoccaceae; | |
| | | | g: Enterococcus; s: | |
| 1145098 | 4 | 0.063492063 | k: Bacteria; p: Firmicutes; | 922 |
| | | | c: Clostridia; o: Clostridiales; | |
| | | | f: Lachnospiraceae; | |
| | | | g: Ruminococcus; | |
| | | | s: gauvreauii | |
| 1503693 | 1 | 0.015873016 | k: Bacteria; p: Firmicutes; | 923 |
| | | | c: Clostridia; o: Clostridiales; | |
| | | | f: Clostridiaceae; g:; s: | |
| 1584173 | 3 | 0.047619048 | k: Bacteria; p: Bacteroidetes; | 924 |
| | | | c: Bacteroidia; o: Bacteroidales; | |
| | | | f: [Barnesiellaceae]; g:; s: | |
| 1602307 | 17 | 0.26984127 | k: Bacteria; p: Proteobacteria; | 925 |
| | | | c: Betaproteobacteria; | |
| | | | o: Burkholderiales; | |
| | | | f: Alcaligenaceae; g: Sutterella; s: | 926 |
| 1624383 | 24 | 0.380952381 | k: Bacteria; p: Firmicutes; | |
| | | | c: Clostridia; o: Clostridiales; | |
| | | | f: Lachnospiraceae; | |
| | | | g: Lachnospira; s: | |
| 1646183 | 1 | 0.015873016 | k: Bacteria; p: Actinobacteria; | 927 |
| | | | c: Coriobacteriia; | |
| | | | o: Coriobacteriales; | |
| | | | f: Coriobacteriaceae; g:; s: | |
| 1657484 | 1 | 0.015873016 | k: Bacteria; p: Firmicutes; | 928 |
| | | | c: Clostridia; o: Clostridiales; | |
| | | | f: Ruminococcaceae; | |
| | | | g: Ruminococcus; s: | |
| 1663575 | 6 | 0.095238095 | k: Bacteria; p: Proteobacteria; | 929 |
| | | | c: Gammaproteobacteria; | |
| | | | o: Enterobacteriales; | |
| | | | f: Enterobacteriaceae; g:; s: | |
| 1719314 | 1 | 0.015873016 | k: Bacteria; p: Firmicutes; | 930 |
| | | | c: Clostridia; o: Clostridiales; | |
| | | | f: Ruminococcaceae; | |
| | | | g: Faecalibacterium; | |
| | | | s: prausnitzii | |
| 1726426 | 1 | 0.015873016 | k: Bacteria; p: Proteobacteria; | 931 |
| | | | c: Gammaproteobacteria; | |
| | | | o: Enterobacteriales; | |
| | | | f: Enterobacteriaceae; | |
| | | | g: Serratia; s: | |
| 1756274 | 1 | 0.015873016 | k: Bacteria; p: Firmicutes; | 932 |
| | | | c: Bacilli; o: | |
| | | | Lactobacillococcaceae; | |
| | | | g: Enterococcus; s: | |
| 1808742 | 2 | 0.031746032 | k: Bacteria; p: Firmicutes; | 933 |
| | | | c: Clostridia; o: Clostridiales; | |
| | | | f: Ruminococcaceae; | |
| | | | g: Faecalibacterium; | |
| | | | s: prausnitzii | |
| 1811927 | 51 | 0.80952381 | k: Bacteria; p: Actinobacteria; | 934 |
| | | | c: Coriobacteriia; | |
| | | | o: Coriobacteriales; | |
| | | | f: Coriobacteriaceae; | |
| | | | g: Collinsella; | |
| | | | s: aerofaciens | |
| 1820513 | 9 | 0.142857143 | k: Bacteria; p: Proteobacteria; | 935 |
| | | | c: Betaproteobacteria; | |
| | | | o: Burkholderiales; | |
| | | | f: Alcaligenaceae; g: Sutterella; s: | |
| 1855954 | 1 | 0.015873016 | k: Bacteria; p: Firmicutes; | 936 |
| | | | c: Clostridia; o: Clostridiales; | |
| | | | f: Clostridiaceae; g: Clostridium; s: | |
| 1910658 | 24 | 0.380952381 | k: Bacteria; p: Bacteroidetes; | 937 |
| | | | c: Bacteroidia; o: Bacteroidales; | |
| | | | f: [Barnesiellaceae]; g:; s: | |
| 1943669 | 3 | 0.047619048 | k: Bacteria; p: Firmicutes; | 938 |
| | | | c: Clostridia; o: Clostridiales; | |
| | | | f: Ruminococcaceae; | |
| | | | s: prausnitzii | |
| 1974536 | 2 | 0.031746032 | k: Bacteria; p: Proteobacteria; | 939 |
| | | | c: Betaproteobacteria; | |
| | | | o: Burkholderiales; | |
| | | | f: Alcaligenaceae; g: Sutterella; s: | |
| 2065341 | 2 | 0.031746032 | k: Bacteria; p: Firmicutes; | 940 |
| | | | c: Clostridia; o: Clostridiales; | |
| | | | f: Lachnospiraceae; | |
| | | | g: Coprococcus; s: | |
| 2127939 | 2 | 0.031746032 | k: Bacteria; p: Actinobacteria; | 941 |
| | | | c: Coriobacteriia; | |
| | | | o: Coriobacteriales; | |
| | | | f: Coriobacteriaceae; g:; s: | |
| 2196877 | 2 | 0.031746032 | k: Bacteria; p: Firmicutes; | 942 |
| | | | c: Clostridia; o: Clostridiales; | |
| | | | f: Ruminococcaceae; | |
| | | | g: Oscillospira; s: | |
| 2201995 | 28 | 0.444444444 | k: Bacteria; p: Proteobacteria; | 943 |
| | | | c: Betaproteobacteria; | |
| | | | o: Burkholderiales; | |
| | | | f: Alcaligenaceae; g: Sutterella; s: | |
| 2203165 | 4 | 0.063492063 | k: Bacteria; p: Firmicutes; | 944 |
| | | | c: Clostridia; o: Clostridiales; | |
| | | | f: Ruminococcaceae; | |
| | | | g: Oscillospira; s: | |
| 2250345 | 9 | 0.142857143 | k: Bacteria; p: Firmicutes; | 945 |
| | | | c: Clostridia; o: Clostridiales; | |
| | | | f: Lachnospiraceae; g: Clostridium; | |
| | | | s: hathewayi | |
| 2256425 | 12 | 0.19047619 | k: Bacteria; p: Firmicutes; | 946 |
| | | | c: Clostridia; o: Clostridiales; | |
| | | | f: Christensenellaceae; g:; s: | |
| 2307779 | 47 | 0.746031746 | k: Bacteria; p: Firmicutes; | 947 |
| | | | c: Clostridia; o: Clostridiales; | |
| | | | f: Ruminococcaceae; | |
| | | | g: Oscillospira; s: | |
| 2313540 | 23 | 0.365079365 | k: Bacteria; p: Firmicutes; | 948 |
| | | | c: Clostridia; o: Clostridiales; | |
| | | | f: Lachnospiraceae; | |
| | | | g: Lachnospira; s: | |
| 2318202 | 7 | 0.111111111 | k: Bacteria; p: Firmicutes; | 949 |
| | | | c: Clostridia; o: Clostridiales; | |
| | | | f: Ruminococcaceae; | |
| | | | g: Oscillospira; s: | |
| 2365690 | 38 | 0.603174603 | k: Bacteria; p: Firmicutes; | |
| | | | c: Clostridia; o: Clostridiales; | 950 |
| | | | f: Ruminococcaceae; | |
| | | | g: Faecalibacterium; | |
| | | | s: prausnitzii | |
| 2423305 | 7 | 0.111111111 | k: Bacteria; p: Actinobacteria; | 951 |
| | | | c: Coriobacteriia; | |
| | | | o: Coriobacteriales; | |
| | | | f: Coriobacteriaceae; g:; s: | 952 |
| 2442706 | 40 | 0.634920635 | k: Bacteria; p: Firmicutes; | |
| | | | c: Clostridia; o: Clostridiales; | |
| | | | f: Christensenellaceae; g:; s: | |
| 2496050 | 2 | 0.031746032 | k: Bacteria; p: Firmicutes; | 953 |
| | | | c: Clostridia; o: Clostridiales; | |
| | | | f: Ruminococcaceae; | |
| | | | g: Ruminococcus; s: | 954 |
| 2532909 | 4 | 0.063492063 | k: Bacteria; p: Firmicutes; | |
| | | | c: Clostridia; o: Clostridiales; | |
| | | | f: Clostridiaceae; g:; s: | |
| 2544615 | 63 | 1 | k: Bacteria; p: Firmicutes; | |
| | | | c: Clostridia; o: Clostridiales; | 955 |
| | | | f: Ruminococcaceae; | |
| | | | g: Faecalibacterium; | |
| | | | s: prausnitzii | |
| 2582263 | 1 | 0.015873016 | k: Bacteria; p: Proteobacteria; | 956 |
| | | | c: Gammaproteobacteria; | |
| | | | o: Enterobacteriales; | |
| | | | f: Enterobacteriaceae; g:; s: | |
| 2617854 | 38 | 0.603174603 | k: Bacteria; p: Bacteroidetes; | 957 |
| | | | c: Bacteroidia; o: Bacteroidales; | |
| | | | f: Rikenellaceae; g:; s: | |
| 2642538 | 5 | 0.079365079 | k: Bacteria; p: Bacteroidetes; | 958 |
| | | | c: Bacteroidia; o: Bacteroidales; | |
| | | | f: [Odoribacteraceae]; | |
| | | | g: Odoribacter; s: | |
| 2710761 | 1 | 0.015873016 | k: Bacteria; p: Firmicutes; | 959 |
| | | | c: Clostridia; o: Clostridiales; | |
| | | | f: Veillonellaceae; g: | |
| | | | Phascolarctobacterium; s: | |
| 2740950 | 1 | 0.015873016 | k: Bacteria; p: Firmicutes; | 960 |
| | | | c: Clostridia; o: Clostridiales; | |
| | | | f: Lachnospiraceae; | |
| | | | g: Coprococcus; s: | |
| 2749126 | 2 | 0.031746032 | k: Bacteria; p: Proteobacteria; | 961 |
| | | | c: Gammaproteobacteria; | |
| | | | o: Enterobacteriales; | |
| | | | f: Enterobacteriaceae; g:; s: | |
| 2781880 | 8 | 0.126984127 | k: Bacteria; p: Firmicutes; | 962 |
| | | | c: Clostridia; o: Clostridiales; | |
| | | | f: Ruminococcaceae; | |
| | | | g: Oscillospira; s: | |
| 2801994 | 1 | 0.015873016 | k: Bacteria; p: Firmicutes; | 963 |
| | | | c: Clostridia; o: Clostridiales; | |
| | | | f: Veillonellaceae; g: | |
| | | | Phascolarctobacterium; s: | |
| 2840201 | 35 | 0.555555556 | k: Bacteria; p: Firmicutes; | 964 |
| | | | c: Clostridia; o: Clostridiales; | |
| | | | f: Clostridiaceae; g: Clostridium; s: | |
| 2943548 | 1 | 0.015873016 | k: Bacteria; p: Firmicutes; | 965 |
| | | | c: Clostridia; o: Clostridiales; | |
| | | | f: Ruminococcaceae; | |
| | | | g: Ruminococcus; s: | |
| 2949920 | 2 | 0.031746032 | k: Bacteria; p: Firmicutes; | 966 |
| | | | c: Clostridia; o: Clostridiales; | |
| | | | f: Clostridiaceae; g:; s: | |
| 2963287 | 3 | 0.047619048 | k: Bacteria; p: Firmicutes; | 967 |
| | | | c: Clostridia; o: Clostridiales; | |
| | | | f: Christensenellaceae; g:; s: | |
| 2978122 | 8 | 0.126984127 | k: Bacteria; p: Firmicutes; | 968 |
| | | | c: Clostridia; o: Clostridiales; | |
| | | | f: Ruminococcaceae; | |
| | | | g: Oscillospira; s: | |
| 2979308 | 43 | 0.682539683 | k: Bacteria; p: Firmicutes; | 969 |
| | | | c: Clostridia; o: Clostridiales; | |
| | | | f: Ruminococcaceae; | |
| | | | g: Ruminococcus; s: | |
| 2985051 | 2 | 0.031746032 | k: Bacteria; p: Actinobacteria; | 970 |
| | | | c: Coriobacteriia; | |
| | | | o: Coriobacteriales; | |
| | | | f: Coriobacteriaceae; g:; s: | |
| 3015509 | 14 | 0.222222222 | k: Bacteria; p: Firmicutes; | 971 |
| | | | c: Clostridia; o: Clostridiales; | |
| | | | f: Ruminococcaceae; | |
| | | | g: Ruminococcus; s: | |
| 3016478 | 6 | 0.095238095 | k: Bacteria; p: Firmicutes; | 972 |
| | | | c: Clostridia; o: Clostridiales; | |
| | | | f: Ruminococcaceae; | |
| | | | g: Ruminococcus; s: | |
| 3023610 | 21 | 0.333333333 | k: Bacteria; p: Firmicutes; | 973 |
| | | | c: Clostridia; o: Clostridiales; | |
| | | | f: Lachnospiraceae; | |
| | | | g: Lachnospira; s: | 974 |
| 3028318 | 7 | 0.111111111 | k: Bacteria; p: Bacteroidetes; | |
| | | | c: Bacteroidia; o: Bacteroidales; | |
| | | | f: Rikenellaceae; g:; s. | |
| 3090117 | 2 | 0.031746032 | k: Bacteria; p: Bacteroidetes; | 975 |
| | | | c: Bacteroidia; o: Bacteroidales; | |
| | | | f: [Barnesiellaceae]; g:; s: | |
| 3123133 | 4 | 0.063492063 | k: Bacteria; p: Proteobacteria; | 976 |
| | | | c: Betaproteobacteria; | |
| | | | o: Burkholderiales; | |
| | | | f: Alcaligenaceae; g: Sutterella; s: | |
| 3138798 | 24 | 0.380952381 | k: Bacteria; p: Firmicutes; | 977 |
| | | | c: Clostridia; o: Clostridiales; | |
| | | | f: Veillonellaceae; g: | |
| | | | Phascolarctobacterium; s: | |
| 3141342 | 2 | 0.031746032 | k: Bacteria; p: Firmicutes; | 978 |
| | | | c: Clostridia; o: Clostridiales; | |
| | | | f: Lachnospiraceae; | |
| | | | g: Coprococcus; s: | |
| 3160267 | 8 | 0.126984127 | k: Bacteria; p: Bacteroidetes; | 979 |
| | | | c: Bacteroidia; o: Bacteroidales; | |
| | | | f: [Barnesiellaceae]; g:; s: | |
| 3190479 | 2 | 0.031746032 | k: Bacteria; p: Firmicutes; | 980 |
| | | | c: Clostridia; o: Clostridiales; | |
| | | | f: Christensenellaceae; g:; s: | |
| 3235048 | 7 | 0.111111111 | k: Bacteria; p: Firmicutes; | 981 |
| | | | c: Clostridia; o: Clostridiales; | |
| | | | f: Ruminococcaceae; | |
| | | | g: Ruminococcus; s: | |
| 3257594 | 1 | 0.015873016 | k: Bacteria; p: Firmicutes; | 982 |
| | | | c: Clostridia; o: Clostridiales; | |
| | | | f: Ruminococcaceae; | |
| | | | g: Oscillospira; s: | |
| 3302039 | 1 | 0.015873016 | k: Bacteria; p: Firmicutes; | 983 |
| | | | c: Clostridia; o: Clostridiales; | |
| | | | f: Ruminococcaceae; | |
| | | | g: Oscillospira; s: | |
| 3325984 | 34 | 0.53968254 | k: Bacteria; p: Firmicutes; | 984 |
| | | | c: Clostridia; o: Clostridiales; | |
| | | | f: Lachnospiraceae; | |
| | | | g: Coprococcus; s: | |
| 3369303 | 2 | 0.031746032 | k: Bacteria; p: Firmicutes; | 985 |
| | | | c: Clostridia; o: Clostridiales; | |
| | | | f: [Mogibacteriaceae]; g:; s: | |
| 3424188 | 1 | 0.015873016 | k: Bacteria; p: Firmicutes; | 986 |
| | | | c: Clostridia; o: Clostridiales; | |
| | | | f: Lachnospiraceae; | |
| | | | g: Lachnospira; s: | |
| 3430935 | 6 | 0.095238095 | k: Bacteria; p: Firmicutes; | 987 |
| | | | c: Clostridia; o: Clostridiales; | |
| | | | f: Ruminococcaceae; | |
| | | | g: Faecalibacterium; | |
| | | | s: prausnitzii | |
| 3443092 | 13 | 0.206349206 | k: Bacteria; p: Firmicutes; | 988 |
| | | | c: Clostridia; o: Clostridiales; | |
| | | | f: Ruminococcaceae; | |
| | | | g: Faecalibacterium; | |
| | | | s: prausnitzii | |
| 3505877 | 2 | 0.031746032 | k: Bacteria; p: Firmicutes; | 989 |
| | | | c: Clostridia; o: Clostridiales; | |
| | | | f: Ruminococcaceae; | |
| | | | g: Faecalibacterium; | |
| | | | s: prausnitzii | |
| 3507351 | 6 | 0.095238095 | k: Bacteria; p: Firmicutes; | 990 |
| | | | c: Clostridia; o: Clostridiales; | |
| | | | f: Veillonellaceae; g: | |
| | | | Phascolarctobacterium; s: | |
| 3522002 | 3 | 0.047619048 | k: Bacteria; p: Firmicutes; | 991 |
| | | | c: Clostridia; o: Clostridiales; | |
| | | | f: [Mogibacteriaceae]; g:; s: | |
| 3568684 | 2 | 0.031746032 | k: Bacteria; p: Firmicutes; | 992 |
| | | | c: Clostridia; o: Clostridiales; | |
| | | | f: Ruminococcaceae; | |
| | | | g: Oscillospira; s: | |
| 3589845 | 3 | 0.047619048 | k: Bacteria; p: Firmicutes; | 993 |
| | | | c: Clostridia; o: Clostridiales; | |
| | | | f: Ruminococcaceae; | |
| | | | g: Ruminococcus; s: | |
| 3613745 | 7 | 0.111111111 | k: Bacteria; p: Firmicutes; | 994 |
| | | | c: Clostridia; o: Clostridiales; | |
| | | | f: Lachnospiraceae; | |
| | | | g: Lachnospira; s: | |
| 3648884 | 2 | 0.031746032 | k: Bacteria; p: Firmicutes; | 995 |
| | | | c: Clostridia; o: Clostridiales; | |
| | | | f: Lachnospiraceae; | |
| | | | g: Coprococcus; s: | |
| 3734841 | 1 | 0.015873016 | k: Bacteria; p: Firmicutes; | 996 |
| | | | c: Clostridia; o: Clostridiales; | |
| | | | f: Clostridiaceae; g:; s: | |
| 3805726 | 4 | 0.063492063 | k: Bacteria; p: Firmicutes; | 997 |
| | | | c: Clostridia; o: Clostridiales; | |
| | | | f: [Mogibacteriaceae]; g:; s: | |
| 3903651 | 54 | 0.857142857 | k: Bacteria; p: Firmicutes; | 998 |
| | | | c: Clostridia; o: Clostridiales; | |
| | | | f: Ruminococcaceae; | |
| | | | g: Oscillospira; s: | |
| 3931537 | 41 | 0.650793651 | k: Bacteria; p: Firmicutes; | 999 |
| | | | c: Clostridia; o: Clostridiales; | |
| | | | f: Clostridiaceae; g: Clostridium; s: | |
| 3991008 | 3 | 0.047619048 | k: Bacteria; p: Firmicutes; | 1000 |
| | | | c: Clostridia; o: Clostridiales; | |
| | | | f: Ruminococcaceae; | |
| | | | g: Faecalibacterium; | |
| | | | s: prausnitzii | |
| 4035247 | 23 | 0.365079365 | k: Bacteria; p: Bacteroidetes; | 1001 |
| | | | c: Bacteroidia; o: Bacteroidales; | |
| | | | f: Rikenellaceae; g:; s. | |
| 4039230 | 25 | 0.396825397 | k: Bacteria; p: Firmicutes; | 1002 |
| | | | c: Clostridia; o: Clostridiales; | |
| | | | f: Lachnospiraceae; | |
| | | | g: Lachnospira; s: | |
| 4060645 | 7 | 0.111111111 | k: Bacteria; p: Firmicutes; | 1003 |
| | | | c: Clostridia; o: Clostridiales; | |
| | | | f: Lachnospiraceae; | |
| | | | g: Lachnospira; s: | |
| 4093791 | 1 | 0.015873016 | k: Bacteria; p: Firmicutes; | 1004 |
| | | | c: Clostridia; o: Clostridiales; | |
| | | | f: [Mogibacteriaceae]; g:; s. | |
| 4095596 | 56 | 0.888888889 | k: Bacteria; p: Proteobacteria; | 1005 |
| | | | c: Betaproteobacteria; | |
| | | | o: Burkholderiales; | |
| | | | f: Alcaligenaceae; g: Sutterella; s: | |
| 4111715 | 7 | 0.111111111 | k: Bacteria; p: Proteobacteria; | 1006 |
| | | | c: Gammaproteobacteria; | |
| | | | o: Enterobacteriales; | |
| | | | f: Enterobacteriaceae; g:; s: | |
| 4120404 | 7 | 0.111111111 | k: Bacteria; p: Proteobacteria; | 1007 |
| | | | c: Betaproteobacteria; | |
| | | | o: Burkholderiales; | |
| | | | f: Alcaligenaceae; g: Sutterella; s: | |
| 4142052 | 1 | 0.015873016 | k: Bacteria; p: Proteobacteria; | 1008 |
| | | | c: Betaproteobacteria; | |
| | | | o: Burkholderiales; | |
| | | | f: Alcaligenaceae; g: Sutterella; s: | |
| 4194837 | 13 | 0.206349206 | k: Bacteria; p: Firmicutes; | 1009 |
| | | | c: Clostridia; o: Clostridiales; | |
| | | | f: Lachnospiraceae; | |
| | | | g: Coprococcus; s: | |
| 4217226 | 1 | 0.015873016 | k: Bacteria; p: Proteobacteria; | 1010 |
| | | | c: Betaproteobacteria; | |
| | | | o: Burkholderiales; | |
| | | | f: Alcaligenaceae; g: Sutterella; s: | 1011 |
| 4238179 | 1 | 0.015873016 | k: Bacteria; p: Firmicutes; | |
| | | | c: Clostridia; o: Clostridiales; | |
| | | | f: Clostridiaceae; g:; s: | |
| 4282363 | 1 | 0.015873016 | k: Bacteria; p: Firmicutes; | |
| | | | c: Clostridia; o: Clostridiales; | 1012 |
| | | | f: Ruminococcaceae; | |
| | | | g: Faecalibacterium; | |
| | | | s: prausnitzii | 1013 |
| 4289858 | 1 | 0.015873016 | k: Bacteria; p: Firmicutes; | |
| | | | c: Clostridia; o: Clostridiales; | |
| | | | f: Clostridiaceae; g:; s: | |
| 4290144 | 1 | 0.015873016 | k: Bacteria; p: Firmicutes; | 1014 |
| | | | c: Bacilli; o: | |
| | | | Lactobacillusococcaceae; | |
| | | | g: Enterococcus; s: | |
| 4296216 | 26 | 0.412698413 | k: Bacteria; p: Firmicutes; | 1015 |
| | | | c: Clostridia; o: Clostridiales; | |
| | | | f: Ruminococcaceae; | |
| | | | g: Ruminococcus; s: | 1016 |
| 4296764 | 3 | 0.047619048 | k: Bacteria; p: Firmicutes; | |
| | | | c: Clostridia; o: Clostridiales; | |
| | | | f: Christensenellaceae; g:; s: | |
| 4296772 | 1 | 0.015873016 | k: Bacteria; p: Firmicutes; | 1017 |
| | | | c: Clostridia; o: Clostridiales; | |
| | | | f: Lachnospiraceae; | |
| | | | g: Coprococcus; s: | |
| 4303851 | 2 | 0.031746032 | k: Bacteria; p: Firmicutes; | 1018 |
| | | | c: Clostridia; o: Clostridiales; | |
| | | | f: Ruminococcaceae; | |
| | | | g: Ruminococcus; s: | |
| 4303855 | 1 | 0.015873016 | k: Bacteria; p: Firmicutes; | 1019 |
| | | | c: Clostridia; o: Clostridiales; | |
| | | | f: Ruminococcaceae; | |
| | | | g: Ruminococcus; s: | |
| 4304843 | 6 | 0.095238095 | k: Bacteria; p: Actinobacteria; | 1020 |
| | | | c: Coriobacteriia; | |
| | | | o: Coriobacteriales; | |
| | | | f: Coriobacteriaceae; g:; s: | |
| 4305261 | 2 | 0.031746032 | k: Bacteria; p: Firmicutes; | 1021 |
| | | | c: Clostridia; o: Clostridiales; | |
| | | | f: [Mogibacteriaceae]; g:; s: | |
| 4308811 | 1 | 0.015873016 | k: Bacteria; p: Actinobacteria; | 1022 |
| | | | c: Coriobacteriia; | |
| | | | o: Coriobacteriales; | |
| | | | f: Coriobacteriaceae; g:; s: | |
| 4311621 | 7 | 0.111111111 | k: Bacteria; p: Firmicutes; | 1023 |
| | | | c: Clostridia; o: Clostridiales; | |
| | | | f: Veillonellaceae; g: | |
| | | | Phascolarctobacterium; s: | |
| 4315688 | 1 | 0.015873016 | k: Bacteria; p: Firmicutes; | 1024 |
| | | | c: Clostridia; o: Clostridiales; | |
| | | | f: Clostridiaceae; g: Clostridium; | |
| | | | s: celatum | |
| 4317896 | 1 | 0.015873016 | k: Bacteria; p: Firmicutes; | 1025 |
| | | | c: Clostridia; o: Clostridiales; | |
| | | | f: Ruminococcaceae; | |
| | | | g: Oscillospira; s: | |
| 4321292 | 2 | 0.031746032 | k: Bacteria; p: Proteobacteria; | 1026 |
| | | | c: Gammaproteobacteria; | |
| | | | o: Enterobacteriales; | |
| | | | f: Enterobacteriaceae; g: Proteus; s: | |
| 4325533 | 15 | 0.238095238 | k: Bacteria; p: Bacteroidetes; | 1027 |
| | | | c: Bacteroidia; o: Bacteroidales; | |
| | | | f: Rikenellaceae; g:; s: | |
| 4325836 | 2 | 0.031746032 | k: Bacteria; p: Firmicutes; | 1028 |
| | | | c: Clostridia; o: Clostridiales; | |
| | | | f: Clostridiaceae; g:; s: | |
| 4326080 | 32 | 0.507936508 | k: Bacteria; p: Firmicutes; | 1029 |
| | | | c: Clostridia; o: Clostridiales; | |
| | | | f: Clostridiaceae; g: Clostridium; s: | |
| 4326869 | 19 | 0.301587302 | k: Bacteria; p: Firmicutes; | 1030 |
| | | | c: Clostridia; o: Clostridiales; | |
| | | | f: Ruminococcaceae; | |
| | | | g: Ruminococcus; s: | |
| 4328189 | 3 | 0.047619048 | k: Bacteria; p: Proteobacteria; | 1031 |
| | | | c: Gammaproteobacteria; | |
| | | | o: Enterobacteriales; | |
| | | | f: Enterobacteriaceae; g: Serratia; s: | |
| 4331723 | 32 | 0.507936508 | k: Bacteria; p: Firmicutes; | 1032 |
| | | | c: Clostridia; o: Clostridiales; | |
| | | | f: Ruminococcaceae; | |
| | | | g: Ruminococcus; s: | |
| 4331760 | 16 | 0.253968254 | k: Bacteria; p: Bacteroidetes; | 1033 |
| | | | c: Bacteroidia; o: Bacteroidales; | |
| | | | f: Rikenellaceae; g: | |
| | | | Alistipes; s: indistinctus | |
| 4332078 | 11 | 0.174603175 | k: Bacteria; p: Firmicutes; | 1034 |
| | | | c: Clostridia; o: Clostridiales; | |
| | | | f: Clostridiaceae; g:; s: | |
| 4333020 | 1 | 0.015873016 | k: Bacteria; p: Proteobacteria; | 1035 |
| | | | c: Gammaproteobacteria; | |
| | | | o: Enterobacteriales; | |
| | | | f: Enterobacteriaceae; g:; s: | |
| 4335402 | 1 | 0.015873016 | k: Bacteria; p: Firmicutes; | 1036 |
| | | | c: Clostridia; o: Clostridiales; | |
| | | | f: Lachnospiraceae; | |
| | | | g: Coprococcus; s: | |
| 4336423 | 3 | 0.047619048 | k: Bacteria; p: Firmicutes; | 1037 |
| | | | c: Clostridia; o: Clostridiales; | |
| | | | f: Ruminococcaceae; | |
| | | | g: Faecalibacterium; | |
| | | | s: prausnitzii | |
| 4336943 | 8 | 0.126984127 | k: Bacteria; p: Bacteroidetes; | 1038 |
| | | | c: Bacteroidia; o: Bacteroidales; | |
| | | | f: Rikenellaceae; g:; s: | |
| 4337970 | 3 | 0.047619048 | k: Bacteria; p: Bacteroidetes; | 1039 |
| | | | c: Bacteroidia; o: Bacteroidales; | |
| | | | f: [Barnesiellaceae]; g:; s: | |
| 4338624 | 7 | 0.111111111 | k: Bacteria; p: Proteobacteria; | 1040 |
| | | | c: Betaproteobacteria; | |
| | | | o: Burkholderiales; | |
| | | | f: Alcaligenaceae; g: Sutterella; s: | |
| 4339145 | 1 | 0.015873016 | k: Bacteria; p: Actinobacteria; | 1041 |
| | | | c: Coriobacteriia; | |
| | | | o: Coriobacteriales; | |
| | | | f: Coriobacteriaceae; g:; s: | |
| 4341497 | 1 | 0.015873016 | k: Bacteria; p: Proteobacteria; | 1042 |
| | | | c: Betaproteobacteria; | |
| | | | o: Burkholderiales; | |
| | | | f: Alcaligenaceae; g: Sutterella; s: | |
| 4349517 | 2 | 0.031746032 | k: Bacteria; p: Firmicutes; | 1043 |
| | | | c: Clostridia; o: Clostridiales; | |
| | | | f: Clostridiaceae; g:; s: | |
| 4353658 | 2 | 0.031746032 | k: Bacteria; p: Firmicutes; | 1044 |
| | | | c: Clostridia; o: Clostridiales; | |
| | | | f: Veillonellaceae; g: | |
| | | | Phascolarctobacterium; s: | |
| 4354477 | 33 | 0.523809524 | k: Bacteria; p: Proteobacteria; | 1045 |
| | | | c: Gammaproteobacteria; | |
| | | | o: Enterobacteriales; | |
| | | | f: Enterobacteriaceae; g:; s: | |
| 4356080 | 13 | 0.206349206 | k: Bacteria; p: Bacteroidetes; | 1046 |
| | | | c: Bacteroidia; o: Bacteroidales; | |
| | | | f: [Barnesiellaceae]; g:; s: | |
| 4357223 | 2 | 0.031746032 | k: Bacteria; p: Firmicutes; | 1047 |
| | | | c: Clostridia; o: Clostridiales; | |
| | | | f: Ruminococcaceae; | |
| | | | g: Ruminococcus; s: | |
| 4359216 | 5 | 0.079365079 | k: Bacteria; p: Firmicutes; | 1048 |
| | | | c: Clostridia; o: Clostridiales; | |
| | | | f: Ruminococcaceae; | |
| | | | g: Faecalibacterium; | |
| | | | s: prausnitzii | |
| 4361768 | 1 | 0.015873016 | k: Bacteria; p: Actinobacteria; | 1049 |
| | | | c: Coriobacteriia; | |
| | | | o: Coriobacteriales; | |
| | | | f: Coriobacteriaceae; g:; s: | |
| 4362724 | 21 | 0.333333333 | k: Bacteria; p: Firmicutes; | 1050 |
| | | | c: Clostridia; o: Clostridiales; | |
| | | | f: Ruminococcaceae; | |
| | | | g: Oscillospira; s: | |
| 4364469 | 2 | 0.031746032 | k: Bacteria; p: Proteobacteria; | 1051 |
| | | | c: Gammaproteobacteria; | |
| | | | o: Enterobacteriales; | |
| | | | f: Enterobacteriaceae; | |
| | | | g: Brenneria; s: alni | |
| 4364747 | 38 | 0.603174603 | k: Bacteria; p: Firmicutes; | 1052 |
| | | | c: Clostridia; o: Clostridiales; | |
| | | | f: Clostridiaceae; g:; s: | |
| 4366089 | 1 | 0.015873016 | k: Bacteria; p: Firmicutes; | 1053 |
| | | | c: Clostridia; o: Clostridiales; | |
| | | | f: Ruminococcaceae; | |
| | | | g: Oscillospira; s: | |
| 4366834 | 1 | 0.015873016 | k: Bacteria; p: Proteobacteria; | 1054 |
| | | | c: Betaproteobacteria; | |
| | | | o: Burkholderiales; | |
| | | | f: Alcaligenaceae; g: Sutterella; s: | |
| 4370657 | 1 | 0.015873016 | k: Bacteria; p: Firmicutes; | 1055 |
| | | | c: Clostridia; o: Clostridiales; | |
| | | | f: Clostridiaceae; g: Clostridium; | |
| | | | s: perfringens | |
| 4371341 | 6 | 0.095238095 | k: Bacteria; p: Firmicutes; | 1056 |
| | | | c: Clostridia; o: Clostridiales; | |
| | | | f: Veillonellaceae; g: | |
| | | | Phascolarctobacterium; s: | |
| 4371724 | 1 | 0.015873016 | k: Bacteria; p: Firmicutes; | 1057 |
| | | | c: Clostridia; o: Clostridiales; | |
| | | | f: Ruminococcaceae; | |
| | | | g: Ruminococcus; s: | |
| 4371786 | 2 | 0.031746032 | k: Bacteria; p: Bacteroidetes; | 1058 |
| | | | c: Bacteroidia; o: Bacteroidales; | |
| | | | f: [Barnesiellaceae]; g:; s: | |
| 4375000 | 2 | 0.031746032 | k: Bacteria; p: Proteobacteria; | 1059 |
| | | | c: Gammaproteobacteria; | |
| | | | o: Enterobacteriales; | |
| | | | f: Enterobacteriaceae; g:; s: | |
| 4379449 | 1 | 0.015873016 | k: Bacteria; p: Firmicutes; | 1060 |
| | | | c: Erysipelotrichi; | |
| | | | o: Erysipelotrichales; | |
| | | | f: Erysipelotrichaceae; | |
| | | | g: Clostridium; s: saccharogumia | |
| 4381430 | 1 | 0.015873016 | k: Bacteria; p: Firmicutes; | 1061 |
| | | | c: Clostridia; o: Clostridiales; | |
| | | | f: Ruminococcaceae; | |
| | | | g: Faecalibacterium; | |
| | | | s: prausnitzii | |
| 4381639 | 4 | 0.063492063 | k: Bacteria; p: Firmicutes; | 1062 |
| | | | c: Clostridia; o: Clostridiales; | |
| | | | f: Ruminococcaceae; | |
| | | | g: Ruminococcus; s: | |
| 4385756 | 1 | 0.015873016 | k: Bacteria; p: Firmicutes; | 1063 |
| | | | c: Clostridia; o: Clostridiales; | |
| | | | f: Clostridiaceae; g:; s: | |
| 4387208 | 1 | 0.015873016 | k: Bacteria; p: Firmicutes; | 1064 |
| | | | c: Bacilli; o: Lactobacillococcaceae; | |
| | | | g: Enterococcus; s: | |
| 4387344 | 6 | 0.095238095 | k: Bacteria; p: Firmicutes; | 1065 |
| | | | c: Clostridia; o: Clostridiales; | |
| | | | f: Ruminococcaceae; | |
| | | | g: Faecalibacterium; | |
| | | | s: prausnitzii | |
| 4391009 | 2 | 0.031746032 | k: Bacteria; p: Proteobacteria; | 1066 |
| | | | c: Betaproteobacteria; | |
| | | | o: Burkholderiales; | |
| | | | f: Alcaligenaceae; g: Sutterella; s: | |
| 4391738 | 1 | 0.015873016 | k: Bacteria; p: Firmicutes; | 1067 |
| | | | c: Bacilli; o: Lactobacillococcaceae; | |
| | | | g: Enterococcus; s: | |
| 4393396 | 8 | 0.126984127 | k: Bacteria; p: Bacteroidetes; | 1068 |
| | | | c: Bacteroidia; o: Bacteroidales; | |
| | | | f: [Barnesiellaceae]; g:; s: | |
| 4397092 | 2 | 0.031746032 | k: Bacteria; p: Actinobacteria; | 1069 |
| | | | c: Coriobacteriia; | |
| | | | o: Coriobacteriales; | |
| | | | f: Coriobacteriaceae; g:; s: | |
| 4400482 | 1 | 0.015873016 | k: Bacteria; p: Firmicutes; | 1070 |
| | | | c: Clostridia; o: Clostridiales; | |
| | | | f: Clostridiaceae; g: Clostridium; | |
| | | | s: butyricum | |
| 4401045 | 2 | 0.031746032 | k: Bacteria; p: Firmicutes; | 1071 |
| | | | c: Clostridia; o: Clostridiales; | |
| | | | f: Clostridiaceae; g: Clostridium; s: | |
| 4402537 | 11 | 0.174603175 | k: Bacteria; p: Actinobacteria; | 1072 |
| | | | c: Coriobacteriia; | |
| | | | o: Coriobacteriales; | |
| | | | f: Coriobacteriaceae; g:; s: | |
| 4402605 | 1 | 0.015873016 | k: Bacteria; p: Firmicutes; | 1073 |
| | | | c: Clostridia; o: Clostridiales; | |
| | | | f: Christensenellaceae; g:; s: | |
| 4403113 | 33 | 0.523809524 | k: Bacteria; p: Firmicutes; | 1074 |
| | | | c: Clostridia; o: Clostridiales; | |
| | | | f: Ruminococcaceae; | |
| | | | g: Faecalibacterium; | |
| | | | s: prausnitzii | |
| 4403259 | 6 | 0.095238095 | k: Bacteria; p: Actinobacteria; | 1075 |
| | | | c: Coriobacteriia; | |
| | | | o: Coriobacteriales; | |
| | | | f: Coriobacteriaceae; g:; s: | |
| 4403632 | 53 | 0.841269841 | k: Bacteria; p: Firmicutes; | 1076 |
| | | | c: Clostridia; o: Clostridiales; | |
| | | | f: Lachnospiraceae; | |
| | | | g: Coprococcus; s: | |
| 4407939 | 5 | 0.079365079 | k: Bacteria; p: Proteobacteria; | 1077 |
| | | | c: Gammaproteobacteria; | |
| | | | o: Enterobacteriales; | |
| | | | f: Enterobacteriaceae; g:; s: | |
| 4410097 | 1 | 0.015873016 | k: Bacteria; p: Firmicutes; | 1078 |
| | | | c: Clostridia; o: Clostridiales; | |
| | | | f: Christensenellaceae; g:; s: | |
| 4414821 | 1 | 0.015873016 | k: Bacteria; p: Firmicutes; | 1079 |
| | | | c: Clostridia; o: Clostridiales; | |
| | | | f: Ruminococcaceae; | |
| | | | g: Ruminococcus; s: | |
| 4416113 | 1 | 0.015873016 | k: Bacteria; p: Proteobacteria; | 1080 |
| | | | c: Gammaproteobacteria; | |
| | | | o: Enterobacteriales; | |
| | | | f: Enterobacteriaceae; g:; s: | |
| 4416646 | 29 | 0.46031746 | k: Bacteria; p: Firmicutes; | 1081 |
| | | | c: Clostridia; o: Clostridiales; | |
| | | | f: Ruminococcaceae; | |
| | | | g: Ruminococcus; s: | |
| 4416951 | 7 | 0.111111111 | k: Bacteria; p: Firmicutes; | 1082 |
| | | | c: Clostridia; o: Clostridiales; | |
| | | | f: Ruminococcaceae; | |
| | | | g: Ruminococcus; s: | 1083 |
| 4419621 | 1 | 0.015873016 | k: Bacteria; p: Proteobacteria; | |
| | | | c: Betaproteobacteria; | |
| | | | o: Burkholderiales; | |
| | | | f: Alcaligenaceae; g: Sutterella; s: | |
| 4419650 | 47 | 0.746031746 | k: Bacteria; p: Firmicutes; | 1084 |
| | | | c: Clostridia; o: Clostridiales; | |
| | | | f: Lachnospiraceae; | |
| | | | g: Coprococcus; s: | |
| 4421273 | 17 | 0.26984127 | k: Bacteria; p: Firmicutes; | 1085 |
| | | | c: Clostridia; o: Clostridiales; | |
| | | | f: Ruminococcaceae; | |
| | | | g: Oscillospira; s: | |
| 4424598 | 6 | 0.095238095 | k: Bacteria; p: Firmicutes; | 1086 |
| | | | c: Clostridia; o: Clostridiales; | |
| | | | f: Lachnospiraceae; | |
| | | | g: Lachnospira; s: | |
| 4425571 | 12 | 0.19047619 | k: Bacteria; p: Proteobacteria; | 1087 |
| | | | c: Gammaproteobacteria; | |
| | | | o: Enterobacteriales; | |
| | | | f: Enterobacteriaceae; g:; s: | |
| 4425669 | 5 | 0.079365079 | k: Bacteria; p: Firmicutes; | 1088 |
| | | | c: Clostridia; o: Clostridiales; | |
| | | | f: Lachnospiraceae; | |
| | | | g: Coprococcus; s: | |
| 4426249 | 1 | 0.015873016 | k: Bacteria; p: Firmicutes; | 1089 |
| | | | c: Clostridia; o: Clostridiales; | |
| | | | f: Ruminococcaceae; | |
| | | | g: Oscillospira; s: | |
| 4427459 | 12 | 0.19047619 | k: Bacteria; p: Firmicutes; | 1090 |
| | | | c: Clostridia; o: Clostridiales; | |
| | | | f: Ruminococcaceae; | |
| | | | g: Ruminococcus; s: | |
| 4428676 | 4 | 0.063492063 | k: Bacteria; p: Firmicutes; | 1091 |
| | | | c: Clostridia; o: Clostridiales; | |
| | | | f: Lachnospiraceae; | |
| | | | g: Coprococcus; s: | |
| 4429986 | 1 | 0.015873016 | k: Bacteria; p: Proteobacteria; | 1092 |
| | | | c: Betaproteobacteria; | |
| | | | o: Burkholderiales; | |
| | | | f: Alcaligenaceae; g: Sutterella; s: | |
| 4433274 | 1 | 0.015873016 | k: Bacteria; p: Proteobacteria; | 1093 |
| | | | c: Gammaproteobacteria; | |
| | | | o: Enterobacteriales; | |
| | | | f: Enterobacteriaceae; g:; s: | |
| 4434334 | 57 | 0.904761905 | k: Bacteria; p: Firmicutes; | 1094 |
| | | | c: Clostridia; o: Clostridiales; | |
| | | | f: Clostridiaceae; g:; s: | |
| 4437359 | 2 | 0.031746032 | k: Bacteria; p: Firmicutes; | 1095 |
| | | | c: Clostridia; o: Clostridiales; | |
| | | | f: Ruminococcaceae; | |
| | | | g: Oscillospira; s: | |
| 4439530 | 3 | 0.047619048 | k: Bacteria; p: Firmicutes; | 1096 |
| | | | c: Clostridia; o: Clostridiales; | |
| | | | f: Clostridiaceae; g:; s: | |
| 4440335 | 1 | 0.015873016 | k: Bacteria; p: Firmicutes; | 1097 |
| | | | c: Clostridia; o: Clostridiales; | |
| | | | f: Christensenellaceae; g:; s: | |
| 4441081 | 2 | 0.031746032 | k: Bacteria; p: Actinobacteria; | 1098 |
| | | | c: Coriobacteriia; | |
| | | | o: Coriobacteriales; | |
| | | | f: Coriobacteriaceae; g:; s: | |
| 4443143 | 7 | 0.111111111 | k: Bacteria; p: Firmicutes; | 1099 |
| | | | c: Clostridia; o: Clostridiales; | |
| | | | f: Ruminococcaceae; | |
| | | | g: Ruminococcus; s: | |
| 4444277 | 2 | 0.031746032 | k: Bacteria; p: Bacteroidetes; | 1100 |
| | | | c: Bacteroidia; o: Bacteroidales; | |
| | | | f: [Odoribacteraceae]; | |
| | | | g: Odoribacter; s: | |
| 4444529 | 1 | 0.015873016 | k: Bacteria; p: Bacteroidetes; | 1101 |
| | | | c: Bacteroidia; o: Bacteroidales; | |
| | | | f: Rikenellaceae; g:; s. | |
| 4444790 | 6 | 0.095238095 | k: Bacteria; p: Firmicutes; | 1102 |
| | | | c: Clostridia; o: Clostridiales; | |
| | | | f: Veillonellaceae; g: | |
| | | | Phascolarctobacterium; s: | |
| 4445226 | 1 | 0.015873016 | k: Bacteria; p: Bacteroidetes; | 1103 |
| | | | c: Bacteroidia; o: Bacteroidales; | |
| | | | f: [Barnesiellaceae]; g:; s: | |
| 4446320 | 25 | 0.396825397 | k: Bacteria; p: Firmicutes; | 1104 |
| | | | c: Clostridia; o: Clostridiales; | |
| | | | f: Clostridiaceae; g:; s: | |
| 4449236 | 1 | 0.015873016 | k: Bacteria; p: Proteobacteria; | 1105 |
| | | | c: Betaproteobacteria; | |
| | | | o: Burkholderiales; | |
| | | | f: Alcaligenaceae; g: Sutterella; s: | |
| 4451901 | 1 | 0.015873016 | k: Bacteria; p: Firmicutes; | 1106 |
| | | | c: Clostridia; o: Clostridiales; | |
| | | | f: [Mogibacteriaceae]; g:; s: | |
| 4452633 | 1 | 0.015873016 | k: Bacteria; p: Firmicutes; | 1107 |
| | | | c: Clostridia; o: Clostridiales; | |
| | | | f: Clostridiaceae; g:; s: | |
| 4453060 | 3 | 0.047619048 | k: Bacteria; p: Firmicutes; | 1108 |
| | | | c: Bacilli; o: | |
| | | | Lactobacillococcaceae; | |
| | | | g: Enterococcus; s: | |
| 4454586 | 53 | 0.841269841 | k: Bacteria; p: Bacteroidetes; | 1109 |
| | | | c: Bacteroidia; o: Bacteroidales; | |
| | | | f: [Odoribacteraceae]; | |
| | | | g: Odoribacter; s: | |
| 4455005 | 1 | 0.015873016 | k: Bacteria; p: Firmicutes; | 1110 |
| | | | c: Clostridia; o: Clostridiales; | |
| | | | f: [Mogibacteriaceae]; g:; s: | |
| 4456702 | 2 | 0.031746032 | k: Bacteria; p: Firmicutes; | 1111 |
| | | | c: Clostridia; o: Clostridiales; | |
| | | | f: Ruminococcaceae; | |
| | | | g: Ruminococcus; s: | |
| 4457064 | 1 | 0.015873016 | k: Bacteria; p: Bacteroidetes; | 1112 |
| | | | c: Bacteroidia; o: Bacteroidales; | |
| | | | f: Rikenellaceae; g:; s: | |
| 4457427 | 44 | 0.698412698 | k: Bacteria; p: Firmicutes; | 1113 |
| | | | c: Clostridia; o: Clostridiales; | |
| | | | f: Lachnospiraceae; | |
| | | | g: Lachnospira; s: | |
| 4459196 | 3 | 0.047619048 | k: Bacteria; p: Firmicutes; | 1114 |
| | | | c: Clostridia; o: Clostridiales; | |
| | | | f: Lachnospiraceae; | |
| | | | g: Lachnospira; s: | |
| 4460847 | 32 | 0.507936508 | k: Bacteria; p: Firmicutes; | 1115 |
| | | | c: Clostridia; o: Clostridiales; | |
| | | | f: Ruminococcaceae; | |
| | | | g: Ruminococcus; s: | |
| 4463709 | 45 | 0.714285714 | k: Bacteria; p: Firmicutes; | 1116 |
| | | | c: Clostridia; o: Clostridiales; | |
| | | | f: Lachnospiraceae; | |
| | | | g: Coprococcus; s: | |
| 4466707 | 1 | 0.015873016 | k: Bacteria; p: Firmicutes; | 1117 |
| | | | c: Clostridia; o: Clostridiales; | |
| | | | f: Lachnospiraceae; | |
| | | | g: Coprococcus; s: | |
| 4468461 | 1 | 0.015873016 | k: Bacteria; p: Firmicutes; | 1118 |
| | | | c: Clostridia; o: Clostridiales; | |
| | | | f: Clostridiaceae; g:; s: | |
| 4470582 | 1 | 0.015873016 | k: Bacteria; p: Firmicutes; | 1119 |
| | | | c: Clostridia; o: Clostridiales; | |
| | | | f: Ruminococcaceae; | |
| | | | g: Faecalibacterium; | |
| | | | s: prausnitzii | |
| 4470870 | 2 | 0.031746032 | k: Bacteria; p: Bacteroidetes; | 1120 |
| | | | c: Bacteroidia; o: Bacteroidales; | |
| | | | f: Barnesiellaceae]; g:; s: | |
| 4471854 | 3 | 0.047619048 | k: Bacteria; p: Firmicutes; | 1121 |
| | | | c: Clostridia; o: Clostridiales; | |
| | | | f: Lachnospiraceae; | |
| | | | g: Coprococcus; s: | |
| 4472091 | 45 | 0.714285714 | k: Bacteria; p: Firmicutes; | 1122 |
| | | | c: Clostridia; o: Clostridiales; | |
| | | | f: Ruminococcaceae; | |
| | | | g: Ruminococcus; s: | |
| 4472721 | 47 | 0.746031746 | k: Bacteria; p: Bacteroidetes; | 1123 |
| | | | c: Bacteroidia; o: Bacteroidales; | |
| | | | f: [Odoribacteraceae]; | |
| | | | g: Odoribacter; s: | 1124 |
| 4473506 | 5 | 0.079365079 | k: Bacteria; p: Proteobacteria; | |
| | | | c: Betaproteobacteria; | |
| | | | o: Burkholderiales; | |
| | | | f: Alcaligenaceae; g: Sutterella; s: | 1125 |
| 4474255 | 1 | 0.015873016 | k: Bacteria; p: Proteobacteria; | |
| | | | c: Betaproteobacteria; | |
| | | | o: Burkholderiales; | 1126 |
| | | | f: Alcaligenaceae; g: Sutterella; s: | |
| 4476561 | 12 | 0.19047619 | k: Bacteria; p: Bacteroidetes; | |
| | | | c: Bacteroidia; o: Bacteroidales; | |
| | | | f: [Barnesiellaceae]; g:; s: | |
| 4476780 | 20 | 0.317460317 | k: Bacteria; p: Bacteroidetes; | 1127 |
| | | | c: Bacteroidia; o: Bacteroidales; | |
| | | | f: Rikenellaceae; g:; s: | |
| 4476877 | 4 | 0.063492063 | k: Bacteria; p: Bacteroidetes; | 1128 |
| | | | c: Bacteroidia; o: Bacteroidales; | |
| | | | f: [Odoribacteraceae]; | |
| | | | g: Odoribacter; s: | |
| 4478840 | 1 | 0.015873016 | k: Bacteria; p: Firmicutes; | 1129 |
| | | | c: Clostridia; o: Clostridiales; | |
| | | | f: Clostridiaceae; g:; s: | |
| 4481131 | 62 | 0.984126984 | k: Bacteria; p: Firmicutes; | 1130 |
| | | | c: Clostridia; o: Clostridiales; | |
| | | | f: Ruminococcaceae; | |
| | | | g: Faecalibacterium; | |
| | | | s: prausnitzii | |
| 4481613 | 2 | 0.031746032 | k: Bacteria; p: Actinobacteria; | 1131 |
| | | | c: Coriobacteriia; | |
| | | | o: Coriobacteriales; | |
| | | | f: Coriobacteriaceae; | |
| | | | g: Collinsella; s: aerofaciens | |
| 4481624 | 2 | 0.031746032 | k: Bacteria; p: Firmicutes; | 1132 |
| | | | c: Clostridia; o: Clostridiales; | |
| | | | f: Clostridiaceae; g:; s: | |
| 4483570 | 1 | 0.015873016 | k: Bacteria; p: Bacteroidetes; | 1133 |
| | | | c: Bacteroidia; o: Bacteroidales; | |
| | | | f: Rikenellaceae; g:; s: | |

¹Operational taxonomic units (OTUs) which cluster bacteria based on 16S rRNA sequence identity.
²In this column, letters refer to phylogenetic classifications (e.g., “k”: refers to “kingdom,” “p:” refers to phylum, and so on).

Table 6 lists exemplary bacterial genera and the number of OTUs identified in GreenGenes database (and recited in Table 5).

### Columns:

A—Taxonomy for that genus
B—Number of OTUs that are found in healthy stool donors that belong to this genus
C—SEQ ID NOs associated with OTUs

TABLE 6
Exemplary Genera Useful in the Present Invention

| A³ | B | C |
|---|---|---|
| k_Bacteria; p_Proteobacteria; c_Gammaproteobacteria; o_Enterobacteriales; | 8 | 1134 to 1141 |
| f_Enterobacteriaceae; g_Proteus | | |
| k_Bacteria; p_Bacteroidetes; c_Bacteroidia; o_Bacteroidales; f_Rikenellaceae; g_ | 34 | 1142 to 1175 |
| k_Bacteria; p_Firmicutes; c_Clostridia; o_Clostridiales; f_Ruminococcaceae; | 1 | 1176 |
| g_Clostridium | | |
| k_Bacteria; p_Firmicutes; c_Clostridia; o_Clostridiales; f_Lachnospiraceae; | 2 | 1177 to 1178 |
| g_Clostridium | | |
| k_Bacteria; p_Proteobacteria; c_Gammaproteobacteria; o_Enterobacteriales; | 291 | 1179 to 1469 |
| f_Enterobacteriaceae; g_ | | |
| k_Bacteria; p_Firmicutes; c_Clostridia; o_Clostridiales; f_Lachnospiraceae; | 248 | 1470 to 1717 |
| g_Coprococcus | | |
| k_Bacteria; p_Firmicutes; c_Clostridia; o_Clostridiales; f_Veillonellaceae; | 28 | 1718 to 1745 |
| g_Phascolarctobacterium | | |
| k_Bacteria; p_Firmicutes; c_Clostridia; o_Clostridiales; f_Lachnospiraceae; | 1 | 1746 |
| g_Ruminococcus | | |
| k_Bacteria; p_Proteobacteria; c_Gammaproteobacteria; o_Enterobacteriales; | 11 | 1747 to 1757 |
| f_Enterobacteriaceae; g_Serratia | | |
| k_Bacteria; p_Bacteroidetes; c_Bacteroidia; o_Bacteroidales; f_Odoribacteraceae; | 14 | 1758 to 1771 |
| g_Odoribacter | | |
| k_Bacteria; p_Firmicutes; c_Clostridia; o_Clostridiales; f_Clostridiaceae; g_ | 322 | 1772 to 2093 |
| k_Bacteria; p_Firmicutes; c_Clostridia; o_Clostridiales; f_Clostridiaceae; | 113 | 2094 to 2206 |
| g_Clostridium | | |
| k_Bacteria; p_Firmicutes; c_Clostridia; o_Clostridiales; f_Mogibacteriaceae; g_ | 47 | 2207 to 2253 |
| k_Bacteria; p_Bacteroidetes; c_Bacteroidia; o_Bacteroidales; f_Rikenellaceae; | 2 | 2254 to 2255 |
| g_Alistipes | | |
| k_Bacteria; p_Firmicutes; c_Clostridia; o_Clostridiales; f_Ruminococcaceae; | 188 | 2256 to 2443 |
| g_Oscillospira | | |
| k_Bacteria; p_Firmicutes; c_Erysipelotrichi; o_Erysipelotrichales; | 2 | 2444 to 2445 |
| f_Erysipelotrichaceae; g_Clostridium | | |
| k_Bacteria; p_Proteobacteria; c_Gammaproteobacteria; o_Enterobacteriales; | 6 | 2446 to 2451 |
| f_Enterobacteriaceae; g_Plesiomonas | | |
| k_Bacteria; p_Firmicutes; c_Bacilli; o_Lactobacillales; f_Enterococcaceae; | 41 | 2452 to 2492 |
| g_Enterococcus | | |
| k_Bacteria; p_Firmicutes; c_Clostridia; o_Clostridiales; f_Lachnospiraceae; | 63 | 2493 to 2555 |
| g_Lachnospira | | |
| k_Bacteria; p_Actinobacteria; c_Coriobacteriia; o_Coriobacteriales; | 83 | 2556 to 2638 |
| f_Coriobacteriaceae; g_ | | |
| k_Bacteria; p_Bacteroidetes; c_Bacteroidia; o_Bacteroidales; f_Barnesiellaceae; g_ | 31 | 2639 to 2669 |
| k_Bacteria; p_Firmicutes; c_Clostridia; o_Clostridiales; f_Ruminococcaceae; | 214 | 2670 to 2883 |
| g_Ruminococcus | | |
| k_Bacteria; p_Proteobacteria; c_Gammaproteobacteria; o_Enterobacteriales; | 28 | 2884 to 2911 |
| f_Enterobacteriaceae; g_Erwinia | | |
| k_Bacteria; p_Proteobacteria; c_Betaproteobacteria; o_Burkholderiales; | 37 | 2912 to 2948 |
| f_Alcaligenaceae; g_Sutterella | | |
| k_Bacteria; p_Proteobacteria; c_Gammaproteobacteria; o_Enterobacteriales; | 10 | 2950 to 2958 |
| f_Enterobacteriaceae; g_Enterobacter | | |
| k_Bacteria; p_Proteobacteria; c_Gammaproteobacteria; o_Enterobacteriales; | 12 | 2959 to 2970 |
| f_Enterobacteriaceae; g_Citrobacter | | |
| k_Bacteria; p_Firmicutes; c_Clostridia; o_Clostridiales; f_Christensenellaceae; g_ | 52 | 2971 to 3022 |
| k_Bacteria; p_Proteobacteria; c_Gammaproteobacteria; o_Enterobacteriales; | 1 | 3023 |
| f_Enterobacteriaceae; g_Xenorhabdus | | |
| k_Bacteria; p_Proteobacteria; c_Gammaproteobacteria; o_Enterobacteriales; | 2 | 3024 to 3025 |
| f_Enterobacteriaceae; g_Brenneria | | |
| k_Bacteria; p_Firmicutes; c_Clostridia; o_Clostridiales; f_Peptostreptococcaceae; | 3 | 3026 to 3028 |
| g_Clostridium | | |
| k_Bacteria; p_Firmicutes; c_Clostridia; o_Clostridiales; f_Ruminococcaceae; | 131 | 3029 to 3159 |
| g_Faecalibacterium | | |
| k_Bacteria; p_Actinobacteria; c_Coriobacteriia; o_Coriobacteriales; | 26 | 3160 to 3185 |
| f_Coriobacteriaceae; g_Collinsella | | |

³In this column, letters refer to phylogenetic classifications (e.g., “k” refers to “kingdom,” “p” refers to phylum, and so on).

In an embodiment, a bacterial strain is included in the pharmaceutical composition of the invention based on its abundance in donors whose stool was used for successful or unsuccessful fecal microbiota transplants (FMTs) in a patient suffering from a gut dysbiosis disorder, e.g., caused by a previous or current anti-cancer therapy.

In an embodiment, a bacterial strain is included in the pharmaceutical composition of the invention based on its presence in the stool samples of donors whose stool was used for FMTs which provided a therapeutically effective result in a GI disorder patient.

In an embodiment, a bacterial strain is included in the pharmaceutical composition of the invention based on its ability to engraft in a recipient. For example, the recipient may be a FMT recipient who received stool transplant from a donor. The bacterial strain is considered to successfully engraft if the strain is abundant in donors and also increased in recipient patients. In some embodiments, the selected bacterial strain exhibits enhanced ability to colonize the mucosa, i.e., is a good mucosal colonizer.

In various embodiments, a bacterial strain may be selected for inclusion in the bacterial mixture based on its ability to directly inhibit a bacterium (e.g., pathogenic bacterium) through production of a secreted product. In some embodiments, a bacterial strain may be selected for inclusion in the bacterial mixture based on its ability to directly compete with the pathogenic bacteria for a niche and/or for nutrients. In embodiments, a bacterial strain may be selected for inclusion in the bacterial mixture based on its ability to decolonize a pathogenic bacterium. In embodiments, a bacterial strain may be selected for inclusion in the bacterial mixture based on its ability to eradicate a pathogenic bacterium.

In an embodiment, a bacterial strain may be selected for inclusion in a bacterial mixture based on its ability to enhance production of SCFAs, which increases the thickness of the mucus layer, maintains the health of colonocytes, increases butyrate levels in the gut, inhibits nitric oxide synthase activity, reduces the concentration of host-derived nitrate levels in the gut, and/or induces IgA production. As used herein, SCFAs refer to fatty acids with an aliphatic tail of less than six carbon atoms. Illustrative SCFAs include, but are not limited to, formic acid, acetic acid, propionic acid, butyric acid, isobutyric acid, valeric acid, and isovaleric acid. Without wishing to be bound by theory, SCFAs are involved in mediation of GI inflammation, and SCFA-producing bacteria are associated with sustained clinical remission in UC. Accordingly, in some embodiments, a bacterial strain is selected based on its ability to produce increased levels of SCFAs. Additionally, in some embodiments, a bacterial strain is selected for its ability to complement the capacity of a functionally deficient microbial community (e.g., the microbial community of a patient infected and/or colonized by a pathogenic bacteria) to produce levels of SCFAs comparable to healthy individuals.

In an embodiment, a bacterial strain may be selected for inclusion in a bacterial mixture based on its ability to promote restoration of mucosal barrier functions. For example, in some embodiments, a bacterial strain may be selected based on its ability to enable mucosal healing, improve mucosal barrier function, and/or to reduce inflammation. Without wishing to be bound by theory, it is believed that inclusion of such bacterial strains reinforces both the structural and chemical barrier functions of the mucosa by displacing pathogenic mucus degrading microorganisms and support mucosal repair. In an embodiment, the inclusion of such bacterial strain impacts numerous inflammatory pathways linked to inappropriate microbial exposure.

Accordingly, in some embodiments, the pharmaceutical composition of the invention includes a bacterial strain that prevents and/or reduces the loss of mucus thickness associated with various GI disorders. In some embodiments, the pharmaceutical composition of the invention includes a bacterial strain that results in a reduction of bacterial penetration or bacterial load in the mucus. In some embodiments, the pharmaceutical composition of the invention includes a bacterial strain that reduces sulfate-reducing bacteria (SRB) in a subject.

Additional criteria that may be utilized for selecting a bacterial strain for inclusion in the pharmaceutical composition of the invention include, but are not limited to, the ability of the bacterial strain to inhibit IgA-degrading bacteria, the ability of the bacterial strain to inhibit serotonin-producing and serotonin-inducing bacteria, the ability of the bacterial strain to enhance tryptophan availability, the ability of the bacterial strain to produce anti-inflammatory zwitterionic polysaccharides, modification of signaling molecules interacting with the Aryl Hydrocarbon Receptor, and/or the ability of the bacterial strain to block the vitamin D receptor (VCD) or vitamin D signaling.

In various embodiments, individual bacterial strains are initially selected from Table 5 or Table 6 and subsequently pooled to form a mixture of bacterial strains. For example, in an embodiment, a mixture of bacterial strains may be formed by including one or more strains that has a 16S rRNA sequence that is at least about 97% identical with the 165 rRNA sequence of any one of the operational taxonomic units provided in Table 5 or Table 6, one or more bacterial strains that directly inhibit a pathogenic bacterium through production of a secreted product, one or more strains that directly compete with the pathogenic bacteria for a niche and/or for nutrients, one or more bacterial strains that decolonize a pathogenic bacterium, and/or one or more bacterial strains that eradicate a pathogenic bacterium.

In some embodiments, the mixtures of bacterial strains treat or prevent an autoimmune disorder including, but not limited to, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & Ill autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis.

In embodiments, the mixtures of bacterial strains treat or prevent diseases or disorders relating to the “gut-brain axis”, including neurodegenerative, neurodevelopmental and neurocognitive disorders, such as anorexia, anxiety, autism-spectrum disorder, depression, Parkinson's, and Schizophrenia.

In embodiments, the mixtures of bacterial strains treat, prevent, or reduce a side effect of an anti-cancer therapy and/or increase efficacy of an anti-cancer therapeutic agent and/or anti-cancer therapy.

In embodiments, the anti-cancer therapy is surgery, radiation therapy, chemotherapy (including hormonal therapy) and/or targeted therapy (including an immunotherapy). Illustrative chemotherapeutics agents are provided elsewhere herein. In embodiments, the immunotherapy binds to and/or recognizes a tumor-cell antigen and/or a cancer-cell antigen, e.g., CTLA-4, PD-1, PD-L1, or PD-L2. In embodiments, the immunotherapy comprises administration of Keytruda (Pembrolizumab), Opdivo (Nivolumab), Yervoy (Ipilimumab), Tecentriq (atezolizumab), Bavencio (avelumab), and Imfinzi (durvalumab).

In embodiments, the subject, e.g., a human, is refractory and/or non-responsive to an anti-cancer therapy (as described herein). In embodiments, the pharmaceutical composition treats a subject that presents a non-curative response, a limited response, or no response to the anti-cancer therapy, or even progress, after 12 weeks or so of receiving the anti-cancer therapy. Thus, a pharmaceutical composition of the present invention can rescue subjects that are refractory and/or non-responsive to the anti-cancer therapy. In embodiments, the subject is refractory and/or non-responsive to a treatment directed to a checkpoint molecule, e.g., CTLA-4, PD-1, PD-L1, and/or PD-L2. In embodiments, the treatment directed to a checkpoint molecule comprises administration of Keytruda (Pembrolizumab), Opdivo (Nivolumab), Yervoy (Ipilimumab), Tecentriq (atezolizumab), Bavencio (avelumab), or Imfinzi (durvalumab).

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogenous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

Representative cancers and/or tumors of the present invention include, but are not limited to, a basal cell carcinoma, biliary tract cancer; bladder cancer; bone cancer; brain and central nervous system cancer; breast cancer (including Triple-Negative Breast Cancer); cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; urothelial carcinoma, vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In various embodiments, the mixtures of bacterial strains may stimulate and/or activate Toll-like receptor activity (e.g., TLR1, and/or TLR2, and/or TLR3, and/or TLR4, and/or TLR5, and/or TLR6, and/or TLR7, and/or TLR8, and/or TLR9, and/or TLR10, and/or TLR11, and/or TLR12, and/or TLR13).

In some embodiments, the mixtures of bacterial strains treat or prevent a diarrheal disease including, but not limited to, acute bloody diarrhea (e.g., dysentery), acute watery diarrhea (e.g., cholera), checkpoint inhibitor-associated colitis, diarrhea due to food poisoning, persistent diarrhea, and traveler's diarrhea.

In some embodiments, the mixtures of bacterial strains treat or prevent an IBD or related disease including, but not limited to, Behcet's disease, collagenous colitis, Crohn's disease, diversion colitis, fulminant colitis, intermediate colitis, left-sided colitis, lymphocytic colitis, pancolitis, pouchitis, proctosigmoiditis, short bowel syndrome, ulcerative colitis, and ulcerative proctitis.

In some embodiments, the mixtures of bacterial strains treat or prevent the various GI disorders disclosed herein and/or as known in the art to be a result of gut dysbiosis.

In some embodiments the mixtures of bacterial strains reduce GI immunoactivation and inflammation.

In various embodiments, the mixtures of bacterial strains treat or prevent various bloodstream infections (BSI).

In some embodiments, the mixtures of bacterial strains treat or prevent catheter or intravascular-line infections (e.g., central-line infections).

In some embodiments, the mixtures of bacterial strains treat or prevent chronic inflammatory diseases.

In some embodiments, the mixtures of bacterial strains treat or prevent meningitis.

In some embodiments, the mixtures of bacterial strains treat or prevent pneumonia, e.g., ventilator-associated pneumonia.

In some embodiments, the mixtures of bacterial strains treat or prevent skin and soft tissue infections.

In some embodiments, the mixtures of bacterial strains treat or prevent surgical-site infections.

In some embodiments, the mixtures of bacterial strains treat or prevent urinary tract infections (e.g., antibiotic-resistant urinary tract infections and catheter-associated urinary tract infections).

In some embodiments, the mixtures of bacterial strains treat or prevent wound infections

In embodiments, the mixtures of bacterial strains treat or prevent other well-known infections: antibiotic-resistant infections and antibiotic-sensitive infections.

In various embodiments, the mixture of bacterial strains includes one or more bacterial strains that interact synergistically for treating or preventing a herein-described disorder, disease, or disorder.

In some embodiments, the mixtures of bacterial strains reduce, ameliorate, or eliminate one or more symptom(s) associated with a herein-described disease, disorder, or condition. Exemplary symptoms include, but are not limited to, diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts.

In some embodiments, the pharmaceutical composition of the invention comprises a bacterial strain derived from any one of the phylum, class, order, family, genus, and/or species listed in Table 5 or Table 6. In exemplary embodiments, the pharmaceutical composition of the invention comprises a bacterial strain belonging to the phylum Bacteroidetes or Firmicutes. In exemplary embodiments, the pharmaceutical composition of the invention comprises a bacterial strain belonging to the class Clostridia, Bacteroidia, or Bacilli. In exemplary embodiments, the pharmaceutical composition of the invention comprises a bacterial strain belonging to the order Bacteroidales, Clostridiales, or Lactobacillales. In exemplary embodiments, the pharmaceutical composition of the invention comprises a bacterial strain belonging to the family Bacteroidaceae, Ruminococcaceae, Lachnospiraceae, or Streptococcacea. In exemplary embodiments, the pharmaceutical composition of the invention comprises a bacterial strain belonging to the genus Bacteroides, Blautia, Faecalibacterium, Coprococcus, Roseburia, Dorea, Clostridium, Eubacterium or Streptococcus. In exemplary embodiments, the pharmaceutical composition of the invention comprises a bacterial strain belonging to the species uniformis, prausnitzii, or faecis.

### Pharmaceutical Compositions, Formulations, and Administration

The present invention provides pharmaceutical compositions comprising the novel mixtures of bacterial strains (and/or additional therapeutic agents) in various formulations. Any pharmaceutical composition (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the pharmaceutical compositions (and/or additional therapeutic agents) may conveniently be presented in unit dosage forms. For example, the dosage forms may be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In one embodiment, the pharmaceutical compositions comprising the novel mixtures of bacterial strains (and/or additional therapeutic agents) described herein are formulated as a composition adapted for a mode of administration described herein.

In various embodiments, the administration of the pharmaceutical compositions (and/or additional therapeutic agents) is any one of oral, intravenous, intraperitoneal, and parenteral. For example, routes of administration include, but are not limited to, oral, intraperitoneal, intravenous, intramuscular, or rectally. In various embodiments, the administration of the pharmaceutical compositions (and/or additional therapeutic agents) is oral, naso-gastric, anterograde gastrointestinal, retrograde gastrointestinal, endoscopic, or enemic.

In one embodiment, the pharmaceutical compositions (and/or additional therapeutic agents) described herein are formulated as a composition adapted for oral administration. Compositions for oral delivery can be in the form of tablets, aqueous or oily suspensions, granules, powders, sprinkles, emulsions, or capsules as examples. Orally administered compositions can comprise one or more agents, for example, sweetening agents such as fructose, aspartame or saccharin; flavoring agents such as peppermint, oil of wintergreen, or cherry; coloring agents; perfuming agents, to mask an odor of a bacterial mixture; and preserving agents, to provide a pharmaceutically palatable preparation. Moreover, when in capsule, tablet, or pill form, the compositions can be coated to delay disintegration to provide a sustained action over an extended period of time. Selectively permeable membranes surrounding an osmotically active agent driving any microbial strain (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, ethacrylic acid and derivative polymers thereof, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Oral administration of the pharmaceutical compositions comprising bacterial mixtures, e.g., via a capsule, is preferable since this route is simpler and more convenient than more conventional, invasive techniques like enema, nasogastric tube, or colonoscopy.

In various embodiments, the pharmaceutical compositions (and/or additional therapeutic agent) are formulated as solid dosage forms such as tablets, dispersible powders, granules, and capsules. In one embodiment, the pharmaceutical compositions (and/or additional therapeutic agent) are formulated as a capsule. In another embodiment, the pharmaceutical compositions (and/or additional therapeutic agent) are formulated as a capsule or tablet. In yet another embodiment, the pharmaceutical compositions (and/or additional therapeutic agent) are formulated as a soft-gel capsule. In a further embodiment, the pharmaceutical compositions (and/or additional therapeutic agent) are formulated as a gelatin capsule.

Dosage forms suitable for parenteral administration (e.g., intravenous, intramuscular, or intraperitoneal injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents.

In various embodiments, the formulations of the invention may additionally comprise a pharmaceutically acceptable carrier or excipient. As one skilled in the art will recognize, the formulations can be in any suitable form appropriate for the desired use and route of administration.

In some dosage forms, the agents described herein can be mixed with at least one inert, pharmaceutically acceptable excipient or carrier such as sodium citrate, dicalcium phosphate, and/or (a) fillers or extenders such as starches, lactose, sucrose, glucose, mannitol, silicic acid, microcrystalline cellulose, and Bakers Special Sugar, (b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., (c) humectants such as glycerol, (d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, glyceryl behenate, and mixtures of such excipients. One of skill in the art will recognize that particular excipients may have two or more functions in the oral dosage form. In the case of an oral dosage form, for example, a capsule or a tablet, the dosage form may also comprise buffering agents.

The formulation can additionally include a surface active agent. Surface active agents suitable for use in the present invention include, but are not limited to, any pharmaceutically acceptable, non-toxic surfactant. Classes of surfactants suitable for use in the compositions of the invention include, but are not limited to polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some embodiments, compositions of the invention may comprise one or more surfactants including, but not limited to, sodium lauryl sulfate, polysorbate 20, polysorbate 40, polysorbate 60, polysorbate 80, and triethyl citrate.

The formulation can also contain pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

The formulation can also include one or more application solvents. Some of the more common solvents that can be used to apply, for example, a delayed-release coating composition include isopropyl alcohol, acetone, methylene chloride and the like.

The formulation can also include one or more alkaline materials. Alkaline material suitable for use in compositions of the invention include, but are not limited to, sodium, potassium, calcium, magnesium and aluminum salts of acids such as phosphoric acid, carbonic acid, citric acid and other aluminum/magnesium compounds. In addition, the alkaline material may be selected from antacid materials such as aluminum hydroxides, calcium hydroxides, magnesium hydroxides and magnesium oxide.

In various embodiments, the pharmaceutical compositions (and/or additional therapeutic agents) are formulated for systemic or local delivery. In an embodiment, administration is systemic. In another embodiment, it may be desirable to administer locally to the area in need of treatment.

Various methods may be used to formulate and/or deliver the agents described herein to a location of interest. For example, the pharmaceutical compositions (and/or additional therapeutic agents) described herein may be formulated for delivery to the GI tract. The GI tract includes organs of the digestive system such as mouth, esophagus, stomach, duodenum, small intestine, large intestine (also referred here to as the “colon”) and rectum and includes all subsections thereof (e.g., the small intestine may include the duodenum, jejunum and ileum; the large intestine may include the colon transversum, colon descendens, colon ascendens, colon sigmoidenum and cecum). For example, the bacterial strains and/or pharmaceutical compositions (and/or additional therapeutic agents) described herein may be formulated for delivery to one or more of the stomach, small intestine, large intestine and rectum and includes all subsections thereof (e.g., duodenum, jejunum and ileum, colon transversum, colon descendens, colon ascendens, colon sigmoidenum and cecum). In some embodiments, the compositions described herein may be formulated to deliver to the upper or lower GI tract. In an embodiment, the bacterial strains and/or pharmaceutical compositions (and/or additional therapeutic agents) may be administered to a subject, by, for example, directly or indirectly contacting the mucosal tissues of the GI tract.

In various embodiments, the administration the pharmaceutical compositions (and/or additional therapeutic agents) is into the GI tract via, for example, oral delivery, nasogastric tube, intestinal intubation (e.g., an enteral tube or feeding tube such as, for example, a jejunal tube or gastro-jejunal tube, etc.), direct infusion (e.g., duodenal infusion), endoscopy, colonoscopy, or enema.

In various embodiments, the administration the pharmaceutical compositions (and/or additional therapeutic agents) is into the female reproductive system, for example by vaginal delivery, e.g., as vaginal suppository.

For example, in various embodiments, the present invention provides modified-release formulations comprising the novel mixtures of bacterial strains (and/or additional therapeutic agents), wherein the formulation releases a substantial amount of the bacterial strains (and/or additional therapeutic agents) into one or more regions of the GI tract. For example, the formulation may release at least about 60% of the bacterial strains after the stomach and into one or more regions of the GI tract.

In various embodiments, the modified-release formulation of the present invention releases at least 60% of the bacterial strains (or additional therapeutic agents) after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial strains (or additional therapeutic agents) in the intestines.

In various embodiments, the modified-release formulation of the present invention releases at least 60% of the bacterial strains (or additional therapeutic agents) in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial strains (or additional therapeutic agents) in the small intestine (e.g., one or more of duodenum, jejunum, ileum, and ileocecal junction).

In various embodiments, the modified-release formulation of the present invention releases at least 60% of the bacterial strains (or additional therapeutic agents) in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial strains (or additional therapeutic agents) in the large intestine (e.g., one or more of cecum, ascending, transverse, descending or sigmoid portions of the colon, and rectum).

In various embodiments, the pharmaceutical composition is formulated for substantially complete delivery prior to the rectum.

In some embodiments, the pharmaceutical composition is formulated for release in the stomach (e.g., so-called reverse enteric formulations). In other embodiments, the pharmaceutical composition is formulated so as to not substantially release the bacterial strains in the stomach.

In certain embodiments, the modified-release formulation releases the bacterial strains (or additional therapeutic agents) at a specific pH. For example, in some embodiments, the modified-release formulation is substantially stable in an acidic environment and substantially unstable (e.g., dissolves rapidly or is physically unstable) in a near neutral to alkaline environment. In some embodiments, stability is indicative of not substantially releasing while instability is indicative of substantially releasing. For example, in some embodiments, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some embodiments, the present formulations are stable in lower pH areas and therefore do not substantially release in, for example, the stomach. In some embodiments, modified-release formulation is substantially stable at a pH of about 1 to about 4 or lower and substantially unstable at pH values that are greater. In these embodiments, the modified-release formulation does not substantially release in the stomach. In these embodiments, the modified-release formulation substantially releases in the small intestine (e.g., one or more of the duodenum, jejunum, and ileum) and/or large intestine (e.g., one or more of the cecum, ascending colon, transverse colon, descending colon, and sigmoid colon). In some embodiments, modified-release formulation is substantially stable at a pH of about 4 to about 5 or lower and consequentially is substantially unstable at pH values that are greater and therefore is not substantially released in the stomach and/or small intestine (e.g., one or more of the duodenum, jejunum, and ileum). In these embodiments, the modified-release formulation substantially releases in the large intestine (e.g., one or more of the cecum, ascending colon, transverse colon, descending colon, and sigmoid colon). In various embodiments, the pH values recited herein may be adjusted as known in the art to account for the state of the subject, e.g., whether in a fasting or postprandial state.

In some embodiments, the modified-release formulation is substantially stable in gastric fluid and substantially unstable in intestinal fluid and, accordingly, is substantially released in the small intestine (e.g., one or more of the duodenum, jejunum, and ileum) and/or large intestine (e.g., one or more of the cecum, ascending colon, transverse colon, descending colon, and sigmoid colon).

In some embodiments, the modified-release formulation is stable in gastric fluid or stable in acidic environments. These modified-release formulations release about 30% or less by weight of the bacterial strains and/or additional therapeutic agent in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of the of the invention may release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the bacterial strains and/or additional therapeutic agent in the modified-release formulation in gastric fluid with a pH of 4-5, or less or simulated gastric fluid with a pH of 4-5 or less, in about 15, or about 30, or about 45, or about 60, or about 90 minutes. Modified-release formulations of the invention may release about 1%, about 2%, about 3%, about 4%, about 5%, about 6%, about 7%, about 8%, about 9%, or about 10% by weight of the total bacterial strains and/or additional therapeutic agent in the modified-release formulation in gastric fluid with a pH of 5 or less, or simulated gastric fluid with a pH of 5 or less, in about 15, or about 30, or about 45, or about 60, or about 90 minutes.

In some embodiments, the modified-release formulation is unstable in intestinal fluid. These modified-release formulations release about 70% or more by weight of the bacterial strains and/or additional therapeutic agent in the modified-release formulation in intestinal fluid or simulated intestinal fluid in about 15, or about 30, or about 45, or about 60, or about 90 minutes. In some embodiments, the modified-release formulation is unstable in near neutral to alkaline environments. These modified-release formulations release about 70% or more by weight of the bacterial strains and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments may release 70% or more by weight of bacterial strains and/or additional therapeutic agent in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various embodiments, the modified-release formulation of the invention is substantially stable in chyme. For example, there is, in some embodiments, a loss of less about 50% or about 40%, or about 30%, or about 20%, or about 10% of bacterial strains activity in about 10, or 9, or 8, or 7, or 6, or 5, or 4, or 3, or 2, or 1 hour from administration.

In various embodiments, the modified-release formulations of the present invention are designed for immediate release (e.g., upon ingestion). In various embodiments, the modified-release formulations may have sustained-release profiles, i.e., slow release of the active ingredient(s) in the body (e.g., GI tract) over an extended period of time. In various embodiments, the modified-release formulations may have a delayed-release profile, i.e., not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various embodiments, the modified-release formulation of the present invention may utilize one or more modified-release coatings such as delayed-release coatings to provide for effective, delayed yet substantial delivery of the bacterial strains to the GI tract together with, optionally, additional therapeutic agents.

In one embodiment, the delayed-release coating includes an enteric agent that is substantially stable in acidic environments and substantially unstable in near neutral to alkaline environments. In an embodiment, the delayed-release coating contains an enteric agent that is substantially stable in gastric fluid. The enteric agent can be selected from, for example, solutions or dispersions of methacrylic acid copolymers, cellulose acetate phthalate (CAP), hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, hypromellose (INN) hydroxypropyl methylcellulose (HPMC), shellac or other suitable enteric coating polymers. Similar polymers include Kollicoat® MAE 30 DP and Kollicoat® MAE 100 P. In various embodiments, the enteric agent may be a combination of the foregoing solutions or dispersions. In embodiments, the enteric agent comprises any EUDRAGIT®-type polymer, derivatives thereof, and copolymers thereof. EUDRAGIT® polymers are available from Evonik Industries AG (Essen, Germany).

In certain embodiments, one or more coating system additives are used with the enteric agent. For example, one or more PlasACRYL™ additives may be used as an anti-tacking agent coating additive. Illustrative PlasACRYL™ additives include, but are not limited to PlasACRYL™ HTP20 and PlasACRYL™ T20.

In another embodiment, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers useful in the present invention include polyvinyl esters, polyvinyl acetals, polyacrylic acid esters, butadiene styrene copolymers, and the like. In one embodiment, colonic delivery is achieved by use of a slowly-eroding wax plug (e.g., various PEGS, including for example, PEG6000).

In some embodiments, an enteric (interior or exterior) coating comprises a polymeric material. Non-limiting examples of suitable polymeric materials include polymethylmethacrylate, poly(N,N-dimethylacrylamide), polyoxamer, polyethylene glycol, polypropylene glycol, polysaccharides (e.g., sucrose, trehalose, glucose, starches such as tapioca and arrowroot, chitosan, alginate, guar gum), polyacrylate, polymethacrylate, polyvinyl alcohol, polyalkylene glycols, polyacrylamide, polyvinylpyrrolidone, polyurethane, polylactide, lactide/glycolide copolymer, polycaprolactone, polydioxanones, polyanhydride, polyhydroxybutyrate, polysiloxane, polytrimethylene carbonate, polyalkylene glycol, and combinations and/or copolymers thereof.

In a further embodiment, the delayed-release coating may be degraded by a microbial enzyme present in the gut flora. In one embodiment, the delayed-release coating may be degraded by a bacteria present in the small intestine. In another embodiment, the delayed-release coating may be degraded by a bacteria present in the large intestine.

Such a coating may comprise a mixture of a first material which is susceptible to attack by colonic bacteria and a second material which has a solubility threshold at about pH 5 or above. The first material may comprise a polysaccharide selected from starch, amylose, amylopectin, chitosan, chondroitin sulfate, cyclodextrin, dextran, pullulan, carrageenan, scleroglucan, chitin, curdulan, and levan. The second material may dissolve in a pH-dependent manner such that it has a “pH threshold” which is the pH below which it is insoluble and at or above which it is soluble. The pH of the surrounding medium triggers dissolution of the second material; thus, little of the second material dissolves below the pH threshold. Once the pH of the surrounding medium reaches (or exceeds) the pH threshold, the second material becomes soluble. In embodiments, the surrounding medium means the medium in the GI tract, such as the gastricjuice or intestinal juice or the in vitro equivalent of the medium in the GI tract. The second material may be a film-forming polymeric material such as an acrylate polymer, a cellulose polymer or a polyvinyl-based polymer. Examples of suitable cellulose polymers include cellulose acetate phthalate (“CAP”), cellulose acetate trimellitate (“CAT”), and hydropropylmethylcellulose acetate succinate. Examples of suitable polyvinyl-based polymers include polyvinyl acetate phthalate (“PVAP”). The second material may be a co-polymer of a (meth)acrylic acid and a (meth)acrylic acid C1-4 alkyl ester, for instance, a copolymer of methacrylic acid and methacrylic acid methyl ester. Such a polymer is known as a poly(methacrylic acid/methyl methacrylate) co-polymer. Examples of such co-polymers are usually anionic and not sustained release polymethacrylates. Examples of anionic poly(methacrylic acid/methyl methacrylate) co-polymers include Eudragit® L, Eudragit® S, and Eudragit® FS. The coating may have an additional layer either between the bacterial mixture core and the layer comprising the delayed release composition described above and/or an outer layer coating the delayed release composition layer as described above.

In embodiments, a capsule comprises an interior enteric coating which has hydrophobic properties which prevents or retards the contact of an aqueous phase (e.g., a drug substance of the present disclosure) with the capsule (or capsule material). In embodiments, the interior enteric coating comprises a hydrophobic coating. The hydrophobic coating may comprise a material selected from the group consisting of shellac, zein, polysaccharides, silk, polycaprolactone, oil, pectin, wax, polymers, shellac, and derivatives thereof, and combinations thereof. Non-limiting examples of suitable polysaccharides include alginate, hyaluronic acid, and chitosan. Non-limiting examples of suitable oils include avocado oil, vegetable oil, castor oil, olive oil, jojoba oil, cocoa butter, coconut oil. Non-limiting examples of suitable waxes include beeswax, carnauba wax, and paraffin wax. In some embodiments, the hydrophobic coating is shellac.

An interior enteric coating may be selected and designed such that it protects the capsule (or capsule material) from an aqueous phase. For example, in some embodiments, the interior enteric coating prevents the aqueous phase (e.g., a mixture of bacterial strains of the present disclosure) from contacting the capsule and/or such that the capsule material is not degraded and/or dissolved by the aqueous phase. In some embodiments, the interior enteric coating protects the capsule from the aqueous phase for greater than or equal to 1 day, greater than or equal to 2 days, greater than or equal to 3 days, greater than or equal to 7 days, greater than or equal to 14 days, greater than or equal to 30 days, greater than or equal to 90 days, or greater than or equal to 180 days at room temperature under ambient conditions. In certain embodiments, the interior enteric coating protects the capsule from the aqueous phase for less than or equal to 365 days, less than or equal to 180 days, less than or equal to 90 days, less than or equal to 30 days, less than or equal to 14 days, less than or equal to 7 days, less than or equal to 3 days, or less than or equal to 2 days at room temperature under ambient conditions. Combinations of the above-referenced ranges are possible (e.g., greater than or equal to 1 day and less than or equal to 365 days). Other ranges are also possible. As such, in some embodiments, the capsule is stable at room temperature under ambient conditions for the times listed above (e.g., greater than or equal to 1 day).

In certain embodiments, the interior enteric coating protects the capsule from the aqueous phase (e.g., the interior enteric coating prevents the aqueous phase from contacting the capsule and/or such that the capsule material is not degraded and/or dissolved by the aqueous phase) for greater than or equal to 1 hour, greater than or equal to 2 hours, greater than or equal to 3 hours, greater than or equal to 6 hours, greater than or equal to 12 hours, greater than or equal to 18 hours, greater than or equal to 24 hours, greater than or equal to 48 hours, or greater than or equal to 96 hours at 37° C. In certain embodiments, the interior enteric coating protects the capsule from the aqueous phase for less than or equal to 168 hours, less than or equal to 96 hours, less than or equal to 48 hours, less than or equal to 24 hours, less than or equal to 18 hours, less than or equal to 12 hours, less than or equal to 6 hours, less than or equal to 3 hours, or less than or equal to 2 hours at 37° C. under ambient conditions. Combinations of the above-referenced ranges are possible (e.g., greater than or equal to 1 hour and less than or equal to 168 hours). As such, in certain embodiments, the capsule is stable at 37° C. under ambient conditions for the times listed above (e.g., greater than or equal to 1 hour).

In various embodiments, the modified release formulation is designed for release in the colon. Various colon-specific delivery approaches may be utilized. For example, the modified release formulation may be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation may be designed as a core tablet with three layers of polymer. The first coating is an acid-soluble polymer (e.g., EUDRAGIT E), the outer coating is enteric, along with a hydroxypropyl methylcellulose barrier layer interposed in between. In another embodiment, colon delivery may be achieved by formulating the bacterial strains (and/or additional therapeutic agent) with specific polymers that degrade in the colon such as, for example, pectin. The pectin may be further gelled or crosslinked with a cation such as a zinc cation. In an embodiment, the formulation is in the form of ionically crosslinked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDS-CT).

In some embodiments, an enteric (interior or exterior) coating comprises an enteric elastomer. In some embodiments, the enteric elastomer comprises a mixture of two or more polymers with carboxyl functionality such that the two or more polymers form hydrogen bonds with one another and has both enteric and elastic properties. In certain embodiments, the enteric elastomer comprises a first polymer comprising a structure as in Formula (I):

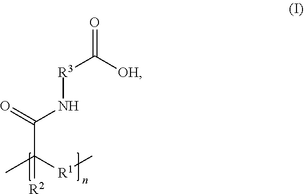

or a pharmaceutically acceptable salt thereof, wherein each R¹ is the same or different and is selected from the group consisting of optionally substituted alkylene, optionally substituted heteroalkylene, optionally substituted arylene, and optionally substituted heteroarylene, each R² is the same or different and is selected from the group consisting of hydrogen, optionally substituted alkyl, and optionally substituted heteroalkyl, each R³ is the same or different and is selected from the group consisting of optionally substituted alkylene and optionally substituted heteroalkylene, n is an integer between 25 and 250,000, and a second polymer comprising a structure as in Formula (II) hydrogen bonded to the first polymer:

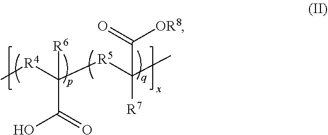

or a pharmaceutically acceptable salt thereof, wherein each R⁴ is the same or different and is selected from the group consisting of optionally substituted alkylene and optionally substituted heteroalkylene, each R⁵ is the same or different and is selected from the group consisting of optionally substituted alkylene and optionally substituted heteroalkylene, each R⁶ is the same or different and is selected from the group consisting of hydrogen, optionally substituted alkyl, and optionally substituted heteroalkyl, each R⁷ is the same or different and is selected from the group consisting of hydrogen, optionally substituted alkyl, and optionally substituted heteroalkyl, each R⁸ is the same or different and is optionally substituted alkyl, p is an integer between 1 and 10, q is an integer between 1 and 10, and z is an integer between 1 and 150,000, provided that (p+q)*z is greater than or equal to 20. Suitable enteric elastomers and methods for making such enteric elastomers are described in more detail in International Patent Publication No. WO2015191922, which is incorporated herein by reference in its entirety for all purposes.

In some embodiments, a capsule comprises a polymeric material. Non-limiting examples of suitable polymeric materials include gelatin, polymethylmethacrylate, poly(N,N-dimethylacrylamide), polyoxamer, polyethylene glycol, polypropylene glycol, polysaccharides (e.g., sucrose, trehalose, glucose, starches such as tapioca and arrowroot, chitosan, alginate, guar gum), polyacrylate, polymethacrylate, polyvinyl alcohol, polyalkylene glycols, polyacrylamide, polyvinylpyrrolidone, polyurethane, polylactide, lactide/glycolide copolymer, polycaprolactone, polydioxanones, polyanhydride, polyhydroxybutyrate, polysiloxane, polytrimethylene carbonate, polyalkylene glycol, and combinations and/or copolymers thereof. In embodiments, the capsule comprises gelatin.

In certain embodiments, the capsule may comprise a bioadherent polymer such as mucin.

Embodiments of dual-coated coated capsules are disclosed in WO2018057747, the contents of which are incorporated by reference in their entirety.

In certain embodiments, the capsule has a particular shape or size. For example, in some cases, the capsule has a shape or size as described in the USP including, but not limited to, #000 capsule, #00 capsule, #0 capsule, #1 capsule, #2 capsule, #3 capsule, #4 capsule, or #5 capsule. Other capsule shapes and/or sizes are also possible.

Formulations for colon specific delivery of the bacterial strains (and/or additional therapeutic agents), as described herein, may be evaluated using, for example, in vitro dissolution tests. For example, parallel dissolution studies in different buffers may be undertaken to characterize the behavior of the formulations at different pH levels. Alternatively, in vitro enzymatic tests may be carried out. For example, the formulations may be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further embodiment, in vivo evaluations may be carried out using animal models such as dogs, guinea pigs, rats, and pigs. Further, clinical evaluation of colon specific drug delivery formulations may be evaluated by calculating drug delivery index (DDI) which considers the relative ratio of RCE (relative colonic tissue exposure to the drug) to RSC (relative amount of drug in blood i.e., that is relative systemic exposure to the drug). Higher drug DDI indicates better colon drug delivery. Absorption of drugs from the colon may be monitored by colonoscopy and intubation.

In various embodiments, the present formulation provides for substantial uniform dissolution of the bacterial strains (and/or additional therapeutic agent) in the area of release in the GI tract. In an embodiment, the present formulation minimizes patchy or heterogeneous release of the bacterial strains.

In various embodiments, the present formulations provide for release of multiple doses of the bacterial strains along the GI tract. For example, the composition and/or formulation can release multiple doses of the bacterial strains at different locations along the intestines, at different times, and/or at different pH. The overall release profile of such a formulation may be adjusted using, for example, multiple particle types or multiple layers. For example, in one embodiment, the first dose of the bacterial strains may be formulated for release in, for example, the small intestine (e.g., one or more of duodenum, jejunum, ileum), whereas the second dose is formulated for delayed release in, for example, the large intestines (e.g., one or more of cecum, ascending, transverse, descending or sigmoid portions of the colon, and rectum). In another example, the first dose of the bacterial strains may be formulated for release in, for example, the small intestine (e.g., one or more of duodenum, jejunum, ileum), whereas the second dose is formulated for delayed release in, for example, another part of the small intestine (e.g., one or more of duodenum, jejunum, ileum). In another embodiment, the first dose of the bacterial strains may be formulated for release in, for example, the large intestine (e.g., one or more of cecum, ascending, transverse, descending or sigmoid portions of the colon, and rectum), whereas the second dose is formulated for delayed release in, for example, another part of the large intestine (e.g., one or more of cecum, ascending, transverse, descending or sigmoid portions of the colon, and rectum). In various embodiments, the composition and/or formulation may release at least one dose, at least two doses, at least three doses, at least four doses, or at least five doses of the bacterial strains at different locations along the intestines, at different times, and/or at different pH.

In some embodiments, the bacterial strains described herein are in the form of live, vegetative cells. In some embodiments, the bacterial strains described herein are in the form of spores. In some embodiments, the bacterial strains described herewith are lyophilized. As used herein, “lyophilization” or “freeze drying” refers to the process of drying a material by first freezing it and then encouraging the ice within it to sublimate in a vacuum environment.

By way of non-limiting example, lyophilization can be via methods known in the art, including those described in U.S. Pat. No. 7,799,328, the contents of which are hereby incorporated by reference in their entirety. In some embodiments, lyophilized bacterial strains described herein are placed in an enterically coated soft gel or capsule.

In one aspect, a pharmaceutical composition comprises a lyophilized formulation further comprising a reducing agent. In certain embodiments, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, Glutathione, Methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a therapeutic composition comprises a cryoprotectant. As used herein, a “cryoprotectant” refers to a substance that is added to a formulation in order to protect an active ingredient during freezing. In an aspect, a cryoprotectant comprises, consists essentially of, or consists of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof. In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehaolse with 0.1% Polysorbate 80.

In another aspect, a therapeutic composition comprises a lyoprotectant. As used herein, a “lyoprotectant” refers to a substance that is added to a formulation in order to protect an active ingredient during the drying stage of a lyophilization (also known as freeze-drying) process. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consists essentially of, or consists of, one or more substances mentioned in this paragraph and the paragraph above.

In one aspect, a cryoprotectant or a lyoprotectant comprise an intracellular agent, e.g., DMSO, Glycerol, or PEG, which penetrates inside the cell preventing the formation of ice crystals that could result in membrane rupture. In another aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In various embodiments, the formulations of the present invention take the form of those as described in one or more of U.S. Pat. Nos. 8,535,713 and 8,911,777 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various embodiments, the formulations of the present invention take the form of those as described in International Patent Publication No. WO 2008/135090, the contents of which are hereby incorporated by reference in their entirety.

In various embodiments, the formulations of the present invention take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

### Administration and Dosage

It will be appreciated that the actual dose of the bacterial strains (and/or additional therapeutic agents) to be administered according to the present invention will vary according to, for example, the particular dosage form and the mode of administration. Many factors that may modify the action of the bacterial strains (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In various embodiments, the dose of the bacterial strains is effective to modulate a patient's microbiome to favor an ecological balance, i.e., treating or preventing a GI disorder described herein.

In various embodiments, the dose of the bacterial strains comprises at least 1×10⁴, 1×10⁵, 1×10⁶, 1×10⁷, 1×10⁸, 1×10⁹, 1×10¹⁰, 1×10¹¹ or greater than 1×10¹¹ colony forming units (CFUs) or bacteria (e.g., germinable bacterial spores).

Individual doses of the bacterial strains (and/or additional therapeutic agents) can be administered in unit dosage forms (e.g., tablets or capsules) containing, for example, from about 0.01 mg to about 5,000 mg, from about 0.01 mg to about 4,000 mg, from about 0.01 mg to about 3,000 mg, from about 0.01 mg to about 2,000 mg, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg of the active ingredient per unit dosage form, or from about 5 mg to about 80 mg per unit dosage form. For example, a unit dosage form can include about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg of the active ingredient, inclusive of all values and ranges therebetween.

In one embodiment, the bacterial strains (and/or additional therapeutic agents) is administered at an amount of from about 0.01 mg to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the bacterial strains (and/or additional therapeutic agents) is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges therebetween.

In some embodiments, a suitable dosage of the bacterial strains (and/or additional therapeutic agents) is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other embodiments, a suitable dosage of the bacterial strains (and/or additional therapeutic agents) in a range of about 0.01 mg/kg to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In an aspect, a therapeutic composition provided here comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

As used herein, “Shannon Diversity Index” refers to a diversity index that accounts for abundance and evenness of species present in a given community using the formula:

$$H = - {\sum }_{i = 1}^{R} ( {p}_{i} ) ( ln ( {p}_{i} ) )$$

where H is Shannon Diversity Index, R is the total number of species in the community, and pi is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117pp.

In accordance with certain embodiments of the invention, the bacterial strains may be administered, for example, more than once daily, about once per day, about every other day, about every third day, about once a week, about once every two weeks, about once every month, about once every two months, about once every three months, about once every six months, or about once every year.

In one aspect, the present disclosure provides a method for treating a disorder in a subject in need thereof, where the method comprises administering to the subject a pharmaceutically active dose of a therapeutic composition described herein. In one aspect, the present disclosure provides a method for treating a disorder in a subject in need thereof, where the method comprises administering daily to the subject a pharmaceutically active dose of a therapeutic composition described herein. In one aspect, a therapeutic composition is administered to a patient in need thereof at least once daily for at least two consecutive days. In one aspect, a therapeutic composition is administered at least once daily for at least 3, 4, 5, 6, 7, 8, 9, 10, 11, 12, 13, 14, or 15 consecutive days. In another aspect, a therapeutic composition is administered at least once daily for at least 1, 2, 3, 4, 5, 6, 7, 8, 9, 10, 11, or 12 consecutive weeks. In another aspect, a therapeutic composition is administered at least twice, three times, four times, or five times per week for at least 1, 2, 3, 4, 5, 6, 7, 8, 9, 10, 11, or 12 consecutive weeks. In one aspect, a therapeutic composition is administered at least once daily for at most 4, 5, 6, 7, 8, 9, 10, 11, 12, 13, 14, 15, 16, 17, 18, 19, or 20 consecutive days or weeks. In another aspect, a therapeutic composition is administered at least once daily for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a patient in need thereof at least twice daily for at least two consecutive days. In one aspect, a therapeutic composition is administered at least twice daily for at least 3, 4, 5, 6, 7, 8, 9, 10, 11, 12, 13, 14, or 15 consecutive days. In another aspect, a therapeutic composition is administered at least twice daily for at least 1, 2, 3, 4, 5, 6, 7, 8, 9, 10, 11, or 12 consecutive weeks. In one aspect, a therapeutic composition is administered at least twice daily for at most 4, 5, 6, 7, 8, 9, 10, 11, 12, 13, 14, 15, 16, 17, 18, 19, or 20 consecutive days or week. In another aspect, a therapeutic composition is administered at least twice daily for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a patient in need thereof at least three times daily for at least two consecutive days. In one aspect, a therapeutic composition is administered at least three times daily for at least 3, 4, 5, 6, 7, 8, 9, 10, 11, 12, 13, 14, or 15 consecutive days. In another aspect, a therapeutic composition is administered at least three times daily for at least 1, 2, 3, 4, 5, 6, 7, 8, 9, 10, 11, or 12 consecutive weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating a disorder in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota in a lyophilized formulation described herein, where the dose is administered at a dosing schedule of at least once or twice daily for at least three consecutive days or weeks. In another aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating a disorder in a subject in need thereof by administering a pharmaceutical composition described herein, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more fold lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

### Additional Therapeutic Agents and Combination Therapy or Co-Formulation

Administration of the present formulations may be combined with additional therapeutic agents. Co-administration of the additional therapeutic agent and the present formulations may be simultaneous or sequential. Further; the present formulations may comprise an additional therapeutic agent (e.g., via co-formulation). For example, the additional therapeutic agent and the bacterial strains may be combined into a single formulation.

In one embodiment, the additional therapeutic agent and the bacterial strains are administered to a subject simultaneously. The term “simultaneously” as used herein, means that the additional therapeutic agent and the bacterial strains are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the bacterial strains can be by simultaneous administration of a single formulation (e.g., a formulation comprising the additional therapeutic agent and the bacterial strains) or of separate formulations (e.g., a first formulation including the additional therapeutic agent and a second formulation including the bacterial strains).

Co-administration does not require the additional therapeutic agents to be administered simultaneously, if the timing of their administration is such that the pharmacological activities of the additional therapeutic agent and the bacterial strains overlap in time. For example, the additional therapeutic agent and the bacterial strains can be administered sequentially. The term “sequentially” as used herein means that the additional therapeutic agent and the bacterial strains are administered with a time separation of more than about 60 minutes. For example, the time between the sequential administration of the additional therapeutic agent and the bacterial strains can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the bacterial strains being administered. Either the additional therapeutic agent or the bacterial strains may be administered first.

In a further embodiment, the additional therapeutic agent and the bacterial strains are administered to a subject simultaneously but the release of additional therapeutic agent and the bacterial strains from their respective dosage forms (or single unit dosage form if co-formulated) in the GI tract occurs sequentially.

Co-administration also does not require the additional therapeutic agents to be administered to the subject by the same route of administration. Rather, each additional therapeutic agent can be administered by any appropriate route, for example, parentally or non-parentally.

In some embodiments, the additional therapeutic agent is an agent used in the current standard-of-care induction therapies for the pathogenic bacteria that the subject is currently infected with and/or is at risk for being infected with, e.g., one or more anti-inflammatory agents, probiotic agents, prebiotic agents, antidiarrheal agents, analgesics, and antibiotic agents.

In some embodiments, the additional therapeutic agent is an anti-inflammatory agent such as steroidal anti-inflammatory agents or non-steroidal anti-inflammatory agents (NSAIDS). Steroids, particularly the adrenal corticosteroids and their synthetic analogues, are well known in the art. Examples of corticosteroids useful in the present invention include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. Additional anti-inflammatory agents are described, for example, in U.S. Pat. No. 4,537,776, the entire contents of which are incorporated by reference herein.

In some embodiments, the additional therapeutic agent is a probiotic. Probiotics suitable for use in the present invention include, but are not limited to, Saccharomyces boulardii; Lactobacillus rhamnosus GG; Lactobacillus plantarum 299v; Clostridium butyricum M588; Clostridium difficile VP20621 (non-toxigenic C. difficile strain); combination of Lactobacillus casei, Lactobacillus acidophilus (Bio-K+CL1285); combination of Lactobacillus casei, Lactobacillus bulgaricus, Streptococcus thermophilus (Actimel); combination of Lactobacillus acidophilus, Bifidobacterium bifidum (Florajen3); combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, and Streptococcus salivarius subsp. thermophilus (VSL #3)).

The compositions and methods of the present invention may further comprise one or more prebiotics.

A prebiotic is a substrate that is selectively used by a host microorganism to produce a health benefit in a subject/patient. Without wishing to be bound by theory, prebiotics are added to nutritionally supplement bacteria in the microbiome and/or in a microbial composition, e.g., to stimulate the growth or activity of one or more strains of beneficial bacteria. Additionally, the prebiotics may be added to prevent “shock” to bacterial strains subsequent to their isolation or purification, freezing, freeze-drying, spray-drying, reconstitution in solution and the like.

Examples of prebiotics include amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

In embodiments, a prebiotic can be added (e.g., in dry or liquid forms) to a microbial composition of the present invention.

Alternately, or additionally, a prebiotic can be included (e.g., in dry or liquid forms) in a distinct pharmaceutical composition which lacks a microbial composition of the present invention.

A prebiotic may be provided to a subject before, contemporaneously with, and/or after a pharmaceutical composition comprising a microbial composition of the present invention is administered, either in a pharmaceutical composition comprising the microbial composition or in a pharmaceutical composition lacking a microbial composition.

A prebiotic may be provided in a single dose or in multiple doses. When provided as a single composition, the single composition may comprise a single prebiotic or a mixture of prebiotics. When provided in multiple compositions, each composition may comprise a single prebiotic or a mixture of prebiotics.

As examples, when multiple doses are provided, a first composition comprising a prebiotic may include one specific prebiotic, e.g., inulin, and a second composition may include a second specific prebiotic, e.g., pectin. Alternately, a first composition may include a mixture of prebiotics, e.g., inulin and pectin and a second composition may include different mixture of prebiotics, e.g., inulin and a FOS. A first composition may include a mixture of prebiotics and a second composition may include one specific prebiotic.

The amount of prebiotic provided to a subject/patient and/or included in a composition depends on the specific prebiotic, the specific bacterial strain of beneficial bacteria, and/or the disease state of the subject/patient. In some embodiments, the additional therapeutic agent is an antidiarrheal agent. Antidiarrheal agents suitable for use in the present invention include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine, thalidomide and traditional antidiarrheal remedies, such as kaolin, pectin, berberine and muscarinic agents.

In some embodiments, the additional therapeutic agent may be an analgesic. Analgesics useful in the compositions and methods of the present invention include, without limitation, morphine, codeine, heroine, methadone and related compounds, thebaine, orpiavine, and their derivatives, buprenorphine, the piperidines, morphinans, benzomorphans, tetrahydroisoquinolines, thiambutanes, benzylamines, tilidine, viminol, nefopam, capsaicin(8-methyl-N-vanillyl-6E-nonenamide), “synthetic” capsaicin (N-vanillylnonamide), and related compounds.

In some embodiments, the additional therapeutic agent is an antibacterial agent, which includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-bacterial agent may be any of the penicillin, cephalosporin, monobactam, and carbapenem antibiotics.

In some embodiments, the additional therapeutic agent includes, but is not limited to, short-chain fatty acids, butyrate, propionate, acetate, IL-2, IL-22, superoxide dismutase (SOD), GLP-2 and analogs, GLP-1, IL-10, IL-27, TGF-β1, TGF-β2, N-acylphosphatidylethanolamines (NAPEs), elafin (also called peptidase inhibitor 3 and SKALP), trefoil factor, melatonin, tryptophan, PGD2, and kynurenic acid, indole metabolites, and other tryptophan metabolites.

Aspects of the present invention relate to pharmaceutical compositions comprising a bacterial mixture and an anti-cancer therapeutic agent. The anti-cancer therapeutic agent may be a chemotherapeutic agent. In a pharmaceutical composition of the present invention, a chemotherapeutic agent that can be formulated for oral administration and the bacterial mixture may be combined and encapsulated together in a capsule. Alternately, the chemotherapeutic agent may be included in a layer coating a capsule which encapsulates the bacterial mixture. In embodiments, the chemotherapeutic agent and the pharmaceutical composition comprising the bacterial mixture are in separate dosage forms. In embodiments, a subject in need thereof has received, is receiving, or will receive chemotherapeutic agent, either with or separate from a bacterial mixture.

In embodiments, any chemotherapeutic agent that can be formulated for oral administration may be used in con. Examples of such chemotherapeutic agents include Afinitor (everolimus), Alecensa (alectinib), Alkeran (melphalan), Alunbrig (brigatinib), Arimidex (anastrozole), Aromasin (exemestane), Bosulif (bosutinib), Cabometyx (cabozantinib), Caprelsa (vandetanib), Casodex (bicalutamide), Cometriq (cabozantinib), Cotellic (cobimetinib), Cyclophosphamide (cyclophosphamide caps), Cytoxan (Cyclophosphamide), Droxia (hydroxyurea), Emcyt (estramustine), Erivedge (vismodegib), etoposide, Fareston (toremifene citrate), Farydak (panobinostat), Femara (letrozole), flutamide, Gilotrif (afatinib), Gleevec (imatinib), Gleostine (lomustine), Hexalen (altretamine), Hycamtin (topotecan), Hydrea (hydroxyurea), Ibrance (palbociclib), Iclusig (ponatinib), Idamycin (Idarubicin), Idhifa (enasidenib), Imbruvica (ibrutinib), Inlyta (axitinib), Iressa (gefitinib), Jakafi (ruxolitinib), Kisqali (ribociclib), Kisqali Femara Co-Pack (ribociclib and letrozole), Lenvima (lenvatinib), leucovorin, Leukeran (chlorambucil), Lonsurf (trifluridine/tipiracil), Lynparza (olaparib), Lysodren (mitotane), Matulane (procarbazine), Megace (megestrol acetate), Mekinist (trametinib), mercaptopurine, Mesnex (mesna), methotrexate, Myleran (busulfan), Navelbine (Vinorelbine), Nerlynx (neratinib), Nexavar (sorafenib), Nilandron (nilutamide), Ninlaro (ixazomib), Odomzo (sonidegib), Pomalyst (pomalidomide), Purixan (mercaptopurine susp), Revlimid (lenalidomide), Rubraca (rucaparib), Rydapt (midostaurin), Soltamox (tamoxifen citrate), Sprycel (dasatinib), Stivarga (regorafenib), Sutent (sunitinib), Tabloid (thioguanine), Tafinlar (dabrafenib), Tagrisso (osimertinib), tamoxifen, Tarceva (erlotinib), Targretin (bexarotene), Tasigna (nilotinib), Temodar (temozolomide), Thalomid (thalidomide), Toposar (Etoposide), tretinoin, Trexall (methotrexate), Tykerb (lapatinib), Venclexta (venetoclax), Votrient (pazopanib), Xalkori (crizotinib), Xatmep (methotrexate soln), Xeloda (capecitabinea), Xtandi (enzalutamide), Zejula (niraparib), Zelboraf (vemurafenib), Zolinza (vorinostat), Zydelig (idelalisib), Zykadia (ceritinib), or Zytiga (abiraterone), and a combination thereof.

In embodiments, a pharmaceutical composition can be in the form of a capsule, tablet, or pill. In embodiments, the capsule, tablet, or pill can be coated or otherwise compounded to provide a dosage form affording the advantage of prolonged action. For example, the capsule, tablet, or pill can comprise an inner dosage (e.g., a bacterial mixture) and an outer dosage component (e.g., a chemotherapeutic agent and/or additional therapeutic agent), the latter being in the form of an envelope over the former. The two components can be separated by an enteric layer which serves to resist disintegration in the stomach and permits the inner component to pass into the duodenum or colon and/or to be delayed in release. A variety of materials can be used for such enteric layers or coatings (as described herein). Exemplary materials include a number of polymeric acids and mixtures of polymeric acids with such materials as shellac, cetyl alcohol, and cellulose acetate.

For all additional therapeutic agent compositions and methods, targeting to various parts of the GI tract may be employed as described herein.

In various embodiments, the patient of the present methods is undergoing treatment with one or more additional therapeutic agents and, by way of non-limitation, such additional therapeutic agents may disrupt the microbiome.

### Methods of Treatment

In various embodiments, the present invention provides methods of modulating a patient's microbiome to provide or restore an ecological balance. For instance, in various embodiments, herein are provided methods for diminishing or inhibiting one or more pathogenic bacteria as described elsewhere herein. In various embodiments, the present mixtures of bacterial strains augment growth of at least one type of bacteria not detectably present in a patient's GI tract prior to administration and, in various embodiments, which are non-pathogenic.

In various embodiments, the present invention provides methods of restoring or enhancing ecological control over gut or genitourinary pathogens or pathobionts in a patient.

In various embodiments, the present invention provides methods of treating or preventing a disease or condition associated with GI dysbiosis, comprising administering an effective amount of a pharmaceutical composition described herein to a subject or a patient need thereof.

In various embodiments, the present invention provides methods of treating or preventing a disease or condition associated with genitourinary dysbiosis, comprising administering an effective amount of a pharmaceutical composition disclosed herein to a subject or a patient need thereof.

Without wishing to be bound by theory, decolonization and eradication of pathogenic bacteria relevant to the present invention likely occurs through, one or more mechanisms. These mechanisms are: (1) Direct inhibition of ARB growth through the production of secreted soluble factors (see, below references 35 to 37 and 70 to 73); (2) Competition for the relevant niche/preferred nutrients (see, below references 8, 38, 39, 74, and 75); (3) Immune activation through host recognition of specific pathogen-associated molecular patterns (PAMPs) (see, below references 40 and 41); and (4) Production of Short Chain Fatty Acid (SCFA) by bacterial strains which enhance the colonic barrier and can induce IgA production (see, below references 40 and 41), and (5) activation of Toll-Like Receptors (TLRs), which modulate the production of antimicrobial peptides, which target many human bacterial pathogens.

In the third and fifth mechanism, various bacterial products stimulate the immune system through the TLR-MyD88 mediated pathway. Stimulation of this pathway results in the upregulation of antimicrobial proteins. Antimicrobial proteins and peptides (AMPs) (e.g., the defensins or cathelicidins) are an important part of the innate immune system and are expressed throughout the body. In the gut, they are produced by the intestinal epithelial cells and Paneth cells to defend against undesired bacterial species (both commensal and pathogenic). AMP produced may be induced by a wide variety of stimuli including but not limited to vitamin D, cytokines, and microbial products. These stimulatory microbial products can include but are not limited to lipopolysaccharide (LPS), peptidoglycan, and flagellin. For example, flagellin is a TLR5 agonist that induces the production of the C-type lectin, RegIIIgamma. RegIIIgamma has roles in killing Gram-positive pathogens, including vancomycin resistant Enterococcus (VRE). There is also evidence that anti-infective AMPs play a role in attenuating host inflammatory responses through downregulation of cytokine production. Induction of AMPs assists the host with a properly modulated immune response to pathogens that accelerates healing of the gut epithelia while eradicating the colonizing pathogen.

In embodiments, the mixture of bacterial strains of the present invention acts to protect patients through one or more mechanisms: A first mechanism is direct inhibition of the pathogenic bacteria through production of secreted products and a second mechanism is through competition for nutrients in the gut.

In various embodiments, the methods of the invention comprise treating or preventing a microbiome-mediated disorder. Illustrative microbiome-mediated disorder includes, but are not limited to, for example, those found in Table 3 of WO 2014/121298, the entire contents of which are incorporated herein by reference.

In various embodiments, the present invention provides methods of treating a patient suffering from a disease or condition associated with GI dysbiosis. In some embodiments, the patient has inflammatory bowel diseases (IBD), for example, Crohn's disease, colitis (e.g., UC or microscopic colitis), or pouchitis. IBD is a group of inflammatory conditions of the large intestine and, in some cases, the small intestine. The main forms of IBD that may be treated by the prevent invention include, but are not limited to, Crohn's disease, UC, pouchitis, collagenous colitis, lymphocytic colitis, ischemic colitis, diversion colitis, Behçet's syndrome, infective colitis, and indeterminate colitis. In an embodiment, the present invention provides methods of treating UC. In another embodiment, the present invention provides methods of treating CD. In a further embodiment, the present invention provides methods of treating pouchitis.

In various embodiments, methods of the invention are utilized for the treatment of UC. UC is one form of IBD. It is a chronic disease of the colon, in which the lining of the colon becomes inflamed and develops tiny open sores, or ulcers, that produce pus and mucous. In some embodiments, methods of the invention ameliorate, reduce, or eliminate the inflammation and/or ulceration associated with UC. In some embodiments, methods of the invention ameliorate, reduce, or eliminate one or more symptoms associated with UC including but not limited to, abdominal discomfort or pain, frequent emptying of the colon, lose and urgent bowel movements, persistent diarrhea, bloody stool, loss of appetite, and weight loss. In some embodiments, methods of the invention may reduce or prevent the delay in growth and development in children afflicted with UC.

In some embodiments, the present invention provides methods of treating IBS. IBS is a common disorder that affects the colon and can cause cramping, abdominal pain, bloating, gas, diarrhea and constipation. IBS is classified based on the predominant symptom of diarrhea (IBS with predominant diarrhea, IBS-D), constipation (IBS with predominant constipation, IBS-C) or mixed symptoms (IBS with alternating constipation and diarrhea, IBS-A). Methods of the invention are effective in treating one or more of IBS-D, IBS-C, and/or IBS-A. In some embodiments, methods of the invention ameliorate, reduce, or eliminate one or more symptoms associated with one or more of IBS-D, IBS-C, and/or IBS-A.

In some embodiments, the present invention provides for compositions and methods for treating or preventing infections (including colonization) by pathogenic bacteria and/or inhibiting the growth or decrease the number of pathogenic bacteria in the GI tract. In an embodiment, the pathogenic bacteria are enterobacteria such as Salmonella. In various embodiments, the present invention provides for compositions and methods that mitigate or prevent the overgrowth of various coliforms in a patient's gut (including coliforms that are virulent and/or antibiotic resistant). Illustrative coliforms include Citrobacter, Enterobacer, Hafnia, Klebsiella, and Escherichia. In some embodiments, the methods and compositions described herein prevent or diminish secondary infections with resistant organisms.

In still other embodiments, the present invention provides methods of treating a patient with an infectious disease of the intestines, for example, CDI and/or a CDAD, nosocomial infection, secondary emergent infection, amebiasis, intestinal tuberculosis, or parasitic disorder. In some embodiments, the present invention provides methods for treating or preventing a CDI and/or a CDAD, comprising administering an effective amount of a composition described herein to a subject or a patient need thereof. In various embodiments, the CDI or CDAD comprises one or more of: C. difficile diarrhea (CDD), C. difficile intestinal inflammatory disease, colitis, pseudomembranous colitis, fever, abdominal pain, dehydration and disturbances in electrolytes, megacolon, peritonitis, and perforation and/or rupture of the colon.

In various embodiments, the disease or condition associated with GI dysbiosis is treated or prevented in the context of initial onset or relapse/recurrence (e.g., due to continued or restarted antibiotic therapy). For example, in a patient that has previously suffered from a GI dysbiosis, the present composition or formulation may be administered upon the first symptoms of recurrence in the patient. By way of non-limiting example, symptoms of recurrence include, in a mild case, about 5 to about 10 watery bowel movements per day, no significant fever, and only mild abdominal cramps while blood tests may show a mild rise in the white blood cell count up to about 15,000 (normal levels are up to about 10,000), and, in a severe case, more than about 12 watery stools per day, nausea, vomiting, high fever (e.g., about 102-104° F.), rectal bleeding, severe abdominal pain (e.g., with tenderness), abdominal distention, and a high white blood count (e.g., of about 15,000 to about 40,000).

In some embodiments, the methods of the present invention are used to treat a subject or patient who is suffering from, or is susceptible to, a disease or condition associated with GI dysbiosis. For example, the patient may be undergoing or has undergone an initial and/or adjunctive therapy that renders the patient susceptible to a disease or condition associated with GI dysbiosis. In some embodiments, the patient is undergoing treatment, or has undergone treatment, with an antibiotic. For example, the patient may have taken an antibiotic during the past about 30 or so days and/or have an immune system that is weak (e.g., from a chronic illness). In another example, the patient may have recently been in the hospital, including in an intensive care unit. Accordingly, in some embodiments, the methods and uses of the present invention treat or prevent a nosocomial infection and/or a secondary emergent infection and/or a hospital acquired infection (HAI).

In various embodiments, the present invention provides methods for treating antibiotic-induced adverse effects in the GI tract, comprising administering an effective amount of a mixture of bacterial strains described herein (and/or additional therapeutic agents) to a subject in need thereof. In another embodiment, the present invention provides methods for preventing an antibiotic-induced adverse effect in the GI tract, comprising administering an effective amount of a mixture of bacterial strains described herein (and/or additional therapeutic agents) to a subject in need thereof.

In various embodiments, the mixtures of bacterial strains as described herein protect the intestinal microbiome from antibiotics-induced damage. In some embodiments, the methods of the invention treat or prevent an antibiotics-associated adverse effect including but not limited to diarrhea, nausea, vomiting, dysgeusia, colitis, and pseudomembranous colitis disease and/or symptoms. In an embodiment, methods of the invention can be used to treat or prevent antibiotic-associated diarrhea (AAD).

Methods for measuring change and/or improvement in GI tract function can include, but are not limited to: endoscopy for direct examination of epithelium and mucosa; histological evaluation and/or tissue procurement for direct evaluation of structural changes and/or immune biomarkers; urine tests for assessment of permeability with non-absorbable sugars and LPS levels; stool tests for assessment of inflammation and/or microbiota changes (for example by PCR); and/or blood tests for assessment of specific markers, including CD4+ cell counts, Th17 cell counts, and/or LPS levels.

In some embodiments, the methods of the present invention treat or prevent a diarrheal disease including, but not limited to, acute bloody diarrhea (e.g., dysentery), acute watery diarrhea (e.g., cholera), checkpoint inhibitor-associated colitis, diarrhea due to food poisoning, persistent diarrhea, and traveler's diarrhea.

In various embodiments, the methods of the present invention treat or prevent an IBD or related disease including, but not limited to, Crohn's disease, ulcerative colitis, collagenous colitis, lymphocytic colitis, diversion colitis, Behcet's disease, intermediate colitis, short bowel syndrome, ulcerative proctitis, proctosigmoiditis, left-sided colitis, pancolitis, and fulminant colitis.

In various embodiments, the methods of the present invention treat or prevent an autoimmune disorder including, but not limited to, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & Ill autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis.

In various embodiments, the methods of the present invention treat or prevent or reduce a side effect associated with an anti-cancer treatment. It is well known in the art that many anti-cancer treatments can affect the gut biosis. For example, chemotherapy can weaken the gut mucosa or change the mucosal-associated microbiota, including reduced diversity in the gut microbiome. Thus; such gut dysbiosis can lead to blood-stream infections. Additionally, the anti-cancer therapy can promote infection of and colonization of antibiotic resistant bacteria. See, e.g., Papanicolas et al., “Not Just Antibiotics: Is Cancer Chemotherapy Driving Antimicrobial Resistance?” Trends Microbiol. 2018 May; 26(5):393-400. Accordingly, bacterial mixtures of the present invention are useful in cancer-related applications, at least in decreasing the severity of a side effect and up to eliminating the side effect, e.g., in part, by promoting the decolonization and/or eradicating various pathogenic bacteria and/or repairing/repopulating his/her gut microbiome after receiving the anti-cancer therapeutic agent and/or anti-cancer therapy.

Moreover, the bacterial mixtures of the present invention are useful in cancer-related applications by increasing efficacy of an anti-cancer therapeutic agent and/or anti-cancer therapy by, at least, promoting the beneficial and desired effects of an anti-cancer therapeutic agent and/or anti-cancer therapy, i.e., killing cancer cells, reducing tumor size, and/or simulating an immune response against a cancer cell or tumor.

In aspects and embodiments, and without wishing to be bound by theory, the bacterial mixtures of the present invention may exert beneficial effects in oncology by maintaining responsiveness of a tumor, allowing an increased treatment dose (of the anti-cancer therapy) than otherwise possible, permitting administration of more frequent treatment doses (e.g., patient would not have to miss a session due to side effects), and/or boosting the immune system (e.g., for checkpoint inhibitor therapy).

In various embodiments, a subject in need thereof has received, is receiving, or will receive an anti-cancer therapeutic agent and/or an anti-cancer therapy.

Aspects of the present invention relate to methods for preventing or treating a cancer comprising administering a pharmaceutical composition comprising a microbial composition (with or without a chemotherapeutic agent that can be formulated for oral administration, as described herein) and administering an anti-cancer therapy.

In embodiments, a pharmaceutical composition is administered simultaneously (as described herein) with the anti-cancer therapy.

Alternately, the pharmaceutical composition and the anti-cancer therapy are administered sequentially. The term “sequentially” as used herein means that the anti-cancer therapy and the pharmaceutical composition are administered with a time separation of more than about 60 minutes. For example, the time between the sequential administration of the anti-cancer therapy and the pharmaceutical composition can be more than about 60 minutes, more than about 2 hours, more than about 5 hours, more than about 10 hours, more than about 1 day, more than about 2 days, more than about 3 days, more than about 1 week apart, more than 1 month apart, or longer. The optimal administration time will depend on the specific anti-cancer therapy and the pharmaceutical composition being administered. Either the anti-cancer therapy or the pharmaceutical composition may be administered first. In embodiments, the subject is administered pharmaceutical composition prior to the anti-cancer therapy, thereby helping ensure that the subject has a healthy gut biome prior to receiving the anti-cancer therapy. Alternately, the subject is administered pharmaceutical composition after the anti-cancer therapy, thereby helping the subject repair/repopulate his/her gut biome after receiving the anti-cancer therapy.

In embodiments, a bacterial mixture is administered enterally, e.g., orally, and the anti-cancer therapy is a chemotherapy or a targeted therapy which is administered parentally.

In aspects and embodiments, the anti-cancer therapy is a radiation therapy.

In aspects and embodiments, the anti-cancer therapy is a surgery, i.e., to excise a tumor or an organ/tissue comprising cancerous cells.

In aspects and embodiments, the anti-cancer therapy comprises a chemotherapy. Examples of chemotherapeutic agents include 5-FU (Fluorouracil), Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, ADE, Adriamycin (Doxorubicin), Afatinib Dimaleate, Afinitor (Everolimus), Afinitor Difsperz (Everolimus), Akynzeo (Netupitant and Palonosetron), Aldara (Imiquimod), Aldesleukin, Alecensa (Alectinib), Alectinib, Alimta (PEMETREXED), Aliqopa (Copanlisib Hydrochloride), Alkeran (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate), Arimidex (Anastrozole), Aromasin (Exemestane), Arranon (Nelarabine), Arsenic Trioxide, Asparaginase Erwinia chrysanthemi, Axicabtagene Ciloleucel, Axitinib, Azacitidine, BEACOPP, Becenum (Carmustine), Beleodaq (Belinostat), Belinostat, Bendamustine Hydrochloride, BEP, Bexarotene, Bicalutamide, BiCNU (Carmustine), Blenoxane (Bleomycin), Bortezomib, Bosulif (Bosutinib), Bosutinib, Brigatinib, BuMel, Busulfan, Busulfex (Busulfan)C, Cabazitaxel, Cabometyx (Cabozantinib), Cabozantinib-S-Malate, CAF, Calquence (Acalabrutinib), Camptosar (Irinotecan Hydrochloride), Capecitabine, CAPOX, Caprelsa (Vandetanib), Carac (Fluorouracil—Topical), Carboplatin, CARBOPLATIN-TAXOL, Carfilzomib, Carmubris (Carmustine), Carmustine, Casodex (Bicalutamide), CeeNU (Lomustine), CEM, Ceritinib, Cerubidine (Daunorubicin), Cervarix (Recombinant HPV Bivalent Vaccine), CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cytarabine, Cytarabine Liposome, Cytosar-U (Cytarabine), Cytoxan (Cyclophosphamide), Cytoxan (Cytoxan), Dabrafenib, Dacarbazine, Dacogen (Decitabine), Dactinomycin, Dasatinib, Daunorubicin Hydrochloride, Daunorubicin Hydrochloride and Cytarabine Liposome, DaunoXome (Daunorubicin Lipid Complex), Decadron (Dexamethasone), Decitabine, Defibrotide Sodium, Defitelio (Defibrotide Sodium), Degarelix, Denileukin Diftitox, DepoCyt (Cytarabine Liposome), Dexamethasone, Dexamethasone Intensol (Dexamethasone), Dexpak Taperpak (Dexamethasone), Dexrazoxane Hydrochloride, Docefrez (Docetaxel), Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), Droxia (Hydroxyurea), DTIC (Decarbazine), DTIC-Dome (Dacarbazine), Efudex (Fluorouracil—Topical), Eligard (Leuprolide), Elitek (Rasburicase), Ellence (Ellence (epirubicin)), Eloxatin (Oxaliplatin), Elspar (Asparaginase), Eltrombopag Olamine, Emcyt (Estramustine), Emend (Aprepitant), Enasidenib Mesylate, Enzalutamide, Epirubicin Hydrochloride, EPOCH, Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Eulexin (Flutamide), Evacet (Doxorubicin Hydrochloride Liposome), Everolimus, Evista (Raloxifene Hydrochloride), Evomela (Melphalan Hydrochloride), Exemestane, Fareston (Toremifene), Farydak (Panobinostat), Faslodex (Fulvestrant), FEC, Femara (Letrozole), Filgrastim, Firmagon (Degarelix), FloPred (Prednisolone), Fludara (Fludarabine), Fludarabine Phosphate, Fluoroplex (Fluorouracil), Fluorouracil, Flutamide, Folex (Methotrexate), Folex PFS (Methotrexate), FOLFIRI, FOLFIRINOX, FOLFOX, Folotyn (Pralatrexate), FUDR (FUDR (floxuridine)), FU-LV, Fulvestrant, Gardasil (Recombinant HPV Quadrivalent Vaccine), Gardasil 9 (Recombinant HPV Nonavalent Vaccine), Gefitinib, Gemcitabine Hydrochloride, GEMCITABINE-CISPLATIN, GEMCITABINE-OXALIPLATIN, Gemzar (Gemcitabine), Gilotrif (Afatinib Dimaleate), Gilotrif (Afatinib), Gleevec (Imatinib Mesylate), Gliadel (Carmustine), Glucarpidase, Goserelin Acetate, Halaven (Eribulin Mesylate), Hemangeol (Propranolol Hydrochloride), Hexalen (Altretamine), HPV Bivalent Vaccine, Recombinant, HPV Nonavalent Vaccine, Recombinant, HPV Quadrivalent Vaccine, Recombinant, Hycamtin (Topotecan Hydrochloride), Hycamtin (Topotecan), Hydrea (Hydroxyurea), Hydroxyurea, Hyper-CVAD, Ibrance (Palbociclib), Ibrutinib, ICE, Iclusig (Ponatinib), Idamycin PFS (Idarubicin), Idarubicin Hydrochloride, Idelalisib, Idhifa (Enasidenib), Ifex (Ifosfamide), Ifosfamide, Ifosfamidum (Ifosfamide), Imatinib Mesylate, Imbruvica (Ibrutinib), Imiquimod, Imlygic (Talimogene Laherparepvec), Inlyta (Axitinib), Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jakafi (Ruxolitinib), JEB, Jevtana (Cabazitaxel), Keoxifene (Raloxifene Hydrochloride), Kepivance (Palifermin), Kisqali (Ribociclib), Kyprolis (Carfilzomib), Lanreotide Acetate, Lanvima (Lenvatinib), Lapatinib Ditosylate, Lenalidomide, Lenvatinib Mesylate, Lenvima (Lenvatinib Mesylate), Letrozole, Leucovorin Calcium, Leukeran (Chlorambucil), Leukine (Sargramostim), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil), Lupron (Leuprolide), Lynparza (Olaparib), Lysodren (Mitotane), Marqibo (Vincristine Sulfate Liposome), Marqibo Kit (Vincristine Lipid Complex), Matulane (Procarbazine), Mechlorethamine Hydrochloride, Megace (Megestrol), Megestrol Acetate, Mekinist (Trametinib), Melphalan, Melphalan Hydrochloride, Mercaptopurine, Mesnex (Mesna), Metastron (Strontium-89 Chloride), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mostarina (Prednimustine), Mozobil (Plerixafor), Mustargen (Mechlorethamine), Mutamycin (Mitomycin), Myleran (Busulfan), Mylosar (Azacitidine), Nanoparticle Paclitaxel (Paclitaxel Albumin-stabilized Nanoparticle Formulation), Navelbine (Vinorelbine), Nelarabine, Neosar (Cyclophosphamide), Neratinib Maleate, Nerlynx (Neratinib), Netupitant and Palonosetron Hydrochloride, Neulasta (filgrastim), Neulasta (pegfilgrastim), Neupogen (filgrastim), Nexavar (Sorafenib), Nilandron (Nilutamide), Nilotinib, Nilutamide, Ninlaro (Ixazomib), Nipent (Pentostatin), Niraparib Tosylate Monohydrate, Nolvadex (Tamoxifen), Novantrone (Mitoxantrone), Nplate (Romiplostim), Odomzo (Sonidegib), OEPA, OFF, Olaparib, Omacetaxine Mepesuccinate, Oncaspar (Pegaspargase), Oncovin (Vincristine), Ondansetron Hydrochloride, Onivyde (Irinotecan Hydrochloride Liposome), Ontak (Denileukin Diftitox), Onxol (Paclitaxel), OPPA, Orapred (Prednisolone), Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panobinostat, Panretin (Alitretinoin), Paraplat (Carboplatin), Pazopanib Hydrochloride, PCV, PEB, Pediapred (Prednisolone), Pegaspargase, Pegfilgrastim, Pemetrexed Disodium, Platinol (Cisplatin), PlatinolAQ (Cisplatin), Plerixafor, Pomalyst (Pomalidomide), Ponatinib Hydrochloride, Pralatrexate, Prednisone, Procarbazine Hydrochloride, Proleukin (Aldesleukin), Promacta (Eltrombopag Olamine), Propranolol Hydrochloride, Purinethol (Mercaptopurine), Purixan (Mercaptopurine), Radium 223 Dichloride, Raloxifene Hydrochloride, Rasburicase, R-CHOP, R-CVP, Reclast (Zoledronic acid), Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Regorafenib, Relistor (Methylnaltrexone Bromide), R-EPOCH, Revlimid (Lenalidomide), Rheumatrex (Methotrexate), Ribociclib, R-ICE, Rolapitant Hydrochloride, Romidepsin, Romiplostim, Rubex (Doxorubicin), Rubidomycin (Daunorubicin Hydrochloride), Rubraca (Rucaparib), Rucaparib Camsylate, Ruxolitinib Phosphate, Rydapt (Midostaurin), Sandostatin (Octreotide), Sandostatin LAR Depot (Octreotide), Sclerosol Intrapleural Aerosol (Talc), Soltamox (Tamoxifen), Somatuline Depot (Lanreotide Acetate), Sonidegib, Sorafenib Tosylate, Sprycel (Dasatinib), STANFORD V, Sterapred (Prednisone), Sterapred DS (Prednisone), Sterile Talc Powder (Talc), Steritalc (Talc), Sterecyst (Prednimustine), Stivarga (Regorafenib), Sunitinib Malate, Supprelin LA (Histrelin), Sutent (Sunitinib Malate), Sutent (Sunitinib), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib), Targretin (Bexarotene), Tasigna (Decarbazine), Tasigna (Nilotinib), Taxol (Paclitaxel), Taxotere (Docetaxel), Temodar (Temozolomide), Temozolomide, Temsirolimus, Tepadina (Thiotepa), Thalidomide, Thalomid (Thalidomide), TheraCys BCG (BCG), Thioguanine, Thioplex (Thiotepa), Thiotepa, TICE BCG (BCG), Tisagenlecleucel, Tolak (Fluorouracil—Topical), Toposar (Etoposide), Topotecan Hydrochloride, Toremifene, Torisel (Temsirolimus), Totect (Dexrazoxane Hydrochloride), TPF, Trabectedin, Trametinib, Treanda (Bendamustine hydrochloride), Trelstar (Triptorelin), Trexall (Methotrexate), Trifluridine and Tipiracil Hydrochloride, Trisenox (Arsenic trioxide), Tykerb (lapatinib), Uridine Triacetate, VAC, Valrubicin, Valstar (Valrubicin Intravesical), Valstar (Valrubicin), VAMP, Vandetanib, Vantas (Histrelin), Varubi (Rolapitant), VeIP, Velban (Vinblastine), Velcade (Bortezomib), Velsar (Vinblastine Sulfate), Vemurafenib, Venclexta (Venetoclax), Vepesid (Etoposide), Verzenio (Abemaciclib), Vesanoid (Tretinoin), Viadur (Leuprolide Acetate), Vidaza (Azacitidine), Vinblastine Sulfate, Vincasar PFS (Vincristine), Vincrex (Vincristine), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib), Vumon (Teniposide), Vyxeos (Daunorubicin Hydrochloride and Cytarabine Liposome), W, Wellcovorin (Leucovorin Calcium), Wellcovorin IV (Leucovorin), Xalkori (Crizotinib), XELIRI, Xeloda (Capecitabine), XELOX, Xofigo (Radium 223 Dichloride), Xtandi (Enzalutamide), Yescarta (Axicabtagene Ciloleucel), Yondelis (Trabectedin), Zaltrap (Ziv-Aflibercept), Zanosar (Streptozocin), Zarxio (Filgrastim), Zejula (Niraparib), Zelboraf (Vemurafenib), Zinecard (Dexrazoxane Hydrochloride), Ziv-Aflibercept, Zofran (Ondansetron Hydrochloride), Zoladex (Goserelin), Zoledronic Acid, Zolinza (Vorinostat), Zometa (Zoledronic acid), Zortress (Everolimus), Zydelig (Idelalisib), Zykadia (Ceritinib), Zytiga (Abiraterone Acetate), and Zytiga (Abiraterone).

In embodiments, the chemotherapy is a hormonal therapy. Illustrative hormone therapeutics include aromatase inhibitors, e.g., Letrozole, anastrozole, exemestane, aminoglutethimide; gonadotropin-releasing hormone (GnRH) analogues, e.g., leuprorelin and goserelin; hormone receptor antagonists, e.g., selective estrogen receptor modulators (as examples, tamoxifen, raloxifene, toremifene and fulvestrant) and antiandrogens, e.g., flutamide and bicalutamide; and hormone supplementation, e.g., megestrol acetate, medroxyprogesterone acetate, fluoxymesterone, diethylstilbestrol, estrace, polyestradiol phosphate, and octreotide.

In aspects and embodiments, the anti-cancer therapy is an immuno-oncology therapy. An immuno-oncology therapy comprises at least one molecule capable of binding and/or recognizing a tumor-cell antigen and/or a cancer-cell antigen. Examples, tumor-cell antigens and/or a cancer-cell antigens include but are not limited to, carbonic anhydrase IX (CAIX), 5T4, CD19, CD20, CD22, CD30, CD33, CD38, CD47, CS1, CD138, Lewis-Y, L1-CAM, MUC16, ROR-1, IL13Ra2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-α), GD2, human epidermal growth factor receptor 2 (HER2), mesothelin, EGFRvIII, fibroblast activation protein (FAP), carcinoembryonic antigen (CEA), and vascular endothelial growth factor receptor 2 (VEGF-R2), as well as other tumor antigens well known in the art. Additional illustrative tumor antigens include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmeli117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2.

In embodiments, the tumor-cell antigen and/or a cancer-cell antigen is a checkpoint molecule. The checkpoint molecule may be a stimulatory checkpoint molecule, e.g., CD27, CD28, CD40, CD122, CD137, OX40, GITR, and ICOS. The checkpoint molecule may be an inhibitory checkpoint molecule, e.g., 2B4, A2AR, B-7 family ligands (including, but are not limited to, B7-1, B7-2, B7-DC, B7-H1, B7-H2, B7-H3, B7-H4, B7-H5, B7-H6 and B7-H7), BTLA, CD115, CD160/By55, CD172a/SIRPα, CD200, CD223, CD244, CEACAM, CHK 1 and CHK2 kinases, CTLA-4, GAL9, HVEM, IDO, KIR, LAG3, PD-1, PD-L1, PD-L2, TIGIT, TIM-3, TMIGD2, and VISTANVSIG8.

In embodiments, the immuno-oncology therapy is protein-based, e.g., antibody, fusion protein, and/or cytokine.

In embodiments, the antibody is Adcetris (Brentuximab Vedotin), Ado-Trastuzumab Emtansine, Alemtuzumab, Arzerra (Ofatumumab), Atezolizumab, Avastin (Bevacizumab), Avelumab, Bavencio (Avelumab), Besponsa (Inotuzumab Ozogamicin), Bevacizumab, Bexxar (Tositumomab), Blinatumomab, Blincyto (Blinatumomab), BMS 936559, Brentuximab Vedotin, Campath (Alemtuzumab), Cetuximab, Cinqair (Reslizumab), Cyramza (Ramucirumab), Daratumumab, Darzalex (Daratumumab), Denosumab, Dinutuximab, Durvalumab, Elotuzumab, Empliciti (Elotuzumab), Erbitux (Cetuximab), Folfiri-Bevacizumab, Folfiri-Cetuximab, Gazyva (Obinutuzumab), Gemtuzumab Ozogamicin, Herceptin (Trastuzumab), Ibritumomab Tiuxetan, Imfinzi (Durvalumab), Inotuzumab Ozogamicin, Ipilimumab, Kadcyla (Ado-trastuzumab Emtansine), Keytruda (Pembrolizumab), Lartruvo (Olaratumab), MK-3475, MPDL3280A, Mylotarg (Gemtuzumab Ozogamicin), Necitumumab, Nivolumab, Obinutuzumab, Ofatumumab, Olaratumab, Opdivo (Nivolumab), Panitumumab, Perjeta (Pertuzumab), Pertuzumab, Pembrolizumab, Pidilizumab, Portrazza (Necitumumab), Prolia (Denosumab), Ramucirumab, Rituxan (Rituximab), Rituximab and Hyaluronidase Human, Siltuximab, Sylvant (Siltuximab), Tecentriq (Atezolizumab), Trastuzumab, Unituxin (Dinutuximab), Vectibix (Panitumumab), Xgeva (Denosumab), Yervoy (Ipilimumab), and Zevalin (Ibritumomab Tiuxetan).

In embodiments, the immuno-oncology therapy includes an engineered protein or a fusion protein. In embodiments, the engineered protein or fusion protein binds to one or more tumor-cell antigens and/or cancer-cell antigens. In embodiments, the fusion protein binds to one or more tumor-cell antigens and/or cancer-cell antigens and is conjugated to a chemotherapeutic agent (as described herein).

In embodiments, the immuno-oncology therapy includes a cytokine, e.g., which binds to one or more tumor-cell antigens and/or cancer-cell antigens. In embodiments, the cytokine is Interferon Alfa-2b, Interleukin-2 (Aldesleukin), Intron A alfab (Interferon alfa-2a), Peginterferon Alfa-2b, PEG-Intron (Peginterferon Alfa-2b), Recombinant Interferon Alfa-2b, RoferonA alfaa (Interferon alfa-2a), and Sylatron (Peginterferon Alfa-2b).

In embodiments, binding and/or recognizing a tumor-cell antigen and/or a cancer-cell antigen blocks and/or prevents downstream signaling of the tumor-cell antigen and/or cancer-cell antigen. Alternately, binding and/or recognizing a tumor-cell antigen and/or a cancer-cell antigen activates and/or stimulates downstream signaling of the tumor-cell antigen and/or cancer-cell antigen.

In embodiments, the immuno-oncology therapy is a cell-based immuno-oncology therapy, e.g., relating to adoptive cell transfer (ACT). The ACT may be autologous or allogenic.

In embodiments, the cell-based immuno-oncology therapy comprises use of Chimeric Antigen Receptor (CAR) T-cell. Exemplary CAR T-cell therapy include, but are not limited to, JCAR014 (Juno Therapeutics), JCAR015 (Juno Therapeutics), JCAR017 (Juno Therapeutics), JCAR018 (Juno Therapeutics), JCAR020 (Juno Therapeutics), JCAR023 (Juno Therapeutics), JCAR024 (Juno Therapeutics), CTL019 (Novartis), Kymriah (or tisagenlecleucel; Novartis), KTE-C19 (Kite Pharma), BPX-401 (Bellicum Pharmaceuticals), BPX-501 (Bellicum Pharmaceuticals), BPX-601 (Bellicum Pharmaceuticals), bb2121 (Bluebird Bio), CD-19 Sleeping Beauty cells (Ziopharm Oncology), UCART19 (Cellectis), UCART123 (Cellectis), UCART38 (Cellectis), UCARTCS1 (Cellectis), OXB-302 (Oxford BioMedica, MB-101 (Mustang Bio), and CAR T-cells developed by Innovative Cellular Therapeutics.

In embodiments, the cell-based immuno-oncology therapy comprises use of an antigen-presenting cell (APC). In embodiments, the APC-related therapy comprises use of dendritic cells or other APCs that express tumor-cell antigens or cancer-cell antigens (as described herein). In one example, the APC is Sipuleucel-T (APC8015, trade name Provenge; Dendreon Corporation).

In embodiments, the cell-based immuno-oncology therapy comprises use of engineered T Cell Receptors (TCR) which recognize tumor-cell antigens or cancer-cell antigens (as described herein).

In embodiments, the cell-based immuno-oncology therapy comprises use of tumor infiltrating lymphocytes (TIL), e.g., adoptive transfer of TILs, which recognize tumor-cell antigens or cancer-cell antigens (as described herein).

In various embodiments, the mixtures of bacterial strains may stimulate and/or activate Toll-like receptor activity (e.g., TLR1, and/or TLR2, and/or TLR3, and/or TLR4, and/or TLR5, and/or TLR6, and/or TLR7, and/or TLR8, and/or TLR9, and/or TLR10, and/or TLR11, and/or TLR12, and/or TLR13).

In various embodiments, the methods of the present invention treat or prevent various bloodstream infections (BSI), catheter or intravascular-line infections (e.g., central-line infections), chronic inflammatory diseases, meningitis, pneumonia, e.g., ventilator-associated pneumonia, skin and soft tissue infections, surgical-site infections, urinary tract infections (e.g., antibiotic-resistant urinary tract infections and catheter-associated urinary tract infections), wound infections, and/or other well-known infections: antibiotic-resistant infections and antibiotic-sensitive infections.

In various embodiments, the methods of the present invention treat or prevent various GI disorders disclosed herein and/or as known in the art to be a result of gut dysbiosis.

In various embodiments, the methods of the present invention reduce GI immunoactivation and inflammation.

In various embodiments, the methods of the present invention reduce, ameliorate, or eliminate one or more symptom(s) associated with a herein-described disease, disorder, or condition. Exemplary symptoms include, but are not limited to, diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts.

In one aspect, a method comprises administering a therapeutic composition orally, by enema, or via rectal suppository. In one aspect, a pharmaceutical composition is formulated as a geltab, pill, microcapsule, capsule, or tablet. In one aspect, a therapeutic composition is formulated as an enteric coated capsule or microcapsule, acid-resistant capsule or microcapsule, or formulated as part of or administered together with a food, a food additive, a dairy-based product, a soy-based product or a derivative thereof, a jelly, or a yogurt. In another aspect, a therapeutic composition is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

Any aspect or embodiment disclosed herein can be combined with any other aspect or embodiment as disclosed herein.

### Definitions

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “microbiota,” and “flora” refer to a community of microbes that live in or on a subject's body, both sustainably and transiently, including eukaryotes, archaea, bacteria, and viruses (including bacterial viruses (i.e., phage)). A non-selected fecal microbiota refers to a community or mixture of fecal microbes derived from a donor's fecal sample without selection and substantially resembling microbial constituents and population structure found in such fecal sample.

As used herein, “gut dysbiosis” refers to an imbalance, maladaptation, and/or reduced diversity in the microbiota in a subject's digestive system. For example, a part of the gut flora is unbalanced, with normally dominating species, i.e., beneficial bacteria, becoming underrepresented (and/or less metabolically active) and outcompeted by contained species, e.g., pathogenic and/or antibiotic-resistant bacteria, which proliferate to fill the void.

As used herein, examples of a “side effect of an anti-cancer therapeutic agent” and a “side effect of an anti-cancer therapy” include abdominal pain, anemia and low blood counts, appetite loss, autoimmune effects, bleeding and bruising (thrombocytopenia), cancer, changes in mood or thinking, colonization by pathogenic bacteria, constipation, cough, dehydration, delirium, diabetes-related symptoms, diarrhea, dry mouth or xerostomia, eating problems, edema, falling, fatigue, fertility issues, fever, flu-like symptoms, fluid in the abdomen or ascites, gastrointestinal (GI) mucositis, gut dysbiosis, hair loss (alopecia), hand-foot syndrome or palmar-plantar erythrodysesthesia, headache, hearing problems, high or low blood pressure, hormone changes, hiccups, hypercalcemia, infection and neutropenia, infection by pathogenic bacteria, inflammatory bowel disease, irritable bowel syndrome, leg cramps, lymphedema, memory or concentration problems, mouth and throat problems, nausea and vomiting, nerve problems (peripheral neuropathy), obesity, osteoporosis, ostomies, pain, seizures, sexual health issues, shortness of breath, sinus congestion, skin and nail changes, sleep problems, stool or urine changes, sweating, swelling, ulcerative colitis, urinary and bladder problems, weight gain from retaining fluid, and/or weakness. In embodiments, a side effect of the anti-cancer therapeutic agent and/or anti-cancer therapy is caused by gut dysbiosis; it has been reported that chemotherapy, for example, is associated with reduced diversity in the gut microbiome. Thus, “treating or preventing or reducing a side effect” refers to decreasing the severity of a side effect and up to eliminating the side effect, e.g., in part, by repairing/repopulating his/her gut microbiome after receiving the anti-cancer therapeutic agent and/or anti-cancer therapy.

As used herein, “increase[ing] efficacy of an anti-cancer therapeutic agent and/or anti-cancer therapy” refers to the ability of a microbial composition or method using same to promote the beneficial and desired effects of an anti-cancer therapeutic agent and/or anti-cancer therapy, i.e., killing cancer cells, reducing tumor size, and/or simulating an immune response against a cancer cell or tumor.

As used herein, “spore” or a population of “spores” includes bacteria (or other single-celled organisms) that are generally viable, more resistant to environmental influences such as heat and bactericidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

In embodiments, the subject, e.g., a human, is refractory and/or non-responsive to a treatment directed to a checkpoint molecule. In embodiments, the treatment directed to a checkpoint molecule comprises administration of Keytruda (Pembrolizumab), Opdivo (Nivolumab), Yervoy (Ipilimumab), Tecentriq (atezolizumab), Bavencio (avelumab), or Imfinzi (durvalumab).

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5%.

In some embodiments, the terms “patient” and “subject” are used interchangeably. In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish.

In various embodiments, methods of the invention are useful in treatment a human subject. In some embodiments, the human is a pediatric human. In other embodiments, the human is an adult human. In other embodiments, the human is a geriatric human. In other embodiments, the human may be referred to as a patient. In some embodiments, the human is a female. In some embodiments, the human is a male.

In certain embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within (plus or minus) 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The terms “one or more”, “at least one”, and the like are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

Conversely, the term “no more than” includes each value less than the stated value.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

The term “greater than” and the like, is understood to include values greater than the stated by at least 1, 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

A stated range is understood to be any value between and at the limits of the stated range. As examples, a range between 1 and 5 includes 1, 2, 3, 4, and 5; a range between 1 and 10 includes 1, 2, 3, 4, 5, 6, 7, 8, 9, and 10; and a range between 1 and 100 includes 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100.

Unless defined otherwise, all technical and scientific terms used herein have the same meaning as commonly understood by one of ordinary skill in the art to which the invention pertains. Although other probes, compositions, methods, and kits similar, or equivalent, to those described herein can be used in the practice of the present invention, the preferred materials and methods are described herein. It is to be understood that the terminology used herein is for the purpose of describing particular embodiments only, and is not intended to be limiting.

### EXAMPLES

### Example 1: Determination of Useful Bacteria in a Bacterial Mixture of the Present Invention

Tables 5 and 6 (above) were created based upon analyses using 16S rRNA sequencing data from public and private sources to identify drivers of ARB decolonization.

Some of the operational taxonomic units (OTUs) were identified because they are present in healthy stool donors and the family or genus they belong to was at least five-fold enriched in stool donors compared to patients pre-FMT, and in a receiver operator curve (ROC) plot, the area under the curve (AUC) was greater than or equal to 0.6, showing that the presence/absence of these families and genera can predict whether a sample is from a donor or from a patient pre-FMT. Some of the OTUs were identified because they are present in healthy stool donors and the family or genus they belong to was enriched in patients who did not become dominated with Enterococcus or pathogenic Gram-negative bacteria compared to those who became dominated, and in a ROC plot, the AUC was greater than or equal to 0.6, showing that the presence/absence of these families and genera can predict whether a patient's gut will become dominated by enteric pathogens. Patients had received or were undergoing stem cell transplantation as part of a treatment for leukemia, lymphoma, multiple myeloma or myelodysplastic syndrome.

Other OTUs were identified because they are present in healthy stool donors and the family or genus they belong to was enriched in people with a C. difficile infection who were not colonized with Extended Spectrum Beta-Lactamase producing Enterobacteriaceae (ESBL-E) compared to those who became colonized, and in a ROC plot, the AUC was greater than or equal to 0.6, showing that the presence/absence of these families and genera can predict whether a patient will become colonized with ESBL-E.

Four datasets were used in these analyses: (1) Taur Y, Xavier J, Lipuma L, Carles Ubeda, Goldberg J, Gobourne A, Lee Y, Dubin K, Socci N, Viale A, Perales M-A, Jenq R, Brink M, Pamer E. Intestinal Domination and the Risk of Bacteremia in Patients Undergoing Allogeneic Hematopoietic Stem Cell Transplantation. Clin Infect Dis. 2012; 55(7):905-914. PMCID: PMC3657523; (2) Gosalbes M J, Vazquez-Castellanos J F, Angebault C, Woerther P-L, Ruppé E, Ferrús M L, Latorre A, Andremont A, Moya A. Carriage of Enterobacteria Producing Extended-Spectrum β-Lactamases and Composition of the Gut Microbiota in an Amerindian Community. Antimicrob Agents Chemother. 2015 Dec. 31; 60(1):507-514. PMCID: PMC4704183; (3) Multicenter retrospective study assessing efficacy of FMT for VRE decolonization in patients with rCDI who were also colonized with VRE; and (4) 16S sequencing was performed on samples from 63 healthy and extensively-screened stool donors.

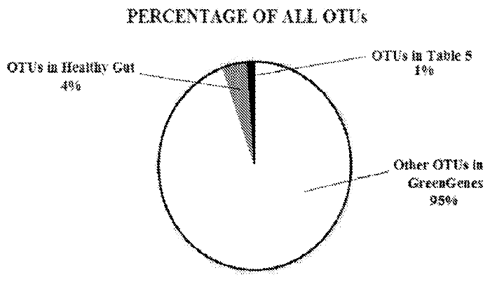

FIG. 1 is a pie chart showing percentage of all OTUs in GreenGenes database that are found in a heathy human gut and that are included in Table 5. The GreenGenes database contains sequence data corresponding to almost 100,000 unique 16S sequences from different bacterial strains that have been sequenced. See, the World Wide Web (www) at greengenes.lbl.gov. FIG. 1 shows the fraction of all OTUs found in the GreenGenes database that are found in the human gut and that are relevant to the present invention. As shown in FIG. 1, 94.6% of all OTUs in the GreenGenes database are not found in the healthy human gut; 4.3% of all OTUs in the GreenGenes database are found in the healthy human gut; and 1.2% of all OTUs in the GreenGenes database that are listed in Table 5.

### Example 2. Development of Bacterial Mixtures

A product candidate is being developed which includes a novel mixture of commensal bacterial strains that can decolonize and/or eradicate and/or reduce the load of antibiotic-resistant enteric pathogens in the gut.

Bacterial strains may be included in a mixture based on their abundance in donors whose stool was used for successful fecal microbiota transplants (FMTs) in a patient suffering from a gut dysbiosis disorder, e.g., caused by a previous or current anti-cancer therapy. Additionally, bacterial strains may be included in a mixture due to their ability to colonize the mucosa, to complement the capacity of a functionally deficient microbial community (e.g., the microbial community of a patient infected and/or colonized by a pathogenic bacteria), to produce levels of SCFAs comparable to healthy individuals, to decolonize a pathogenic bacterium, to directly compete with the ARB for a niche and/or for nutrients, to directly compete with the pathogenic bacteria for a niche and/or for nutrients, to directly inhibit a pathogenic bacterium through production of a secreted product, to directly inhibit an antibiotic-resistant bacterium (ARB) through production of a secreted product, to enable mucosal healing, improve mucosal barrier function, and/or to reduce inflammation, to enhance production of SCFAs, to eradicate a pathogenic bacterium, to improve mucosal barrier function, and/or, to promote restoration of mucosal barrier functions, and/or to reduce inflammation.

Certain bacterial strains are included in a mixture based upon their 16S rRNA sequence identity. For example, the mixture includes one or more bacterial strains having a 16S rRNA sequence that is at least about 80% identical to the 16S rRNA sequence of any one of the operational taxonomic units (OTUs) provided in Table 5 or Table 6. For example, the mixture may include one or more bacterial strains having a 16S rRNA sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical with the 16S rRNA sequence of any one of the operational taxonomic units (OTUs) provided in Table 5 or Table 6.

Certain mixtures of bacterial strains are substantially complete or non-selected fecal microbiota preparations (e.g., from a single donor), which generally comprises a full complement of functional microorganisms found in feces of one or more healthy humans. Such mixtures of bacterial strains may be supplemented with one or more strains listed in Table 5 or Table 6 and/or one or more strains having a 16S rRNA sequence that is at least about 95% identical with the 16S rRNA sequence of any one of the strains listed in Table 5 or Table 6.

Other mixtures of bacterial strains comprise “less than the full complement” of functional microorganisms found in feces of one healthy human or in feces of more than one healthy human donor. These bacterial mixtures omit at least one bacterial strain from the full complement. Such mixtures of bacterial strains may be supplemented with one or more strains listed in Table 5 or Table 6 and/or one or more strains having a 16S rRNA sequence that is at least about 95% identical with the 16S rRNA sequence of any one of the strains listed in Table 5 or Table 6. Some bacterial strains in a mixture are directly obtained from human feces (i.e., from a suitable and healthy donor); Some of those strains may isolated or purified from its source material, i.e., separated from at least some of the components with which they were associated when initially produced (e.g., nature (from feces)) Some bacterial strains in a mixture are indirectly obtained from human feces and/or are obtained independent of human feces (e.g., from a bacterial cell bank or from a laboratory stock).

### Example 3: Production of a Pharmaceutical Composition

Strains selected in Example 1 and/or Example 2 may be independently cultured and mixed together before administration. Cultured strains are independently grown in supportive media, e.g. at 37° C., pH 7, in a GMM or other animal-products-free medium, pre-reduced with 1 g/L cysteine-HCl. After each strain reaches a sufficient biomass, it is optionally preserved for banking by adding 15% glycerol and then frozen at −80° C. in 1 ml cryotubes. Each strain may then be cultivated to a concentration of about 10¹⁰ CFU/mL, then concentrated 20-fold by tangential flow microfiltration; the spent medium is exchanged by diafiltering with a preservative medium consisting of 2% gelatin, 100 mM trehalose, and 10 mM sodium phosphate buffer, or other suitable preservative medium. The suspension is freeze-dried to a powder and titrated.

After drying, the powder is blended with microcrystalline cellulose and magnesium stearate and formulated into a 250 mg gelatin capsule containing 10 mg of lyophilized powder (10⁸ to 10¹¹ bacteria), 160 mg microcrystalline cellulose, 77.5 mg gelatin, and 2.5 mg magnesium stearate.

### Example 4: Methods of Treatment

A subject having gut dysbiosis is administered a pharmaceutical composition comprising a bacterial mixture of the present invention to treat the gut dysbiosis.

For subjects who have gut dysbiosis as a side effect of an anti-cancer therapeutic agent and/or a side effect of an anti-cancer therapy, the pharmaceutical composition helps reduce or treating the side effect.

For subjects who have undergone or are undergoing an anti-cancer therapeutic agent and/or a side effect of an anti-cancer therapy, the pharmaceutical composition increases the efficacy of the anti-cancer therapeutic agent and/or anti-cancer therapy.

### Example 5: Methods of Prevention

A subject at risk for gut dysbiosis is administered a pharmaceutical composition comprising a bacterial mixture of the present invention to prevent gut dysbiosis.

For subjects who are at risk for gut dysbiosis as a side effect of an anti-cancer therapeutic agent and/or a side effect of an anti-cancer therapy, the pharmaceutical composition helps prevents the likelihood of getting the side effect.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### REFERENCES

1. Antibiotic Resistance Threats in the United States, 2013 [Internet]. US Department of Health and Human Services, Centers for Disease Control and Prevention; 2013. Available from: the World Wide Web (www) at cdc.gov/drugresistance/pdf/ar-threats-2013-508.pdf
2. Stone P W. Economic burden of healthcare-associated infections: an American perspective. Expert Rev Pharmacoecon Outcomes Res. 2009 October; 9(5):417-422. PMCID: PMC2827870
3. Pamer E G. Resurrecting the intestinal microbiota to combat antibiotic-resistant pathogens. Science. 2016; 352(6285):535-8. PMCID: PMC4984266
4. Abt M, Pamer E. Commensal bacteria mediated defenses against pathogens. Curr Opin Immunol. 2014; 29:16-22. PMCID: PMC4132187
5. Becattini S, Taur Y, Pamer E G. Antibiotic-Induced Changes in the Intestinal Microbiota and Disease. Trends Mol Med. 2016; 22(6):458-78. PMCID: PMC4885777
6. Carlet J. The gut is the epicentre of antibiotic resistance. Antimicrob Resist Infect Control. 2012; 1:39.
7. Buffie C, Pamer E. Microbiota-mediated colonization resistance against intestinal pathogens. Nat Rev Immunol. 2013; 13(11):790-801. PMCID: PMC4194195
8. Martina S-C, Raffatellu M. No Vacancy: How Beneficial Microbes Cooperate with Immunity To Provide Colonization Resistance to Pathogens. J Immunol. 2015; 194(9):4081-4087. PMCID: PMC4402713
9. Ubeda C, Taur Y, Jenq R, Equinda M, Son T, Samstein M, Viale A, Socci N, Brink M, Kamboj M, Pamer E. Vancomycin-resistant Enterococcus domination of intestinal microbiota is enabled by antibiotic treatment in mice and precedes bloodstream invasion in humans. J Clin Invest. 2010; 120(12):4332-4341. PMCID: PMC2993598
10. Ziakas P D, Thapa R, Rice L B, Mylonakis E. Trends and significance of VRE colonization in the ICU: a meta-analysis of published studies. PloS One. 2013; 8(9):e75658. PMCID: PMC3785502
11. Hendrix C W, Hammond J M, Swoboda S M, Merz W G, Harrington S M, Perl T M, Dick J D, Borschel D M, Halczenko P W, Pelz R K, Rocco L E, Conway J E, Brower R G, Lipsett P A. Surveillance strategies and impact of vancomycin-resistant enterococcal colonization and infection in critically ill patients. Ann Surg. 2001 February; 233(2):259-265. PMCID: PMC1421209
12. Horwitz D, McCue T, Mapes A C, Ajami N J, Petrosino J F, Ramig R F, Trautner B W. Decreased microbiota diversity associated with urinary tract infection in a trial of bacterial interference. J Infect. 2015 September; 71(3):358-367. PMCID: PMC4529357
13. Safdar N, Sengupta S, Musuuza J S, Juthani-Mehta M, Drees M, Abbo L M, Milstone A M, Furuno J P, Varman M, Anderson D J, Morgan D J, Miller L G, Snyder G M, and the SHEA Research Committee. Status of the Prevention of Multidrug-Resistant Organisms in International Settings: A Survey of the Society for Healthcare Epidemiology of America Research Network. Infect Control Hosp Epidemiol. 2016 Nov. 7; 1-8. PMID: 27817759
14. Abad C, Fearday A, Safdar N. Adverse effects of isolation in hospitalised patients: a systematic review. J Hosp Infect. 2010; 76(2):97-102. PMID: 20619929
15. Bowling J E, Taylor B S. Isolation Precautions for Hospitalized Patients: The Challenges of Identifying Unintended Individual Consequences and Measuring the Prevention of Community Harm. J Gen Intern Med. 2016 Nov. 18; 1-3.
16. Guilley-Lerondeau B, Bourigault C, Buttes A-CG des, Birgand G, Lepelletier D. Adverse effects of isolation: a prospective matched cohort study including 90 direct interviews of hospitalized patients in a French University Hospital. Eur J Clin Microbiol Infect Dis. 2017 Jan. 1; 36(1):75-80.
17. Kassam Z, Lee C H, Yuan Y, Hunt R H. Fecal microbiota transplantation for Clostridium difficile infection: systematic review and meta-analysis. Am J Gastroenterol. 2013 April; 108(4):500-508. PMID: 23511459
18. Fischer M, Sipe B, Cheng Y-W, Phelps E, Rogers N, Sagi S, Bohm M, Xu H, Kassam Z. Fecal microbiota transplant in severe and severe-complicated Clostridium difficile: A promising treatment approach. Gut Microbes. 2016 Dec. 21; 1-14. PMID: 28001467
19. Singh R, Nood E, Nieuwdorp M, Dam B, Berge I, Geerlings S, Bemelman F. Donor feces infusion for eradication of Extended Spectrum beta-Lactamase producing Escherichia coli in a patient with end stage renal disease. Clin Microbiol Infec. 2014; 20(11):0977-0978. PMID: 24845223
20. Garcia-Fernandez S, Morosini M-I, Cobo M, Foruny J R, Lopez-Sanroman A, Cobo J, Romero J, Canton R, Del Campo R. Gut eradication of VIM-1 producing ST9 Klebsiella oxytoca after fecal microbiota transplantation for diarrhea caused by a Clostridium difficile hypervirulent R027 strain. Diagn Microbiol Infect Dis. 2016 December; 86(4):470-471. PMID: 27712927
21. Millan B, Park H, Hotte N, Mathieu O, Burguiere P, Tompkins T, Kao D, Madsen K. Fecal Microbial Transplants Reduce Antibiotic-resistant Genes in Patients With Recurrent Clostridium difficile Infection. Clin Infect Dis. 2016; 62(12):1479-1486. PMID: 27025836
22. Lagier J, Million M, Fournier P, Brouqui P, Raoult D. Faecal microbiota transplantation for stool decolonization of OXA-48 carbapenemase-producing Klebsiella pneumoniae. J Hosp Infect. 2015; 90(2):173-4. PMID: 25913649
23. Bilinski J, Grzesiowski P, Muszynski J, Wroblewska M, Madry K, Robak K, Dzieciatkowski T, Wieslaw W-J, Basak G. Fecal Microbiota Transplantation Inhibits Multidrug-Resistant Gut Pathogens: Preliminary Report Performed in an Immunocompromised Host. Arch Immunol Ther Ex. 2016; 64(3):255-258. PMID: 26960790
24. Bilinski J, Grzesiowski P, Sorensen N, Madry K, Muszynski J, Robak K, Wroblewska M, Dzieciatkowski T, Dulny G, Dwilewicz-Trojaczek J, Wiktor-Jedrzejczak W, Basak G W. Fecal Microbiota Transplantation in Patients with Blood Disorders Inhibits Gut Colonization with Antibiotic-Resistant Bacteria: Results of a Prospective, Single-Center Study. Clin Infect Dis Off Publ Infect Dis Soc Am. 2017 Mar. 24; PMID: 28369341
25. Freedman A. Use of Stool Transplant to Clear Fecal Colonization with Carbapenem-Resistant Enterobacteraciae (CRE): Proof of Concept. Idsa; 2014 [cited 2017 Mar. 30]. Available from: https://idsa.confex.com/idsa/2014/webprogram/Paper47124.html
26. Davido B, Batista R, Michelon H, Lepainteur M, Bouchand F, Lepeule R, Salomon J, Vittecoq D, Duran C, Escaut L, Sobhani I, Paul M, Lawrence C, Perronne C, Chast F, Dinh A. Is faecal microbiota transplantation an option to eradicate highly drug-resistant enteric bacteria carriage? J Hosp Infect. 2017 Apr. 1; 95(4):433-437. PMID: 28237504
27. Sohn K M, Cheon S, Kim Y-S. Can Fecal Microbiota Transplantation (FMT) Eradicate Fecal Colonization With Vancomycin-Resistant Enterococci (VRE)? Infect Control Amp Hosp Epidemiol. 2016 January; 1-2.
28. Jouhten H, Mattila E, Arkkila P, Satokari R. Reduction of Antibiotic Resistance Genes in Intestinal Microbiota of Patients With Recurrent Clostridium difficile Infection After Fecal Microbiota Transplantation. Clin Infect Dis. 2016; 63(5):710-1. PMID: 27317794
29. Dubberke E R, Mullane K M, Gerding D N, Lee C H, Louie T J, Guthertz H, Jones C. Clearance of Vancomycin-Resistant Enterococcus Concomitant With Administration of a Microbiota-Based Drug Targeted at Recurrent Clostridium difficile Infection. Open Forum Infect Dis. 2016; 3(3):ofw133.
30. Smith M. Clearance of Vancomycin-Resistant Enterococcus Colonization with Fecal Microbiota Transplantation among Patients with Recurrent Clostridium difficile Infection. Idsa; 2016 [cited 2016 Oct. 21]. Available from: https://idsa.confex.com/idsa/2016/webprogram/Paper59960.html
31. Khanna S, Pardi D S, Kelly C R, Kraft C S, Dhere T, Henn M R, Lombardo M-J J, Vulic M, Ohsumi T, Winkler J, Pindar C, H M Barbara, Pomerantz R J, Aunins J G, Cook D N, Hohmann E L. A Novel Microbiome Therapeutic Increases Gut Microbial Diversity and Prevents Recurrent Clostridium difficile Infection. J Infect Dis. 2016; 214(2):173-81. PMID: 26908752
32. Jang M-O, An J, Jung S-I, Park K-H. Refractory Clostridium difficile Infection Cured With Fecal Microbiota Transplantation in Vancomycin-Resistant Enterococcus Colonized Patient. Intest Res. 2014; 13(1):80-84. PMCID: PMC4316227
33. Nancy C-C, Sullivan E, Gonzalo B-L. Fecal Microbiota Transplantation and Successful Resolution of Multidrug-Resistant-Organism Colonization. J Clin Microbiol. 2015; 53(6):1986-1989. PMID: 25878340
34. Stripling J, Kumar R, Baddley J W, Nellore A, Dixon P, Howard D, Ptacek T, Lefkowitz E J, Tallaj J A, Benjamin W H, Morrow C D, Rodriguez J. Loss of Vancomycin-Resistant Enterococcus Fecal Dominance in an Organ Transplant Patient With Clostridium difficile Colitis After Fecal Microbiota Transplant. Open Forum Infect Dis. 2015; 2(2):ofv078. PMCID: PMC4498259
35. Gilmore M, Rauch M, Ramsey M, Himes P, Varahan S, Manson J, Lebreton F, Hancock L. Pheromone killing of multidrug-resistant Enterococcus faecalis V583 by native commensal strains. P Natl Acad Sci Usa. 2015; 112(23):7273-8. PMCID: PMC4466700
36. Gaca A, Gilmore M. Killing of VRE Enterococcus faecalis by commensal strains: Evidence for evolution and accumulation of mobile elements in the absence of competition. Gut Microbes. 2016; 7(1):90-96. PMCID: PMC4856443
37. Sassone-Corsi M, Nuccio S-P, Liu H, Hernandez D, Vu C T, Takahashi A A, Edwards R A, Raffatellu M. Microcins mediate competition among Enterobacteriaceae in the inflamed gut. Nature. 2016 Dec. 8; 540(7632):280-283.
38. Hecht A L, Casterline B W, Earley Z M, Goo Y A, Goodlett D R, Bubeck Wardenburg J. Strain competition restricts colonization of an enteric pathogen and prevents colitis. EMBO Rep. 2016; 17(9):1281-91. PMCID: PMC5007561
39. McKenney E S, Kendall M M. Microbiota and pathogen “pas de deux”: setting up and breaking down barriers to intestinal infection. Pathog Dis. 2016; 74(5). PMID: 27252177
40. Ubeda C, Bucci V, Caballero S, Djukovic A, Toussaint N, Equinda M, Lipuma L, Ling L, Gobourne A, No D, Taur Y, Jenq R, Brink M, Xavier J, Pamer E. Intestinal Microbiota Containing Barnesiella Species Cures Vancomycin-Resistant Enterococcus faecium Colonization. Infect Immun. 2013; 81(3):965-973. PMCID: PMC3584866
41. Kinnebrew M, Ubeda C, Zenewicz L, Smith N, Flavell R, Pamer E. Bacterial flagellin stimulates Toll-like receptor 5-dependent defense against vancomycin-resistant Enterococcus infection. J Infect Dis. 2010; 201(4):534-43. PMCID: PMC2811237
42. Desai M S, Seekatz A M, Koropatkin N M, Kamada N, Hickey C A, Wolter M, Pudlo N A, Kitamoto S, Terrapon N, Muller A, Young V B, Henrissat B, Wilmes P, Stappenbeck T S, NOhez G, Martens E C. A Dietary Fiber-Deprived Gut Microbiota Degrades the Colonic Mucus Barrier and Enhances Pathogen Susceptibility. Cell. 2016 Nov. 17; 167(5):1339-1353.e21.
43. Earle K A, Billings G, Sigal M, Lichtman J S, Hansson G C, Elias J E, Amieva M R, Huang K C, Sonnenburg J L. Quantitative Imaging of Gut Microbiota Spatial Organization. Cell Host Microbe. 2015 Oct. 14; 18(4):478-488. PMID: 26439864
44. Goverse G, Molenaar R, Macia L, Tan J, Erkelens M N, Konijn T, Knippenberg M, Cook E C L, Hanekamp D, Veldhoen M, Hartog A, Roeselers G, Mackay C R, Mebius R E. Diet-Derived Short Chain Fatty Acids Stimulate Intestinal Epithelial Cells To Induce Mucosal Tolerogenic Dendritic Cells. J Immunol. 2017 Jan. 18; 1600165. PMID: 28100682
45. Wu W, Sun M, Chen F, Cao A T, Liu H, Zhao Y, Huang X, Xiao Y, Yao S, Zhao Q, Liu Z, Cong Y. Microbiota metabolite short-chain fatty acid acetate promotes intestinal IgA response to microbiota which is mediated by GPR43. Mucosal Immunol [Internet]. 2016 Dec. 14 [cited 2017 Jan. 24]; Available from: the World Wide Web (www) at nature.com/doifinder/10.1038/mi.2016.114
46. Olsan E E et al. Colonization resistance: the deconvolution of a complex trait. 2017. Journal of Biological Chemistry 292(21): 8577-8581.
47. Meynell G G. Antibacterial mechanisms of the mouse gut. II. The role of Eh and volative fatty acids in the normal gut. 1963. British journal of experimental pathology 44: 209-219.
48. Winter S E et al. The dynamics of gut-associated microbial communities during inflammation. 2013. EMPO Rep 14: 319-327.
49. Winter S E et al. Host-derived nitrate boosts growth of E. coli in the inflamed gut. 2013. Science 339: 708-711.
50. Spees A M et al. Streptomycin-induced inflammation enhances Escherichia coli gut colonization through nitrate respiration. 2013. MBio 4: e00430
51. Garner C D et al. Perturbation of the small intestine microbial ecology by streptomycin alters pathology in a Salmonella enterica serovar typhimurium murine model of infection. 2009. Infection and Immunity 77: 2691-2702.
52. Smith P M et al. The microbial metabolites, short-chain fatty acids, regulate colonic Treg cell homeostasis. 2013. Science 341: 569-573.
53. Rivera-Chavez F et al. Depletion of butyrate-producing Clostridia from the gut microbiota drives an aerobic luminal expansion of Salmonella. 2016. Cell Host and Microbe 19: 443-454.
54. Itoh K and Freter R. Control of Escherichia coli populations by a combination of indigenous Clostridia and Lactobacilli in gnotobiotic mice and continuous-flow cultures. 1989. Infection and Immunity 57: 559-565.
55. Donohoe D R et al. Microbial regulation of glucose metabolism and cell-cycle progression in mammalian colonocytes. 2012. PLoS One 7: e46589.
56. Kelly C J et al. Crosstalk between microbiota-derived short-chain fatty acids and intestinal epithelial HIF augments tissue barrier function. 2015. Cell Host and Microbe 17: 662-671.
57. Jones S A et al. Anaerobic respiration of Escherichia coli in the mouse intestine. 2011. Infection and Immunity 79: 4218-4226.
58. Kinnebrew M et al. Bacterial flagellin stimulates TLR5-dependent defense against vancomycin-resistant Enterococcus infection. 2010. Journal of Infectious Disease 201(4): 534-543.
59. Artis D. Epithelial-cell recognition of commensal bacteria and maintenance of immune homeostasis in the gut. 2008. Nature Reviews Immunology 8:411-420.
60. Vaishnava S et al. Paneth cells directly sense gut commensals and maintain homeostasis at the intestinal host-microbial interface. 2008. PNAS.
61. Macpherson A J et al. Interactions between commensal intestinal bacteria and the immune system. 2004. Nature Reviews Immunology 4: 478-485.
62. Rakoff-Nahoum S et al. Recognition of commensal microflora by toll-like receptors is required for intestinal homeostasis. 2004. Cell 118:229-241.
63. Pamer E G. Immune responses to commensal and environmental microbes. 2007. Nature Immunology 8:1173-1178.
64. Cash H L et al. Symbiotic bacteria direct expression of an intestinal bactericidal lectin. 2006. Science 313: 1126-1130.
65. Ayabe T et al. Secretion of microbicidal alpha-defensins by intestinal Paneth cells in response to bacteria. 2000. Nature Immunology 1:113-118.
66. Vora P et al. Beta-defensin-2 expression is regulated by TLR signaling in intestinal epithelial cells. 2004. Journal of Immunology 173:5398-5405.
67. Kolls J K et al. Cytokine-mediated regulation of antimicrobial proteins. 2008. Nature Reviews Immunology 8: 829-835.
68. Brandl K et al. MyD88-mediated signals induce the bactericidal lectin RegIIIgamma and protect mice against intestinal Listeria monocytogenes infection. 2007. Journal of Experimental Medicine 204: 1891-1900.
69. Brandl K et al. Vancomycin-resistant enterococci exploit antibiotic-induced innate immune deficits 2008. Nature 455: 804-807.
70. Charlier C, Cretenet M, Even S, Le Loir Y (2009) Interactions between Staphylococcus aureus and lactic acid bacteria: an old story with new perspectives. Int J Food Microbiol 131:30-39
71. Reid G, Kim S O, Ko“hler G A (2006) Selecting, testing and understanding probiotic microorganisms. FEMS Immunol Med Microbiol 46:149-157
72. Matu M N, Orinda G O, Njagi E N M, Cohen C R, Bukusi E A (2010) In vitro inhibitory activity of human vaginal lactobacilli against pathogenic bacteria associated with bacterial vaginosis in Kenyan women. Anaerobe 16:210-215
73. Karaoglu S, A, Aydin F, Kilic, SS, Kilic, AO (2002) Antimicrobial activity and characteristics of bacteriocins produced by vaginal lactobacilli. Turk J Med Sci 33:7-13
74. Zarate G, Nader-Macias M E (2006) Influence of probiotic vaginal lactobacilli on in vitro adhesion of urogenital pathogens to vaginal epithelial cells. Lett Appl Microbiol 43:174-180
75. Boris S, Suarez J E, Vazquez F, Barbes C (1998) Adherence of human vaginal lactobacilli to vaginal epithelial cells and interaction with uropathogens. Infect Immun 66:1985-1989

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1. A pharmaceutical composition comprising a bacterial mixture wherein at least one bacterial strain in the bacterial mixture comprises a 16S rRNA sequence that is greater than about 97% identical to the 16S rRNA sequence of any one of SEQ ID NOs 1-3185.

2.-11. (canceled)

12. The pharmaceutical composition of claim 1, wherein the at least one bacterial strain is isolated and/or purified from its source material prior to forming the bacterial mixture.

13. The pharmaceutical composition of claim 1, wherein the at least one bacterial strain is cultured prior to forming the bacterial mixture.

14. The pharmaceutical composition of claim 1, wherein the at least one bacterial strain is not cultured prior to forming the bacterial mixture.

15. The pharmaceutical composition of claim 1, wherein the at least one bacterial strain is not isolated and/or purified from its source material prior to forming the bacterial mixture.

16.-25. (canceled)

26. The pharmaceutical composition of claim 1, wherein the bacterial mixture comprises a plurality of at least about fifty bacterial strains, wherein each bacterial strain of the plurality comprises a 16S rRNA sequence that is greater than about 97% identical to the 16S rRNA sequence of any one of SEO ID NOs 1-3185.

27. The pharmaceutical composition of claim 1, wherein the bacterial mixture comprises a plurality of at least one hundred bacterial strains, and wherein each bacterial strain of the plurality comprises a 16S rRNA sequence that is greater than about 97% identical to the 16S rRNA sequence of any one of SEQ ID NOs 1-3185.

28. The pharmaceutical composition of claim 1, wherein the bacterial mixture comprises a plurality of between about five and about one hundred bacterial strains, and wherein each bacterial strain of the in the plurality comprises a 16S rRNA sequence that is greater than about 97% identical to the 16S rRNA sequence of any one of SEQ ID NOs 1-3185.

29.-51. (canceled)

52. The pharmaceutical composition of claim 1, wherein at least one bacterial strain is included in the bacterial mixture due to its ability to directly inhibit an antibiotic-resistant bacterium (ARB) through production of a secreted product and/or its capability to directly compete with the ARB for a niche and/or for nutrients.

53. (canceled)

54. The pharmaceutical composition of claim 1, wherein the bacterial mixture is lyophilized and formulated into a capsule further comprising microcrystalline cellulose, gelatin, and magnesium stearate.

55. The pharmaceutical composition of a claim 1, wherein the pharmaceutical composition is formulated to be delivered orally, via a nasogastric tube, intravaginally, or colonoscopically.

56.-69. (canceled)

70. A method for treating an infection in a subject by one or more pathogens, comprising administering an effective amount of a pharmaceutical composition comprising a bacterial mixture wherein at least one bacterial strain in the bacterial mixture comprises a 16S rRNA sequence that is greater than about 97% identical to the 16S rRNA sequence of any one of SEQ ID NOs 1-3185.

71. (canceled)

72. (canceled)

73. The method of a claim 70, wherein administering an effective amount of the pharmaceutical composition decolonizes the one or more pathogens and/or eradicates the one or more pathogens.

74. The method of claim 70, wherein the one or more pathogens includes one or more of Aeromonas hydrophila, Bacillus, Bifidobacterium, Bordetella, Borrelia, Brucella, Burkholderia, Clostridium Campylobacter, Chlamydia, Chlamydophila, Corynebacterium, Coxiella, Ehrlichia, Enterobacteriaceae, Enterococcus, Escherichia Francisella, Haemophilus, Helicobacter, Klebsiella, Legionella, Leptospira, Listeria, Morganella, Mycobacterium, Mycoplasma, Neisseria, Orientia, Plesiomonas shigelloides, Antibiotic-resistant Proteobacteria, Proteus, Pseudomonas, Rickettsia, Salmonella, Shigella, Staphylococcus, Streptococcus, Treponema, Vibrio, and Yersinia.

75. The method of claim 70, wherein at least one of the one or more pathogens is an antibiotic-resistant bacterium (ARB).

76. (canceled)

77. The method of claim 70, wherein the subject in need thereof has received or is receiving, an anti-cancer therapeutic agent and/or an anti-cancer therapy.

78.-100. (canceled)

101. A method of increasing efficacy of an anti-cancer therapeutic agent and/or anti-cancer therapy comprising administering an effective amount of the pharmaceutical composition claim 1.

102.-105. (canceled)

106. The method of claim 101, wherein the anti-cancer therapy is a targeted therapy directed to a checkpoint molecule and the subject is refractory and/or non-responsive to the treatment directed to the checkpoint molecule.

107. The method of claim 106, wherein the treatment directed to the checkpoint molecule comprises administration Pembrolizumab, Nivolumab, Ipilimumab, Atezolizumab, Avelumab, or Durvalumab.

108. A method for manufacturing a pharmaceutical composition, wherein the pharmaceutical composition comprises a bacterial mixture wherein at least one bacterial strain in the bacterial mixture comprises a 16S rRNA sequence that is greater than about 97% identical to the 16S rRNA sequence of any one of SEQ ID NOs 1-3185.
